SEQUENCE LISTING (253 sequences)

SEQ ID NO: 1 - protein, 7 aa, synthetic construct
SSSVAWN

SEQ ID NO: 2 - protein, 18 aa, synthetic construct
RTYFRSKWYYDYAVSVKS

SEQ ID NO: 3 - protein, 8 aa, synthetic construct
GIVGALDY

SEQ ID NO: 4 - protein, 16 aa, synthetic construct
RSSQSLLFTNGYDSLD

SEQ ID NO: 5 - protein, 7 aa, synthetic construct
LVSNRAS

SEQ ID NO: 6 - protein, 9 aa, synthetic construct
MQGIRTPFT

SEQ ID NO: 7 - protein, 9 aa, synthetic construct
GDSVSSSSV

SEQ ID NO: 8 - protein, 7 aa, synthetic construct
YFRSKWY

SEQ ID NO: 9 - protein, 12 aa, synthetic construct
GDSVSSSSVAWN

SEQ ID NO: 10 - protein, 11 aa, synthetic construct
RTYFRSKWYYD

SEQ ID NO: 11 - protein, 8 aa, synthetic construct
SSSSVAWN

SEQ ID NO: 12 - protein, 14 aa, synthetic construct
WLGRTYFRSKWYYD

SEQ ID NO: 13 - protein, 9 aa, synthetic construct
SRGIVGALD

SEQ ID NO: 14 - protein, 12 aa, synthetic construct
LFTNGYDSLDWY

SEQ ID NO: 15 - protein, 10 aa, synthetic construct
LLIYLVSNRA

SEQ ID NO: 16 - protein, 8 aa, synthetic construct
MQGIRTPF

SEQ ID NO: 17 - protein, 10 aa, synthetic construct
GDSVSSSSVA

SEQ ID NO: 18 - protein, 9 aa, synthetic construct
TYFRSKWYY

SEQ ID NO: 19 - protein, 10 aa, synthetic construct
SRGIVGALDY

SEQ ID NO: 20 - protein, 11 aa, synthetic construct
QSLLFTNGYDS

SEQ ID NO: 21 - skipped

SEQ ID NO: 22 - protein, 120 aa, synthetic construct
QVQLQQSGPGLVMSSQTLSLTCAISGDSVSSSSVAWNWIRQSPSRGLQWLGRTYFRSKWYYDYAVSVKSRITINPDTSKNQFSLQLNSVTPEDTAVYYCSRGIVGALDYWGQGTLVTVSS

SEQ ID NO: 23 - protein, 112 aa, synthetic construct
DIVMTQSPLSLPVTPGEPASISCRSSQSLLFTNGYDSLDWYLQKPGQSPQLLIYLVSNRASGVPDRFSGSGSGTEFTLKISRVEADDVGVYFCMQGIRTPFTFGPGTKLEIK

SEQ ID NO: 24 - protein, 7 aa, synthetic construct
SNSVAWH

SEQ ID NO: 25 - protein, 18 aa, synthetic construct
RTYHRSKWYNDYAVSVKS

SEQ ID NO: 26 - protein, 8 aa, synthetic construct
QILGALDY

SEQ ID NO: 27 - protein, 15 aa, synthetic construct
RSSQSLQFGNGNYLD

SEQ ID NO: 28 - protein, 7 aa, synthetic construct
LGSNRAS

SEQ ID NO: 29 - protein, 9 aa, synthetic construct
MQVLQIPFT

SEQ ID NO: 30 - protein, 9 aa, synthetic construct
GDSVSSNSV

SEQ ID NO: 31 - protein, 7 aa, synthetic construct
YYRSKWY

SEQ ID NO: 32 - protein, 12 aa, synthetic construct
GDSVSSNSVAWH

SEQ ID NO: 33 - protein, 11 aa, synthetic construct
RTYHRSKWYND

SEQ ID NO: 34 - protein, 8 aa, synthetic construct
SSNSVAWH

SEQ ID NO: 35 - protein, 14 aa, synthetic construct
WLGRTYHRSKWYND

SEQ ID NO: 36 - protein, 9 aa, synthetic construct
ARQILGALD

SEQ ID NO: 37 - protein, 11 aa, synthetic construct
QFGNGNYLDWY

SEQ ID NO: 38 - protein, 10 aa, synthetic construct
LLIYLGSNRA

SEQ ID NO: 39 - protein, 8 aa, synthetic construct
MQVLQIPF

SEQ ID NO: 40 - protein, 10 aa, synthetic construct
GDSVSSNSVA

SEQ ID NO: 41 - protein, 9 aa, synthetic construct
TYHRSKWYN

SEQ ID NO: 42 - protein, 10 aa, synthetic construct
ARQILGALDY

SEQ ID NO: 43 - protein, 10 aa, synthetic construct
QSLQFGNGNY

SEQ ID NO: 44 - skipped

SEQ ID NO: 45 - protein, 120 aa, synthetic construct
QVQLQQSGPGLVKPSQTLSLTCAISGDSVSSNSVAWHWIRQSPSRGLEWLGRTYHRSKWYNDYAVSVKSRITINPDTSKNQFSLQLNSVTPEDTAVYYCARQILGALDYWGQGTLVTVSS

SEQ ID NO: 46 - protein, 111 aa, synthetic construct
DIVMTQTPLSLPVTPGEPASISCRSSQSLQFGNGNYLDWYLQKPGQSPQLLIYLGSNRASGVPDRFSGSGSGTDFTLKISRVEAEDVGVYYCMQVLQIPFTFGPGTKVDIK

SEQ ID NO: 47 - protein, 7 aa, synthetic construct
SNRVAWN

SEQ ID NO: 48 - protein, 18 aa, synthetic construct
RTYYRSKWYNDFALSVKS

SEQ ID NO: 49 - protein, 16 aa, synthetic construct
RSSQSLLFSNGYNSLD

SEQ ID NO: 50 - protein, 9 aa, synthetic construct
MQALQTPFT

SEQ ID NO: 51 - protein, 12 aa, synthetic construct
GDSVSSNRVAWN

SEQ ID NO: 52 - protein, 11 aa, synthetic construct
RTYYRSKWYND

SEQ ID NO: 53 - protein, 8 aa, synthetic construct
SSNRVAWN

SEQ ID NO: 54 - protein, 14 aa, synthetic construct
WLGRTYYRSKWYND

SEQ ID NO: 55 - protein, 9 aa, synthetic construct
ARGIVGALD

SEQ ID NO: 56 - protein, 12 aa, synthetic construct
LFSNGYNSLDWY

SEQ ID NO: 57 - protein, 8 aa, synthetic construct
MQALQTPF

SEQ ID NO: 58 - protein, 10 aa, synthetic construct
GDSVSSNRVA

SEQ ID NO: 59 - protein, 9 aa, synthetic construct
TYYRSKWYN

SEQ ID NO: 60 - protein, 10 aa, synthetic construct
ARGIVGALDY

SEQ ID NO: 61 - protein, 11 aa, synthetic construct
QSLLFSNGYNS

SEQ ID NO: 62 - protein, 120 aa, synthetic construct
QVQLQQSGPGLVKPSQTLSLTCAISGDSVSSNRVAWNWIRQSPSRGLEWLGRTYYRSKWYNDFALSVKSRININADTSKSQFSLQLNSVTPEDTAVYYCARGIVGALDYWGQGTTVTVSS

SEQ ID NO: 63 - protein, 112 aa, synthetic construct
DIVMTQSPLSLPVTPGEPASISCRSSQSLLFSNGYNSLDWYLQKPGQSPQLLIYLGSNRASGVPDRFSGSESGTDFTLKISRVEAEDVGVYYCMQALQTPFTFGPGTKLEIK

SEQ ID NO: 64 - protein, 7 aa, synthetic construct
SNSAAWN

SEQ ID NO: 65 - protein, 18 aa, synthetic construct
RTYYRSKWYNDYAVSVKS

SEQ ID NO: 66 - protein, 10 aa, synthetic construct
RDYSSGGFDY

SEQ ID NO: 67 - protein, 17 aa, synthetic construct
KSSQSVLYSSDNKNYLA

SEQ ID NO: 68 - protein, 7 aa, synthetic construct
WASTRES

SEQ ID NO: 69 - protein, 9 aa, synthetic construct
QQYYSTPFT

SEQ ID NO: 70 - protein, 9 aa, synthetic construct
GDSVSSNSA

SEQ ID NO: 71 - protein, 12 aa, synthetic construct
GDSVSSNSAAWN

SEQ ID NO: 72 - protein, 8 aa, synthetic construct
SSNSAAWN

SEQ ID NO: 73 - protein, 11 aa, synthetic construct
ARRDYSSGGFD

SEQ ID NO: 74 - protein, 13 aa, synthetic construct
LYSSDNKNYLAWY

SEQ ID NO: 75 - protein, 10 aa, synthetic construct
LLIYWASTRE

SEQ ID NO: 76 - protein, 8 aa, synthetic construct
QQYYSTPF

SEQ ID NO: 77 - protein, 10 aa, synthetic construct
GDSVSSNSAA

SEQ ID NO: 78 - protein, 12 aa, synthetic construct
ARRDYSSGGFDY

SEQ ID NO: 79 - protein, 12 aa, synthetic construct
QSVLYSSDNKNY

SEQ ID NO: 80 - skipped

SEQ ID NO: 81 - protein, 122 aa, synthetic construct
QVQLVESGPGQVKPSQTLSLTCAISGDSVSSNSAAWNWIRQSPSRGLEWLGRTYYRSKWYNDYAVSVKSRITINPDTSKNQFSLQLNSVTPEDTAVYYCARRDYSSGGFDYWGQGTTVTVSS

SEQ ID NO: 82 - protein, 113 aa, synthetic construct
EIVLTQSPDSLAVSLGERATINCKSSQSVLYSSDNKNYLAWYQQKPGQPPNLLIYWASTRESGVPDRFSGSGSGTDFTLTINSLQAEDVAVYYCQQYYSTPFTFGPGTKVEIK

SEQ ID NO: 83 - protein, 7 aa, synthetic construct
SNSVAWN

SEQ ID NO: 84 - protein, 18 aa, synthetic construct
RTYYRSKWYNDYALSVKS

SEQ ID NO: 85 - protein, 8 aa, synthetic construct
GVVGAVDY

SEQ ID NO: 86 - protein, 16 aa, synthetic construct
RSSQNLLYSNGYNSLD

SEQ ID NO: 87 - protein, 9 aa, synthetic construct
MQGLRTPLT

SEQ ID NO: 88 - protein, 8 aa, synthetic construct
SSNSVAWN

SEQ ID NO: 89 - protein, 9 aa, synthetic construct
ARGVVGAVD

SEQ ID NO: 90 - protein, 12 aa, synthetic construct
LYSNGYNSLDWY

SEQ ID NO: 91 - protein, 8 aa, synthetic construct
MQGLRTPL

SEQ ID NO: 92 - protein, 10 aa, synthetic construct
ARGVVGAVDY

SEQ ID NO: 93 - protein, 11 aa, synthetic construct
QNLLYSNGYNS

SEQ ID NO: 94 - protein, 120 aa, synthetic construct
EVQLVQSGPGLVKPSQTLSLTCAISGDSVSSNSVAWNWIRQSPSRGLEWLGRTYYRSKWYNDYALSVKSRITINPDTSKNQFSLQLNSVTPEDTAVYYCARGVVGAVDYWGQGTLVTVSS

SEQ ID NO: 95 - protein, 112 aa, synthetic construct
DIVMTQSPLSLPVTPGEPASISCRSSQNLLYSNGYNSLDWYLQKPGQSPQLLIYLGSNRASGVPDRFSGSGSGTDFTLKISRVEAEDVGVYYCMQGLRTPLTFGGGTKVEIK

SEQ ID NO: 96 - protein, 18 aa, synthetic construct
RTYYRSKWYNDYAISVRS

SEQ ID NO: 97 - protein, 8 aa, synthetic construct
GVVGALDY

SEQ ID NO: 98 - protein, 16 aa, synthetic construct
RSSQSLLFTNGYNSLD

SEQ ID NO: 99 - protein, 9 aa, synthetic construct
MQALRTPYT

SEQ ID NO: 100 - protein, 9 aa, synthetic construct
SRGVVGALD

SEQ ID NO: 101 - protein, 12 aa, synthetic construct
LFTNGYNSLDWY

SEQ ID NO: 102 - protein, 10 aa, synthetic construct
FLIYLVSNRA

SEQ ID NO: 103 - protein, 8 aa, synthetic construct
MQALRTPY

SEQ ID NO: 104 - protein, 10 aa, synthetic construct
SRGVVGALDY

SEQ ID NO: 105 - protein, 11 aa, synthetic construct
QSLLFTNGYNS

SEQ ID NO: 106 - protein, 120 aa, synthetic construct
QVQLQQSGPGLVKPSQTLSLTCGISGDSVSSSSVAWNWIRQSPSRGLEWLGRTYYRSKWYNDYAISVRSRVTINPDTSKNQFSLQLNSVTPEDTAVYYCSRGVVGALDYWGQGTLVTVSS

SEQ ID NO: 107 - protein, 112 aa, synthetic construct
DIVMTQSPLSLTVTPGEPASISCRSSQSLLFTNGYNSLDWYLQKPGQSPQFLIYLVSNRASGVPDRFSGSGSGTDFTLKISRVEAEDVGVYYCMQALRTPYTFGQGTKVEIK

SEQ ID NO: 108 - protein, 7 aa, synthetic construct
STRAAWN

SEQ ID NO: 109 - protein, 18 aa, synthetic construct
RTYYRSKWYNDYTVSVKS

SEQ ID NO: 110 - protein, 8 aa, synthetic construct
GNVGRVDF

SEQ ID NO: 111 - protein, 16 aa, synthetic construct
RSSQSLLHSDGYNYLD

SEQ ID NO: 112 - protein, 9 aa, synthetic construct
GDSVSSTRA

SEQ ID NO: 113 - protein, 12 aa, synthetic construct
GDSVSSTRAAWN

SEQ ID NO: 114 - protein, 8 aa, synthetic construct
SSTRAAWN

SEQ ID NO: 115 - protein, 9 aa, synthetic construct
ARGNVGRVD

SEQ ID NO: 116 - protein, 12 aa, synthetic construct
LHSDGYNYLDWY

SEQ ID NO: 117 - protein, 10 aa, synthetic construct
GDSVSSTRAA

SEQ ID NO: 118 - protein, 10 aa, synthetic construct
ARGNVGRVDF

SEQ ID NO: 119 - protein, 11 aa, synthetic construct
QSLLHSDGYNY

SEQ ID NO: 120 - protein, 120 aa, synthetic construct
QVQLQQSGPGLVKPSQTLSLTCAISGDSVSSTRAAWNWIRQSPSRGLEWLGRTYYRSKWYNDYTVSVKSRITINPDTSKNQFSLHLNSVTPEDSAVYFCARGNVGRVDFWGQGTLVTVSS

SEQ ID NO: 121 - protein, 112 aa, synthetic construct
EIVMTQSPLSLPVTPGEPASISCRSSQSLLHSDGYNYLDWYLQKPGQSPQLLIYLGSNRASGVPDRFSGSGSDTDFTLKISRVEAEDVGVYYCMQGLRTPLTFGPGTKVDIK

SEQ ID NO: 122 - protein, 7 aa, synthetic construct
STSVAWN

SEQ ID NO: 123 - protein, 16 aa, synthetic construct
RSSQSLMYSNGYNHLD

SEQ ID NO: 124 - protein, 7 aa, synthetic construct
LGSNRVS

SEQ ID NO: 125 - protein, 9 aa, synthetic construct
GDSVSSTSV

SEQ ID NO: 126 - protein, 12 aa, synthetic construct
GDSVSSTSVAWN

SEQ ID NO: 127 - protein, 8 aa, synthetic construct
SSTSVAWN

SEQ ID NO: 128 - protein, 12 aa, synthetic construct
MYSNGYNHLDWY

SEQ ID NO: 129 - protein, 10 aa, synthetic construct
FLIHLGSNRV

SEQ ID NO: 130 - protein, 10 aa, synthetic construct
GDSVSSTSVA

SEQ ID NO: 131 - protein, 11 aa, synthetic construct
QSLMYSNGYNH

SEQ ID NO: 132 - protein, 120 aa, synthetic construct
QVQLQQSGPGLVKPSQTLSLTCAISGDSVSSTSVAWNWIRQSPSGGLEWLGRTYYRSKWYNDYALSVKSRMTINPDTASNQFSLQLNSVTPEDTAVYYCSRGIVGALDYWGQGTLVTVSS

SEQ ID NO: 133 - protein, 112 aa, synthetic construct
DIVMTQTPLSLPVTPGGPASISCRSSQSLMYSNGYNHLDWYLQKPGQSPQFLIHLGSNRVSGVPDRFSGSGSGTDFTLKISRVEAEDVGVYYCMQALRTPYTFGQGTKVDIK

SEQ ID NO: 134 - protein, 18 aa, synthetic construct
RIYYRSKWYNDYALSVKS

SEQ ID NO: 135 - protein, 11 aa, synthetic construct
RIYYRSKWYND

SEQ ID NO: 136 - protein, 14 aa, synthetic construct
WLGRIYYRSKWYND

SEQ ID NO: 137 - protein, 9 aa, synthetic construct
IYYRSKWYN

SEQ ID NO: 138 - protein, 120 aa, synthetic construct
QVQLQQSGPGLVKPSQTLSLTCAISGDSVSSTSVAWNWIRQSPSGGLEWLGRIYYRSKWYNDYALSVKSRMTINPDTASNQFSLQLNSVTPEDTAVYYCSRGIVGALDYWGQGTLVTVSS

SEQ ID NO: 139 - protein, 112 aa, synthetic construct
EIVMTQSPLSLPVTPGEPASISCRSSQSLLHSDGYNYLDWYLQKPGQSPQLLIYLGSNRASGVPDRFSGSGSDTDFTLKISRVEAEDVGVYYCMQGLRTPLTFGPGTKLEIK

SEQ ID NO: 140 - protein, 112 aa, synthetic construct
DIVMTQSPLSLTVTPGEPASISCRSSQSLLFTNGYNSLDWYLQKPGQSPQFLIYLVSNRASGVPDRFSGSGSGTDFTLKISRVEAEDVGVYYCMQALRTPYTFGQGTKVDIK

SEQ ID NO: 141 - protein, 10 aa, synthetic construct
FLIHLGSNRA

SEQ ID NO: 142 - protein, 112 aa, synthetic construct
DIVMTQSPLSLPVTPGEPASISCRSSQSLMYSNGYNHLDWYLQKPGQSPQFLIHLGSNRASGVPDRFSGSGSGTDFTLKISRVEAEDVGVYYCMQALRTPYTFGQGTKVEIK

SEQ ID NO: 143 - protein, 5 aa, synthetic construct
RYNMN

SEQ ID NO: 144 - protein, 17 aa, synthetic construct
SISTSSNYIYYADSVKG

SEQ ID NO: 145 - protein, 7 aa, synthetic construct
GWGPFDY

SEQ ID NO: 146 - protein, 11 aa, synthetic construct
RARQSIGTAIH

SEQ ID NO: 147 - protein, 7 aa, synthetic construct
YASESIS

SEQ ID NO: 148 - protein, 9 aa, synthetic construct
QQSGSWPYT

SEQ ID NO: 149 - protein, 7 aa, synthetic construct
GFTFSRY

SEQ ID NO: 150 - protein, 6 aa, synthetic construct
STSSNY

SEQ ID NO: 151 - protein, 6 aa, synthetic construct
GWGPFD

SEQ ID NO: 152 - protein, 7 aa, synthetic construct
RQSIGTA

SEQ ID NO: 153 - skipped

SEQ ID NO: 154 - protein, 6 aa, synthetic construct
SGSWPY

SEQ ID NO: 155 - protein, 8 aa, synthetic construct
GFTFSRYN

SEQ ID NO: 156 - protein, 8 aa, synthetic construct
ISTSSNYI

SEQ ID NO: 157 - protein, 9 aa, synthetic construct
TRGWGPFDY

SEQ ID NO: 158 - protein, 6 aa, synthetic construct
QSIGTA

SEQ ID NO: 159 - protein, 116 aa, synthetic construct
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 160 - protein, 107 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 161 - protein, 243 aa, synthetic construct
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 162 - protein, 243 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 163 - protein, 475 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 452 aa, synthetic construct
QVQLVESGPGQVKPSQTLSLTCAISGDSVSSNSAAWNWIRQSPSRGLEWLGRTYYRSKWYNDYAVSVKSRITINPDTSKNQFSLQLNSVTPEDTAVYYCARRDYSSGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 165 - protein, 220 aa, synthetic construct
EIVLTQSPDSLAVSLGERATINCKSSQSVLYSSDNKNYLAWYQQKPGQPPNLLIYWASTRESGVPDRFSGSGSGTDFTLTINSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 20 aa, synthetic construct
GGSEGKSSGSGSESKSTGGS

SEQ ID NO: 167 - protein, 8 aa, synthetic construct
GGGSGGGS

SEQ ID NO: 168 - protein, 12 aa, synthetic construct
GGGSGGGSGGGS

SEQ ID NO: 169 - protein, 16 aa, synthetic construct
GGGSGGGSGGGSGGGS

SEQ ID NO: 170 - protein, 20 aa, synthetic construct
GGGSGGGSGGGSGGGSGGGS

SEQ ID NO: 171 - protein, 15 aa, synthetic construct
GGGGSGGGGSGGGGS

SEQ ID NO: 172 - protein, 20 aa, synthetic construct
GGGGSGGGGSGGGGSGGGGS

SEQ ID NO: 173 - protein, 25 aa, synthetic construct
GGGGSGGGGSGGGGSGGGGSGGGGS

SEQ ID NO: 174 - protein, 18 aa, synthetic construct
GSTSGSGKPGSGEGSTKG

SEQ ID NO: 175 - protein, 14 aa, synthetic construct
IRPRAIGGSKPRVA

SEQ ID NO: 176 - protein, 15 aa, synthetic construct
GKGGSGKGGSGKGGS

SEQ ID NO: 177 - protein, 15 aa, synthetic construct
GGKGSGGKGSGGKGS

SEQ ID NO: 178 - protein, 15 aa, synthetic construct
GGGKSGGGKSGGGKS

SEQ ID NO: 179 - protein, 15 aa, synthetic construct
GKGKSGKGKSGKGKS

SEQ ID NO: 180 - protein, 15 aa, synthetic construct
GGGKSGGKGSGKGGS

SEQ ID NO: 181 - protein, 15 aa, synthetic construct
GKPGSGKPGSGKPGS

SEQ ID NO: 182 - protein, 20 aa, synthetic construct
GKPGSGKPGSGKPGSGKPGS

SEQ ID NO: 183 - protein, 20 aa, synthetic construct
GKGKSGKGKSGKGKSGKGKS

SEQ ID NO: 184 - protein, 14 aa, synthetic construct
STAGDTHLGGEDFD

SEQ ID NO: 185 - protein, 15 aa, synthetic construct
GEGGSGEGGSGEGGS

SEQ ID NO: 186 - protein, 15 aa, synthetic construct
GGEGSGGEGSGGEGS

SEQ ID NO: 187 - protein, 15 aa, synthetic construct
GEGESGEGESGEGES

SEQ ID NO: 188 - protein, 15 aa, synthetic construct
GGGESGGEGSGEGGS

SEQ ID NO: 189 - protein, 20 aa, synthetic construct
GEGESGEGESGEGESGEGES

SEQ ID NO: 190 - protein, 18 aa, synthetic construct
GSTSGSGKPGSGEGSTKG

SEQ ID NO: 191 - protein, 19 aa, synthetic construct
PRGASKSGSASQTGSAPGS

SEQ ID NO: 192 - protein, 19 aa, synthetic construct
GTAAAGAGAAGGAAAGAAG

SEQ ID NO: 193 - protein, 19 aa, synthetic construct
GTSGSSGSGSGGSGSGGGG

SEQ ID NO: 194 - protein, 20 aa, synthetic construct
GKPGSGKPGSGKPGSGKPGS

SEQ ID NO: 195 - protein, 4 aa, synthetic construct
GSGS

SEQ ID NO: 196 - protein, 10 aa, synthetic construct
APAPAPAPAP

SEQ ID NO: 197 - protein, 20 aa, synthetic construct
APAPAPAPAPAPAPAPAPAP

SEQ ID NO: 198 - protein, 32 aa, synthetic construct
AEAAAKEAAAKEAAAAKEAAAAKEAAAAKAAA

SEQ ID NO: 199 - protein, 16 aa, synthetic construct
GTEGKSSGSGSESKST

SEQ ID NO: 200 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 201 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 202 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 203 - protein, 12 aa, Homo sapiens
LVLDLSMQEALS

SEQ ID NO: 204 - protein, 7 aa, synthetic construct
NNNAAWS

SEQ ID NO: 205 - protein, 18 aa, synthetic construct
RTYYRSKWLYDYAVSVKS

SEQ ID NO: 206 - protein, 8 aa, synthetic construct
GYSSSFDY

SEQ ID NO: 207 - protein, 14 aa, synthetic construct
TGTSSNIGTYKFVS

SEQ ID NO: 208 - protein, 7 aa, synthetic construct
EVSKRPS

SEQ ID NO: 209 - protein, 10 aa, synthetic construct
VSYAGSGTLL

SEQ ID NO: 210 - protein, 5 aa, synthetic construct
RSTMH

SEQ ID NO: 211 - protein, 17 aa, synthetic construct
YINPSSAYTNYNQKFQG

SEQ ID NO: 212 - protein, 12 aa, synthetic construct
PQVHYDYAGFPY

SEQ ID NO: 213 - protein, 10 aa, synthetic construct
SASSSVSYMN

SEQ ID NO: 214 - protein, 7 aa, synthetic construct
DSSKLAS

SEQ ID NO: 215 - protein, 9 aa, synthetic construct
QQWSRNPPT

SEQ ID NO: 216 - protein, 5 aa, synthetic construct
RSTMH

SEQ ID NO: 217 - protein, 17 aa, synthetic construct
YINPSSAYTNYNQKFQG

SEQ ID NO: 218 - protein, 12 aa, synthetic construct
PQVHYDYAGFPY

SEQ ID NO: 219 - protein, 10 aa, synthetic construct
SASSSVSYMN

SEQ ID NO: 220 - protein, 7 aa, synthetic construct
DSSKLAS

SEQ ID NO: 221 - protein, 9 aa, synthetic construct
QQWSRNPPT

SEQ ID NO: 222 - protein, 7 aa, synthetic construct
GYTFTRS

SEQ ID NO: 223 - protein, 6 aa, synthetic construct
NPSSAY

SEQ ID NO: 224 - protein, 11 aa, synthetic construct
PQVHYDYAGFP

SEQ ID NO: 225 - protein, 6 aa, synthetic construct
SSSVSY

SEQ ID NO: 226 - skipped

SEQ ID NO: 227 - protein, 6 aa, synthetic construct
WSRNPP

SEQ ID NO: 228 - protein, 8 aa, synthetic construct
GYTFTRST

SEQ ID NO: 229 - protein, 8 aa, synthetic construct
INPSSAYT

SEQ ID NO: 230 - protein, 14 aa, synthetic construct
ASPQVHYDYAGFPY

SEQ ID NO: 231 - protein, 5 aa, synthetic construct
SSVSY

SEQ ID NO: 232 - protein, 9 aa, synthetic construct
QQWSRNPPT

SEQ ID NO: 233 - protein, 120 aa, synthetic construct
QVQLQQSGPGLVKPSQTLSLTCAISGDSVFNNNAAWSWIRQSPSRGLEWLGRTYYRSKWLYDYAVSVKSRITINPDTSKNQFSLQLNSVTPEDTAVYYCARGYSSSFDYWGQGTLVTVSS

SEQ ID NO: 234 - protein, 110 aa, synthetic construct
QSALTQPASVSGSPGQSITISCTGTSSNIGTYKFVSWYQQHPGKAPKVMIYEVSKRPSGVSNRFSGSKSGNTASLTISGLQAEDEADYYCVSYAGSGTLLFGGGTKLTVL

SEQ ID NO: 235 - protein, 121 aa, synthetic construct
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSS

SEQ ID NO: 236 - protein, 106 aa, synthetic construct
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIK

SEQ ID NO: 237 - protein, 121 aa, synthetic construct
QVQLVQSGAEVKKPGSSVKVSCKASGYTFTRSTMHWVKQAPGQGLEWMGYINPSSAYTNYNQKFQGRVTLTADKSTSTAYMELSSLRSEDTAVYYCASPQVHYDYAGFPYWGQGTLVTVSS

SEQ ID NO: 238 - protein, 106 aa, synthetic construct
EIVLTQSPATLSASPGERVTLSCSASSSVSYMNWYQQKPGQAPRRLIYDSSKLASGVPARFSGSGSGRDYTLTISSLEPEDFAVYYCQQWSRNPPTFGGGTKVEIK

SEQ ID NO: 239 - DNA, 1356 bp, synthetic construct
  source 1..1356: mol_type = other DNA
caggtgcagctggtggaatctggacctggccaagtgaagccctctcagaccctgtctctgacctgtgccatctccggcgactccgtgtcctctaattctgccgcctggaactggatccggcagtctcctagtagaggcctggaatggctgggcagaacctactaccggtccaagtggtacaacgactacgccgtgtccgtgaagtcccggatcaccatcaatcccgacacctccaagaaccagttctccctgcagctgaacagcgtgacccctgaggataccgccgtgtactactgcgccagacgggactattcttccggcggctttgattactggggccagggcacaacagtgaccgtgtcatctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 240 - DNA, 660 bp, synthetic construct
  source 1..660: mol_type = other DNA
gagatcgtgctgacccagtctcctgactctctggctgtgtctctgggcgagagagccaccatcaactgcaagtcctctcagtccgtgctgtactcctccgacaacaagaactacctggcttggtatcagcagaagcctggtcagcctcctaacctgctgatctactgggcctccaccagagagtctggtgtgcccgatcgcttttccggctctggctctggcaccgacttcaccctgaccatcaattccctgcaggccgaggatgtggccgtgtactactgccagcagtactacagcacccctttcaccttcggacccggcaccaaggtggaaatcaagcgtactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 241 - protein, 247 aa, synthetic construct
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSGGSEGKSSGSGSESKSTGGSEIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIK

SEQ ID NO: 242 - protein, 247 aa, synthetic construct
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSS

SEQ ID NO: 243 - protein, 247 aa, synthetic construct
QVQLVQSGAEVKKPGSSVKVSCKASGYTFTRSTMHWVKQAPGQGLEWMGYINPSSAYTNYNQKFQGRVTLTADKSTSTAYMELSSLRSEDTAVYYCASPQVHYDYAGFPYWGQGTLVTVSSGGSEGKSSGSGSESKSTGGSEIVLTQSPATLSASPGERVTLSCSASSSVSYMNWYQQKPGQAPRRLIYDSSKLASGVPARFSGSGSGRDYTLTISSLEPEDFAVYYCQQWSRNPPTFGGGTKVEIK

SEQ ID NO: 244 - protein, 247 aa, synthetic construct
EIVLTQSPATLSASPGERVTLSCSASSSVSYMNWYQQKPGQAPRRLIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWMGYINPSSAYTNYNQKFQGRVTLTADKSTSTAYMELSSLRSEDTAVYYCASPQVHYDYAGFPYWGQGTLVTVSS

SEQ ID NO: 245 - protein, 338 aa, synthetic construct
HHHHHHGLNDIFEAQKIEWHEENLYFQSGSDAEQKACPPGKEPYKVDEDLIFYQNWELEACVDGTMLARQMDLVNEIPFTYEQLSIFKHKLDKTYPQGYPESLIQQLGHFFRYVSPEDIHQWNVTSPDTVKTLLKVSKGQKMNAQAIALVACYLRGGGQLDEDMVKALGDIPLSYLCDFSPQDLHSVPSSVMWLVGPQDLDKCSQRHLGLLYQKACSAFQNVSGLEYFEKIKTFLGGASVKDLRALSQHNVSMDIATFKRLQVDSLVGLSVAEVQKLLGPHVEGLKAEERHRPVRDWILRQRQDDLDTLGLGLQGGIPNGYLVLDLSMQEALSGGGGS

SEQ ID NO: 246 - protein, 338 aa, synthetic construct
HHHHHHGLNDIFEAQKIEWHEENLYFQSGSDAEQKACPPGKEPYKVDEDLIFYQNWELEACVDGTMLARQMDLVNEIPFTYEQLSIFKHKLDKTYPQGYPESLIQQLGHFFRYVSPEDIHQWNVTSPDTVKTLLKVSKGQKMNAQAIALVACYLRGGGQLDEDMVKALGDIPLSYLCDFSPQDLHSVPSSVMWLVGPQDLDKCSQRHLGLLYQKACSAFQNVSGLEYFEKIKTFLGGASVKDLRALSQHNVSMDIATFKRLQVDSLVGLSVAEVQKLLGPNIVDLKTEEDKSPVRDWLFRQHQKDLDRLGLGLQGGIPNGYLVLDLSMQEALSGGGGS

SEQ ID NO: 247 - protein, 311 aa, synthetic construct
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGSHHHHHH

SEQ ID NO: 248 - DNA, 1425 bp, synthetic construct
  source 1..1425: mol_type = other DNA
gacatccagatgacccagtctccatcctctctgtccgcctctgtgggcgacagagtgaccattacctgccgggccagacagtctatcggcaccgctatccactggtatcagcagaagcctggcaaggcccctaagctgctgattaagtacgcctccgagtccatctccggcgtgccctccagattttctggctctggatctggcaccgactttaccctgacaatctccagcctgcagcctgaggacttcgccacctactactgtcagcagtccggctcttggccttacacctttggtcagggcaccaagctggaaatcaagggcggatctgagggaaagtccagcggctccggcagcgaaagcaagtccaccggcggaagcgaggtgcagctggttgaatctggcggaggactggttaagcctggcggctctctgagactgtcttgtgctgcttctggcttcaccttcagccggtacaacatgaactgggtccgacaggctcctggcaaaggcctggaatgggtgtcctccatctccacctccagcaactacatctactacgccgactccgtgaagggcagattcaccttctccagagacaacgccaagaactccctggacctgcagatgtctggcctgagagctgaggacaccgctatctactactgcaccagaggctggggacccttcgattattggggccagggaaccctggtcaccgtgtcatctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 249 - protein, 6 aa, synthetic construct
HHHHHH

SEQ ID NO: 250 - protein, 12 aa, synthetic construct
LVLDLSVQEALS

SEQ ID NO: 251 - protein, 9 aa, synthetic construct
GDSVSSNRV

SEQ ID NO: 252 - protein, 7 aa, synthetic construct
YHRSKWY

SEQ ID NO: 253 - protein, 12 aa, synthetic construct
GDSVSSNSVAWN

(19) United States
(12) Patent Application Publication
(10) Pub. No.: US 2024/0166737 A1
(43) Pub. Date: May 23, 2024

(54) MSLN AND CD3 BINDING AGENTS AND METHODS OF USE THEREOF

(71) Applicant: Janssen Biotech, Inc., Horsham, PA (US)

(72) Inventors: Karine Smans, Antwerp (BE); Thomas C. Nesspor, Collegeville, PA (US); Winnie Chan, West Chester, PA (US); Isha Taneja, Spring House, PA (US); Stuart W. J. Ember, Eagleville, PA (US)

(73) Assignee: Janssen Biotech, Inc., Horsham, PA (US)

(21) Appl. No.: 18/493,367

(22) Filed: Oct. 24, 2023

Related U.S. Application Data
(60) Provisional application No. 63/419,174, filed on Oct. 25, 2022.

(51) Int. Cl. C07K 16/28; A61K 39/00; A61P 35/00; C12N 15/63; G01N 33/574

(52) U.S. Cl. CPC C07K 16/28; A61P 35/00; C07K 16/2809; C12N 15/63; G01N 33/57407; A61K 2039/505; C07K 2317/31; C07K 2317/524; C07K 2317/526; C07K 2317/55; C07K 2317/565; C07K 2317/622

(57) ABSTRACT

The present disclosure provides MSLN binding agents in the monospecific and multispecific formats, including bispecific MSLN×CD3 binding proteins, and related compositions and methods of use.

### 1. CROSS-REFERENCE TO RELATED APPLICATIONS

This application claims the benefit of U.S. Provisional Patent Application No. 63/419,174, filed Oct. 25, 2022, the disclosure of which is herein incorporated by reference in its entirety.

### 2. REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6711USNP1_SL.xml”, creation date of Oct. 11, 2023, and a size of 223 kilobytes (KB). This sequence listing submitted is part of the specification and is herein incorporated by reference in its entirety.

### 3. FIELD

The present disclosure generally relates to a T cell engager that binds mesothelin (MSLN) and CD3, compositions comprising same and methods of using same.

### 4. BACKGROUND

Mesothelin (MSLN) is a lineage-restricted cell surface protein that originates from the MSLN gene. MSLN encodes a 71 kDa precursor protein that is processed intracellularly by furin cleavage into a secreted 31 kDa protein megakaryocyte promoting factor (MPF) and the membrane expressed 40 kDa protein MSLN (Ho et al., 2011, “A novel high-affinity human monoclonal antibody to mesothelin,” Int. J. Cancer). MSLN is a glycosylated and GPI-anchored glycoprotein.

Therapeutic antibodies have been developed against MSLN but were found to have medium to low efficacy. There exists a need for new and more effective therapeutic molecules targeting MSLN. The present disclosure meets this and other needs.

### 5. SUMMARY

In one aspect, provided herein is a binding agent comprising an antigen binding region that (a) binds to an epitope of MSLN recognized by an antibody comprising a heavy chain variable (VH) domain comprising a VH complementarity determining region 1 (HCDR1), a HCDR2, and a HCDR3 having an amino acid sequence of a HCDR1, a HCDR2, and a HCDR3, respectively, of a VH domain having an amino acid sequence of SEQ ID NO:81 and a light chain variable (VL) domain comprising a LCDR1, a LCDR2, and a LCDR3 having an amino acid sequence of a LCDR1, a LCDR2, and a LCDR3, respectively, of a VL domain having an amino acid sequence of SEQ ID NO:82; (b) competes for the binding to MSLN with an antibody comprising a VH domain comprising a HCDR1, a HCDR2, and a HCDR3 having an amino acid sequence of a HCDR1, a HCDR2, and a HCDR3, respectively, of a VH domain having an amino acid sequence of SEQ ID NO:81 and a VL domain comprising a LCDR1, a LCDR2, and a LCDR3 having an amino acid sequence of a LCDR1, a LCDR2, and a LCDR3, respectively, of a VL domain having an amino acid sequence of SEQ ID NO:82; or (c) binds to an epitope of MSLN in the membrane-restricted region of MSLN.

In one aspect, provided herein is a binding agent comprising an antigen binding region that binds to MSLN, wherein the antigen binding region comprises: (a) a heavy chain variable (VH) domain comprising VH complementarity determining region 1 (HCDR1), HCDR2, and HCDR3 of any one of antibodies MSNB457, MSNB71, MSNB124, MSNB133, MSNB459, MSNB568, MSNB569, MSNB570, MSNB571, MSNB590, MSNB591, or MSNB592 as set forth in Tables 1 to 12; and/or (b) a light chain variable (VL) domain comprising LCDR1, LCDR2, and LCDR3 of any one of antibodies MSNB457, MSNB71, MSNB124, MSNB133, MSNB459, MSNB568, MSNB569, MSNB570, MSNB571, MSNB590, MSNB591, or MSNB592 as set forth in Tables 1 to 12.

In some embodiments, the antigen binding region comprises a VH domain comprising a HCDR1, HCDR2, and HCDR3 as set forth in SEQ ID NO:81, and a VL domain comprising a LCDR1, LCDR2, and LCDR3 as set forth in SEQ ID NO:82.

In some embodiments, the antigen binding region comprises a VH domain comprising a HCDR1, HCDR2, and HCDR3 as set forth in SEQ ID NO:22, and a VL domain comprising a LCDR1, LCDR2, and LCDR3 as set forth in SEQ ID NO:23.

In some embodiments, the antigen binding region comprises a VH domain comprising a HCDR1, HCDR2, and HCDR3 as set forth in SEQ ID NO:45, and a VL domain comprising a LCDR1, LCDR2, and LCDR3 as set forth in SEQ ID NO:46.

In some embodiments, the antigen binding region comprises a VH domain comprising a HCDR1, HCDR2, and HCDR3 as set forth in SEQ ID NO:62, and a VL domain comprising a LCDR1, LCDR2, and LCDR3 as set forth in SEQ ID NO:63.

In some embodiments, the antigen binding region comprises a VH domain comprising a HCDR1, HCDR2, and HCDR3 as set forth in SEQ ID NO:94, and a VL domain comprising a LCDR1, LCDR2, and LCDR3 as set forth in SEQ ID NO:95.

In some embodiments, the antigen binding region comprises a VH domain comprising a HCDR1, HCDR2, and HCDR3 as set forth in SEQ ID NO:106, and a VL domain comprising a LCDR1, LCDR2, and LCDR3 as set forth in SEQ ID NO: 107.

In some embodiments, the antigen binding region comprises a VH domain comprising a HCDR1, HCDR2, and HCDR3 as set forth in SEQ ID NO: 120, and a VL domain comprising a LCDR1, LCDR2, and LCDR3 as set forth in SEQ ID NO:121.

In some embodiments, the antigen binding region comprises a VH domain comprising a HCDR1, HCDR2, and HCDR3 as set forth in SEQ ID NO: 132, and a VL domain comprising a LCDR1, LCDR2, and LCDR3 as set forth in SEQ ID NO:133.

In some embodiments, the antigen binding region comprises a VH domain comprising a HCDR1, HCDR2, and HCDR3 as set forth in SEQ ID NO: 138, and a VL domain comprising a LCDR1, LCDR2, and LCDR3 as set forth in SEQ ID NO:133.

In some embodiments, the antigen binding region comprises a VH domain comprising a HCDR1, HCDR2, and HCDR3 as set forth in SEQ ID NO: 120, and a VL domain comprising a LCDR1, LCDR2, and LCDR3 as set forth in SEQ ID NO: 139.

In some embodiments, the antigen binding region comprises a VH domain comprising a HCDR1, HCDR2, and HCDR3 as set forth in SEQ ID NO: 106, and a VL domain comprising a LCDR1, LCDR2, and LCDR3 as set forth in SEQ ID NO: 140.

In some embodiments, the antigen binding region comprises a VH domain comprising a HCDR1, HCDR2, and HCDR3 as set forth in SEQ ID NO:132, and a VL domain comprising a LCDR1, LCDR2, and LCDR3 as set forth in SEQ ID NO:142.

In some embodiments, provided herein is a binding agent comprising an antigen binding region that binds to MSLN, wherein the antigen binding region comprises: (a) a VH domain comprising HCDR1, HCDR2, and HCDR3; and/or a VL domain comprising LCDR1, LCDR2, and LCDR3.

In particular embodiments, HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In particular embodiments, HCDR1 comprises an amino acid sequence of SEQ ID NO:70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In particular embodiments, HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In particular embodiments, HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76.

In particular embodiments, HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69.

In particular embodiments, HCDR1 comprises an amino acid sequence of SEQ ID NO:1; HCDR2 comprises an amino acid sequence of SEQ ID NO:2; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6.

In particular embodiments, HCDR1 comprises an amino acid sequence of SEQ ID NO:7; HCDR2 comprises an amino acid sequence of SEQ ID NO:8; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6.

In particular embodiments, HCDR1 comprises an amino acid sequence of SEQ ID NO:9; HCDR2 comprises an amino acid sequence of SEQ ID NO:10; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6.

In particular embodiments, HCDR1 comprises an amino acid sequence of SEQ ID NO:11; HCDR2 comprises an amino acid sequence of SEQ ID NO: 12; HCDR3 comprises an amino acid sequence of SEQ ID NO: 13; LCDR1 comprises an amino acid sequence of SEQ ID NO:14; LCDR2 comprises an amino acid sequence of SEQ ID NO:15; and LCDR3 comprises an amino acid sequence of SEQ ID NO: 16.

In particular embodiments, HCDR1 comprises an amino acid sequence of SEQ ID NO:17; HCDR2 comprises an amino acid sequence of SEQ ID NO: 18; HCDR3 comprises an amino acid sequence of SEQ ID NO: 19; LCDR1 comprises an amino acid sequence of SEQ ID NO:20; LCDR2 comprises an amino acid sequence of LVS; and LCDR3 comprises an amino acid sequence of SEQ ID NO:6.

In particular embodiments, HCDR1 comprises an amino acid sequence of SEQ ID NO:24; HCDR2 comprises an amino acid sequence of SEQ ID NO:25; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29.

In particular embodiments, HCDR1 comprises an amino acid sequence of SEQ ID NO:30; HCDR2 comprises an amino acid sequence of SEQ ID NO:252; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29.

In particular embodiments, HCDR1 comprises an amino acid sequence of SEQ ID NO:32; HCDR2 comprises an amino acid sequence of SEQ ID NO:33; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29.

In particular embodiments, HCDR1 comprises an amino acid sequence of SEQ ID NO:34; HCDR2 comprises an amino acid sequence of SEQ ID NO:65; HCDR3 comprises an amino acid sequence of SEQ ID NO:36; LCDR1 comprises an amino acid sequence of SEQ ID NO:37; LCDR2 comprises an amino acid sequence of SEQ ID NO:38; and LCDR3 comprises an amino acid sequence of SEQ ID NO:39.

In particular embodiments, HCDR1 comprises an amino acid sequence of SEQ ID NO:40; HCDR2 comprises an amino acid sequence of SEQ ID NO:41; HCDR3 comprises an amino acid sequence of SEQ ID NO:42; LCDR1 comprises an amino acid sequence of SEQ ID NO:43; LCDR2 comprises an amino acid sequence of LGS; and LCDR3 comprises an amino acid sequence of SEQ ID NO:29.

In particular embodiments, HCDR1 comprises an amino acid sequence of SEQ ID NO:47; HCDR2 comprises an amino acid sequence of SEQ ID NO:48; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50.

In particular embodiments, HCDR1 comprises an amino acid sequence of SEQ ID NO:251; HCDR2 comprises an amino acid sequence of SEQ ID NO:31; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50.

In particular embodiments, HCDR1 comprises an amino acid sequence of SEQ ID NO:51; HCDR2 comprises an amino acid sequence of SEQ ID NO:52; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50.

In particular embodiments, HCDR1 comprises an amino acid sequence of SEQ ID NO:53; HCDR2 comprises an amino acid sequence of SEQ ID NO:54; HCDR3 comprises an amino acid sequence of SEQ ID NO:55; LCDR1 comprises an amino acid sequence of SEQ ID NO:56; LCDR2 comprises an amino acid sequence of SEQ ID NO:38; and LCDR3 comprises an amino acid sequence of SEQ ID NO:57.

In particular embodiments, HCDR1 comprises an amino acid sequence of SEQ ID NO:58; HCDR2 comprises an amino acid sequence of SEQ ID NO:59; HCDR3 comprises an amino acid sequence of SEQ ID NO:60; LCDR1 comprises an amino acid sequence of SEQ ID NO:61; LCDR2 comprises an amino acid sequence of LGS; and LCDR3 comprises an amino acid sequence of SEQ ID NO:50.

In particular embodiments, HCDR1 comprises an amino acid sequence of SEQ ID NO:83; HCDR2 comprises an amino acid sequence of SEQ ID NO:84; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:30; HCDR2 comprises an amino acid sequence of SEQ ID NO:31; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:253; HCDR2 comprises an amino acid sequence of SEQ ID NO:52; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:88; HCDR2 comprises an amino acid sequence of SEQ ID NO:54; HCDR3 comprises an amino acid sequence of SEQ ID NO:89; LCDR1 comprises an amino acid sequence of SEQ ID NO:90; LCDR2 comprises an amino acid sequence of SEQ ID NO:38; and LCDR3 comprises an amino acid sequence of SEQ ID NO:91.

In particular embodiments, HCDR1 comprises an amino acid sequence of SEQ ID NO:40; HCDR2 comprises an amino acid sequence of SEQ ID NO:59; HCDR3 comprises an amino acid sequence of SEQ ID NO:92; LCDR1 comprises an amino acid sequence of SEQ ID NO:93; LCDR2 comprises an amino acid sequence of LGS; and LCDR3 comprises an amino acid sequence of SEQ ID NO:87.

In particular embodiments, HCDR1 comprises an amino acid sequence of SEQ ID NO:1; HCDR2 comprises an amino acid sequence of SEQ ID NO:96; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99.

In particular embodiments, HCDR1 comprises an amino acid sequence of SEQ ID NO:7; HCDR2 comprises an amino acid sequence of SEQ ID NO:31; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99.

In particular embodiments, HCDR1 comprises an amino acid sequence of SEQ ID NO:9; HCDR2 comprises an amino acid sequence of SEQ ID NO:52; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99.

In particular embodiments, HCDR1 comprises an amino acid sequence of SEQ ID NO:11; HCDR2 comprises an amino acid sequence of SEQ ID NO:54; HCDR3 comprises an amino acid sequence of SEQ ID NO: 100; LCDR1 comprises an amino acid sequence of SEQ ID NO:101; LCDR2 comprises an amino acid sequence of SEQ ID NO:102; and LCDR3 comprises an amino acid sequence of SEQ ID NO: 103.

In particular embodiments, HCDR1 comprises an amino acid sequence of SEQ ID NO:17; HCDR2 comprises an amino acid sequence of SEQ ID NO:59; HCDR3 comprises an amino acid sequence of SEQ ID NO: 104; LCDR1 comprises an amino acid sequence of SEQ ID NO:105; LCDR2 comprises an amino acid sequence of LVS; and LCDR3 comprises an amino acid sequence of SEQ ID NO:99.

In particular embodiments, HCDR1 comprises an amino acid sequence of SEQ ID NO:108; HCDR2 comprises an amino acid sequence of SEQ ID NO: 109; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:112; HCDR2 comprises an amino acid sequence of SEQ ID NO:31; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:113; HCDR2 comprises an amino acid sequence of SEQ ID NO:52; HCDR3 comprises an amino acid sequence of SEQ ID NO: 110; LCDR1 comprises an amino acid sequence of SEQ ID NO:111; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:114; HCDR2 comprises an amino acid sequence of SEQ ID NO:54; HCDR3 comprises an amino acid sequence of SEQ ID NO: 115; LCDR1 comprises an amino acid sequence of SEQ ID NO:116; LCDR2 comprises an amino acid sequence of SEQ ID NO:38; and LCDR3 comprises an amino acid sequence of SEQ ID NO:91.

In particular embodiments, HCDR1 comprises an amino acid sequence of SEQ ID NO:117; HCDR2 comprises an amino acid sequence of SEQ ID NO:59; HCDR3 comprises an amino acid sequence of SEQ ID NO:118; LCDR1 comprises an amino acid sequence of SEQ ID NO:119; LCDR2 comprises an amino acid sequence of LGS; and LCDR3 comprises an amino acid sequence of SEQ ID NO:87.

In particular embodiments, HCDR1 comprises an amino acid sequence of SEQ ID NO:122; HCDR2 comprises an amino acid sequence of SEQ ID NO:84; HCDR3 comprises an amino acid sequence of SEQ ID NO:3; LCDR1 comprises an amino acid sequence of SEQ ID NO:123; LCDR2 comprises an amino acid sequence of SEQ ID NO: 124; and LCDR3 comprises an amino acid sequence of SEQ ID NO:99.

In particular embodiments, HCDR1 comprises an amino acid sequence of SEQ ID NO:125; HCDR2 comprises an amino acid sequence of SEQ ID NO:31; HCDR3 comprises an amino acid sequence of SEQ ID NO:3; LCDR1 comprises an amino acid sequence of SEQ ID NO:123; LCDR2 comprises an amino acid sequence of SEQ ID NO:124; and LCDR3 comprises an amino acid sequence of SEQ ID NO:99.

In particular embodiments, HCDR1 comprises an amino acid sequence of SEQ ID NO:126; HCDR2 comprises an amino acid sequence of SEQ ID NO:52; HCDR3 comprises an amino acid sequence of SEQ ID NO:3; LCDR1 comprises an amino acid sequence of SEQ ID NO:123; LCDR2 comprises an amino acid sequence of SEQ ID NO:124; and LCDR3 comprises an amino acid sequence of SEQ ID NO:99.

In particular embodiments, HCDR1 comprises an amino acid sequence of SEQ ID NO:127; HCDR2 comprises an amino acid sequence of SEQ ID NO:54; HCDR3 comprises an amino acid sequence of SEQ ID NO: 13; LCDR1 comprises an amino acid sequence of SEQ ID NO:128; LCDR2 comprises an amino acid sequence of SEQ ID NO: 129; and LCDR3 comprises an amino acid sequence of SEQ ID NO: 103.

In particular embodiments, HCDR1 comprises an amino acid sequence of SEQ ID NO:130; HCDR2 comprises an amino acid sequence of SEQ ID NO:59; HCDR3 comprises an amino acid sequence of SEQ ID NO: 19; LCDR1 comprises an amino acid sequence of SEQ ID NO:131; LCDR2 comprises an amino acid sequence of LGS; and LCDR3 comprises an amino acid sequence of SEQ ID NO:99.

In particular embodiments, HCDR1 comprises an amino acid sequence of SEQ ID NO:122; HCDR2 comprises an amino acid sequence of SEQ ID NO:134; HCDR3 comprises an amino acid sequence of SEQ ID NO:3; LCDR1 comprises an amino acid sequence of SEQ ID NO:123; LCDR2 comprises an amino acid sequence of SEQ ID NO: 124; and LCDR3 comprises an amino acid sequence of SEQ ID NO:99.

In particular embodiments, HCDR1 comprises an amino acid sequence of SEQ ID NO:125; HCDR2 comprises an amino acid sequence of SEQ ID NO:31; HCDR3 comprises an amino acid sequence of SEQ ID NO:3; LCDR1 comprises an amino acid sequence of SEQ ID NO:123; LCDR2 comprises an amino acid sequence of SEQ ID NO:124; and LCDR3 comprises an amino acid sequence of SEQ ID NO:99.

In particular embodiments, HCDR1 comprises an amino acid sequence of SEQ ID NO:126; HCDR2 comprises an amino acid sequence of SEQ ID NO: 135; HCDR3 comprises an amino acid sequence of SEQ ID NO:3; LCDR1 comprises an amino acid sequence of SEQ ID NO:123; LCDR2 comprises an amino acid sequence of SEQ ID NO: 124; and LCDR3 comprises an amino acid sequence of SEQ ID NO:99.

In particular embodiments, HCDR1 comprises an amino acid sequence of SEQ ID NO:127; HCDR2 comprises an amino acid sequence of SEQ ID NO: 136; HCDR3 comprises an amino acid sequence of SEQ ID NO: 13; LCDR1 comprises an amino acid sequence of SEQ ID NO:128; LCDR2 comprises an amino acid sequence of SEQ ID NO:129; and LCDR3 comprises an amino acid sequence of SEQ ID NO: 103.

In particular embodiments, HCDR1 comprises an amino acid sequence of SEQ ID NO:130; HCDR2 comprises an amino acid sequence of SEQ ID NO:137; HCDR3 comprises an amino acid sequence of SEQ ID NO:19; LCDR1 comprises an amino acid sequence of SEQ ID NO:131; LCDR2 comprises an amino acid sequence of LGS; and LCDR3 comprises an amino acid sequence of SEQ ID NO:99.

In particular embodiments, HCDR1 comprises an amino acid sequence of SEQ ID NO:108; HCDR2 comprises an amino acid sequence of SEQ ID NO: 109; HCDR3 comprises an amino acid sequence of SEQ ID NO: 110; LCDR1 comprises an amino acid sequence of SEQ ID NO:111; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:112; HCDR2 comprises an amino acid sequence of SEQ ID NO:31; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:113; HCDR2 comprises an amino acid sequence of SEQ ID NO:52; HCDR3 comprises an amino acid sequence of SEQ ID NO: 110; LCDR1 comprises an amino acid sequence of SEQ ID NO:111; LCDR2 comprises an amino acid sequence of SEQ ID NO:28; and LCDR3 comprises an amino acid sequence of SEQ ID NO:87.

In particular embodiments, HCDR1 comprises an amino acid sequence of SEQ ID NO:114; HCDR2 comprises an amino acid sequence of SEQ ID NO:54; HCDR3 comprises an amino acid sequence of SEQ ID NO:115; LCDR1 comprises an amino acid sequence of SEQ ID NO:116; LCDR2 comprises an amino acid sequence of SEQ ID NO:38; and LCDR3 comprises an amino acid sequence of SEQ ID NO:91.

In particular embodiments, HCDR1 comprises an amino acid sequence of SEQ ID NO:117; HCDR2 comprises an amino acid sequence of SEQ ID NO:59; HCDR3 comprises an amino acid sequence of SEQ ID NO:118; LCDR1 comprises an amino acid sequence of SEQ ID NO:119; LCDR2 comprises an amino acid sequence of LGS; and LCDR3 comprises an amino acid sequence of SEQ ID NO:87.

In particular embodiments, HCDR1 comprises an amino acid sequence of SEQ ID NO:1; HCDR2 comprises an amino acid sequence of SEQ ID NO:96; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99.

In particular embodiments, HCDR1 comprises an amino acid sequence of SEQ ID NO:7; HCDR2 comprises an amino acid sequence of SEQ ID NO:31; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99.

In particular embodiments, HCDR1 comprises an amino acid sequence of SEQ ID NO:9; HCDR2 comprises an amino acid sequence of SEQ ID NO:52; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99.

In particular embodiments, HCDR1 comprises an amino acid sequence of SEQ ID NO:11; HCDR2 comprises an amino acid sequence of SEQ ID NO:54; HCDR3 comprises an amino acid sequence of SEQ ID NO:100; LCDR1 comprises an amino acid sequence of SEQ ID NO:101; LCDR2 comprises an amino acid sequence of SEQ ID NO:102; and LCDR3 comprises an amino acid sequence of SEQ ID NO: 103.

In particular embodiments, HCDR1 comprises an amino acid sequence of SEQ ID NO:17; HCDR2 comprises an amino acid sequence of SEQ ID NO:59; HCDR3 comprises an amino acid sequence of SEQ ID NO:104; LCDR1 comprises an amino acid sequence of SEQ ID NO:105; LCDR2 comprises an amino acid sequence of LVS; and LCDR3 comprises an amino acid sequence of SEQ ID NO:99.

In particular embodiments, HCDR1 comprises an amino acid sequence of SEQ ID NO:122; HCDR2 comprises an amino acid sequence of SEQ ID NO:84; HCDR3 comprises an amino acid sequence of SEQ ID NO:3; LCDR1 comprises an amino acid sequence of SEQ ID NO:123; LCDR2 comprises an amino acid sequence of SEQ ID NO:28; and LCDR3 comprises an amino acid sequence of SEQ ID NO:99.

In particular embodiments, HCDR1 comprises an amino acid sequence of SEQ ID NO:125; HCDR2 comprises an amino acid sequence of SEQ ID NO:31; HCDR3 comprises an amino acid sequence of SEQ ID NO:3; LCDR1 comprises an amino acid sequence of SEQ ID NO:123; LCDR2 comprises an amino acid sequence of SEQ ID NO:28; and LCDR3 comprises an amino acid sequence of SEQ ID NO:99.

In particular embodiments, HCDR1 comprises an amino acid sequence of SEQ ID NO:126; HCDR2 comprises an amino acid sequence of SEQ ID NO:52; HCDR3 comprises an amino acid sequence of SEQ ID NO:3; LCDR1 comprises an amino acid sequence of SEQ ID NO:123; LCDR2 comprises an amino acid sequence of SEQ ID NO:28; and LCDR3 comprises an amino acid sequence of SEQ ID NO:99.

In particular embodiments, HCDR1 comprises an amino acid sequence of SEQ ID NO:127; HCDR2 comprises an amino acid sequence of SEQ ID NO:54; HCDR3 comprises an amino acid sequence of SEQ ID NO:13; LCDR1 comprises an amino acid sequence of SEQ ID NO:128; LCDR2 comprises an amino acid sequence of SEQ ID NO:141; and LCDR3 comprises an amino acid sequence of SEQ ID NO:103.

In particular embodiments, HCDR1 comprises an amino acid sequence of SEQ ID NO:130; HCDR2 comprises an amino acid sequence of SEQ ID NO:59; HCDR3 comprises an amino acid sequence of SEQ ID NO:19; LCDR1 comprises an amino acid sequence of SEQ ID NO:131; LCDR2 comprises an amino acid sequence of LGS; and LCDR3 comprises an amino acid sequence of SEQ ID NO:99.

In some embodiments, provided herein is a binding agent comprising an antigen binding region that binds to MSLN, wherein the antigen binding region comprises a VH and/or a VL domain.

In particular embodiments, the antigen binding region comprises a VH comprising an amino acid sequence of SEQ ID NO:81. In particular embodiments, the antigen binding region comprises a VH comprising an amino acid sequence of SEQ ID NO:22. In particular embodiments, the antigen binding region comprises a VH comprising an amino acid sequence of SEQ ID NO:45. In particular embodiments, the antigen binding region comprises a VH comprising an amino acid sequence of SEQ ID NO:62. In particular embodiments, the antigen binding region comprises a VH comprising an amino acid sequence of SEQ ID NO:94. In particular embodiments, the antigen binding region comprises a VH comprising an amino acid sequence of SEQ ID NO: 106. In particular embodiments, the antigen binding region comprises a VH comprising an amino acid sequence of SEQ ID NO:120. In particular embodiments, the antigen binding region comprises a VH comprising an amino acid sequence of SEQ ID NO:132. In particular embodiments, the antigen binding region comprises a VH comprising an amino acid sequence of SEQ ID NO: 138.

In particular embodiments, the antigen binding region comprises a VL comprising an amino acid sequence of SEQ ID NO:82. In particular embodiments, the antigen binding region comprises a VL comprising an amino acid sequence of SEQ ID NO:23. In particular embodiments, the antigen binding region comprises a VL comprising an amino acid sequence of SEQ ID NO:46. In particular embodiments, the antigen binding region comprises a VL comprising an amino acid sequence of SEQ ID NO:63. In particular embodiments, the antigen binding region comprises a VL comprising an amino acid sequence of SEQ ID NO:82. In particular embodiments, the antigen binding region comprises a VL comprising an amino acid sequence of SEQ ID NO:95. In particular embodiments, the antigen binding region comprises a VL comprising an amino acid sequence of SEQ ID NO: 107. In particular embodiments, the antigen binding region comprises a VL comprising an amino acid sequence of SEQ ID NO:121. In particular embodiments, the antigen binding region comprises a VL comprising an amino acid sequence of SEQ ID NO:133. In particular embodiments, the antigen binding region comprises a VL comprising an amino acid sequence of SEQ ID NO: 139. In particular embodiments, the antigen binding region comprises a VL comprising an amino acid sequence of SEQ ID NO:140. In particular embodiments, the antigen binding region comprises a VL comprising an amino acid sequence of SEQ ID NO: 142. In particular embodiments, the antigen binding region comprises a VH domain comprising an amino acid sequence of SEQ ID NO:81, and a VL domain comprising an amino acid sequence of SEQ ID NO:82.

In particular embodiments, the antigen binding region comprises a VH domain comprising an amino acid sequence of SEQ ID NO:22, and a VL domain comprising an amino acid sequence of SEQ ID NO:23. In particular embodiments, the antigen binding region comprises a VH domain comprising an amino acid sequence of SEQ ID NO:45, and a VL domain comprising an amino acid sequence of SEQ ID NO:46. In particular embodiments, the antigen binding region comprises a VH domain comprising an amino acid sequence of SEQ ID NO:62, and a VL domain comprising an amino acid sequence of SEQ ID NO:63. In particular embodiments, the antigen binding region comprises a VH domain comprising an amino acid sequence of SEQ ID NO:94, and a VL domain comprising an amino acid sequence of SEQ ID NO:95. In particular embodiments, the antigen binding region comprises a VH domain comprising an amino acid sequence of SEQ ID NO:106, and a VL domain comprising an amino acid sequence of SEQ ID NO:107. In particular embodiments, the antigen binding region comprises a VH domain comprising an amino acid sequence of SEQ ID NO: 120, and a VL domain comprising an amino acid sequence of SEQ ID NO: 121. In particular embodiments, the antigen binding region comprises a VH domain comprising an amino acid sequence of SEQ ID NO:132, and a VL domain comprising an amino acid sequence of SEQ ID NO: 133. In particular embodiments, the antigen binding region comprises a VH domain comprising an amino acid sequence of SEQ ID NO:138, and a VL domain comprising an amino acid sequence of SEQ ID NO:133. In particular embodiments, the antigen binding region comprises a VH domain comprising an amino acid sequence of SEQ ID NO: 120, and a VL domain comprising an amino acid sequence of SEQ ID NO: 139. In particular embodiments, the antigen binding region comprises a VH domain comprising an amino acid sequence of SEQ ID NO: 106, and a VL domain comprising an amino acid sequence of SEQ ID NO: 140. In particular embodiments, the antigen binding region comprises a VH domain comprising an amino acid sequence of SEQ ID NO:132, and a VL domain comprising an amino acid sequence of SEQ ID NO:142.

In various embodiments of the binding agent described herein, the antigen binding region that binds to MSLN is a scFv, a (scFv)2, a Fv, a Fab, a F(ab′)2, a Fd, or a dAb. In some embodiments, the antigen binding region that binds to MSLN is the Fab.

In some embodiments, the antigen binding region that binds to MSLN is the scFv. In some embodiments, the scFv comprises, from the N- to C-terminus, a VH, a first linker (L1) and a VL (VH-L1-VL) or the VL, the L1 and the VH (VL-L1-VH). In some embodiments, the L1 comprises (a) about 5 to 50 amino acids; (b) about 5 to 40 amino acids; (c) about 10 to 30 amino acids; or (d) about 10 to 20 amino acids. In some embodiments, the L1 comprises the amino acid sequence of SEQ ID NO: 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199.

In some embodiments, the binding agent is a multi-specific protein. In some embodiments, the multispecific protein is a bispecific protein. In some embodiments, the multispecific protein is a trispecific protein.

In some embodiments, the binding agent further comprises an immunoglobulin (Ig) constant region of or a fragment of the Ig constant region. In some embodiments, the fragment of the Ig constant region comprises a Fc region. In some embodiments, the fragment of the Ig constant region comprises a CH2 domain. In some embodiments, the fragment of the Ig constant region comprises a CH3 domain. In some embodiments, the fragment of the Ig constant region comprises a CH2 domain and a CH3 domain. In some embodiments, the fragment of the Ig constant region comprises at least portion of a hinge, a CH2 domain and a CH3 domain. In some embodiments, the fragment of the Ig constant region comprises a hinge, a CH2 domain and a CH3 domain.

In some embodiments, the antigen binding region that binds MSLN is fused or conjugated to the N-terminus of the Ig constant region or the fragment of the Ig constant region. In some embodiments, the antigen binding region that binds MSLN is fused or conjugated to the C-terminus of the Ig constant region or the fragment of the Ig constant region.

In some embodiments, the antigen binding region that binds MSLN is fused or conjugated to the Ig constant region or the fragment of the Ig constant region via a second linker (L2). In some embodiments, the L2 comprises the amino acid sequence of SEQ ID NO:166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199.

In some embodiments, the Ig constant region or the fragment of the Ig constant region is of an IgG1, an IgG2, an IgG3 or an IgG4 isotype.

In some embodiments, the Ig constant region or the fragment of the Ig constant region comprises at least one mutation that results in reduced binding of the protein to a Fcγ receptor (FcγR). In some embodiments, the at least one mutation that results in reduced binding of the binding agent to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the FcγR is FcγRI, FcγRIIA, FcγRIIB or FcγRIII, or any combination thereof.

In some embodiments, the protein comprises at least one mutation in a CH3 domain of the Ig constant region or the fragment of the Ig constant region. In some embodiments, the at least one mutation in the CH3 domain is selected from the group consisting of T350V, L351Y, F405A, Y407V, T366Y, T366W, T366L, F405W, T394W, K392L, T394S, Y407T, Y407A, T366S/L368A/Y407V, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index. In some embodiments, at least one mutation in the CH3 domain is selected from the group consisting of H435R, Y436F and H435R/L436F.

In some embodiments, the antigen binding region specifically binds to membrane-associated MSLN and does not bind to a soluble MSLN isoform or shed MSLN. In some embodiments, when bound to MSLN, the antigen binding region binds to at least one of residues 587-598 within an amino acid sequence of SEQ ID NO:200. In some embodiments, when bound to MSLN, the antigen binding region binds to at least one residue selected from the group consisting of L589, D590, M593, V588, S592, L597, E595, and A596 with an amino acid sequence of SEQ ID NO:200. In some embodiments, when bound to MSLN, the antigen binding region binds to at least one residue selected from the group consisting of L589, D590, and M593 with an amino acid sequence of SEQ ID NO:200. In some embodiments, when bound to MSLN, the antigen binding region binds to at least one residue selected from the group consisting of V588, S592, E595, and L597 with an amino acid sequence of SEQ ID NO:200. In some embodiments, when bound to MSLN, the antigen binding region binds to at least one residue selected from the group consisting of V588, S592, and L597 with an amino acid sequence of SEQ ID NO:200.

In some embodiments, the multispecific protein comprises an antigen binding region that binds a second antigen other than MSLN. In some embodiments, the second antigen is cluster of differentiation 38 (CD3ε).

In one aspect, provided herein is a binding agent comprising a first antigen binding region that binds to MSLN and a second antigen binding region that binds to CD3ε, wherein (i) the first antigen binding region comprises a VH domain comprising a HCDR1, HCDR2, and HCDR3 as set forth in SEQ ID NO:81, and a VL domain comprising a LCDR1, LCDR2, and LCDR3 as set forth in SEQ ID NO:82; and (ii) the second antigen binding region a VH domain comprising a HCDR1, HCDR2, and HCDR3 as set forth in SEQ ID NO: 159, and a VL domain comprising a LCDR1, LCDR2, and LCDR3 as set forth in SEQ ID NO:160.

In some embodiments, wherein (i) in the first antigen binding region that binds to MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO: 143; HCDR2 comprises an amino acid sequence of SEQ ID NO:144; HCDR3 comprises an amino acid sequence of SEQ ID NO:145; LCDR1 comprises an amino acid sequence of SEQ ID NO:146; LCDR2 comprises an amino acid sequence of SEQ ID NO:147; and LCDR3 comprises an amino acid sequence of SEQ ID NO:148.

In some embodiments, wherein (i) in the first antigen binding region that binds to MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO: 149; HCDR2 comprises an amino acid sequence of SEQ ID NO:150; HCDR3 comprises an amino acid sequence of SEQ ID NO:151; LCDR1 comprises an amino acid sequence of SEQ ID NO: 152; LCDR2 comprises an amino acid sequence of YAS; and LCDR3 comprises an amino acid sequence of SEQ ID NO:154.

In some embodiments, wherein (i) in the first antigen binding region that binds to MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO: 155; HCDR2 comprises an amino acid sequence of SEQ ID NO:156; HCDR3 comprises an amino acid sequence of SEQ ID NO:157; LCDR1 comprises an amino acid sequence of SEQ ID NO:158; LCDR2 comprises an amino acid sequence of YAS; and LCDR3 comprises an amino acid sequence of SEQ ID NO:148.

In some embodiments, wherein (i) in the first antigen binding region that binds to MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO:143; HCDR2 comprises an amino acid sequence of SEQ ID NO: 144; HCDR3 comprises an amino acid sequence of SEQ ID NO:145; LCDR1 comprises an amino acid sequence of SEQ ID NO: 146; LCDR2 comprises an amino acid sequence of SEQ ID NO:147; and LCDR3 comprises an amino acid sequence of SEQ ID NO: 148.

In some embodiments, wherein (i) in the first antigen binding region that binds to MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO:149; HCDR2 comprises an amino acid sequence of SEQ ID NO:150; HCDR3 comprises an amino acid sequence of SEQ ID NO:151; LCDR1 comprises an amino acid sequence of SEQ ID NO:152; LCDR2 comprises an amino acid sequence of YAS; and LCDR3 comprises an amino acid sequence of SEQ ID NO:154.

In some embodiments, wherein (i) in the first antigen binding region that binds to MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO:155; HCDR2 comprises an amino acid sequence of SEQ ID NO:156; HCDR3 comprises an amino acid sequence of SEQ ID NO:157; LCDR1 comprises an amino acid sequence of SEQ ID NO:158; LCDR2 comprises an amino acid sequence of YAS; and LCDR3 comprises an amino acid sequence of SEQ ID NO:148.

In some embodiments, wherein (i) in the first antigen binding region that binds to MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO: 143; HCDR2 comprises an amino acid sequence of SEQ ID NO:144; HCDR3 comprises an amino acid sequence of SEQ ID NO:145; LCDR1 comprises an amino acid sequence of SEQ ID NO: 146; LCDR2 comprises an amino acid sequence of SEQ ID NO:147; and LCDR3 comprises an amino acid sequence of SEQ ID NO: 148.

In some embodiments, wherein (i) in the first antigen binding region that binds to MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO:149; HCDR2 comprises an amino acid sequence of SEQ ID NO:150; HCDR3 comprises an amino acid sequence of SEQ ID NO:151; LCDR1 comprises an amino acid sequence of SEQ ID NO:152; LCDR2 comprises an amino acid sequence of YAS; and LCDR3 comprises an amino acid sequence of SEQ ID NO:154.

In some embodiments, wherein (i) in the first antigen binding region that binds to MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69; and (ii) in the second antigen binding region that binds to CD3ε, HCDR1 comprises an amino acid sequence of SEQ ID NO:155; HCDR2 comprises an amino acid sequence of SEQ ID NO:156; HCDR3 comprises an amino acid sequence of SEQ ID NO:157; LCDR1 comprises an amino acid sequence of SEQ ID NO:158; LCDR2 comprises an amino acid sequence of YAS; and LCDR3 comprises an amino acid sequence of SEQ ID NO:148.

In some embodiments, wherein (i) in the first antigen binding region that binds to MSLN, HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76; and (ii) in the second antigen binding region that binds to CD3ε, HCDR1 comprises an amino acid sequence of SEQ ID NO:143; HCDR2 comprises an amino acid sequence of SEQ ID NO:144; HCDR3 comprises an amino acid sequence of SEQ ID NO:145; LCDR1 comprises an amino acid sequence of SEQ ID NO: 146; LCDR2 comprises an amino acid sequence of SEQ ID NO:147; and LCDR3 comprises an amino acid sequence of SEQ ID NO:148.

In some embodiments, wherein (i) in the first antigen binding region that binds to MSLN, HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76; and (ii) in the second antigen binding region that binds to CD3ε, HCDR1 comprises an amino acid sequence of SEQ ID NO:149; HCDR2 comprises an amino acid sequence of SEQ ID NO:150; HCDR3 comprises an amino acid sequence of SEQ ID NO:151; LCDR1 comprises an amino acid sequence of SEQ ID NO:152; LCDR2 comprises an amino acid sequence of YAS; and LCDR3 comprises an amino acid sequence of SEQ ID NO:154.

In some embodiments, wherein (i) in the first antigen binding region that binds to MSLN, HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76; and (ii) in the second antigen binding region that binds to CD3ε, HCDR1 comprises an amino acid sequence of SEQ ID NO: 155; HCDR2 comprises an amino acid sequence of SEQ ID NO:156; HCDR3 comprises an amino acid sequence of SEQ ID NO:157; LCDR1 comprises an amino acid sequence of SEQ ID NO: 158; LCDR2 comprises an amino acid sequence of YAS; and LCDR3 comprises an amino acid sequence of SEQ ID NO:148.

In some embodiments, wherein (i) in the first antigen binding region that binds to MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69; and (ii) in the second antigen binding region that binds to CD3ε, HCDR1 comprises an amino acid sequence of SEQ ID NO: 143; HCDR2 comprises an amino acid sequence of SEQ ID NO:144; HCDR3 comprises an amino acid sequence of SEQ ID NO:145; LCDR1 comprises an amino acid sequence of SEQ ID NO: 146; LCDR2 comprises an amino acid sequence of SEQ ID NO:147; and LCDR3 comprises an amino acid sequence of SEQ ID NO:148.

In some embodiments, wherein (i) in the first antigen binding region that binds to MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69; and (ii) in the second antigen binding region that binds to CD3ε, HCDR1 comprises an amino acid sequence of SEQ ID NO: 149; HCDR2 comprises an amino acid sequence of SEQ ID NO:150; HCDR3 comprises an amino acid sequence of SEQ ID NO: 151; LCDR1 comprises an amino acid sequence of SEQ ID NO:152; LCDR2 comprises an amino acid sequence of YAS; and LCDR3 comprises an amino acid sequence of SEQ ID NO:154.

In some embodiments, wherein (i) in the first antigen binding region that binds to MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69; and (ii) in the second antigen binding region that binds to CD3ε, HCDR1 comprises an amino acid sequence of SEQ ID NO: 155; HCDR2 comprises an amino acid sequence of SEQ ID NO:156; HCDR3 comprises an amino acid sequence of SEQ ID NO:157; LCDR1 comprises an amino acid sequence of SEQ ID NO:158; LCDR2 comprises an amino acid sequence of YAS; and LCDR3 comprises an amino acid sequence of SEQ ID NO: 148.

In some embodiments, wherein (i) the first binding region comprises a VH domain comprising an amino acid sequence of SEQ ID NO:81, and a VL domain comprising an amino acid sequence of SEQ ID NO:82; and (ii) the second binding region comprises a VH domain comprising an amino acid sequence of SEQ ID NO:159, and a VL domain comprising an amino acid sequence of SEQ ID NO: 160.

In some embodiments, the first antigen binding region comprises a Fab, and the second antigen binding region comprises a scFv. In some embodiments, the scFv comprises an amino acid sequence of SEQ ID NO:161 or SEQ ID NO:162.

In some embodiments, the binding agent further comprises an immunoglobulin (Ig) constant region of or a fragment of the Ig constant region. In some embodiments, the fragment of the Ig constant region comprises a Fc region.

In some embodiments, the binding agent comprises: (i) a first polypeptide comprising the second antigen binding region as a scFv, a CH2 domain and a CH3 domain; (ii) a second polypeptide comprising the VH domain of the first antigen binding region, a CH2 domain and a CH3 domain; and (iii) a third polypeptide comprising the VL domain of the first antigen binding region, wherein the VH domain and the VL domain of the first antigen binding region form a Fab, and the first polypeptide and the second polypeptide forms a Fc region.

In some embodiments, the first polypeptide comprising an amino acid sequence of SEQ ID NO: 163, a second polypeptide comprising an amino acid sequence of SEQ ID NO:164, and a third polypeptide comprising an amino acid sequence of SEQ ID NO: 165.

In some embodiments, the binding agent is a bispecific antibody. In some embodiments, the binding agent is a monoclonal antibody. In some embodiments, the binding agent is a humanized antibody.

In one aspect, provided herein is a binding agent comprises (i) a first polypeptide comprising a scFv that binds CD3ε, a CH2 domain and a CH3 domain; (ii) a second polypeptide comprising a VH domain that binds MSLN, a CH2 domain and a CH3 domain; and (iii) a third polypeptide comprising a VL domain that binds MSLN, wherein the scFv that binds CD3ε comprises a VH domain comprising a HCDR1, HCDR2, and HCDR3 as set forth in SEQ ID NO:159, and a VL domain comprising a LCDR1, LCDR2, and LCDR3 as set forth in SEQ ID NO:160; and wherein the VH domain that binds MSLN comprises a HCDR1, HCDR2, and HCDR3 as set forth in SEQ ID NO:81, and the VL domain that binds MSLN comprises a LCDR1, LCDR2, and LCDR3 as set forth in SEQ ID NO:82.

In some embodiments, wherein (i) the scFv that binds CD3ε comprises HCDR1 comprising an amino acid sequence of SEQ ID NO:143; HCDR2 comprising an amino acid sequence of SEQ ID NO: 144; HCDR3 comprising an amino acid sequence of SEQ ID NO: 145; LCDR1 comprising an amino acid sequence of SEQ ID NO: 146; LCDR2 comprising an amino acid sequence of SEQ ID NO: 147; and LCDR3 comprising an amino acid sequence of SEQ ID NO:148; and (ii) in the VH domain that binds MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:143; HCDR2 comprising an amino acid sequence of SEQ ID NO: 144; HCDR3 comprising an amino acid sequence of SEQ ID NO:145; LCDR1 comprising an amino acid sequence of SEQ ID NO:146; LCDR2 comprising an amino acid sequence of SEQ ID NO: 147; and LCDR3 comprising an amino acid sequence of SEQ ID NO:148; and (ii) in the VH domain that binds MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:143; HCDR2 comprising an amino acid sequence of SEQ ID NO: 144; HCDR3 comprising an amino acid sequence of SEQ ID NO:145; LCDR1 comprising an amino acid sequence of SEQ ID NO:146; LCDR2 comprising an amino acid sequence of SEQ ID NO: 147; and LCDR3 comprising an amino acid sequence of SEQ ID NO:148; and (ii) in the VH domain that binds MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:143; HCDR2 comprising an amino acid sequence of SEQ ID NO: 144; HCDR3 comprising an amino acid sequence of SEQ ID NO: 145; LCDR1 comprising an amino acid sequence of SEQ ID NO:146; LCDR2 comprising an amino acid sequence of SEQ ID NO: 147; and LCDR3 comprising an amino acid sequence of SEQ ID NO:148; and (ii) in the VH domain that binds MSLN, HCDR1 comprises an amino acid sequence of SEQ ID NO: 72; HCDR2 comprises an amino acid sequence of SEQ ID NO:54; HCDR3 comprises an amino acid sequence of SEQ ID NO:73; and in the VL domain that binds MSLN, LCDR1 comprises an amino acid sequence of SEQ ID NO:74; LCDR2 comprises an amino acid sequence of SEQ ID NO:75; and LCDR3 comprises an amino acid sequence of SEQ ID NO:76.

In some embodiments, wherein (i) the scFv that binds CD3ε comprises HCDR1 comprising an amino acid sequence of SEQ ID NO: 143; HCDR2 comprising an amino acid sequence of SEQ ID NO: 144; HCDR3 comprising an amino acid sequence of SEQ ID NO:145; LCDR1 comprising an amino acid sequence of SEQ ID NO:146; LCDR2 comprising an amino acid sequence of SEQ ID NO: 147; and LCDR3 comprising an amino acid sequence of SEQ ID NO:148; and (ii) in the VH domain that binds MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; and in the VL domain that binds MSLN, LCDR1 comprises an amino acid sequence of SEQ ID NO:79; LCDR2 comprises an amino acid sequence of WAS; and LCDR3 comprises an amino acid sequence of SEQ ID NO:69.

In some embodiments, wherein (i) the scFv that binds CD3ε comprises HCDR1 comprising an amino acid sequence of SEQ ID NO: 149; HCDR2 comprising an amino acid sequence of SEQ ID NO: 150; HCDR3 comprising an amino acid sequence of SEQ ID NO:151; LCDR1 comprising an amino acid sequence of SEQ ID NO:152; LCDR2 comprising an amino acid sequence of YAS; and LCDR3 comprising an amino acid sequence of SEQ ID NO: 154; and (ii) in the VH domain that binds MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:149; HCDR2 comprising an amino acid sequence of SEQ ID NO:150; HCDR3 comprising an amino acid sequence of SEQ ID NO: 151; LCDR1 comprising an amino acid sequence of SEQ ID NO: 152; LCDR2 comprising an amino acid sequence of YAS; and LCDR3 comprising an amino acid sequence of SEQ ID NO: 154; and (ii) in the VH domain that binds MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO: 149; HCDR2 comprising an amino acid sequence of SEQ ID NO: 150; HCDR3 comprising an amino acid sequence of SEQ ID NO:151; LCDR1 comprising an amino acid sequence of SEQ ID NO:152; LCDR2 comprising an amino acid sequence of YAS; and LCDR3 comprising an amino acid sequence of SEQ ID NO:154; and (ii) in the VH domain that binds MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:149; HCDR2 comprising an amino acid sequence of SEQ ID NO:150; HCDR3 comprising an amino acid sequence of SEQ ID NO:151; LCDR1 comprising an amino acid sequence of SEQ ID NO:152; LCDR2 comprising an amino acid sequence of YAS; and LCDR3 comprising an amino acid sequence of SEQ ID NO: 154; and (ii) in the VH domain that binds MSLN, HCDR1 comprises an amino acid sequence of SEQ ID NO:72; HCDR2 comprises an amino acid sequence of SEQ ID NO:54; HCDR3 comprises an amino acid sequence of SEQ ID NO:73; and in the VL domain that binds MSLN, LCDR1 comprises an amino acid sequence of SEQ ID NO:74; LCDR2 comprises an amino acid sequence of SEQ ID NO:75; and LCDR3 comprises an amino acid sequence of SEQ ID NO:76.

In some embodiments, wherein (i) the scFv that binds CD3ε comprises HCDR1 comprising an amino acid sequence of SEQ ID NO:149; HCDR2 comprising an amino acid sequence of SEQ ID NO: 150; HCDR3 comprising an amino acid sequence of SEQ ID NO:151; LCDR1 comprising an amino acid sequence of SEQ ID NO:152; LCDR2 comprising an amino acid sequence of YAS; and LCDR3 comprising an amino acid sequence of SEQ ID NO: 154; and (ii) in the VH domain that binds MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; and in the VL domain that binds MSLN, LCDR1 comprises an amino acid sequence of SEQ ID NO:79; LCDR2 comprises an amino acid sequence of WAS; and LCDR3 comprises an amino acid sequence of SEQ ID NO:69.

In some embodiments, wherein (i) the scFv that binds CD3ε comprises HCDR1 comprising an amino acid sequence of SEQ ID NO: 155; HCDR2 comprising an amino acid sequence of SEQ ID NO: 156; HCDR3 comprising an amino acid sequence of SEQ ID NO:157; LCDR1 comprising an amino acid sequence of SEQ ID NO:158; LCDR2 comprising an amino acid sequence of YAS; and LCDR3 comprising an amino acid sequence of SEQ ID NO: 148; and (ii) in the VH domain that binds MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:155; HCDR2 comprising an amino acid sequence of SEQ ID NO: 156; HCDR3 comprising an amino acid sequence of SEQ ID NO:157; LCDR1 comprising an amino acid sequence of SEQ ID NO:158; LCDR2 comprising an amino acid sequence of YAS; and LCDR3 comprising an amino acid sequence of SEQ ID NO: 148; and (ii) in the VH domain that binds MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:155; HCDR2 comprising an amino acid sequence of SEQ ID NO: 156; HCDR3 comprising an amino acid sequence of SEQ ID NO:157; LCDR1 comprising an amino acid sequence of SEQ ID NO:158; LCDR2 comprising an amino acid sequence of YAS; and LCDR3 comprising an amino acid sequence of SEQ ID NO: 148; and (ii) in the VH domain that binds MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69.

In some embodiments, wherein (i) the scFv that binds CD3ε comprises HCDR1 comprising an amino acid sequence of SEQ ID NO:155; HCDR2 comprising an amino acid sequence of SEQ ID NO: 156; HCDR3 comprising an amino acid sequence of SEQ ID NO:157; LCDR1 comprising an amino acid sequence of SEQ ID NO:158; LCDR2 comprising an amino acid sequence of YAS; and LCDR3 comprising an amino acid sequence of SEQ ID NO:148; and (ii) in the VH domain that binds MSLN, HCDR1 comprises an amino acid sequence of SEQ ID NO:72; HCDR2 comprises an amino acid sequence of SEQ ID NO:54; HCDR3 comprises an amino acid sequence of SEQ ID NO:73; and in the VL domain that binds MSLN, LCDR1 comprises an amino acid sequence of SEQ ID NO:74; LCDR2 comprises an amino acid sequence of SEQ ID NO: 75; and LCDR3 comprises an amino acid sequence of SEQ ID NO:76.

In some embodiments, wherein (i) the scFv that binds CD3ε comprises HCDR1 comprising an amino acid sequence of SEQ ID NO:155; HCDR2 comprising an amino acid sequence of SEQ ID NO: 156; HCDR3 comprising an amino acid sequence of SEQ ID NO: 157; LCDR1 comprising an amino acid sequence of SEQ ID NO:158; LCDR2 comprising an amino acid sequence of YAS; and LCDR3 comprising an amino acid sequence of SEQ ID NO: 148; and (ii) in the VH domain that binds MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; and in the VL domain that binds MSLN, LCDR1 comprises an amino acid sequence of SEQ ID NO:79; LCDR2 comprises an amino acid sequence of WAS; and LCDR3 comprises an amino acid sequence of SEQ ID NO:69.

In some embodiments, the scFv that binds CD3ε comprises a VH domain comprising an amino acid sequence of SEQ ID NO:159, and a VL domain comprising an amino acid sequence of SEQ ID NO: 160; the VH domain that binds MSLN comprises an amino acid sequence of SEQ ID NO:81, and the VL domain that binds MSLN comprises an amino acid sequence of SEQ ID NO:82. In some embodiments, the scFv comprises an amino acid sequence of SEQ ID NO: 161 or SEQ ID NO:162.

In one aspect, provided herein is a composition comprising any of binding agent described herein, and a pharmaceutically acceptable carrier.

In one aspect, provided herein is a polynucleotide comprising nucleotide sequences encoding a VH, a VL, or both a VH and a VL of any of the binding agent described herein.

In one aspect, provided herein a polynucleotide comprising nucleotide sequences encoding the first polypeptide, the second polypeptide, and/or the third polypeptide of a binding agent described herein. In some embodiments, the polynucleotide is operably linked to a promoter.

In one aspect, provided herein is a vector comprising any of the polynucleotide described herein.

In one aspect, provided herein is a cell comprising any of the polynucleotide described herein. In one aspect, provided herein is a cell comprising any of the vector described herein. In one aspect, provided herein is an isolated cell producing any of the binding agent described herein.

In one aspect, provided herein is a kit comprising any of the binding agent described herein.

In one aspect, provided herein is a method of making a binding agent which binds to a membrane-restricted epitope of MSLN, comprising culturing the cell descried herein to express the binding agent. In one aspect, provided herein is a method of making a binding agent which binds to a membrane-restricted epitope of MSLN, comprising expressing a polynucleotide described herein.

In one aspect, provided herein is a method of directing a T cell to a target cell expressing MSLN, comprising contacting the T cell with an effective amount of a MSLN×CD3 binding agent described herein or a composition comprising the MSLN×CD3 binding agent, wherein the antigen binding region that binds to CD3εbinds the T cell and the antigen binding region that binds to MSLN binds to the target cell.

In some embodiments, the T cell induces apoptosis in the target cell. In some embodiments, the apoptosis is induced via T-cell dependent cytotoxicity (TDCC) of the directed T cell.

In some embodiments, the method induces differential cytokine releases by the directed T cell; optionally wherein the cytokine is IL-1β, IL-2, IL-4, IL-6, IL-8, IL-12, IL-13, TNF-α, IFN-γ, or any combination thereof.

In some embodiments, the target cell expresses MSLN at a level higher than a reference expression level of MSLN; optionally wherein the reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects.

In some embodiments, the target cell is a cancer or tumor cell. In some embodiments, the cancer or tumor is selected from mesothelioma, serous ovarian carcinoma, and pancreatic cancer. In some embodiments, the cancer is mesothelioma, serous ovarian carcinoma, and pancreatic cancer.

In some embodiments, the target cell comprises a single nucleotide polymorphism (SNP) in the MSLN gene that result in Met593Val substitution in the encoded MSLN protein, and wherein the target cell is not homozygous for the SNP.

In one aspect, provided herein is a method of activating a population of T cells, comprising contacting the population of T cells with an effective amount of a MSLN×CD3 binding agent described herein or a composition comprising the MSLN×CD3 binding agent, and wherein said binding agent activates the population of T cells upon binding to CD3ε.

In some embodiments, activation of the population of T cells is measured by an increase in expression of T cell activation marker(s) CD25, CD69, or a combination thereof. In some embodiments, the percentage of CD25+ T cells, the percentage of CD69+ T cells, and/or the percentage of CD25+/CD69+ T cells in the population of T cells is increased. In some embodiments, activation of the population of T cells is measured by an increase in secretion of a cytokine by the population of T cells; optionally wherein the cytokine is IL-1β, IL-2, IL-4, IL-6, IL-8, IL-12, IL-13, TNF-α, IFN-γ, or any combination thereof.

In one aspect, provided herein is a method of killing or inhibiting the proliferation of a cancer or tumor cell, comprising contacting the cancer or tumor cell with an effective amount of a MSLN×CD3 binding agent described herein or a composition comprising the MSLN×CD3 binding agent. In some embodiments, the cancer or tumor is selected from mesothelioma, serous ovarian carcinoma, and pancreatic cancer. In some embodiments, the cancer is mesothelioma, serous ovarian carcinoma, and pancreatic cancer. In some embodiments, the cancer or tumor cell expresses MSLN at a level higher than a reference expression level of MSLN; optionally wherein the reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects.

In one aspect, provided herein is a method of treating a cancer or tumor in a subject in need thereof, comprising administering an effective amount of a MSLN×CD3 binding agent described herein or a composition comprising the MSLN×CD3 binding agent to the subject. In some embodiments, the cancer or tumor is selected from mesothelioma, serous ovarian carcinoma, and pancreatic cancer. In some embodiments, the cancer is mesothelioma, serous ovarian carcinoma, and pancreatic cancer. In some embodiments, the administering is intravenous administration. In some embodiments, the administering is subcutaneous administration. In some embodiments, the subject is not homozygous for an SNP in the MLSN gene that results in Met593 Val substitution in the encoded MSLN.

In one aspect, provided herein is a method of diagnosing and treating a subject having a MSLN-expressing cancer or tumor, comprising (a) detecting presence or absence of a SNP in the MSLN gene in the subject that results in Met593 Val substitution in the encoded MSLN protein; (b) diagnosing the subject as likely responsive to the treatment of a binding agent comprising a first antigen binding region that binds to MSLN and a second antigen binding region that binds to CD3ε if the subject is not homozygous for the SNP; and (c) administering or provide for administration of an effective amount of a MSLN×CD3 binding agent described herein or a composition comprising the MSLN×CD3 binding agent to the subject if the subject is diagnosed as likely responsive in step (b).

In some embodiments, the cancer or tumor is selected from mesothelioma, serous ovarian carcinoma, and pancreatic cancer.

### 6. BRIEF DESCRIPTION OF THE DRAWINGS

FIG. 1 shows cleavage of full-length membrane-bound MSLN in the membrane-restricted stub region and shed MSLN released from the cell membrane.

FIGS. 2A-D shows quantification of shed MSLN in healthy and cancer serum (FIG. 2A), serosal fluids (FIG. 2B), full-length MSLN in patient-derived xenografts (FIG. 2C), full-length MSLN in NSCLC adenocarcinoma tumors (FIG. 2D).

FIG. 3 shows a graphic depiction of the antigens used to raise antibodies against the C-terminus of human MSLN. MSNW20 is shown in SEQ ID NO:245; MSNW23 is shown in SEQ ID NO:246.

FIGS. 4A-B shows a H838 selectivity assay using MSNB124 (FIG. 4A) and MSNB110 (FIG. 4B).

FIG. 5 shows staining of smooth muscle and cardiac muscle using MSNB71.

FIG. 6 shows the uncorrected total intensity in the cell area as a function of antibody concentration in C2C12 cells.

FIG. 7 shows the uncorrected total intensity in the cell area as a function of antibody concentration in KELLY cells.

FIG. 8 shows the uncorrected total intensity in the cell area as a function of antibody concentration in OCVAR8 cells.

FIG. 9 shows bispecific antibodies binding to the MSLN expressing tumor cell lines.

FIG. 10 shows T-cell cytotoxicity assessment of MSNB457×CD3 bispecific antibodies.

FIG. 11 shows OVCAR-8 xenograft in vivo assessment of MNC3B55 and MNC3B130.

FIG. 12 shows T-cell cytotoxicity assessment of MSNB457×CD3 bispecific antibodies.

FIG. 13 shows a Bipod cytotoxicity comparison.

FIG. 14 shows Bipod and 2+1 format glyphs.

FIG. 15 shows Bipod and 2+1 T-cell binding

FIG. 16 shows Bipod and 2+1 cytotoxicity comparison.

FIG. 17 shows an illustration of the bispecific antibody MNC3B304.

FIG. 18 shows the amino acid sequences of the heavy and light chains of the MNC3B304 antibody.

FIG. 19 shows a time course 37° C. binding of the MNC3B304 antibody to -OVCAR-8 MSLN^{HI+}, -OVCAR-8 K1/C8 MSLN^{MED+}, and SK-OV-3SK-OV-3 MSLN^{LO+} cells.

FIG. 20 shows a competition experiment of binding of membrane-restricted antibody MSNB457.003 or non-membrane-restricted antibody MSNB90.003 in OVCAR-8 cells in the presence or absence of MSLN. Membrane-restricted mAb MSNB457.003 and non-membrane-restricted mAb MSNB90.003 binding was tested in -OVCAR-8 cells in the presence or absence of 1 μM shed MSLN (MSNW11.002), FL-MSLN (MSNW10.002), or 12 aa stub region peptide (LVLDLSMQEALS (SEQ ID NO: 203)). MSNB457.003 is the parental antibody of MNC3B304.

FIG. 21 shows the 12-amino acid C-terminal stub-region sequence alignment of human MSLN compared to SNP-containing MSLN. The SNP rs1135210 sequence variant is aligned with the wild-type sequence.

FIG. 22 shows a FACS-based binding in OVCAR 8 SNP^{A|A}, CAPAN 2 SNP^{A|G}, and BxPC3 SNP^{G|G} cells using membrane-restricted MSNB457.003 and non-membrane-restricted MSLN MSNB90.003 binding.

FIG. 23 shows Incucyte-based cytotoxicity in -OVCAR-8 SNP^{A|A}, CAPAN 2 SNP^{A|G}, and BxPC3 and NCI-H322 SNP^{G|G} cells. Incucyte cytotoxicity was determined at 72 hours, E:T 1:1, using Annexin V or Caspase 3/7 as a measure of apoptosis-induced cell death by membrane-restricted MNC3B130 (mrMSLN-MSNB457×W245LH alternative heterodimerization scaffold) or MNC3B304 (mrMSLN-MSNB457×W245LH, KiH), and non-membrane-restricted MNC3B70 (nmrMSLN-MSNB9×W245LH alternative heterodimerization scaffold), or MNC3B232 (nmrMSLN-MSNB9×W245LH, KiH). MSNB9 is the parental Ab for the MSLN Fab used in BsAb MNC3B232 and is a high-affinity MSLN IgG1 mAb (K_{D}=0.05 nM). MNC3B58 and MNC3B53 are the respective alternative heterodimerization scaffold and KiH W245LH×Null controls.

FIG. 24 shows T-cell-mediated cytotoxicity in 17-cell-line panel with different receptor densities: MNC3B304 versus reference bispecific antibody binding to membrane-proximal mesothelin. Cytotoxicity was measured by Incucyte. Caspase 3/7 signal over background was used as measure of cancer cell death. Two donors, 17 cell lines, each cell line and E:T ratio was run in duplicate or triplicate. Killing EC₅₀ values were determined at 48 and 72 hours.

FIG. 25A-B shows in vitro cytotoxicity in models with respectively, low, medium, and high MSLN expression. Incucyte using Caspase 3/7 as a measure of cancer cell death. E:T 1:1. Graphs represent killing at 72 hours. (FIG. 25A) Dose-response curves of MNC3B304 and MNC3B232. Tested models with different MSLN expression levels: SKOV3SK-OV-3 MSLN^{LO+} (low), OVCAR-8 K1/C8 MSLN^{MED+} (medium), and OVCAR-8 MSLN^{HI+} (high). (FIG. 25B) Overview of killing EC₅₀ values. MNC3B232 represents a control bispecific binding to membrane-proximal mesothelin (K_{D})=0.05 nM).

FIG. 26 shows an immunohistochemistry (IHC) using MNC3B30 (membrane restricted) and 5B2 (non-membrane restricted) antibodies in CDX models with respectively, low, medium, and high MSLN expression. Tested models with different MSLN expression levels: SK-OV-3 MSLN (LO) (low), OVCAR-8 K1/C8 MSLN (MED) (medium), and OVCAR-8 MSLN (HI) (high).

FIG. 27 shows in vitro cytotoxicity with membrane-restricted MNC3B304 and non-membrane-restricted MNC3B232 in the presence or absence of shed MSLN using models with medium and high MSLN expression.

FIG. 28A-C shows the effect of MNC3B304 on MSLN^{LO+}, MSLN^{MED+}, and MSLN^{HI+} xenografts in T-cell humanized mice (Studies ONC2021-083, P764S2, P764B). T-cell-humanized NSG mice bearing established SC (FIG. 28A) SK-OV-3−, (FIG. 28B) OVCAR-8 K1/C8, or (FIG. 28C) OVCAR-8− tumors were IP dosed with MNC3B304 at the indicated doses. MNC3B232 was dosed at 0.1 mg/kg. Control mice received PBS. Group tumor volumes are graphed as mean±SEM. Tumor cells were implanted on Day 0. (FIG. 28A) T-cells were implanted on Day 22, followed by treatment on Day 23, 27, 30, 34, 38, 41, 44, 48, (FIG. 28B) T-cells were implanted on Day 34, followed by treatment on Day 35, 39, 42, 46, 49, 53, or (FIG. 28C) T-cells were implanted on Day 24, followed by treatment on Day 25, 28, 32, 35, 39, 42 (dosing indicated by bar beneath the X-axis). * Denotes significant difference of MNC3B304-treated groups on (FIG. 28A) Day 52 versus control group (p<0.05, n=10/group), (FIG. 28B) Day 57 versus control group (p<0.0001, n=10/group), or (FIG. 28C) on Day 45 versus control group (p<0.0001, n=10/group).

FIG. 29 shows determination of the minimal efficacious dose of MNC3B304 on established OVCAR-8 K1/C8 MSLN^{MED+} tumors in T-cell humanized mice. T-cell-humanized NSG mice bearing established SC OVCAR-8 K1/C8 tumors were IP dosed with MNC3B304 at 0.5, 0.1, and 0.05 mg/kg. Control mice received PBS. Group tumor volumes are graphed as mean±SEM. Tumor cells were implanted on Day 0, T-cells implanted on Day 27, followed by treatment on Day 28, 32, 35, 39, 42, 46, 49, 53 (dosing indicated by bar beneath the X-axis). * Denotes significant difference of MNC3B304-treated groups on Day 56 versus control group (p<0.0001, n=10/group).

### 7. DETAILED DESCRIPTION

Methothelin (MSLN) is a lineage-restricted cell surface protein that originates from the MSLN gene. Despite identification of MSLN in 1992 with the mAb K1 that was generated by the immunization of mice with human ovarian carcinoma (OVCAR-3) cells, its biological function remains largely unknown (Chang et al., 1992, “Isolation and characterization of a monoclonal antibody, K1, reactive with ovarian cancers and normal mesothelium,” Int. J. Cancer). MSLN knockout mice do not show a detectable phenotype (Bera et al., 2000, “Mesothelin is not required for normal mouse development or reproduction,” Cell Biol.). It has been suggested that MSLN plays a role in tumor adhesion and metastasis, based on evidence that MSLN can bind to the ovarian tumor-associated antigen MUC16/CA125. MUC16/CA125 binding to MSLN may contribute to metastasis of ovarian cancer to the peritoneum by initiating cell attachment to the mesothelial cell lining (Gubbels et al., 2006, “Mesothelin-MUC16 binding is a high affinity, N-glycan dependent interaction that facilitates peritoneal metastasis of ovarian tumors,” Mol Cancer).

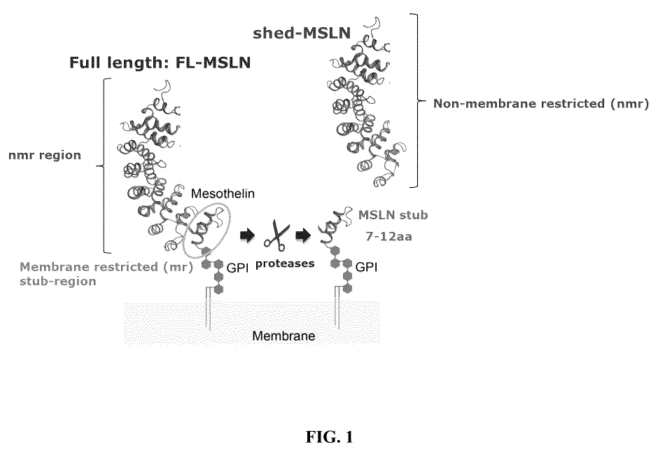

MSLN has been shown to be shed from the membrane in ovarian carcinoma patients due to the sheddase TACE, which is a member of the matrix metalloproteinase (MMP) disintegrin and metalloprotease family. FIG. 1 shows that TACE cleaves MSLN at 7 to 12 amino acids distal from the GPI anchor, releasing shed MSLN into the circulation. Further, other proteases including disintegrin and metalloproteinase (ADAM)10, ADAM17, b-secretase (BACE)2, BACE1, and MMP15 have also been shown to cleave MSLN close to the cell membrane and more than one sheddase can catalyze MSLN release in the same cell.

Without being bound by any theory, it is contemplated that shed MSLN fragments can compete with cell membrane-bound MSLN for binding with an anti-MSLN antibody, thereby impairing biological and therapeutic effects of the antibody. Provided herein are anti-MSLN antibodies specifically targeting the membrane-restricted non-shed region (“stub”) of MSLN thus avoiding the soluble “sink.” This novel approach to an extensively studied target led to antibody-mediated T-cell redirection to cancer types (e.g., mesothelioma, ovarian, and pancreatic cancers) with important unmet medical needs.

7.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

7.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

Unless otherwise defined herein, technical and scientific terms used in the present description have the meanings that are commonly understood by those of ordinary skill in the art. Whenever appropriate, terms used in the singular will also include the plural and vice versa. In the event that any description of a term set forth conflicts with any document incorporated herein by reference, the description of the term set forth below shall control.

The term “binding agent” as used herein refers to a molecule that binds a specific antigen or target (e.g., MSLN and/or CD3). A binding agent may comprise a protein, peptide, nucleic acid, carbohydrate, lipid, or small molecular weight compound. In some embodiments, a binding agent comprises a full-length antibody. In some embodiments, a binding agent is an antigen binding fragment of an antibody. In some embodiments, a binding agent comprises an alternative protein scaffold or artificial scaffold (e.g., a non-immunoglobulin backbone). In some embodiments, a binding agent is a fusion protein comprising an antigen-binding site. In some embodiments, a binding agent is a bispecific molecule comprising at least two antigen-binding sites. In some embodiments, a binding agent is a multispecific molecule comprising at least three antigen-binding sites.

The terms “mesothelin” and “MSLN” “MSLN protein” and “MSLN polypeptide” encompass a polypeptide (“polypeptide” and “protein” are used interchangeably herein), including any native polypeptide, from any vertebrate source, including mammals such as primates (e.g., humans and cynomolgus monkeys (cynomolgus)), dogs, and rodents (e.g., mice and rats), unless otherwise indicated. In certain embodiments, the terms include “related MSLN polypeptides,” including SNP variants thereof. The term “MSLN” also encompasses “full-length,” unprocessed MSLN as well as any form that results from processing. Without being bound by any theory, it is contemplated that MSLN is actively shed from cells due to the cleavage of full-length membrane-associated protein close to the cell membrane, e.g., by the sheddase TACE (which is a member of the matrix metalloproteinase (MMP) disintegrin and metalloprotease family), or cleavage by other proteases including disintegrin and metalloproteinase (ADAM)10, ADAM17, b-secretase (BACE)2, BACE1, and MMP15 at location(s) close to the cell membrane, and more than one sheddase can catalyze MSLN release in the same cell. Accordingly, the term “shed MSLN” as used herein refers to the fragment or fragments of the MSLN protein that are shed and released from the cell membrane, and the terms “membrane-restricted portion” or “membrane-restricted region” of MSLN refer to the fragment of the MSLN protein that remains associated with the cell membrane after shedding.

Three MSLN variants have been described co-existing within the same cell. The National Center for Biotechnology Information (NCBI) database refers to 3 reported transcripts for MSLN/MPF. Variant 1 (GenBank accession NM_005823) encodes the main MSLN/MPF isoform. Variant 2 (NM_013404) encodes a rare MSLN isoform containing an 8-amino-acid insertion following amino acid 410. Variant 3 (AF180951) is a partial complementary DNA (cDNA) encoding MSLN with an alternatively spliced C-terminus. Alternative splicing in Variant 3 leads to a frame shift and an altered C-terminus lacking the GPI anchor site. This isoform, therefore, is not expressed at the cell membrane but directly secreted from the cell as a soluble protein (Muminova et al., 2004, “Characterization of human mesothelin transcripts in ovarian and pancreatic cancer,” BMC Cancer). Additionally, MSLN has also been described to contain single nucleotide polymorphisms (SNPs). For example, rs1135210 (GRCh38-chromosome 16:768559) is a SNP that codes for an adenosine to guanine change resulting in replacement of methionine to valine in the C-terminal MSLN region (MSLN:p.Met593 Val). An exemplary amino acid sequence of the human MSLN protein is provided below, where the 587-LVLDLSMQEALS-598 (SEQ ID NO:203) membrane-restricted stub region is underlined and the Met593 residue is shown in boldface:

(SEQ ID NO: 200)
MALPTARPLLGSCGTPALGSLLFLLFSLGWVQPSRTLAGETGQEA
APLDGVLANPPNISSLSPRQLLGFPCAEVSGLSTERVRELAVALA
QKNVKLSTEQLRCLAHRLSEPPEDLDALPLDLLLFLNPDAFSGPQ
ACTRFFSRITKANVDLLPRGAPERQRLLPAALACWGVRGSLLSEA
DVRALGGLACDLPGRFVAESAEVLLPRLVSCPGPLDQDQQEAARA
ALQGGGPPYGPPSTWSVSTMDALRGLLPVLGQPIIRSIPQGIVAA
WRQRSSRDPSWRQPERTILRPRFRREVEKTACPSGKKAREIDESL
IFYKKWELEACVDAALLATQMDRVNAIPFTYEQLDVLKHKLDELY
PQGYPESVIQHLGYLFLKMSPEDIRKWNVTSLETLKALLEVNKGH
EMSPQVATLIDRFVKGRGQLDKDTLDTLTAFYPGYLCSLSPEELS
SVPPSSIWAVRPQDLDTCDPRQLDVLYPKARLAFQNMNGSEYFVK
IQSFLGGAPTEDLKALSQQNVSMDLATFMKLRTDAVLPLTVAEVQ
KLLGPHVEGLKAEERHRPVRDWILRQRQDDLDTLGLGLQGGIPNG
YLVLDLSMQEALSGTPCLLGPGPVLTVLALLLASTLA

The term “Cluster of Differentiation 3 E” or “CD3E” refers to a known protein which is also called “T-cell surface glycoprotein CD3 epsilon chain,” or “T3E.” CD3ε, together with CD3-gamma, -delta and -zeta, and the T-cell receptor alpha/beta and gamma/delta heterodimers, forms the T-cell receptor-CD3 complex. This complex plays an important role in coupling antigen recognition to several intracellular signal-transduction pathways. The CD3 complex mediates signal transduction, resulting in T cell activation and proliferation. CD3 is required for the immune response. The amino acid sequence of a full length CD3ε is shown in SEQ ID NO: 201. The amino acid sequence of the extracellular domain (ECD) of CD3ε is shown in SEQ ID NO: 202. Throughout the specification, “CD3ε-specific” or “specifically binds CD3ε” or “anti-CD3ε antibody” refers to antibodies that bind specifically to the CD3ε polypeptide (SEQ ID NO: 201), including antibodies that bind specifically to the CD3ε extracellular domain (ECD) (SEQ ID NO: 202);

Human CD3 epsilon:
(SEQ ID NO: 201)
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRORGONKERP
PPVPNPDYEPIRKGQRDLYSGLNQRRI;
Human CD3 epsilon extracellular domain:
(SEQ ID NO: 202)
DGNEEMGGITQTPYKVSISGTTVILTCPQYPGSEILWOHNDKNIG
GDEDDKNIGSDEDHLSLKEFSELEQSGYYVCYPRGSKPEDANFYL
YLRARVCENCMEMD.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D})) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full-length monoclonal antibodies), antibody compositions with polyepitopic or monoepitopic specificity, recombinantly produced antibodies, single domain (e.g., VHH) antibodies, monospecific antibodies, multispecific antibodies (including bispecific antibodies), synthetic antibodies, chimeric antibodies, humanized antibodies, or human versions of antibodies having full-length heavy and/or light chains. Antibodies also include antibody fragments (and/or polypeptides that comprise antibody fragments) that retain binding characteristics of their parental antibodies. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable domain antibody, single variable domain, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) variable domains. For example, antibodies also include tetrameric antibodies comprising two heavy chain and two light chain molecules, an antibody light chain monomer, and an antibody heavy chain monomer. Thus, for example, the V region domain may be dimeric and contain VHH-VHH, VH-VH, VH-VL, or VL-VL dimers that bind NKG2A. If desired, the VH and VL may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated, for example, into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). In some embodiments, antibodies comprising a VH and/or VL contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions. A “functional fragment,” “binding fragment,” or “antigen binding fragment” of a therapeutic antibody will exhibit at least one if not some or all of the biological functions attributed to the intact antibody, the function comprising at least binding to the target antigen (e.g., a MSLN binding fragment or fragment that binds to MSLN).

A typical 4-chain antibody unit is a heterotetrametric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a ß sheet configuration, connected by three hypervariable regions, which form loops connecting, and in some cases form part of, the ß sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat,” and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (a), delta (8), epsilon (¿), gamma (Y), and mu (u), based on the amino acid sequence of the heavy chain constant region. The distinct heavy chains differ in size: α, δ, and γ contain approximately 450 amino acids, while u and & contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in the table below.

Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26-- | H30--H35B | H27--H38 |
| | (Kabat | | H32 . . . 34 | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 | H93--H101 | H105-H117 |
| | | | or H95-- | | |
| | | | H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “antigen binding domain” or “antigen binding region” refers to a binding agent or a portion of a binding agent as described herein (such as a protein or an antibody or fragment thereof) that binds an antigen. In some embodiments, an antigen binding region can comprise one or more fragments or portions of an intact antibody as described herein. The term “antigen binding domain” or “antigen binding region” can be an antibody fragment as described above.

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

The term “(scFv)₂” or “tandem scFv” or “bis-scFv” refers to a fusion protein comprising two light chain variable region (VL) and two heavy chain variable region (VH), wherein the two VL and the two VH are contiguously linked via polypeptide linkers, and capable of being expressed as a single chain polypeptide. The two VL and two VH are fused by peptide linkers to form a bivalent molecule VL_{A}-linker-VH_{A}-linker-VL_{B}-linker-VH_{B} to form two binding sites, capable of binding two different antigens or epitopes concurrently.

The term “multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

The term “bispecific” refers to a molecule (such as a protein or an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The terms “bispecific anti-MSLN/anti-CD3 antibody,” “MSLN/CD3 antibody,” “MSLN×CD3 antibody,” “anti-MSLN/anti-CD3 protein,” and the like refer to an antibody that binds MSLN and CD3, i.e., comprising at least one binding domain specifically binding MSNL and at least one binding domain specifically binding CD3. The domains specifically binding MSLN and CD3 are typically V_{H}/V_{L} pairs. The bispecific anti-MSLN×CD3 antibody may be monovalent in terms of its binding to either MSLN or CD3.

The term “monoclonal antibody” as used herein refers to a substantially homogenous antibody population involved in the highly specific recognition and binding of a single antigenic determinant or epitope. The term “monoclonal antibody” encompasses intact and full-length antibodies as well as antibody fragments (e.g., Fab, Fab′, F(ab′)₂, Fv), single chain antibodies, scFv, fusion proteins comprising an antigen-binding antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of publicly available bioinformatic software tools. X-ray crystallography may be used to characterize an epitope on a target protein by analyzing the amino acid residue interactions of an antigen/antibody complex.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR amino acid residues are replaced by residues from corresponding CDRs from a non-human antibody (e.g., mouse, rat, rabbit, or non-human primate), wherein the non-human antibody has the desired specificity, affinity, and/or activity. In some embodiments, one or more framework region amino acid residues of the human heavy chain or light chain variable regions are replaced by corresponding residues from the non-human antibody. Furthermore, humanized antibodies can comprise amino acid residues that are not found in the human antibody or in the non-human antibody. In some embodiments, these modifications are made to further refine and/or optimize antibody characteristics. In some embodiments, the humanized antibody comprises at least a portion of a human immunoglobulin constant region (e.g., CH1, CH2, CH3, Fc, and/or hinge region).

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60, at least about 60-80 nucleotides or amino acids in length, or any integral value there between. In some embodiments, identity exists over a longer region than 60-80 nucleotides or amino acids, such as at least about 80-100 nucleotides or amino acids, and in some embodiments the sequences are substantially identical over the full length of the sequences being compared, for example, (i) the coding region of a nucleotide sequence or (ii) an amino acid sequence.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like.

The term “excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle. In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990). In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution. In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc. Compositions, including pharmaceutical compounds, may contain a prophylactically or therapeutically effective amount of a NKG2A binding agent (e.g., an antibody), for example, in isolated or purified form, together with a suitable amount of excipient so as to provide the form for proper administration to the subject (e.g., patient). The formulation should suit the mode of administration.

The term “pharmaceutical composition” or “pharmaceutical formulation” as used herein refers to a preparation that is in such form as to permit the biological activity of the binding agent to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to therapeutic measures that aim to cure, slow down, lessen symptoms of, and/or halt progression of a pathologic condition or disorder. Thus, those in need of treatment include those already with the disorder.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, dendritic cells, etc.

As used herein, reference to “about” or “approximately” a value or parameter includes (and describes) embodiments that are directed to that value or parameter. For example, a description referring to “about X” includes description of “X.”

As used in the present disclosure and claims, the singular forms “a,” “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

7.3 MSLN Binding Agents

In one aspect, provided herein is a binding agent comprising an antigen binding region that binds MSLN (e.g., human MSLN). In some embodiments, the present binding agent comprises at least one portion that is a polypeptide. In some embodiments, the present binding agent comprises at least one portion that is not a polypeptide. In some embodiments, the present binding agents are MSLN binding proteins.

In some embodiments, the present disclosure provides binding agents (e.g., antibodies or proteins) that bind MSLN. In some embodiments, the MSLN binding agent binds a MSLN protein or a fragment thereof of a mammalian origin. In some embodiments, the MSLN binding agent binds a human MSLN protein or a fragment thereof. In some embodiments, the MSLN binding agent binds a MSLN protein or a fragment thereof originated from a non-human mammalian species. In some embodiments, the non-human mammalian species is a rodent (e.g., mice and rats). In some embodiments, the non-human mammalian species is a dog. In some embodiments, the non-human mammalian species is a cynomolgus monkeys (cynomolgus).

In some embodiments, the binding agent comprises an antigen binding region that binds to MSLN. In some embodiments, the MSLN binding region in the present binding protein is an antibody or a binding domain derived from an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In some embodiments, the antibody comprises an IgG heavy chain. In some embodiments, the antibody comprises an IgG1 heavy chain. In some embodiments, the antibody comprises an IgG2 heavy chain. In some embodiments, the antibody comprises an IgG4 heavy chain. In some embodiments, the antibody comprises a kappa light chain. In some embodiments, the antibody comprises a kappa light chain constant region. In some embodiments, the antibody comprises a lambda light chain. In some embodiments, the antibody comprises a lambda light chain constant region. In some embodiments, the antibody is an antibody fragment comprising an antigen-binding site. In some embodiments, the antibody is an scFv. In some embodiments, the antibody is a disulfide-linked scFv. In some embodiments, the antibody is a disulfide-linked sc(Fv)₂. In some embodiments, the antibody is a Fab, Fab′, or a F(ab)₂ antibody. In some embodiments, the antibody is a diabody. In some embodiments, the antibody is a nanobody. In some embodiments, the antibody is a monospecific antibody. In some embodiments, the antibody is a bispecific antibody. In some embodiments, the antibody is a trispecific antibody. In some embodiments, the antibody is a multispecific antibody. In some embodiments, the antibody is a monovalent antibody. In some embodiments, the antibody is a multivalent antibody. In some embodiments, the antibody is a bivalent antibody. In some embodiments, the antibody is a trivalent antibody. In some embodiments, the antibody is a tetravalent antibody.

In some embodiments, the MSLN binding region provided herein binds to MSLN (e.g., human MSLN) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In some embodiments, the MSLN binding region provided herein binds to MSLN with a dissociation constant of ≤0.1 nM. In some embodiments, the MSLN binding region provided herein binds to MSLN with a dissociation constant of ≤0.2 nM. In some embodiments, the MSLN binding region provided herein binds to MSLN with a dissociation constant of ≤0.3 nM. In some embodiments, the MSLN binding region provided herein binds to MSLN with a dissociation constant of ≤0.8 nM. In some embodiments, the MSLN binding region provided herein binds to MSLN with a dissociation constant of ≤3 nM. In some embodiments, the MSLN binding region provided herein binds to MSLN with a dissociation constant of ≤9 nM. A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®-2000 or a Biacore®-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®-3000, or the Biacore®-8000 system.

In one aspect, provided herein is a binding agent that binds MSLN. In one embodiment, the binding agent comprises a MSLN binding region. In some embodiments, the MSLN binding region is any one of those in Tables 1-12.

In some embodiments, the MSLN binding region provided herein comprises one or more CDR sequences of the VH or VL having the amino acid sequence set forth in any one of SEQ ID NO:22, SEQ ID NO:23, SEQ ID NO:45, SEQ ID NO:46, SEQ ID NO:62, SEQ ID NO:63, SEQ ID NO:81, SEQ ID NO:82, SEQ ID NO:94, SEQ ID NO:95, SEQ ID NO: 106, SEQ ID NO: 107, SEQ ID NO: 120, SEQ ID NO: 121, SEQ ID NO: 132, SEQ ID NO: 133, SEQ ID NO:138, SEQ ID NO:139, SEQ ID NO: 140, and SEQ ID NO: 142.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:22. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:23. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:22, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:23. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 1, 7, 9, 11, and 17; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:2, 8, 10, 12, and 18, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:3, 13, and 19; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO:4, 14 and SEQ ID NO:20; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:5, 15, and 21; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO:6 and 16. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 1.

In some embodiments, the MSLN binding region provided herein comprises an HCDR1 comprising an amino acid sequence of any of SEQ ID NOs: 1, 7, 9, 11, and 17; (ii) an HCDR2 comprising an amino acid sequence of any of SEQ ID NOs:2, 8, 10, 12, and 18, (iii) an HCDR3 comprising an amino acid sequence of SEQ ID NOs:3, 13, and 19; (iv) a LCDR1 comprising an amino acid sequence of SEQ ID NOs:4, 14, and 20; (v) a LCDR2 comprising an amino acid sequence of SEQ ID NOs:5, 15, and 21; and/or (vi) a LCDR3 comprising an amino acid sequence of SEQ ID NOs:6 and 16.

In some specific embodiments, in the MSLN binding region provided herein, the HCDR1 comprises the amino acid sequence of SEQ ID NO: 1, the HCDR2 comprises the amino acid sequence of SEQ ID NO:2, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the MSLN binding region provided herein, the HCDR1 comprises the amino acid sequence of SEQ ID NO:7, the HCDR2 comprises the amino acid sequence of SEQ ID NO:8, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the MSLN binding region provided herein, the HCDR1 comprises the amino acid sequence of SEQ ID NO:9, the HCDR2 comprises the amino acid sequence of SEQ ID NO: 10, the HCDR3 comprises the amino acid sequence of SEQ ID NO:3, the LCDR1 comprises the amino acid sequence of SEQ ID NO:4, the LCDR2 comprises the amino acid sequence of SEQ ID NO:5, and the LCDR3 comprises the amino acid sequence of SEQ ID NO:6.

In some specific embodiments, in the MSLN binding region provided herein, the HCDR1 comprises the amino acid sequence of SEQ ID NO:11, the HCDR2 comprises the amino acid sequence of SEQ ID NO:12, the HCDR3 comprises the amino acid sequence of SEQ ID NO:13, the LCDR1 comprises the amino acid sequence of SEQ ID NO: 14, the LCDR2 comprises the amino acid sequence of SEQ ID NO: 15, and the LCDR3 comprises the amino acid sequence of SEQ ID NO: 16.

In some specific embodiments, in the MSLN binding region provided herein, the HCDR1 comprises the amino acid sequence of SEQ ID NO: 17, the HCDR2 comprises the amino acid sequence of SEQ ID NO: 18, the HCDR3 comprises the amino acid sequence of SEQ ID NO:19, the LCDR1 comprises the amino acid sequence of SEQ ID NO:20, the LCDR2 comprises the amino acid sequence of LVS, and the LCDR3 comprises the amino acid sequence of SEQ ID NO:6.

In one embodiment, provided herein is a binding region that binds MSLN, comprising a VH comprising a HCDR1, a HCDR2, and a HCDR3 having an amino acid sequence of SEQ ID NOs:1, 2, and 3, respectively. In another embodiment, provided herein is a binding region that binds MSLN, comprising a VL comprising a LCDR1, a LCDR2, and a LCDR3 having an amino acid sequence of SEQ ID NOs:4, 5, and 6, respectively. In another embodiment, provided herein is a binding region that binds MSLN, comprising: (i) a VH comprising a HCDR1, a HCDR2, and a HCDR3 having an amino acid sequence of SEQ ID NOs: 1, 2, and 3, respectively, and (ii) a VL comprising a LCDR1, a LCDR2, and a LCDR3 having an amino acid sequence of SEQ ID NOs:4, 5, and 6, respectively.

In one embodiment, provided herein is a binding region that binds MSLN, comprising a VH comprising a HCDR1, a HCDR2, and a HCDR3 having an amino acid sequence of SEQ ID NOs:7, 8, and 3, respectively. In another embodiment, provided herein is a binding region that binds MSLN, comprising a VL comprising a LCDR1, a LCDR2, and a LCDR3 having an amino acid sequence of SEQ ID NOs:4, 5, and 6, respectively. In another embodiment, provided herein is a binding region that binds MSLN, comprising: (i) a VH comprising a HCDR1, a HCDR2, and a HCDR3 having an amino acid sequence of SEQ ID NOs:7, 8, and 3, respectively, and (ii) a VL comprising a LCDR1, a LCDR2, and a LCDR3 having an amino acid sequence of SEQ ID NOs:4, 5, and 6, respectively.

In one embodiment, provided herein is a binding region that binds MSLN, comprising a VH comprising a HCDR1, a HCDR2, and a HCDR3 having an amino acid sequence of SEQ ID NOs:9, 10, and 3, respectively. In another embodiment, provided herein is a binding region that binds MSLN, comprising a VL comprising a LCDR1, a LCDR2, and a LCDR3 having an amino acid sequence of SEQ ID NOs:4, 5, and 6, respectively. In another embodiment, provided herein is a binding region that binds MSLN, comprising: (i) a VH comprising a HCDR1, a HCDR2, and a HCDR3 having an amino acid sequence of SEQ ID NOs:9, 10, and 3, respectively, and (ii) a VL comprising a LCDR1, a LCDR2, and a LCDR3 having an amino acid sequence of SEQ ID NOs:4, 5, and 6, respectively.

In one embodiment, provided herein is a binding region that binds MSLN, comprising a VH comprising a HCDR1, a HCDR2, and a HCDR3 having an amino acid sequence of SEQ ID NOs:11, 12, and 13, respectively. In another embodiment, provided herein is a binding region that binds MSLN, comprising a VL comprising a LCDR1, a LCDR2, and a LCDR3 having an amino acid sequence of SEQ ID NOs: 14, 15, and 16, respectively. In another embodiment, provided herein is a binding region that binds MSLN, comprising: (i) a VH comprising a HCDR1, a HCDR2, and a HCDR3 having an amino acid sequence of SEQ ID NOs: 11, 12, and 13, respectively, and (ii) a VL comprising a LCDR1, a LCDR2, and a LCDR3 having an amino acid sequence of SEQ ID NOs: 14, 15, and 16, respectively.

In one embodiment, provided herein is a binding region that binds MSLN, comprising a VH comprising a HCDR1, a HCDR2, and a HCDR3 having an amino acid sequence of SEQ ID NOs: 17, 18, and 19, respectively. In another embodiment, provided herein is a binding region that binds MSLN, comprising a VL comprising a LCDR1, a LCDR2, and a LCDR3 having an amino acid sequence of SEQ ID NOs:20, 21, and 6, respectively. In another embodiment, provided herein is a binding region that binds MSLN, comprising: (i) a VH comprising a HCDR1, a HCDR2, and a HCDR3 having an amino acid sequence of SEQ ID NOs:17, 18, and 19, respectively, and (ii) a VL comprising a LCDR1, a LCDR2, and a LCDR3 having an amino acid sequence of SEQ ID NOs:20, 21, and 6, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:45. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:46. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:45, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:46. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 24, 30, 32, 34, and 40; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:25, 252, 33, 35, and 41, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:26, 36, and 42; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:27, 37, and 43; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:28, 38, and 44; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:29 and 39. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 2.

In some embodiments, the MSLN binding region provided herein comprises an HCDR1 comprising an amino acid sequence of any of SEQ ID NOs:24, 30, 32, 34, and 40; (ii) an HCDR2 comprising an amino acid sequence of any of SEQ ID NOs:25, 252, 33, 35, and 41, (iii) an HCDR3 comprising an amino acid sequence of SEQ ID NOs:26, 36, and 42; (iv) a LCDR1 comprising an amino acid sequence of SEQ ID NOs:27, 37, and 43; (v) a LCDR2 comprising an amino acid sequence of SEQ ID NOs:28, 38, and 44; and/or (vi) a LCDR3 comprising an amino acid sequence of SEQ ID NOs:29 and 39.

In some specific embodiments, in the MSLN binding region provided herein, the HCDR1 comprises the amino acid sequence of SEQ ID NO:24, the HCDR2 comprises the amino acid sequence of SEQ ID NO:25, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29.

In some specific embodiments, in the MSLN binding region provided herein, the HCDR1 comprises the amino acid sequence of SEQ ID NO:30, the HCDR2 comprises the amino acid sequence of SEQ ID NO:252, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29.

In some specific embodiments, in the MSLN binding region provided herein, the HCDR1 comprises the amino acid sequence of SEQ ID NO:32, the HCDR2 comprises the amino acid sequence of SEQ ID NO:33, the HCDR3 comprises the amino acid sequence of SEQ ID NO:26, the LCDR1 comprises the amino acid sequence of SEQ ID NO:27, the LCDR2 comprises the amino acid sequence of SEQ ID NO:28, and the LCDR3 comprises the amino acid sequence of SEQ ID NO:29.

In some specific embodiments, in the MSLN binding region provided herein, the HCDR1 comprises the amino acid sequence of SEQ ID NO:34, the HCDR2 comprises the amino acid sequence of SEQ ID NO:35, the HCDR3 comprises the amino acid sequence of SEQ ID NO:36, the LCDR1 comprises the amino acid sequence of SEQ ID NO:37, the LCDR2 comprises the amino acid sequence of SEQ ID NO:38, and the LCDR3 comprises the amino acid sequence of SEQ ID NO:39.

In some specific embodiments, in the MSLN binding region provided herein, the HCDR1 comprises the amino acid sequence of SEQ ID NO:40, the HCDR2 comprises the amino acid sequence of SEQ ID NO:41, the HCDR3 comprises the amino acid sequence of SEQ ID NO:42, the LCDR1 comprises the amino acid sequence of SEQ ID NO:43, the LCDR2 comprises the amino acid sequence of LGS, and the LCDR3 comprises the amino acid sequence of SEQ ID NO:29.

In one embodiment, provided herein is a binding region that binds MSLN, comprising a VH comprising a HCDR1, a HCDR2, and a HCDR3 having an amino acid sequence of SEQ ID NOs:24, 25, and 26, respectively. In another embodiment, provided herein is a binding region that binds MSLN, comprising a VL comprising a LCDR1, a LCDR2, and a LCDR3 having an amino acid sequence of SEQ ID NOs:27, 28, and 29, respectively. In another embodiment, provided herein is a binding region that binds MSLN, comprising: (i) a VH comprising a HCDR1, a HCDR2, and a HCDR3 having an amino acid sequence of SEQ ID NOs:24, 25, and 26, respectively, and (ii) a VL comprising a LCDR1, a LCDR2, and a LCDR3 having an amino acid sequence of SEQ ID NOs:27, 28, and 29, respectively.

In one embodiment, provided herein is a binding region that binds MSLN, comprising a VH comprising a HCDR1, a HCDR2, and a HCDR3 having an amino acid sequence of SEQ ID NOs:30, 252, and 26, respectively. In another embodiment, provided herein is a binding region that binds MSLN, comprising a VL comprising a LCDR1, a LCDR2, and a LCDR3 having an amino acid sequence of SEQ ID SEQ ID NOs:27, 28, and 29, respectively. In another embodiment, provided herein is a binding region that binds MSLN, comprising: (i) a VH comprising a HCDR1, a HCDR2, and a HCDR3 having an amino acid sequence of SEQ ID NOs:30, 252, and 26, respectively, and (ii) a VL comprising a LCDR1, a LCDR2, and a LCDR3 having an amino acid sequence of SEQ ID SEQ ID NOs:27, 28, and 29, respectively.

In one embodiment, provided herein is a binding region that binds MSLN, comprising a VH comprising a HCDR1, a HCDR2, and a HCDR3 having an amino acid sequence of SEQ ID NOs:32, 33, and 26, respectively. In another embodiment, provided herein is a binding region that binds MSLN, comprising a VL comprising a LCDR1, a LCDR2, and a LCDR3 having an amino acid sequence of SEQ ID NOs:27, 28, and 29, respectively. In another embodiment, provided herein is a binding region that binds MSLN, comprising: (i) a VH comprising a HCDR1, a HCDR2, and a HCDR3 having an amino acid sequence of SEQ ID NOs:32, 33, and 26, respectively, and (ii) a VL comprising a LCDR1, a LCDR2, and a LCDR3 having an amino acid sequence of SEQ ID NOs:27, 28, and 29, respectively.

In one embodiment, provided herein is a binding region that binds MSLN, comprising a VH comprising a HCDR1, a HCDR2, and a HCDR3 having an amino acid sequence of SEQ ID NOs:34, 35, and 36, respectively. In another embodiment, provided herein is a binding region that binds MSLN, comprising a VL comprising a LCDR1, a LCDR2, and a LCDR3 having an amino acid sequence of SEQ ID NOs:37, 38, and 39, respectively. In another embodiment, provided herein is a binding region that binds MSLN, comprising: (i) a VH comprising a HCDR1, a HCDR2, and a HCDR3 having an amino acid sequence of SEQ ID NOs:34, 35, and 36, respectively, and (ii) a VL comprising a LCDR1, a LCDR2, and a LCDR3 having an amino acid sequence of SEQ ID NOs:37, 38, and 39, respectively.

In one embodiment, provided herein is a binding region that binds MSLN, comprising a VH comprising a HCDR1, a HCDR2, and a HCDR3 having an amino acid sequence of SEQ ID NOs:40, 41, and 42, respectively. In another embodiment, provided herein is a binding region that binds MSLN, comprising a VL comprising a LCDR1, a LCDR2, and a LCDR3 having an amino acid sequence of SEQ ID NOs:43, 44, and 29, respectively. In another embodiment, provided herein is a binding region that binds MSLN, comprising: (i) a VH comprising a HCDR1, a HCDR2, and a HCDR3 having an amino acid sequence of SEQ ID NOs:40, 41, and 42, respectively, and (ii) a VL comprising a LCDR1, a LCDR2, and a LCDR3 having an amino acid sequence of SEQ ID NOs:43, 44, and 29, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:62. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:63. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:62, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:63. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:47, 251, 51, 53, and 58; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:48, 31, 52, 54, and 59, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:3, 55, and 60; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:49, 56, and 61; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:28, 38, and 44; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:50 and 57. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 3.

In some embodiments, the MSLN binding region provided herein comprises an HCDR1 comprising an amino acid sequence of any of SEQ ID NOs:47, 251, 51, 53, and 58; (ii) an HCDR2 comprising an amino acid sequence of any of SEQ ID NOs:48, 31, 52, 54, and 59, (iii) an HCDR3 comprising an amino acid sequence of SEQ ID NOs:3, 55, and 60; (iv) a LCDR1 comprising an amino acid sequence of SEQ ID NOs:49, 56, and 61; (v) a LCDR2 comprising an amino acid sequence of SEQ ID NOs:28, 38, and 44; and/or (vi) a LCDR3 comprising an amino acid sequence of SEQ ID NOs:50 and 57.

In some specific embodiments, in the MSLN binding region provided herein, the HCDR1 comprises the amino acid sequence of SEQ ID NO:47, the HCDR2 comprises the amino acid sequence of SEQ ID NO:48, the HCDR3 comprises the amino acid sequence of SEQ ID NO:3, the LCDR1 comprises the amino acid sequence of SEQ ID NO:49, the LCDR2 comprises the amino acid sequence of SEQ ID NO:28, and the LCDR3 comprises the amino acid sequence of SEQ ID NO:50.

In some specific embodiments, in the MSLN binding region provided herein, the HCDR1 comprises the amino acid sequence of SEQ ID NO:251, the HCDR2 comprises the amino acid sequence of SEQ ID NO:31, the HCDR3 comprises the amino acid sequence of SEQ ID NO:3, the LCDR1 comprises the amino acid sequence of SEQ ID NO:49, the LCDR2 comprises the amino acid sequence of SEQ ID NO:28, and the LCDR3 comprises the amino acid sequence of SEQ ID NO:50.

In some specific embodiments, in the MSLN binding region provided herein, the HCDR1 comprises the amino acid sequence of SEQ ID NO:51, the HCDR2 comprises the amino acid sequence of SEQ ID NO:52, the HCDR3 comprises the amino acid sequence of SEQ ID NO:3, the LCDR1 comprises the amino acid sequence of SEQ ID NO:49, the LCDR2 comprises the amino acid sequence of SEQ ID NO:28, and the LCDR3 comprises the amino acid sequence of SEQ ID NO:50.

In some specific embodiments, in the MSLN binding region provided herein, the HCDR1 comprises the amino acid sequence of SEQ ID NO:53, the HCDR2 comprises the amino acid sequence of SEQ ID NO:54, the HCDR3 comprises the amino acid sequence of SEQ ID NO:55, the LCDR1 comprises the amino acid sequence of SEQ ID NO:56, the LCDR2 comprises the amino acid sequence of SEQ ID NO:38, and the LCDR3 comprises the amino acid sequence of SEQ ID NO:57.

In some specific embodiments, in the MSLN binding region provided herein, the HCDR1 comprises the amino acid sequence of SEQ ID NO:58, the HCDR2 comprises the amino acid sequence of SEQ ID NO:59, the HCDR3 comprises the amino acid sequence of SEQ ID NO:60, the LCDR1 comprises the amino acid sequence of SEQ ID NO:61, the LCDR2 comprises the amino acid sequence of LGS, and the LCDR3 comprises the amino acid sequence of SEQ ID NO:50.

In one embodiment, provided herein is a binding region that binds MSLN, comprising a VH comprising a HCDR1, a HCDR2, and a HCDR3 having an amino acid sequence of SEQ ID NOs:47, 48, and 3, respectively. In another embodiment, provided herein is a binding region that binds MSLN, comprising a VL comprising a LCDR1, a LCDR2, and a LCDR3 having an amino acid sequence of SEQ ID NOs:49, 28, and 50, respectively. In another embodiment, provided herein is a binding region that binds MSLN, comprising: (i) a VH comprising a HCDR1, a HCDR2, and a HCDR3 having an amino acid sequence of SEQ ID NOs:47, 48, and 3, respectively, and (ii) a VL comprising a LCDR1, a LCDR2, and a LCDR3 having an amino acid sequence of SEQ ID NOs:49, 28, and 50, respectively.

In one embodiment, provided herein is a binding region that binds MSLN, comprising a VH comprising a HCDR1, a HCDR2, and a HCDR3 having an amino acid sequence of SEQ ID NOs:251, 31, and 3, respectively. In another embodiment, provided herein is a binding region that binds MSLN, comprising a VL comprising a LCDR1, a LCDR2, and a LCDR3 having an amino acid sequence of SEQ ID NOs:49, 28, and 50, respectively. In another embodiment, provided herein is a binding region that binds MSLN, comprising: (i) a VH comprising a HCDR1, a HCDR2, and a HCDR3 having an amino acid sequence of SEQ ID NOs:251, 31, and 3, respectively, and (ii) a VL comprising a LCDR1, a LCDR2, and a LCDR3 having an amino acid sequence of SEQ ID NOs:49, 28, and 50, respectively.

In one embodiment, provided herein is a binding region that binds MSLN, comprising a VH comprising a HCDR1, a HCDR2, and a HCDR3 having an amino acid sequence of SEQ ID NOs:51, 52, and 3, respectively. In another embodiment, provided herein is a binding region that binds MSLN, comprising a VL comprising a LCDR1, a LCDR2, and a LCDR3 having an amino acid sequence of SEQ ID NOs:49, 28, and 50, respectively. In another embodiment, provided herein is a binding region that binds MSLN, comprising: (i) a VH comprising a HCDR1, a HCDR2, and a HCDR3 having an amino acid sequence of SEQ ID NOs:51, 52, and 3, respectively, and (ii) a VL comprising a LCDR1, a LCDR2, and a LCDR3 having an amino acid sequence of SEQ ID NOs:49, 28, and 50, respectively.

In one embodiment, provided herein is a binding region that binds MSLN, comprising a VH comprising a HCDR1, a HCDR2, and a HCDR3 having an amino acid sequence of SEQ ID NOs:53, 54, and 55, respectively. In another embodiment, provided herein is a binding region that binds MSLN, comprising a VL comprising a LCDR1, a LCDR2, and a LCDR3 having an amino acid sequence of SEQ ID NOs:56, 38, and 57, respectively. In another embodiment, provided herein is a binding region that binds MSLN, comprising: (i) a VH comprising a HCDR1, a HCDR2, and a HCDR3 having an amino acid sequence of SEQ ID NOs:53, 54, and 55, respectively, and (ii) a VL comprising a LCDR1, a LCDR2, and a LCDR3 having an amino acid sequence of SEQ ID NOs:56, 38, and 57, respectively.

In one embodiment, provided herein is a binding region that binds MSLN, comprising a VH comprising a HCDR1, a HCDR2, and a HCDR3 having an amino acid sequence of SEQ ID NOs:58, 59, and 60, respectively. In another embodiment, provided herein is a binding region that binds MSLN, comprising a VL comprising a LCDR1, a LCDR2, and a LCDR3 having an amino acid sequence of SEQ ID NOs:61, 44, and 50, respectively. In another embodiment, provided herein is a binding region that binds MSLN, comprising: (i) a VH comprising a HCDR1, a HCDR2, and a HCDR3 having an amino acid sequence of SEQ ID NOs:58, 59, and 60, respectively, and (ii) a VL comprising a LCDR1, a LCDR2, and a LCDR3 having an amino acid sequence of SEQ ID NOs:61, 44, and 50, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:81. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:82. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:81, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:82. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:64, 70, 71, 72, and 77; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:65, 31, 52, 54, and 59, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:66, 73, and 78; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:67, 74, and 79; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:68, 75, and 80; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:69 and 76. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 4.

In some embodiments, the MSLN binding region provided herein comprises an HCDR1 comprising an amino acid sequence of any of SEQ ID NOs:64, 70, 71, 72, and 77; (ii) an HCDR2 comprising an amino acid sequence of any of SEQ ID NOs:65, 31, 52, 54, and 59, (iii) an HCDR3 comprising an amino acid sequence of SEQ ID NOs:66, 73, and 78; (iv) a LCDR1 comprising an amino acid sequence of SEQ ID NOs:67, 74, and 79; (v) a LCDR2 comprising an amino acid sequence of SEQ ID NOs:68, 75, and 80; and/or (vi) a LCDR3 comprising an amino acid sequence of SEQ ID NOs:69 and 76.

In some specific embodiments, in the MSLN binding region provided herein, the HCDR1 comprises the amino acid sequence of SEQ ID NO:64, the HCDR2 comprises the amino acid sequence of SEQ ID NO:65, the HCDR3 comprises the amino acid sequence of SEQ ID NO:66, the LCDR1 comprises the amino acid sequence of SEQ ID NO:67, the LCDR2 comprises the amino acid sequence of SEQ ID NO:68, and the LCDR3 comprises the amino acid sequence of SEQ ID NO:69.

In some specific embodiments, in the MSLN binding region provided herein, the HCDR1 comprises the amino acid sequence of SEQ ID NO:70, the HCDR2 comprises the amino acid sequence of SEQ ID NO:31, the HCDR3 comprises the amino acid sequence of SEQ ID NO:66, the LCDR1 comprises the amino acid sequence of SEQ ID NO:67, the LCDR2 comprises the amino acid sequence of SEQ ID NO:68, and the LCDR3 comprises the amino acid sequence of SEQ ID NO:69.

In some specific embodiments, in the MSLN binding region provided herein, the HCDR1 comprises the amino acid sequence of SEQ ID NO:71, the HCDR2 comprises the amino acid sequence of SEQ ID NO:52, the HCDR3 comprises the amino acid sequence of SEQ ID NO:66, the LCDR1 comprises the amino acid sequence of SEQ ID NO:67, the LCDR2 comprises the amino acid sequence of SEQ ID NO:68, and the LCDR3 comprises the amino acid sequence of SEQ ID NO:69.

In some specific embodiments, in the MSLN binding region provided herein, the HCDR1 comprises the amino acid sequence of SEQ ID NO: 72, the HCDR2 comprises the amino acid sequence of SEQ ID NO:54, the HCDR3 comprises the amino acid sequence of SEQ ID NO:73, the LCDR1 comprises the amino acid sequence of SEQ ID NO:74, the LCDR2 comprises the amino acid sequence of SEQ ID NO:75, and the LCDR3 comprises the amino acid sequence of SEQ ID NO:76.

In some specific embodiments, in the MSLN binding region provided herein, the HCDR1 comprises the amino acid sequence of SEQ ID NO:77, the HCDR2 comprises the amino acid sequence of SEQ ID NO:59, the HCDR3 comprises the amino acid sequence of SEQ ID NO:78, the LCDR1 comprises the amino acid sequence of SEQ ID NO: 79, the LCDR2 comprises the amino acid sequence of WAS, and the LCDR3 comprises the amino acid sequence of SEQ ID NO:69.

In one embodiment, provided herein is a binding region that binds MSLN, comprising a VH comprising a HCDR1, a HCDR2, and a HCDR3 having an amino acid sequence of SEQ ID NOs:64, 65, and 66, respectively. In another embodiment, provided herein is a binding region that binds MSLN, comprising a VL comprising a LCDR1, a LCDR2, and a LCDR3 having an amino acid sequence of SEQ ID NOs:67, 68, and 69, respectively. In another embodiment, provided herein is a binding region that binds MSLN, comprising: (i) a VH comprising a HCDR1, a HCDR2, and a HCDR3 having an amino acid sequence of SEQ ID NOs:64, 65, and 66, respectively, and (ii) a VL comprising a LCDR1, a LCDR2, and a LCDR3 having an amino acid sequence of SEQ ID NOs:67, 68, and 69, respectively.

In one embodiment, provided herein is a binding region that binds MSLN, comprising a VH comprising a HCDR1, a HCDR2, and a HCDR3 having an amino acid sequence of SEQ ID NOs:70, 31, and 66, respectively. In another embodiment, provided herein is a binding region that binds MSLN, comprising a VL comprising a LCDR1, a LCDR2, and a LCDR3 having an amino acid sequence of SEQ ID NOs:67, 68, and 69, respectively. In another embodiment, provided herein is a binding region that binds MSLN, comprising: (i) a VH comprising a HCDR1, a HCDR2, and a HCDR3 having an amino acid sequence of SEQ ID NOs:70, 31, and 66, respectively, and (ii) a VL comprising a LCDR1, a LCDR2, and a LCDR3 having an amino acid sequence of SEQ ID NOs:67, 68, and 69, respectively.

In one embodiment, provided herein is a binding region that binds MSLN, comprising a VH comprising a HCDR1, a HCDR2, and a HCDR3 having an amino acid sequence of SEQ ID NOs:71, 52, and 66, respectively. In another embodiment, provided herein is a binding region that binds MSLN, comprising a VL comprising a LCDR1, a LCDR2, and a LCDR3 having an amino acid sequence of SEQ ID NOs:67, 68, and 69, respectively. In another embodiment, provided herein is a binding region that binds MSLN, comprising: (i) a VH comprising a HCDR1, a HCDR2, and a HCDR3 having an amino acid sequence of SEQ ID NOs:71, 52, and 66, respectively, and (ii) a VL comprising a LCDR1, a LCDR2, and a LCDR3 having an amino acid sequence of SEQ ID NOs:67, 68, and 69, respectively.

In one embodiment, provided herein is a binding region that binds MSLN, comprising a VH comprising a HCDR1, a HCDR2, and a HCDR3 having an amino acid sequence of SEQ ID NOs:72, 54, and 73, respectively. In another embodiment, provided herein is a binding region that binds MSLN, comprising a VL comprising a LCDR1, a LCDR2, and a LCDR3 having an amino acid sequence of SEQ ID NOs: 74, 75, and 76, respectively. In another embodiment, provided herein is a binding region that binds MSLN, comprising: (i) a VH comprising a HCDR1, a HCDR2, and a HCDR3 having an amino acid sequence of SEQ ID NOs:72, 54, and 73, respectively, and (ii) a VL comprising a LCDR1, a LCDR2, and a LCDR3 having an amino acid sequence of SEQ ID NOs: 74, 75, and 76, respectively.

In one embodiment, provided herein is a binding region that binds MSLN, comprising a VH comprising a HCDR1, a HCDR2, and a HCDR3 having an amino acid sequence of SEQ ID NOs:77, 59, and 78, respectively. In another embodiment, provided herein is a binding region that binds MSLN, comprising a VL comprising a LCDR1, a LCDR2, and a LCDR3 having an amino acid sequence of SEQ ID NOs:79, 80, and 69, respectively. In another embodiment, provided herein is a binding region that binds MSLN, comprising: (i) a VH comprising a HCDR1, a HCDR2, and a HCDR3 having an amino acid sequence of SEQ ID NOs:77, 59, and 78, respectively, and (ii) a VL comprising a LCDR1, a LCDR2, and a LCDR3 having an amino acid sequence of SEQ ID NOs: 79, 80, and 69, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:94. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:95. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:94, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:95. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 30, 40, 253, 83, and 88; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 52, 54, 59, and 84 (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:85, 89, and 92; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:86, 90, and 93; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:28, 38, and 44; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:87 and 91. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 5.

In some embodiments, the MSLN binding region provided herein comprises an HCDR1 comprising an amino acid sequence of any of SEQ ID NOs:30, 40, 253, 83, and 88; (ii) an HCDR2 comprising an amino acid sequence of any of SEQ ID NOs:31, 52, 54, 59, and 84, (iii) an HCDR3 comprising an amino acid sequence of SEQ ID NOs:85, 89, and 92; (iv) a LCDR1 comprising an amino acid sequence of SEQ ID NOs:86, 90, and 93; (v) a LCDR2 comprising an amino acid sequence of SEQ ID NOs:28, 38, and 44; and/or (vi) a LCDR3 comprising an amino acid sequence of SEQ ID NOs:87 and 91.

In some specific embodiments, in the MSLN binding region provided herein, the HCDR1 comprises the amino acid sequence of SEQ ID NO:83, the HCDR2 comprises the amino acid sequence of SEQ ID NO:84, the HCDR3 comprises the amino acid sequence of SEQ ID NO:85, the LCDR1 comprises the amino acid sequence of SEQ ID NO:86, the LCDR2 comprises the amino acid sequence of SEQ ID NO:28 and the LCDR3 comprises the amino acid sequence of SEQ ID NO:87.

In some specific embodiments, in the MSLN binding region provided herein, the HCDR1 comprises the amino acid sequence of SEQ ID NO:30, the HCDR2 comprises the amino acid sequence of SEQ ID NO:31, the HCDR3 comprises the amino acid sequence of SEQ ID NO:85, the LCDR1 comprises the amino acid sequence of SEQ ID NO:86, the LCDR2 comprises the amino acid sequence of SEQ ID NO:28, and the LCDR3 comprises the amino acid sequence of SEQ ID NO:87.

In some specific embodiments, in the MSLN binding region provided herein, the HCDR1 comprises the amino acid sequence of SEQ ID NO:253, the HCDR2 comprises the amino acid sequence of SEQ ID NO:52, the HCDR3 comprises the amino acid sequence of SEQ ID NO:85, the LCDR1 comprises the amino acid sequence of SEQ ID NO:86 the LCDR2 comprises the amino acid sequence of SEQ ID NO:28, and the LCDR3 comprises the amino acid sequence of SEQ ID NO:87.

In some specific embodiments, in the MSLN binding region provided herein, the HCDR1 comprises the amino acid sequence of SEQ ID NO:88, the HCDR2 comprises the amino acid sequence of SEQ ID NO:54, the HCDR3 comprises the amino acid sequence of SEQ ID NO:89, the LCDR1 comprises the amino acid sequence of SEQ ID NO:90, the LCDR2 comprises the amino acid sequence of SEQ ID NO:38, and the LCDR3 comprises the amino acid sequence of SEQ ID NO:91.

In some specific embodiments, in the MSLN binding region provided herein, the HCDR1 comprises the amino acid sequence of SEQ ID NO:40, the HCDR2 comprises the amino acid sequence of SEQ ID NO:59, the HCDR3 comprises the amino acid sequence of SEQ ID NO:92, the LCDR1 comprises the amino acid sequence of SEQ ID NO:93, the LCDR2 comprises the amino acid sequence of LGS, and the LCDR3 comprises the amino acid sequence of SEQ ID NO:87.

In one embodiment, provided herein is a binding region that binds MSLN, comprising a VH comprising a HCDR1, a HCDR2, and a HCDR3 having an amino acid sequence of SEQ ID NOs:83, 84, and 85, respectively. In another embodiment, provided herein is a binding region that binds MSLN, comprising a VL comprising a LCDR1, a LCDR2, and a LCDR3 having an amino acid sequence of SEQ ID NOs:86, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:83, 84, and 85, respectively, and (ii) a VL comprising a LCDR1, a LCDR2, and a LCDR3 having an amino acid sequence of SEQ ID NOs:86, 28, and 87, respectively.

In one embodiment, provided herein is a binding region that binds MSLN, comprising a VH comprising a HCDR1, a HCDR2, and a HCDR3 having an amino acid sequence of SEQ ID NOs:30, 31, and 85, respectively. In another embodiment, provided herein is a binding region that binds MSLN, comprising a VL comprising a LCDR1, a LCDR2, and a LCDR3 having an amino acid sequence of SEQ ID NOs:86, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:30, 31, and 85, respectively, and (ii) a VL comprising a LCDR1, a LCDR2, and a LCDR3 having an amino acid sequence of SEQ ID NOs:86, 28, and 87, respectively.

In one embodiment, provided herein is a binding region that binds MSLN, comprising a VH comprising a HCDR1, a HCDR2, and a HCDR3 having an amino acid sequence of SEQ ID NOs:253, 52, and 85, respectively. In another embodiment, provided herein is a binding region that binds MSLN, comprising a VL comprising a LCDR1, a LCDR2, and a LCDR3 having an amino acid sequence of SEQ ID NOs:86, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:253, 52, and 85, respectively, and (ii) a VL comprising a LCDR1, a LCDR2, and a LCDR3 having an amino acid sequence of SEQ ID NOs:86, 28, and 87, respectively.

In one embodiment, provided herein is a binding region that binds MSLN, comprising a VH comprising a HCDR1, a HCDR2, and a HCDR3 having an amino acid sequence of SEQ ID NOs:88, 54, and 89, respectively. In another embodiment, provided herein is a binding region that binds MSLN, comprising a VL comprising a LCDR1, a LCDR2, and a LCDR3 having an amino acid sequence of SEQ ID NOs:90, 38, and 91, respectively. In another embodiment, provided herein is a binding region that binds MSLN, comprising: (i) a VH comprising a HCDR1, a HCDR2, and a HCDR3 having an amino acid sequence of SEQ ID NOs:88, 54, and 89, respectively, and (ii) a VL comprising a LCDR1, a LCDR2, and a LCDR3 having an amino acid sequence of SEQ ID NOs:90, 38, and 91, respectively.

In one embodiment, provided herein is a binding region that binds MSLN, comprising a VH comprising a HCDR1, a HCDR2, and a HCDR3 having an amino acid sequence of SEQ ID NOs:40, 59, and 92, respectively. In another embodiment, provided herein is a binding region that binds MSLN, comprising a VL comprising a LCDR1, a LCDR2, and a LCDR3 having an amino acid sequence of SEQ ID NOs:93, 44, and 87, respectively. In another embodiment, provided herein is a binding region that binds MSLN, comprising: (i) a VH comprising a HCDR1, a HCDR2, and a HCDR3 having an amino acid sequence of SEQ ID NOs:40, 59, and 92, respectively, and (ii) a VL comprising a LCDR1, a LCDR2, and a LCDR3 having an amino acid sequence of SEQ ID NOs:93, 44, and 87, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:106. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:107. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 106, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:107. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 1, 7, 9, 11, and 17; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 52, 54, 59, and 96, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:97, 100, and 104; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:98, 101, and 105; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:5, 21, and 102; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:99 and 103. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 6.

In some embodiments, the MSLN binding region provided herein comprises an HCDR1 comprising an amino acid sequence of any of SEQ ID NOs:1, 7, 9, 11, and 17; (ii) an HCDR2 comprising an amino acid sequence of any of SEQ ID NOs:31, 52, 54, 59, and 96, (iii) an HCDR3 comprising an amino acid sequence of SEQ ID NOs:97, 100, and 104; (iv) a LCDR1 comprising an amino acid sequence of SEQ ID NOs:98, 101, and 105; (v) a LCDR2 comprising an amino acid sequence of SEQ ID NOs 5, 21, and 102; and/or (vi) a LCDR3 comprising an amino acid sequence of SEQ ID NOs:99 and 103.

In some specific embodiments, in the MSLN binding region provided herein, the HCDR1 comprises the amino acid sequence of SEQ ID NO:1, the HCDR2 comprises the amino acid sequence of SEQ ID NO:96, the HCDR3 comprises the amino acid sequence of SEQ ID NO:97, the LCDR1 comprises the amino acid sequence of SEQ ID NO:98, the LCDR2 comprises the amino acid sequence of SEQ ID NO:5, and the LCDR3 comprises the amino acid sequence of SEQ ID NO:99.

In some specific embodiments, in the MSLN binding region provided herein, the HCDR1 comprises the amino acid sequence of SEQ ID NO:7, the HCDR2 comprises the amino acid sequence of SEQ ID NO:31, the HCDR3 comprises the amino acid sequence of SEQ ID NO:97, the LCDR1 comprises the amino acid sequence of SEQ ID NO:98, the LCDR2 comprises the amino acid sequence of SEQ ID NO:5, and the LCDR3 comprises the amino acid sequence of SEQ ID NO:99.

In some specific embodiments, in the MSLN binding region provided herein, the HCDR1 comprises the amino acid sequence of SEQ ID NO:9, the HCDR2 comprises the amino acid sequence of SEQ ID NO:52, the HCDR3 comprises the amino acid sequence of SEQ ID NO:97, the LCDR1 comprises the amino acid sequence of SEQ ID NO:98, the LCDR2 comprises the amino acid sequence of SEQ ID NO:5, and the LCDR3 comprises the amino acid sequence of SEQ ID NO:99.

In some specific embodiments, in the MSLN binding region provided herein, the HCDR1 comprises the amino acid sequence of SEQ ID NO: 11, the HCDR2 comprises the amino acid sequence of SEQ ID NO:54, the HCDR3 comprises the amino acid sequence of SEQ ID NO:100, the LCDR1 comprises the amino acid sequence of SEQ ID NO: 101, the LCDR2 comprises the amino acid sequence of SEQ ID NO: 102, and the LCDR3 comprises the amino acid sequence of SEQ ID NO:103.

In some specific embodiments, in the MSLN binding region provided herein, the HCDR1 comprises the amino acid sequence of SEQ ID NO:17, the HCDR2 comprises the amino acid sequence of SEQ ID NO:59, the HCDR3 comprises the amino acid sequence of SEQ ID NO: 104, the LCDR1 comprises the amino acid sequence of SEQ ID NO: 105, the LCDR2 comprises the amino acid sequence of LVS, and the LCDR3 comprises the amino acid sequence of SEQ ID NO:99.

In one embodiment, provided herein is a binding region that binds MSLN, comprising a VH comprising a HCDR1, a HCDR2, and a HCDR3 having an amino acid sequence of SEQ ID NOs: 1, 96, and 97, respectively. In another embodiment, provided herein is a binding region that binds MSLN, comprising a VL comprising a LCDR1, a LCDR2, and a LCDR3 having an amino acid sequence of SEQ ID NOs:98, 5, and 99, respectively. In another embodiment, provided herein is a binding region that binds MSLN, comprising: (i) a VH comprising a HCDR1, a HCDR2, and a HCDR3 having an amino acid sequence of SEQ ID NOs: 1, 96, and 97, respectively, and (ii) a VL comprising a LCDR1, a LCDR2, and a LCDR3 having an amino acid sequence of SEQ ID NOs:98, 5, and 99, respectively.

In one embodiment, provided herein is a binding region that binds MSLN, comprising a VH comprising a HCDR1, a HCDR2, and a HCDR3 having an amino acid sequence of SEQ ID NOs:7, 31, and 97, respectively. In another embodiment, provided herein is a binding region that binds MSLN, comprising a VL comprising a LCDR1, a LCDR2, and a LCDR3 having an amino acid sequence of SEQ ID NOs:98, 5, and 99, respectively. In another embodiment, provided herein is a binding region that binds MSLN, comprising: (i) a VH comprising a HCDR1, a HCDR2, and a HCDR3 having an amino acid sequence of SEQ ID NOs:7, 31, and 97, respectively, and (ii) a VL comprising a LCDR1, a LCDR2, and a LCDR3 having an amino acid sequence of SEQ ID NOs:98, 5, and 99, respectively.

In one embodiment, provided herein is a binding region that binds MSLN, comprising a VH comprising a HCDR1, a HCDR2, and a HCDR3 having an amino acid sequence of SEQ ID NOs:9, 52, and 97, respectively. In another embodiment, provided herein is a binding region that binds MSLN, comprising a VL comprising a LCDR1, a LCDR2, and a LCDR3 having an amino acid sequence of SEQ ID NOs:98, 5, and 99, respectively. In another embodiment, provided herein is a binding region that binds MSLN, comprising: (i) a VH comprising a HCDR1, a HCDR2, and a HCDR3 having an amino acid sequence of SEQ ID NOs:9, 52, and 97, respectively, and (ii) a VL comprising a LCDR1, a LCDR2, and a LCDR3 having an amino acid sequence of SEQ ID NOs:98, 5, and 99, respectively.

In one embodiment, provided herein is a binding region that binds MSLN, comprising a VH comprising a HCDR1, a HCDR2, and a HCDR3 having an amino acid sequence of SEQ ID NOs: 11, 54, and 100, respectively. In another embodiment, provided herein is a binding region that binds MSLN, comprising a VL comprising a LCDR1, a LCDR2, and a LCDR3 having an amino acid sequence of SEQ ID NOs:101, 102, and 103, respectively. In another embodiment, provided herein is a binding region that binds MSLN, comprising: (i) a VH comprising a HCDR1, a HCDR2, and a HCDR3 having an amino acid sequence of SEQ ID NOs: 11, 54, and 100, respectively, and (ii) a VL comprising a LCDR1, a LCDR2, and a LCDR3 having an amino acid sequence of SEQ ID NOs: 101, 102, and 103, respectively.

In one embodiment, provided herein is a binding region that binds MSLN, comprising a VH comprising a HCDR1, a HCDR2, and a HCDR3 having an amino acid sequence of SEQ ID NOs: 17, 59, and 104, respectively. In another embodiment, provided herein is a binding region that binds MSLN, comprising a VL comprising a LCDR1, a LCDR2, and a LCDR3 having an amino acid sequence of SEQ ID NOs: 105, 21, and 99, respectively. In another embodiment, provided herein is a binding region that binds MSLN, comprising: (i) a VH comprising a HCDR1, a HCDR2, and a HCDR3 having an amino acid sequence of SEQ ID NOs:17, 59, and 104, respectively, and (ii) a VL comprising a LCDR1, a LCDR2, and a LCDR3 having an amino acid sequence of SEQ ID NOs: 105, 21, and 99, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:120. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO: 121. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 120, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:121. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 108, 112, 113, 114, and 117; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 52, 54, 59, and 109 (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:110, 115, and 118; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 111, 116, and 119; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:28, 38, and 44; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:87 and 91. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 7.

In some embodiments, the MSLN binding region provided herein comprises an HCDR1 comprising an amino acid sequence of any of SEQ ID NOs: 108, 112, 113, 114, and 117; (ii) an HCDR2 comprising an amino acid sequence of any of SEQ ID NOs:31, 52, 54, 59, and 109 (iii) an HCDR3 comprising an amino acid sequence of SEQ ID NOs:110, 115, and 118; (iv) a LCDR1 comprising an amino acid sequence of SEQ ID NOs:111, 116, and 119; (v) a LCDR2 comprising an amino acid sequence of SEQ ID NOs:28, 38, and 44; and/or (vi) a LCDR3 comprising an amino acid sequence of SEQ ID NOs:87 and 91.

In some specific embodiments, in the MSLN binding region provided herein, the HCDR1 comprises the amino acid sequence of SEQ ID NO: 108, the HCDR2 comprises the amino acid sequence of SEQ ID NO: 109, the HCDR3 comprises the amino acid sequence of SEQ ID NO:110, the LCDR1 comprises the amino acid sequence of SEQ ID NO:111, the LCDR2 comprises the amino acid sequence of SEQ ID NO:28, and the LCDR3 comprises the amino acid sequence of SEQ ID NO:87.

In some specific embodiments, in the MSLN binding region provided herein, the HCDR1 comprises the amino acid sequence of SEQ ID NO: 112, the HCDR2 comprises the amino acid sequence of SEQ ID NO:31, the HCDR3 comprises the amino acid sequence of SEQ ID NO:110, the LCDR1 comprises the amino acid sequence of SEQ ID NO: 111, the LCDR2 comprises the amino acid sequence of SEQ ID NO:28, and the LCDR3 comprises the amino acid sequence of SEQ ID NO:87.

In some specific embodiments, in the MSLN binding region provided herein, the HCDR1 comprises the amino acid sequence of SEQ ID NO: 113, the HCDR2 comprises the amino acid sequence of SEQ ID NO:52, the HCDR3 comprises the amino acid sequence of SEQ ID NO:110, the LCDR1 comprises the amino acid sequence of SEQ ID NO: 111, the LCDR2 comprises the amino acid sequence of SEQ ID NO:28, and the LCDR3 comprises the amino acid sequence of SEQ ID NO:38.

In some specific embodiments, in the MSLN binding region provided herein, the HCDR1 comprises the amino acid sequence of SEQ ID NO: 114, the HCDR2 comprises the amino acid sequence of SEQ ID NO:54, the HCDR3 comprises the amino acid sequence of SEQ ID NO:115, the LCDR1 comprises the amino acid sequence of SEQ ID NO:116, the LCDR2 comprises the amino acid sequence of SEQ ID NO:38, and the LCDR3 comprises the amino acid sequence of SEQ ID NO:91.

In some specific embodiments, in the MSLN binding region provided herein, the HCDR1 comprises the amino acid sequence of SEQ ID NO: 117, the HCDR2 comprises the amino acid sequence of SEQ ID NO:59, the HCDR3 comprises the amino acid sequence of SEQ ID NO:118, the LCDR1 comprises the amino acid sequence of SEQ ID NO: 119, the LCDR2 comprises the amino acid sequence of LGS, and the LCDR3 comprises the amino acid sequence of SEQ ID NO:87.

In one embodiment, provided herein is a binding region that binds MSLN, comprising a VH comprising a HCDR1, a HCDR2, and a HCDR3 having an amino acid sequence of SEQ ID NOs:108, 109, and 110, respectively. In another embodiment, provided herein is a binding region that binds MSLN, comprising a VL comprising a LCDR1, a LCDR2, and a LCDR3 having an amino acid sequence of SEQ ID NOs: 111, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:108, 109, and 110, respectively, and (ii) a VL comprising a LCDR1, a LCDR2, and a LCDR3 having an amino acid sequence of SEQ ID NOs:111, 28, and 87, respectively.

In one embodiment, provided herein is a binding region that binds MSLN, comprising a VH comprising a HCDR1, a HCDR2, and a HCDR3 having an amino acid sequence of SEQ ID NOs: 112, 31, and 110, respectively. In another embodiment, provided herein is a binding region that binds MSLN, comprising a VL comprising a LCDR1, a LCDR2, and a LCDR3 having an amino acid sequence of SEQ ID NOs: 111, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:112, 31, and 110, respectively, and (ii) a VL comprising a LCDR1, a LCDR2, and a LCDR3 having an amino acid sequence of SEQ ID NOs:111, 28, and 87, respectively.

In one embodiment, provided herein is a binding region that binds MSLN, comprising a VH comprising a HCDR1, a HCDR2, and a HCDR3 having an amino acid sequence of SEQ ID NOs:113, 52, and 110, respectively. In another embodiment, provided herein is a binding region that binds MSLN, comprising a VL comprising a LCDR1, a LCDR2, and a LCDR3 having an amino acid sequence of SEQ ID NOs:111, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:113, 52, and 110, respectively, and (ii) a VL comprising a LCDR1, a LCDR2, and a LCDR3 having an amino acid sequence of SEQ ID NOs:111, 28, and 87, respectively.

In one embodiment, provided herein is a binding region that binds MSLN, comprising a VH comprising a HCDR1, a HCDR2, and a HCDR3 having an amino acid sequence of SEQ ID NOs: 114, 54, and 115, respectively. In another embodiment, provided herein is a binding region that binds MSLN, comprising a VL comprising a LCDR1, a LCDR2, and a LCDR3 having an amino acid sequence of SEQ ID NOs: 116, 38, and 91, respectively. In another embodiment, provided herein is a binding region that binds MSLN, comprising: (i) a VH comprising a HCDR1, a HCDR2, and a HCDR3 having an amino acid sequence of SEQ ID NOs:114, 54, and 115, respectively, and (ii) a VL comprising a LCDR1, a LCDR2, and a LCDR3 having an amino acid sequence of SEQ ID NOs: 116, 38, and 91, respectively.

In one embodiment, provided herein is a binding region that binds MSLN, comprising a VH comprising a HCDR1, a HCDR2, and a HCDR3 having an amino acid sequence of SEQ ID NOs:117, 59, and 118, respectively. In another embodiment, provided herein is a binding region that binds MSLN, comprising a VL comprising a LCDR1, a LCDR2, and a LCDR3 having an amino acid sequence of SEQ ID NOs: 119, 44, and 87, respectively. In another embodiment, provided herein is a binding region that binds MSLN, comprising: (i) a VH comprising a HCDR1, a HCDR2, and a HCDR3 having an amino acid sequence of SEQ ID NOs: 117, 59, and 118, respectively, and (ii) a VL comprising a LCDR1, a LCDR2, and a LCDR3 having an amino acid sequence of SEQ ID NOs: 119, 44, and 87, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:132. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:133. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 132, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:133. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 122, 125, 126, 127, and 130; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 52, 54, 59, and 84, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:3, 13, and 19; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 123, 128, and 131; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:44, 124, and 129; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:99 and 103. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 8.

In some embodiments, the MSLN binding region provided herein comprises an HCDR1 comprising an amino acid sequence of any of SEQ ID NOs:122, 125, 126, 127 and 130; (ii) an HCDR2 comprising an amino acid sequence of any of SEQ ID NOs:31, 52, 54, 59, and 84, (iii) an HCDR3 comprising an amino acid sequence of SEQ ID NOs:3, 13, and 19; (iv) a LCDR1 comprising an amino acid sequence of SEQ ID NOs: 123, 128, and 131; (v) a LCDR2 comprising an amino acid sequence of SEQ ID NOs:44, 124, and 129; and/or (vi) a LCDR3 comprising an amino acid sequence of SEQ ID NOs:99 and 103.

In some specific embodiments, in the MSLN binding region provided herein, the HCDR1 comprises the amino acid sequence of SEQ ID NO:122, the HCDR2 comprises the amino acid sequence of SEQ ID NO:84, the HCDR3 comprises the amino acid sequence of SEQ ID NO:3, the LCDR1 comprises the amino acid sequence of SEQ ID NO: 123, the LCDR2 comprises the amino acid sequence of SEQ ID NO: 124, and the LCDR3 comprises the amino acid sequence of SEQ ID NO:99.

In some specific embodiments, in the MSLN binding region provided herein, the HCDR1 comprises the amino acid sequence of SEQ ID NO: 125, the HCDR2 comprises the amino acid sequence of SEQ ID NO:31, the HCDR3 comprises the amino acid sequence of SEQ ID NO:3, the LCDR1 comprises the amino acid sequence of SEQ ID NO: 123, the LCDR2 comprises the amino acid sequence of SEQ ID NO: 124, and the LCDR3 comprises the amino acid sequence of SEQ ID NO:99.

In some specific embodiments, in the MSLN binding region provided herein, the HCDR1 comprises the amino acid sequence of SEQ ID NO: 126, the HCDR2 comprises the amino acid sequence of SEQ ID NO:52, the HCDR3 comprises the amino acid sequence of SEQ ID NO:3, the LCDR1 comprises the amino acid sequence of SEQ ID NO: 123, the LCDR2 comprises the amino acid sequence of SEQ ID NO:124, and the LCDR3 comprises the amino acid sequence of SEQ ID NO:99.

In some specific embodiments, in the MSLN binding region provided herein, the HCDR1 comprises the amino acid sequence of SEQ ID NO: 127, the HCDR2 comprises the amino acid sequence of SEQ ID NO:54, the HCDR3 comprises the amino acid sequence of SEQ ID NO:13, the LCDR1 comprises the amino acid sequence of SEQ ID NO: 128, the LCDR2 comprises the amino acid sequence of SEQ ID NO: 129, and the LCDR3 comprises the amino acid sequence of SEQ ID NO: 103.

In some specific embodiments, in the MSLN binding region provided herein, the HCDR1 comprises the amino acid sequence of SEQ ID NO: 130, the HCDR2 comprises the amino acid sequence of SEQ ID NO:59, the HCDR3 comprises the amino acid sequence of SEQ ID NO: 19, the LCDR1 comprises the amino acid sequence of SEQ ID NO:131, the LCDR2 comprises the amino acid sequence of LGS, and the LCDR3 comprises the amino acid sequence of SEQ ID NO:99.

In one embodiment, provided herein is a binding region that binds MSLN, comprising a VH comprising a HCDR1, a HCDR2, and a HCDR3 having an amino acid sequence of SEQ ID NOs:122, 84, and 3, respectively. In another embodiment, provided herein is a binding region that binds MSLN, comprising a VL comprising a LCDR1, a LCDR2, and a LCDR3 having an amino acid sequence of SEQ ID NOs: 123, 124, and 99, respectively. In another embodiment, provided herein is a binding region that binds MSLN, comprising: (i) a VH comprising a HCDR1, a HCDR2, and a HCDR3 having an amino acid sequence of SEQ ID NOs: 122, 84, and 3, respectively, and (ii) a VL comprising a LCDR1, a LCDR2, and a LCDR3 having an amino acid sequence of SEQ ID NOs: 123, 124, and 99, respectively.

In one embodiment, provided herein is a binding region that binds MSLN, comprising a VH comprising a HCDR1, a HCDR2, and a HCDR3 having an amino acid sequence of SEQ ID NOs: 125, 31, and 3, respectively. In another embodiment, provided herein is a binding region that binds MSLN, comprising a VL comprising a LCDR1, a LCDR2, and a LCDR3 having an amino acid sequence of SEQ ID NOs: 123, 124, and 99, respectively. In another embodiment, provided herein is a binding region that binds MSLN, comprising: (i) a VH comprising a HCDR1, a HCDR2, and a HCDR3 having an amino acid sequence of SEQ ID NOs:125, 31, and 3, respectively, and (ii) a VL comprising a LCDR1, a LCDR2, and a LCDR3 having an amino acid sequence of SEQ ID NOs: 123, 124, and 99, respectively.

In one embodiment, provided herein is a binding region that binds MSLN, comprising a VH comprising a HCDR1, a HCDR2, and a HCDR3 having an amino acid sequence of SEQ ID NOs: 126, 52, and 3, respectively. In another embodiment, provided herein is a binding region that binds MSLN, comprising a VL comprising a LCDR1, a LCDR2, and a LCDR3 having an amino acid sequence of SEQ ID NOs: 123, 124, and 99, respectively. In another embodiment, provided herein is a binding region that binds MSLN, comprising: (i) a VH comprising a HCDR1, a HCDR2, and a HCDR3 having an amino acid sequence of SEQ ID NOs: 126, 52, and 3, respectively, and (ii) a VL comprising a LCDR1, a LCDR2, and a LCDR3 having an amino acid sequence of SEQ ID NOs: 123, 124, and 99, respectively.

In one embodiment, provided herein is a binding region that binds MSLN, comprising a VH comprising a HCDR1, a HCDR2, and a HCDR3 having an amino acid sequence of SEQ ID NOs:127, 54, and 13, respectively. In another embodiment, provided herein is a binding region that binds MSLN, comprising a VL comprising a LCDR1, a LCDR2, and a LCDR3 having an amino acid sequence of SEQ ID NOs: 128, 129, and 103, respectively. In another embodiment, provided herein is a binding region that binds MSLN, comprising: (i) a VH comprising a HCDR1, a HCDR2, and a HCDR3 having an amino acid sequence of SEQ ID NOs:127, 54, and 13, respectively, and (ii) a VL comprising a LCDR1, a LCDR2, and a LCDR3 having an amino acid sequence of SEQ ID NOs: 128, 129, and 103, respectively.

In one embodiment, provided herein is a binding region that binds MSLN, comprising a VH comprising a HCDR1, a HCDR2, and a HCDR3 having an amino acid sequence of SEQ ID NOs: 130, 59, and 19, respectively. In another embodiment, provided herein is a binding region that binds MSLN, comprising a VL comprising a LCDR1, a LCDR2, and a LCDR3 having an amino acid sequence of SEQ ID NOs: 131, 44, and 99, respectively. In another embodiment, provided herein is a binding region that binds MSLN, comprising: (i) a VH comprising a HCDR1, a HCDR2, and a HCDR3 having an amino acid sequence of SEQ ID NOs:130, 59, and 19, respectively, and (ii) a VL comprising a LCDR1, a LCDR2, and a LCDR3 having an amino acid sequence of SEQ ID NOs: 131, 44, and 99, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:138. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO: 133. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 138, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:133. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 122, 125, 126, 127, and 130; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 134, 135, 136, and 137, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:3, 13, and 19; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 123, 128, and 131; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:44, 124, and 129; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:99 and 103. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 9.

In some embodiments, the MSLN binding region provided herein comprises an HCDR1 comprising an amino acid sequence of any of SEQ ID NOs: 122, 125, 126, 127 and 130; (ii) an HCDR2 comprising an amino acid sequence of any of SEQ ID NOs:31, 134, 135, 136, and 137, (iii) an HCDR3 comprising an amino acid sequence of SEQ ID NOs:3, 13, and 19; (iv) a LCDR1 comprising an amino acid sequence of SEQ ID NOs: 123, 128, and 131; (v) a LCDR2 comprising an amino acid sequence of SEQ ID NOs:44, 124, and 129; and/or (vi) a LCDR3 comprising an amino acid sequence of SEQ ID NOs:99 and 103.

In some specific embodiments, in the MSLN binding region provided herein, the HCDR1 comprises the amino acid sequence of SEQ ID NO: 122, the HCDR2 comprises the amino acid sequence of SEQ ID NO:134, the HCDR3 comprises the amino acid sequence of SEQ ID NO:3, the LCDR1 comprises the amino acid sequence of SEQ ID NO:123, the LCDR2 comprises the amino acid sequence of SEQ ID NO: 124, and the LCDR3 comprises the amino acid sequence of SEQ ID NO:99.

In some specific embodiments, in the MSLN binding region provided herein, the HCDR1 comprises the amino acid sequence of SEQ ID NO: 125, the HCDR2 comprises the amino acid sequence of SEQ ID NO:31, the HCDR3 comprises the amino acid sequence of SEQ ID NO:3, the LCDR1 comprises the amino acid sequence of SEQ ID NO: 123, the LCDR2 comprises the amino acid sequence of SEQ ID NO: 124, and the LCDR3 comprises the amino acid sequence of SEQ ID NO:99.

In some specific embodiments, in the MSLN binding region provided herein, the HCDR1 comprises the amino acid sequence of SEQ ID NO: 126, the HCDR2 comprises the amino acid sequence of SEQ ID NO: 135, the HCDR3 comprises the amino acid sequence of SEQ ID NO:3, the LCDR1 comprises the amino acid sequence of SEQ ID NO: 123, the LCDR2 comprises the amino acid sequence of SEQ ID NO: 124, and the LCDR3 comprises the amino acid sequence of SEQ ID NO:99.

In some specific embodiments, in the MSLN binding region provided herein, the HCDR1 comprises the amino acid sequence of SEQ ID NO: 127, the HCDR2 comprises the amino acid sequence of SEQ ID NO: 136, the HCDR3 comprises the amino acid sequence of SEQ ID NO:13, the LCDR1 comprises the amino acid sequence of SEQ ID NO: 128, the LCDR2 comprises the amino acid sequence of SEQ ID NO: 129, and the LCDR3 comprises the amino acid sequence of SEQ ID NO: 103.

In some specific embodiments, in the MSLN binding region provided herein, the HCDR1 comprises the amino acid sequence of SEQ ID NO: 130, the HCDR2 comprises the amino acid sequence of SEQ ID NO: 137, the HCDR3 comprises the amino acid sequence of SEQ ID NO: 19, the LCDR1 comprises the amino acid sequence of SEQ ID NO: 131, the LCDR2 comprises the amino acid sequence of LGS, and the LCDR3 comprises the amino acid sequence of SEQ ID NO:99.

In one embodiment, provided herein is a binding region that binds MSLN, comprising a VH comprising a HCDR1, a HCDR2, and a HCDR3 having an amino acid sequence of SEQ ID NOs: 122, 134, and 3, respectively. In another embodiment, provided herein is a binding region that binds MSLN, comprising a VL comprising a LCDR1, a LCDR2, and a LCDR3 having an amino acid sequence of SEQ ID NOs: 123, 124, and 99, respectively. In another embodiment, provided herein is a binding region that binds MSLN, comprising: (i) a VH comprising a HCDR1, a HCDR2, and a HCDR3 having an amino acid sequence of SEQ ID NOs:122, 134, and 3, respectively, and (ii) a VL comprising a LCDR1, a LCDR2, and a LCDR3 having an amino acid sequence of SEQ ID NOs: 123, 124, and 99, respectively.

In one embodiment, provided herein is a binding region that binds MSLN, comprising a VH comprising a HCDR1, a HCDR2, and a HCDR3 having an amino acid sequence of SEQ ID NOs: 125, 31, and 3, respectively. In another embodiment, provided herein is a binding region that binds MSLN, comprising a VL comprising a LCDR1, a LCDR2, and a LCDR3 having an amino acid sequence of SEQ ID NOs: 123, 124, and 99, respectively. In another embodiment, provided herein is a binding region that binds MSLN, comprising: (i) a VH comprising a HCDR1, a HCDR2, and a HCDR3 having an amino acid sequence of SEQ ID NOs: 125, 31, and 3, respectively, and (ii) a VL comprising a LCDR1, a LCDR2, and a LCDR3 having an amino acid sequence of SEQ ID NOs: 123, 124, and 99, respectively.

In one embodiment, provided herein is a binding region that binds MSLN, comprising a VH comprising a HCDR1, a HCDR2, and a HCDR3 having an amino acid sequence of SEQ ID NOs:126, 135, and 3, respectively. In another embodiment, provided herein is a binding region that binds MSLN, comprising a VL comprising a LCDR1, a LCDR2, and a LCDR3 having an amino acid sequence of SEQ ID NOs: 123, 124, and 99, respectively. In another embodiment, provided herein is a binding region that binds MSLN, comprising: (i) a VH comprising a HCDR1, a HCDR2, and a HCDR3 having an amino acid sequence of SEQ ID NOs:126, 135, and 3, respectively, and (ii) a VL comprising a LCDR1, a LCDR2, and a LCDR3 having an amino acid sequence of SEQ ID NOs: 123, 124, and 99, respectively.

In one embodiment, provided herein is a binding region that binds MSLN, comprising a VH comprising a HCDR1, a HCDR2, and a HCDR3 having an amino acid sequence of SEQ ID NOs:127, 136, and 13, respectively. In another embodiment, provided herein is a binding region that binds MSLN, comprising a VL comprising a LCDR1, a LCDR2, and a LCDR3 having an amino acid sequence of SEQ ID NOs: 128, 129, and 103, respectively. In another embodiment, provided herein is a binding region that binds MSLN, comprising: (i) a VH comprising a HCDR1, a HCDR2, and a HCDR3 having an amino acid sequence of SEQ ID NOs: 127, 136, and 13, respectively, and (ii) a VL comprising a LCDR1, a LCDR2, and a LCDR3 having an amino acid sequence of SEQ ID NOs: 128, 129, and 103, respectively.

In one embodiment, provided herein is a binding region that binds MSLN, comprising a VH comprising a HCDR1, a HCDR2, and a HCDR3 having an amino acid sequence of SEQ ID NOs: 130, 137, and 19, respectively. In another embodiment, provided herein is a binding region that binds MSLN, comprising a VL comprising a LCDR1, a LCDR2, and a LCDR3 having an amino acid sequence of SEQ ID NOs: 131, 44, and 99, respectively. In another embodiment, provided herein is a binding region that binds MSLN, comprising: (i) a VH comprising a HCDR1, a HCDR2, and a HCDR3 having an amino acid sequence of SEQ ID NOs:130, 137, and 19, respectively, and (ii) a VL comprising a LCDR1, a LCDR2, and a LCDR3 having an amino acid sequence of SEQ ID NOs: 131, 44, and 99, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:120. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO: 139. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 120, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO: 139. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 108, 112, 113, 114, and 117; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 52, 54, 59, and 109, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:110, 115, and 118; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 111, 116, and 119; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:28, 38, and 44; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:87 and 91. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 10.

In some embodiments, the MSLN binding region provided herein comprises an HCDR1 comprising an amino acid sequence of any of SEQ ID NOs: 108, 112, 113, 114, and 117; (ii) an HCDR2 comprising an amino acid sequence of any of SEQ ID NOs:31, 52, 54, 59, and 109, (iii) an HCDR3 comprising an amino acid sequence of SEQ ID NOs: 110, 115, and 118; (iv) a LCDR1 comprising an amino acid sequence of SEQ ID NOs:111, 116, and 119; (v) a LCDR2 comprising an amino acid sequence of SEQ ID NOs:28, and 38, and 44; and/or (vi) a LCDR3 comprising an amino acid sequence of SEQ ID NOs:87 and 91.

In some specific embodiments, in the MSLN binding region provided herein, the HCDR1 comprises the amino acid sequence of SEQ ID NO: 108, the HCDR2 comprises the amino acid sequence of SEQ ID NO:109, the HCDR3 comprises the amino acid sequence of SEQ ID NO:110, the LCDR1 comprises the amino acid sequence of SEQ ID NO:111, the LCDR2 comprises the amino acid sequence of SEQ ID NO:28, and the LCDR3 comprises the amino acid sequence of SEQ ID NO:87.

In some specific embodiments, in the MSLN binding region provided herein, the HCDR1 comprises the amino acid sequence of SEQ ID NO: 112, the HCDR2 comprises the amino acid sequence of SEQ ID NO:31, the HCDR3 comprises the amino acid sequence of SEQ ID NO:110, the LCDR1 comprises the amino acid sequence of SEQ ID NO: 111, the LCDR2 comprises the amino acid sequence of SEQ ID NO:28, and the LCDR3 comprises the amino acid sequence of SEQ ID NO:87.

In some specific embodiments, in the MSLN binding region provided herein, the HCDR1 comprises the amino acid sequence of SEQ ID NO:113, the HCDR2 comprises the amino acid sequence of SEQ ID NO:52, the HCDR3 comprises the amino acid sequence of SEQ ID NO:110, the LCDR1 comprises the amino acid sequence of SEQ ID NO:111, the LCDR2 comprises the amino acid sequence of SEQ ID NO:28, and the LCDR3 comprises the amino acid sequence of SEQ ID NO:87.

In some specific embodiments, in the MSLN binding region provided herein, the HCDR1 comprises the amino acid sequence of SEQ ID NO: 114, the HCDR2 comprises the amino acid sequence of SEQ ID NO:54, the HCDR3 comprises the amino acid sequence of SEQ ID NO:115, the LCDR1 comprises the amino acid sequence of SEQ ID NO:116, the LCDR2 comprises the amino acid sequence of SEQ ID NO:38, and the LCDR3 comprises the amino acid sequence of SEQ ID NO:91.

In some specific embodiments, in the MSLN binding region provided herein, the HCDR1 comprises the amino acid sequence of SEQ ID NO: 117, the HCDR2 comprises the amino acid sequence of SEQ ID NO:59, the HCDR3 comprises the amino acid sequence of SEQ ID NO:118, the LCDR1 comprises the amino acid sequence of SEQ ID NO: 119, the LCDR2 comprises the amino acid sequence of LGS, and the LCDR3 comprises the amino acid sequence of SEQ ID NO:87.

In one embodiment, provided herein is a binding region that binds MSLN, comprising a VH comprising a HCDR1, a HCDR2, and a HCDR3 having an amino acid sequence of SEQ ID NOs:108, 109, and 110, respectively. In another embodiment, provided herein is a binding region that binds MSLN, comprising a VL comprising a LCDR1, a LCDR2, and a LCDR3 having an amino acid sequence of SEQ ID NOs: 111, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs: 108, 109, and 110, respectively, and (ii) a VL comprising a LCDR1, a LCDR2, and a LCDR3 having an amino acid sequence of SEQ ID NOs: 111, 28, and 87, respectively.

In one embodiment, provided herein is a binding region that binds MSLN, comprising a VH comprising a HCDR1, a HCDR2, and a HCDR3 having an amino acid sequence of SEQ ID NOs:112, 31, and 110, respectively. In another embodiment, provided herein is a binding region that binds MSLN, comprising a VL comprising a LCDR1, a LCDR2, and a LCDR3 having an amino acid sequence of SEQ ID NOs: 111, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:112, 31, and 110, respectively, and (ii) a VL comprising a LCDR1, a LCDR2, and a LCDR3 having an amino acid sequence of SEQ ID NOs: 111, 28, and 87, respectively.

In one embodiment, provided herein is a binding region that binds MSLN, comprising a VH comprising a HCDR1, a HCDR2, and a HCDR3 having an amino acid sequence of SEQ ID NOs: 113, 52, and 110, respectively. In another embodiment, provided herein is a binding region that binds MSLN, comprising a VL comprising a LCDR1, a LCDR2, and a LCDR3 having an amino acid sequence of SEQ ID NOs: 111, 28, and 87, respectively. In another embodiment, provided herein is a binding region that binds MSLN, comprising: (i) a VH comprising a HCDR1, a HCDR2, and a HCDR3 having an amino acid sequence of SEQ ID NOs:113, 52, and 110, respectively, and (ii) a VL comprising a LCDR1, a LCDR2, and a LCDR3 having an amino acid sequence of SEQ ID NOs: 111, 28, and 87, respectively.

In one embodiment, provided herein is a binding region that binds MSLN, comprising a VH comprising a HCDR1, a HCDR2, and a HCDR3 having an amino acid sequence of SEQ ID NOs: 114, 54, and 115, respectively. In another embodiment, provided herein is a binding region that binds MSLN, comprising a VL comprising a LCDR1, a LCDR2, and a LCDR3 having an amino acid sequence of SEQ ID NOs: 116, 38, and 91, respectively. In another embodiment, provided herein is a binding region that binds MSLN, comprising: (i) a VH comprising a HCDR1, a HCDR2, and a HCDR3 having an amino acid sequence of SEQ ID NOs:114, 54, and 115, respectively, and (ii) a VL comprising a LCDR1, a LCDR2, and a LCDR3 having an amino acid sequence of SEQ ID SEQ ID NOs: 116, 38, and 91, respectively.

In one embodiment, provided herein is a binding region that binds MSLN, comprising a VH comprising a HCDR1, a HCDR2, and a HCDR3 having an amino acid sequence of SEQ ID NOs:117, 59, and 118, respectively. In another embodiment, provided herein is a binding region that binds MSLN, comprising a VL comprising a LCDR1, a LCDR2, and a LCDR3 having an amino acid sequence of SEQ ID NOs:119, 44, and 87, respectively. In another embodiment, provided herein is a binding region that binds MSLN, comprising: (i) a VH comprising a HCDR1, a HCDR2, and a HCDR3 having an amino acid sequence of SEQ ID NOs:117, 59, and 118, respectively, and (ii) a VL comprising a LCDR1, a LCDR2, and a LCDR3 having an amino acid sequence of SEQ ID NOs: 119, 44, and 87, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO:106. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO: 140. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO:106, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:140. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 1, 7, 9, 11, and 17; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 52, 54, 59, and 96, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:97, 100, and 104; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:98, 101, and 105; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:5, 21, and 102; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:99 and 103. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 11.

In some embodiments, the MSLN binding region provided herein comprises an HCDR1 comprising an amino acid sequence of any of SEQ ID NOs:1, 7, 9, 11, and 17; (ii) an HCDR2 comprising an amino acid sequence of any of SEQ ID NOs:31, 52, 54, 59, and 96, (iii) an HCDR3 comprising an amino acid sequence of SEQ ID NOs:97, 100, and 104; (iv) a LCDR1 comprising an amino acid sequence of SEQ ID NOs:98, 101, and 105; (v) a LCDR2 comprising an amino acid sequence of SEQ ID NOs:5, 21, and 102; and/or (vi) a LCDR3 comprising an amino acid sequence of SEQ ID NOs:99 and 103.

In some specific embodiments, in the MSLN binding region provided herein, the HCDR1 comprises the amino acid sequence of SEQ ID NO:1, the HCDR2 comprises the amino acid sequence of SEQ ID NO:96, the HCDR3 comprises the amino acid sequence of SEQ ID NO:97, the LCDR1 comprises the amino acid sequence of SEQ ID NO:98, the LCDR2 comprises the amino acid sequence of SEQ ID NO:5, and the LCDR3 comprises the amino acid sequence of SEQ ID NO:99.

In some specific embodiments, in the MSLN binding region provided herein, the HCDR1 comprises the amino acid sequence of SEQ ID NO:7, the HCDR2 comprises the amino acid sequence of SEQ ID NO:31, the HCDR3 comprises the amino acid sequence of SEQ ID NO:97, the LCDR1 comprises the amino acid sequence of SEQ ID NO:98, the LCDR2 comprises the amino acid sequence of SEQ ID NO:5, and the LCDR3 comprises the amino acid sequence of SEQ ID NO:99.

In some specific embodiments, in the MSLN binding region provided herein, the HCDR1 comprises the amino acid sequence of SEQ ID NO:9, the HCDR2 comprises the amino acid sequence of SEQ ID NO:52, the HCDR3 comprises the amino acid sequence of SEQ ID NO:97, the LCDR1 comprises the amino acid sequence of SEQ ID NO:98, the LCDR2 comprises the amino acid sequence of SEQ ID NO:5, and the LCDR3 comprises the amino acid sequence of SEQ ID NO:99.

In some specific embodiments, in the MSLN binding region provided herein, the HCDR1 comprises the amino acid sequence of SEQ ID NO:11, the HCDR2 comprises the amino acid sequence of SEQ ID NO:54, the HCDR3 comprises the amino acid sequence of SEQ ID NO:100, the LCDR1 comprises the amino acid sequence of SEQ ID NO: 101, the LCDR2 comprises the amino acid sequence of SEQ ID NO: 102, and the LCDR3 comprises the amino acid sequence of SEQ ID NO:103.

In some specific embodiments, in the MSLN binding region provided herein, the HCDR1 comprises the amino acid sequence of SEQ ID NO:17, the HCDR2 comprises the amino acid sequence of SEQ ID NO:59, the HCDR3 comprises the amino acid sequence of SEQ ID NO:104, the LCDR1 comprises the amino acid sequence of SEQ ID NO: 105, the LCDR2 comprises the amino acid sequence of LVS, and the LCDR3 comprises the amino acid sequence of SEQ ID NO:99.

In one embodiment, provided herein is a binding region that binds MSLN, comprising a VH comprising a HCDR1, a HCDR2, and a HCDR3 having an amino acid sequence of SEQ ID NOs:1, 96, and 97, respectively. In another embodiment, provided herein is a binding region that binds MSLN, comprising a VL comprising a LCDR1, a LCDR2, and a LCDR3 having an amino acid sequence of SEQ ID NOs:98, 5, and 99, respectively. In another embodiment, provided herein is a binding region that binds MSLN, comprising: (i) a VH comprising a HCDR1, a HCDR2, and a HCDR3 having an amino acid sequence of SEQ ID NOs: 1, 96, and 97, respectively, and (ii) a VL comprising a LCDR1, a LCDR2, and a LCDR3 having an amino acid sequence of SEQ ID NOs:98, 5, and 99, respectively.

In one embodiment, provided herein is a binding region that binds MSLN, comprising a VH comprising a HCDR1, a HCDR2, and a HCDR3 having an amino acid sequence of SEQ ID NOs:7, 31, and 97, respectively. In another embodiment, provided herein is a binding region that binds MSLN, comprising a VL comprising a LCDR1, a LCDR2, and a LCDR3 having an amino acid sequence of SEQ ID NOs:98, 5, and 99, respectively. In another embodiment, provided herein is a binding region that binds MSLN, comprising: (i) a VH comprising a HCDR1, a HCDR2, and a HCDR3 having an amino acid sequence of SEQ ID NOs:7, 31, and 97, respectively, and (ii) a VL comprising a LCDR1, a LCDR2, and a LCDR3 having an amino acid sequence of SEQ ID NOs:98, 5, and 99, respectively.

In one embodiment, provided herein is a binding region that binds MSLN, comprising a VH comprising a HCDR1, a HCDR2, and a HCDR3 having an amino acid sequence of SEQ ID NOs:9, 52, and 97, respectively. In another embodiment, provided herein is a binding region that binds MSLN, comprising a VL comprising a LCDR1, a LCDR2, and a LCDR3 having an amino acid sequence of SEQ ID NOs:98, 5, and 99, respectively. In another embodiment, provided herein is a binding region that binds MSLN, comprising: (i) a VH comprising a HCDR1, a HCDR2, and a HCDR3 having an amino acid sequence of SEQ ID NOs:9, 52, and 97, respectively, and (ii) a VL comprising a LCDR1, a LCDR2, and a LCDR3 having an amino acid sequence of SEQ ID NOs:98, 5, and 99, respectively.

In one embodiment, provided herein is a binding region that binds MSLN, comprising a VH comprising a HCDR1, a HCDR2, and a HCDR3 having an amino acid sequence of SEQ ID NOs: 11, 54, and 100, respectively. In another embodiment, provided herein is a binding region that binds MSLN, comprising a VL comprising a LCDR1, a LCDR2, and a LCDR3 having an amino acid sequence of SEQ ID NOs: 101, 102, and 103, respectively. In another embodiment, provided herein is a binding region that binds MSLN, comprising: (i) a VH comprising a HCDR1, a HCDR2, and a HCDR3 having an amino acid sequence of SEQ ID NOs:11, 54, and 100, respectively, and (ii) a VL comprising a LCDR1, a LCDR2, and a LCDR3 having an amino acid sequence of SEQ ID NOs: 101, 102, and 103, respectively.

In one embodiment, provided herein is a binding region that binds MSLN, comprising a VH comprising a HCDR1, a HCDR2, and a HCDR3 having an amino acid sequence of SEQ ID NOs: 17, 59, and 104, respectively. In another embodiment, provided herein is a binding region that binds MSLN, comprising a VL comprising a LCDR1, a LCDR2, and a LCDR3 having an amino acid sequence of SEQ ID NOs: 105, 21, and 99, respectively. In another embodiment, provided herein is a binding region that binds MSLN, comprising: (i) a VH comprising a HCDR1, a HCDR2, and a HCDR3 having an amino acid sequence of SEQ ID NOs: 17, 59, and 104, respectively, and (ii) a VL comprising a LCDR1, a LCDR2, and a LCDR3 having an amino acid sequence of SEQ ID NOs: 105, 21, and 99, respectively.

In one embodiment, the MSLN binding region comprises a VH comprising a HCDR1, a HCDR2, and a HCDR3 having an amino acid sequence of a HCDR1, a HCDR2, and a HCDR3, respectively, of a VH having an amino acid sequence of SEQ ID NO: 132. In another embodiment, the MSLN binding region comprises a VL comprising a LCDR1, a LCDR2, and a LCDR3 having an amino acid sequence of a LCDR1, a LCDR2, and a LCDR3, respectively, of a VL having an amino acid sequence of SEQ ID NO:142. In yet another embodiment, the MSLN binding region comprises: (i) a VH comprising a HCDR1, a HCDR2, and a HCDR3 having an amino acid sequence of a HCDR1, a HCDR2, and a HCDR3, respectively, of a VH having an amino acid sequence of SEQ ID NO: 132, and (ii) a VL comprising a LCDR1, a LCDR2, and a LCDR3 having an amino acid sequence of a LCDR1, a LCDR2, and a LCDR3, respectively, of a VL having an amino acid sequence of SEQ ID NO:142. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the MSLN binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 122, 125, 126, 127, and 130; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs:31, 52, 54, 59, and 84, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:3, 13, and 19; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 123, 128, and 131; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs:28, 44, and 141; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:99 and 103. In some embodiments, the MSLN binding region is humanized. In some embodiments, the MSLN binding region comprises an acceptor human framework, e.g., a human immunoglobulin framework or a human consensus framework.

In some specific embodiments, the MSLN binding region provided herein comprises one or more CDRs in Table 12.

In some embodiments, the MSLN binding region provided herein comprises an HCDR1 comprising an amino acid sequence of any of SEQ ID NOs: 122, 125, 126, 127, and 130; (ii) an HCDR2 comprising an amino acid sequence of any of SEQ ID NOs:31, 52, 54, 59, and 84, (iii) an HCDR3 comprising an amino acid sequence of SEQ ID NOs:3, 13, and 19; (iv) a LCDR1 comprising an amino acid sequence of SEQ ID NOs: 123, 128, and 131; (v) a LCDR2 comprising an amino acid sequence of SEQ ID NOs:28, 44, and 141; and/or (vi) a LCDR3 comprising an amino acid sequence of SEQ ID NOs:99 and 103.

In some specific embodiments, in the MSLN binding region provided herein, the HCDR1 comprises the amino acid sequence of SEQ ID NO: 122, the HCDR2 comprises the amino acid sequence of SEQ ID NO:84, the HCDR3 comprises the amino acid sequence of SEQ ID NO:3, the LCDR1 comprises the amino acid sequence of SEQ ID NO: 123, the LCDR2 comprises the amino acid sequence of SEQ ID NO:28, and the LCDR3 comprises the amino acid sequence of SEQ ID NO:99.

In some specific embodiments, in the MSLN binding region provided herein, the HCDR1 comprises the amino acid sequence of SEQ ID NO: 125 the HCDR2 comprises the amino acid sequence of SEQ ID NO:31, the HCDR3 comprises the amino acid sequence of SEQ ID NO:3, the LCDR1 comprises the amino acid sequence of SEQ ID NO:123, the LCDR2 comprises the amino acid sequence of SEQ ID NO:28, and the LCDR3 comprises the amino acid sequence of SEQ ID NO:99.

In some specific embodiments, in the MSLN binding region provided herein, the HCDR1 comprises the amino acid sequence of SEQ ID NO: 126, the HCDR2 comprises the amino acid sequence of SEQ ID NO:52, the HCDR3 comprises the amino acid sequence of SEQ ID NO:3, the LCDR1 comprises the amino acid sequence of SEQ ID NO: 123, the LCDR2 comprises the amino acid sequence of SEQ ID NO:28, and the LCDR3 comprises the amino acid sequence of SEQ ID NO:99.

In some specific embodiments, in the MSLN binding region provided herein, the HCDR1 comprises the amino acid sequence of SEQ ID NO: 127, the HCDR2 comprises the amino acid sequence of SEQ ID NO:54, the HCDR3 comprises the amino acid sequence of SEQ ID NO: 13, the LCDR1 comprises the amino acid sequence of SEQ ID NO:128, the LCDR2 comprises the amino acid sequence of SEQ ID NO: 141, and the LCDR3 comprises the amino acid sequence of SEQ ID NO: 103.

In some specific embodiments, in the MSLN binding region provided herein, the HCDR1 comprises the amino acid sequence of SEQ ID NO:130, the HCDR2 comprises the amino acid sequence of SEQ ID NO:59, the HCDR3 comprises the amino acid sequence of SEQ ID NO: 19, the LCDR1 comprises the amino acid sequence of SEQ ID NO: 131, the LCDR2 comprises the amino acid sequence of LGS, and the LCDR3 comprises the amino acid sequence of SEQ ID NO:99.

In one embodiment, provided herein is a binding region that binds MSLN, comprising a VH comprising a HCDR1, a HCDR2, and a HCDR3 having an amino acid sequence of SEQ ID NOs: 122, 84, and 3, respectively. In another embodiment, provided herein is a binding region that binds MSLN, comprising a VL comprising a LCDR1, a LCDR2, and a LCDR3 having an amino acid sequence of SEQ ID NOs: 123, 28, and 99, respectively. In another embodiment, provided herein is a binding region that binds MSLN, comprising: (i) a VH comprising a HCDR1, a HCDR2, and a HCDR3 having an amino acid sequence of SEQ ID NOs: 122, 84, and 3, respectively, and (ii) a VL comprising a LCDR1, a LCDR2, and a LCDR3 having an amino acid sequence of SEQ ID NOs: 123, 28, and 99, respectively.

In one embodiment, provided herein is a binding region that binds MSLN, comprising a VH comprising a HCDR1, a HCDR2, and a HCDR3 having an amino acid sequence of SEQ ID NOs: 125, 31, and 3, respectively. In another embodiment, provided herein is a binding region that binds MSLN, comprising a VL comprising a LCDR1, a LCDR2, and a LCDR3 having an amino acid sequence of SEQ ID NOs: 123, 28, and 99, respectively. In another embodiment, provided herein is a binding region that binds MSLN, comprising: (i) a VH comprising a HCDR1, a HCDR2, and a HCDR3 having an amino acid sequence of SEQ ID NOs: 125, 31, and 3, respectively, and (ii) a VL comprising a LCDR1, a LCDR2, and a LCDR3 having an amino acid sequence of SEQ ID NOs: 123, 28, and 99, respectively.

In one embodiment, provided herein is a binding region that binds MSLN, comprising a VH comprising a HCDR1, a HCDR2, and a HCDR3 having an amino acid sequence of SEQ ID NOs: 126, 53, and 3, respectively. In another embodiment, provided herein is a binding region that binds MSLN, comprising a VL comprising a LCDR1, a LCDR2, and a LCDR3 having an amino acid sequence of SEQ ID NOs: 123, 28, and 99, respectively. In another embodiment, provided herein is a binding region that binds MSLN, comprising: (i) a VH comprising a HCDR1, a HCDR2, and a HCDR3 having an amino acid sequence of SEQ ID NOs:126, 53, and 3, respectively, and (ii) a VL comprising a LCDR1, a LCDR2, and a LCDR3 having an amino acid sequence of SEQ ID NOs: 123, 28, and 99, respectively.

In one embodiment, provided herein is a binding region that binds MSLN, comprising a VH comprising a HCDR1, a HCDR2, and a HCDR3 having an amino acid sequence of SEQ ID NOs: 127, 54, and 13, respectively. In another embodiment, provided herein is a binding region that binds MSLN, comprising a VL comprising a LCDR1, a LCDR2, and a LCDR3 having an amino acid sequence of SEQ ID NOs: 128, 141, and 103, respectively. In another embodiment, provided herein is a binding region that binds MSLN, comprising: (i) a VH comprising a HCDR1, a HCDR2, and a HCDR3 having an amino acid sequence of SEQ ID NOs: 127, 54, and 13, respectively, and (ii) a VL comprising a LCDR1, a LCDR2, and a LCDR3 having an amino acid sequence of SEQ ID NOs: 128, 141, and 103, respectively.

In one embodiment, provided herein is a binding region that binds MSLN, comprising a VH comprising a HCDR1, a HCDR2, and a HCDR3 having an amino acid sequence of SEQ ID NOs: 130, 59, and 19, respectively. In another embodiment, provided herein is a binding region that binds MSLN, comprising a VL comprising a LCDR1, a LCDR2, and a LCDR3 having an amino acid sequence of SEQ ID NOs: 131, 44, and 99, respectively. In another embodiment, provided herein is a binding region that binds MSLN, comprising: (i) a VH comprising a HCDR1, a HCDR2, and a HCDR3 having an amino acid sequence of SEQ ID NOs:130, 59, and 19, respectively, and (ii) a VL comprising a LCDR1, a LCDR2, and a LCDR3 having an amino acid sequence of SEQ ID NOs: 131, 44, and 99, respectively.

In some embodiments, the MSLN binding region further comprises one or more framework regions of the VH or VL having the amino acid sequence of any one of SEQ ID NO:22, SEQ ID NO:23, SEQ ID NO:45, SEQ ID NO:46, SEQ ID NO:62, SEQ ID NO:63, SEQ ID NO:81, SEQ ID NO:82, SEQ ID NO:94, SEQ ID NO:95, SEQ ID NO: 106, SEQ ID NO: 107, SEQ ID NO: 120, SEQ ID NO: 121, SEQ ID NO: 132, SEQ ID NO: 133, SEQ ID NO:138, SEQ ID NO: 139, SEQ ID NO: 140, and SEQ ID NO:142. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FRI is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In one embodiment, provided herein is a binding region that binds MSLN, comprising a VH having an amino acid sequence of SEQ ID NO:22. In another embodiment, provided herein is a binding region that binds MSLN, comprising a VL having an amino acid sequence of SEQ ID NO:23. In another embodiment, provided herein is a binding region that binds MSLN, comprising: (i) a VH having an amino acid sequence of SEQ ID NO:22, and (ii) a VL having an amino acid sequence of SEQ ID NO:23.

In one embodiment, provided herein is a binding region that binds MSLN, comprising a VH having an amino acid sequence of SEQ ID NO:45. In another embodiment, provided herein is a binding region that binds MSLN, comprising a VL having an amino acid sequence of SEQ ID NO:46. In another embodiment, provided herein is a binding region that binds MSLN, comprising: (i) a VH having an amino acid sequence of SEQ ID NO:45, and (ii) a VL having an amino acid sequence of SEQ ID NO:46.

In one embodiment, provided herein is a binding region that binds MSLN, comprising a VH having an amino acid sequence of SEQ ID NO:62. In another embodiment, provided herein is a binding region that binds MSLN, comprising a VL having an amino acid sequence of SEQ ID NO:63. In another embodiment, provided herein is a binding region that binds MSLN, comprising: (i) a VH having an amino acid sequence of SEQ ID NO:62, and (ii) a VL having an amino acid sequence of SEQ ID NO:63.

In one embodiment, provided herein is a binding region that binds MSLN, comprising a VH having an amino acid sequence of SEQ ID NO:81. In another embodiment, provided herein is a binding region that binds MSLN, comprising a VL having an amino acid sequence of SEQ ID NO:82. In another embodiment, provided herein is a binding region that binds MSLN, comprising: (i) a VH having an amino acid sequence of SEQ ID NO:81, and (ii) a VL having an amino acid sequence of SEQ ID NO:82.

In one embodiment, provided herein is a binding region that binds MSLN, comprising a VH having an amino acid sequence of SEQ ID NO:94. In another embodiment, provided herein is a binding region that binds MSLN, comprising a VL having an amino acid sequence of SEQ ID NO:95. In another embodiment, provided herein is a binding region that binds MSLN, comprising: (i) a VH having an amino acid sequence of SEQ ID NO:94, and (ii) a VL having an amino acid sequence of SEQ ID NO:95.

In one embodiment, provided herein is a binding region that binds MSLN, comprising a VH having an amino acid sequence of SEQ ID NO:106. In another embodiment, provided herein is a binding region that binds MSLN, comprising a VL having an amino acid sequence of SEQ ID NO:107. In another embodiment, provided herein is a binding region that binds MSLN, comprising: (i) a VH having an amino acid sequence of SEQ ID NO:106, and (ii) a VL having an amino acid sequence of SEQ ID NO: 107.

In one embodiment, provided herein is a binding region that binds MSLN, comprising a VH having an amino acid sequence of SEQ ID NO:120. In another embodiment, provided herein is a binding region that binds MSLN, comprising a VL having an amino acid sequence of SEQ ID NO: 121. In another embodiment, provided herein is a binding region that binds MSLN, comprising: (i) a VH having an amino acid sequence of SEQ ID NO: 120, and (ii) a VL having an amino acid sequence of SEQ ID NO: 121.

In one embodiment, provided herein is a binding region that binds MSLN, comprising a VH having an amino acid sequence of SEQ ID NO:132. In another embodiment, provided herein is a binding region that binds MSLN, comprising a VL having an amino acid sequence of SEQ ID NO:133. In another embodiment, provided herein is a binding region that binds MSLN, comprising: (i) a VH having an amino acid sequence of SEQ ID NO: 132, and (ii) a VL having an amino acid sequence of SEQ ID NO:133.

In one embodiment, provided herein is a binding region that binds MSLN, comprising a VH having an amino acid sequence of SEQ ID NO:138. In another embodiment, provided herein is a binding region that binds MSLN, comprising a VL having an amino acid sequence of SEQ ID NO:133. In another embodiment, provided herein is a binding region that binds MSLN, comprising: (i) a VH having an amino acid sequence of SEQ ID NO:138, and (ii) a VL having an amino acid sequence of SEQ ID NO:133.

In one embodiment, provided herein is a binding region that binds MSLN, comprising a VH having an amino acid sequence of SEQ ID NO:120. In another embodiment, provided herein is a binding region that binds MSLN, comprising a VL having an amino acid sequence of SEQ ID NO: 139. In another embodiment, provided herein is a binding region that binds MSLN, comprising: (i) a VH having an amino acid sequence of SEQ ID NO:120, and (ii) a VL having an amino acid sequence of SEQ ID NO: 139.

In one embodiment, provided herein is a binding region that binds MSLN, comprising a VH having an amino acid sequence of SEQ ID NO:106. In another embodiment, provided herein is a binding region that binds MSLN, comprising a VL having an amino acid sequence of SEQ ID NO:140. In another embodiment, provided herein is a binding region that binds MSLN, comprising: (i) a VH having an amino acid sequence of SEQ ID NO: 106, and (ii) a VL having an amino acid sequence of SEQ ID NO: 140.

In one embodiment, provided herein is a binding region that binds MSLN, comprising a VH having an amino acid sequence of SEQ ID NO: 132. In another embodiment, provided herein is a binding region that binds MSLN, comprising a VL having an amino acid sequence of SEQ ID NO:142. In another embodiment, provided herein is a binding region that binds MSLN, comprising: (i) a VH having an amino acid sequence of SEQ ID NO: 132, and (ii) a VL having an amino acid sequence of SEQ ID NO:142.

In certain embodiments, the MSLN binding region provided herein comprises amino acid sequences with certain percent identity relative to any MSLN binding region provided herein (such as in Table 1, Table 2, Table 3, Table 4, Table 5, Table 6, Table 7, Table 8, Table 9, Table 10, Table 11, and Table 12).

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the MSLN binding region provide herein contains substitutions (e.g., conservative substitutions), insertions, or deletions relative to the reference sequence, but the MSLN binding region comprising that sequence retains the ability to bind to MSLN. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the MSLN binding region provided herein includes post-translational modifications of a reference sequence.

In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:22, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:23.

In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:45, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:46. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:62, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:63. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:81, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:82. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:94, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:95. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 106, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 107. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 120, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:121. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:132, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 133. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:138, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:133. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 120, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 139. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 106, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:140. In some embodiments, the MSLN binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 132, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:142.

In some embodiments, the MSLN binding region provided herein binds to the same epitope as an MSLN binding region comprising a VH comprising the amino acid sequence of SEQ ID NO:22, and a VL comprising the amino acid sequence of SEQ ID NO:23. In some embodiments, the MSLN binding region provided herein binds to the same epitope as an MSLN binding region comprising a VH comprising the amino acid sequence of SEQ ID NO:45, and a VL comprising the amino acid sequence of SEQ ID NO:46. In some embodiments, the MSLN binding region provided herein binds to the same epitope as an MSLN binding region comprising a VH comprising the amino acid sequence of SEQ ID NO:62, and a VL comprising the amino acid sequence of SEQ ID NO:63. In some embodiments, the MSLN binding region provided herein binds to the same epitope as an MSLN binding region comprising a VH comprising the amino acid sequence of SEQ ID NO:81, and a VL comprising the amino acid sequence of SEQ ID NO:82. In some embodiments, the MSLN binding region provided herein binds to the same epitope as an MSLN binding region comprising a VH comprising the amino acid sequence of SEQ ID NO:94, and a VL comprising the amino acid sequence of SEQ ID NO:95. In some embodiments, the MSLN binding region provided herein binds to the same epitope as an MSLN binding region comprising a VH comprising the amino acid sequence of SEQ ID NO: 106, and a VL comprising the amino acid sequence of SEQ ID NO: 107. In some embodiments, the MSLN binding region provided herein binds to the same epitope as an MSLN binding region comprising a VH comprising the amino acid sequence of SEQ ID NO:120, and a VL comprising the amino acid sequence of SEQ ID NO: 121. In some embodiments, the MSLN binding region provided herein binds to the same epitope as an MSLN binding region comprising a VH comprising the amino acid sequence of SEQ ID NO: 132, and a VL comprising the amino acid sequence of SEQ ID NO:133. In some embodiments, the MSLN binding region provided herein binds to the same epitope as an MSLN binding region comprising a VH comprising the amino acid sequence of SEQ ID NO:138, and a VL comprising the amino acid sequence of SEQ ID NO:133. In some embodiments, the MSLN binding region provided herein binds to the same epitope as an MSLN binding region comprising a VH comprising the amino acid sequence of SEQ ID NO:120, and a VL comprising the amino acid sequence of SEQ ID NO: 139. In some embodiments, the MSLN binding region provided herein binds to the same epitope as an MSLN binding region comprising a VH comprising the amino acid sequence of SEQ ID NO: 106, and a VL comprising the amino acid sequence of SEQ ID NO: 140. In some embodiments, the MSLN binding region provided herein binds to the same epitope as an MSLN binding region comprising a VH comprising the amino acid sequence of SEQ ID NO: 132, and a VL comprising the amino acid sequence of SEQ ID NO: 142.

In some embodiments, the MSLN binding region provided herein specifically binds to MSLN competitively with an MSLN binding region comprising a VH comprising the amino acid sequence of SEQ ID NO:22, and a VL comprising the amino acid sequence of SEQ ID NO:23. In some embodiments, the MSLN binding region provided herein specifically binds to MSLN competitively with an MSLN binding region comprising a VH comprising the amino acid sequence of SEQ ID NO:45, and a VL comprising the amino acid sequence of SEQ ID NO:46. In some embodiments, the MSLN binding region provided herein specifically binds to MSLN competitively with an MSLN binding region comprising a VH comprising the amino acid sequence of SEQ ID NO:62, and a VL comprising the amino acid sequence of SEQ ID NO:63. In some embodiments, the MSLN binding region provided herein specifically binds to MSLN competitively with an MSLN binding region comprising a VH comprising the amino acid sequence of SEQ ID NO:81, and a VL comprising the amino acid sequence of SEQ ID NO:82. In some embodiments, the MSLN binding region provided herein specifically binds to MSLN competitively with an MSLN binding region comprising a VH comprising the amino acid sequence of SEQ ID NO:94, and a VL comprising the amino acid sequence of SEQ ID NO:95. In some embodiments, the MSLN binding region provided herein specifically binds to MSLN competitively with an MSLN binding region comprising a VH comprising the amino acid sequence of SEQ ID NO: 106, and a VL comprising the amino acid sequence of SEQ ID NO:107. In some embodiments, the MSLN binding region provided herein specifically binds to MSLN competitively with an MSLN binding region comprising a VH comprising the amino acid sequence of SEQ ID NO: 120, and a VL comprising the amino acid sequence of SEQ ID NO:121. In some embodiments, the MSLN binding region provided herein specifically binds to MSLN competitively with an MSLN binding region comprising a VH comprising the amino acid sequence of SEQ ID NO: 132, and a VL comprising the amino acid sequence of SEQ ID NO: 133 In some embodiments, the MSLN binding region provided herein specifically binds to MSLN competitively with an MSLN binding region comprising a VH comprising the amino acid sequence of SEQ ID NO:138, and a VL comprising the amino acid sequence of SEQ ID NO:133. In some embodiments, the MSLN binding region provided herein specifically binds to MSLN competitively with an MSLN binding region comprising a VH comprising the amino acid sequence of SEQ ID NO: 120, and a VL comprising the amino acid sequence of SEQ ID NO:139. In some embodiments, the MSLN binding region provided herein specifically binds to MSLN competitively with an MSLN binding region comprising a VH comprising the amino acid sequence of SEQ ID NO: 106, and a VL comprising the amino acid sequence of SEQ ID NO:140. In some embodiments, the MSLN binding region provided herein specifically binds to MSLN competitively with an MSLN binding region comprising a VH comprising the amino acid sequence of SEQ ID NO: 132, and a VL comprising the amino acid sequence of SEQ ID NO:142.

In some embodiments, the MSLN binding region provided herein specifically binds to a discontinuous epitope on MSLN comprising 585-594 and 596-597 amino acids of MSLN (SEQ ID NO: 200). In some embodiments, the MSLN binding region provided herein specifically binds to a discontinuous epitope on MSLN having 585-594 and 596-597 amino acids of MSLN (SEQ ID NO: 200). In some embodiments, the MSLN binding region provided herein specifically binds to a discontinuous epitope on MSLN having G585, Y586, L587, V588, L589, D590, L591, S592, M593, Q594, A596, and L597.

In some embodiments, the MSLN binding region provided herein specifically binds to an epitope on MSLN comprising 587-593 amino acid residues of MSLN (SEQ ID NO: 200). In some embodiments, the MSLN binding region provided herein specifically binds to an epitope on MSLN having 587-593 amino acid residues of MSLN (SEQ ID NO: 200). In some embodiments, the MSLN binding region provided herein specifically binds to an epitope on MSLN having L587, V588, L589, D590, L591, S592, M593.

TABLE 1
MSNB71 Binding Region Sequences

| | Kabat | Chothia | AbM | Contact | IMGT |
|---|---|---|---|---|---|
| Heavy | SSSV | GDSV | GDSV | SSSS | GDSV |
| Chain | AWN | SSSS | SSSS | VAWN | SSSS |
| variable | (SEQ | V | VAWN | (SEQ | VA |
| region | ID | (SEQ | (SEQ | ID | (SEQ |
| CDR1 | NO: | ID | ID | NO: | ID |
| | 1) | NO: | NO: | 11) | NO: |
| | | 7) | 9) | | 17) |
| Heavy | RTYF | YFRS | RTYF | WLGR | TYFR |
| Chain | RSKW | KWY | RSKW | TYFR | SKWY |
| variable | YYDY | (SEQ | YYD | SKWY | Y |
| region | AVSV | ID | (SEQ | YD | (SEQ |
| CDR2 | KS | NO: | ID | (SEQ | ID |
| | (SEQ | 8) | NO: | ID | NO: |
| | ID | | 10) | NO: | 18) |
| | NO: | | | 12) | |
| | 2) | | | | |
| Heavy | GIVG | GIVG | GIVG | SRGI | SRGI |
| Chain | ALDY | ALDY | ALDY | VGAL | VGAL |
| variable | (SEQ | (SEQ | (SEQ | D | DY |
| region | ID | ID | ID | (SEQ | (SEQ |
| CDR3 | NO: | NO: | NO: | ID | ID |
| | 3) | 3) | 3) | NO: | NO: |
| | | | | 13) | 19) |
| Light | RSSQ | RSSQ | RSSQ | LFTN | QSLL |
| Chain | SLLF | SLLF | SLLF | GYDS | FTNG |
| variable | TNGY | TNGY | TNGY | LDWY | YDS |
| region | DSLD | DSLD | DSLD | (SEQ | (SEQ |
| CDR1 | (SEQ | (SEQ | (SEQ | ID | ID |
| | ID | ID | ID | NO: | NO: |
| | NO: | NO: | NO: | 14) | 20) |
| | 4) | 4) | 4) | | |
| Light | LVSN | LVSN | LVSN | LLIY | LVS |
| Chain | RAS | RAS | RAS | LVSN | |
| variable | (SEQ | (SEQ | (SEQ | RA | |
| region | ID | ID | ID | (SEQ | |
| CDR2 | NO: | NO: | NO: | ID | |
| | 5) | 5) | 5) | NO: | |
| | | | | 15) | |
| Light | MQGI | MQGI | MQGI | MQGI | MQGI |
| Chain | RTPF | RTPF | RTPF | RTPF | RTPF |
| variable | T | T | T | (SEQ | T |
| region | (SEQ | (SEQ | (SEQ | ID | (SEQ |
| CDR3 | ID | ID | ID | NO: | ID |
| | NO: | NO: | NO: | 16) | NO: |
| | 6) | 6) | 6) | | 6) |

VH (SEQ ID NO: 22):
QVQLQQSGPGLVMSSQTLSLTCAISGDSVSSSSVAWNWIR
QSPSRGLQWLGRTYFRSKWYYDYAVSVKSRITINPDTSKN
QFSLQLNSVTPEDTAVYYCSRGIVGALDYWGQGTLVTVSS
VL (SEQ ID NO: 23):
DIVMTQSPLSLPVTPGEPASISCRSSQSLLFTNGYDSLDW
YLQKPGQSPQLLIYLVSNRASGVPDRFSGSGSGTEFTLKI
SRVEADDVGVYFCMQGIRTPFTFGPGTKLEIK

TABLE 2
MSNB124 Binding Region Sequences

| | Kabat | Chothia | AbM | Contact | IMGT |
|---|---|---|---|---|---|
| Heavy | SNSV | GDSV | GDSV | SSNS | GDSV |
| Chain | AWH | SSNS | SSNS | VAWH | SSNS |
| variable | (SEQ | V | VAWH | (SEQ | VA |
| region | ID | (SEQ | (SEQ | ID | (SEQ |
| CDR1 | NO: | ID | ID | NO: | ID |
| | 24) | NO: | NO: | 34) | NO: |
| | | 30) | 32) | | 40) |
| Heavy | RTYH | YHRS | RTYH | WLGR | TYHR |
| Chain | RSKW | KWY | RSKW | TYHR | SKWY |
| variable | YNDY | (SEQ | YND | SKWY | N |
| region | AVSV | ID | (SEQ | ND | (SEQ |
| CDR2 | KS | NO: | ID | (SEQ | ID |
| | (SEQ | 252) | NO: | ID | NO: |
| | ID | | 33) | NO: | 41) |
| | NO: | | | 35) | |
| | 25) | | | | |
| Heavy | QILG | QILG | QILG | ARQI | ARQI |
| Chain | ALDY | ALDY | ALDY | LGAL | LGAL |
| variable | (SEQ | (SEQ | (SEQ | D | DY |
| region | ID | ID | ID | (SEQ | (SEQ |
| CDR3 | NO: | NO: | NO: | ID | ID |
| | 26) | 26) | 26) | NO: | NO: |
| | | | | 36) | 42) |
| Light | RSSQ | RSSQ | RSSQ | QFGN | QSLQ |
| Chain | SLQF | SLQF | SLQF | GNYL | FGNG |
| variable | GNGN | GNGN | GNGN | DWY | NY |
| region | YLD | YLD | YLD | (SEQ | (SEQ |
| CDR1 | (SEQ | (SEQ | (SEQ | ID | ID |
| | ID | ID | ID | NO: | NO: |
| | NO: | NO: | NO: | 37) | 43) |
| | 27) | 27) | 27) | | |
| Light | LGSN | LGSN | LGSN | LLIY | LGS |
| Chain | RAS | RAS | RAS | LGSN | |
| variable | (SEQ | (SEQ | (SEQ | RA | |
| region | ID | ID | ID | (SEQ | |
| CDR2 | NO: | NO: | NO: | ID | |
| | 28) | 28) | 28) | NO: | |
| | | | | 38) | |
| Light | MQVL | MQVL | MQVL | MQVL | MQVL |
| Chain | QIPF | QIPF | QIPF | QIPF | QIPF |
| variable | T | T | T | (SEQ | T |
| region | (SEQ | (SEQ | (SEQ | ID | (SEQ |
| CDR3 | ID | ID | ID | NO: | ID |
| | NO: | NO: | NO: | 39) | NO: |
| | 29) | 29) | 29) | | 29) |

VH (SEQ ID NO: 45):
QVQLQQSGPGLVKPSQTLSLTCAISGDSVSSNSVAWHWIR
QSPSRGLEWLGRTYHRSKWYNDYAVSVKSRITINPDTSKN
QFSLQLNSVTPEDTAVYYCARQILGALDYWGQGTLVTVSS
VL (SEQ ID NO: 46):
DIVMTQTPLSLPVTPGEPASISCRSSQSLQFGNGNYLDWY
LQKPGQSPQLLIYLGSNRASGVPDRFSGSGSGTDFTLKIS
RVEAEDVGVYYCMQVLQIPFTFGPGTKVDIK

TABLE 3
MSNB133 Binding Region Sequences

| | Kabat | Chothia | AbM | Contact | IMGT |
|---|---|---|---|---|---|
| Heavy | SNRV | GDSV | GDSV | SSNR | GDSV |
| Chain | AWN | SSNR | SSNR | VAWN | SSNR |
| variable | (SEQ | V | VAWN | (SEQ | VA |
| region | ID | (SEQ | (SEQ | ID | (SEQ |
| CDR1 | NO: | ID | ID | NO: | ID |
| | 47) | NO: | NO: | 53) | NO: |
| | | 251) | 51) | | 58) |
| Heavy | RTYY | YYRS | RTYY | WLGR | TYYR |
| Chain | RSKW | KWY | RSKW | TYYR | SKWY |
| variable | YNDF | (SEQ | YND | SKWY | N |
| region | ALSV | ID | (SEQ | ND | (SEQ |
| CDR2 | KS | NO: | ID | (SEQ | ID |
| | (SEQ | 31) | NO: | ID | NO: |
| | ID | | 52) | NO: | 59) |
| | NO: | | | 54) | |
| | 48) | | | | |
| Heavy | GIVG | GIVG | GIVG | ARGI | ARGI |
| Chain | ALDY | ALDY | ALDY | VGAL | VGAL |
| variable | (SEQ | (SEQ | (SEQ | D | DY |
| region | ID | ID | ID | (SEQ | (SEQ |
| CDR3 | NO: | NO: | NO: | ID | ID |
| | 3) | 3) | 3) | NO: | NO: |
| | | | | 55) | 60) |
| Light Chain | RSSQ | RSSQ | RSSQ | LFSN | QSLL |
| variable | SLLF | SLLF | SLLF | GYNS | FSNG |
| region | SNGY | SNGY | SNGY | LDWY | YNS |
| CDR1 | NSLD | NSLD | NSLD | (SEQ | (SEQ |
| | (SEQ | (SEQ | (SEQ | ID | ID |
| | ID | ID | ID | NO: | NO: |
| | NO: | NO: | NO: | 56) | 61) |
| | 49) | 49) | 49) | | |
| Light Chain | LGSN | LGSN | LGSN | LLIY | LGS |
| variable | RAS | RAS | RAS | LGSN | |
| region | (SEQ | (SEQ | (SEQ | RA | |
| CDR2 | ID | ID | ID | (SEQ | |
| | NO: | NO: | NO: | ID | |
| | 28) | 28) | 28) | NO: | |
| | | | | 38) | |
| Light Chain | MQAL | MQAL | MQAL | MQAL | MQAL |
| variable | QTPF | QTPF | QTPF | QTPF | QTPF |
| region | T | T | T | (SEQ | T |
| CDR3 | (SEQ | (SEQ | (SEQ | ID | (SEQ |
| | ID | ID | ID | NO: | ID |
| | NO: | NO: | NO: | 57) | NO: |
| | 50) | 50) | 50) | | 50) |

VH (SEQ ID NO: 62):
QVQLQQSGPGLVKPSQTLSLTCAISGDSVSSNRVAWNWIR
QSPSRGLEWLGRTYYRSKWYNDFALSVKSRININADTSKS
QFSLQLNSVTPEDTAVYYCARGIVGALDYWGQGTTVTVSS
VL (SEQ ID NO: 63):
DIVMTQSPLSLPVTPGEPASISCRSSQSLLFSNGYNSLDW
YLQKPGQSPQLLIYLGSNRASGVPDRFSGSESGTDFTLKI
SRVEAEDVGVYYCMQALQTPFTFGPGTKLEIK

TABLE 4
MSNB457 Binding Region Sequences

| | Kabat | Chothia | AbM | Contact | IMGT |
|---|---|---|---|---|---|
| Heavy | SNSA | GDSV | GDSV | SSNS | GDSV |
| Chain | AWN | SSNS | SSNS | AAWN | SSNS |
| variable | (SEQ | A | AAWN | (SEQ | AA |
| region | ID | (SEQ | (SEQ | ID | (SEQ |
| CDR1 | NO: | ID | ID | NO: | ID |
| | 64) | NO: | NO: | 72) | NO: |
| | | 70) | 71) | | 77) |
| Heavy | RTYY | YYRS | ND | WLGR | TYYR |
| Chain | RSKW | KWY | (SEQ | TYYR | SKWY |
| variable | YNDY | (SEQ | ID | SKWY | N |
| region | AVSV | ID | NO: | ND | (SEQ |
| CDR2 | KS | NO: | 52)RTYY | (SEQ | ID |
| | (SEQ | 31) | RSKW | ID | NO: |
| | ID | | Y | NO: | 59) |
| | NO: | | | 54) | |
| | 65) | | | | |
| Heavy | RDYS | RDYS | RDYS | ARRD | ARRD |
| Chain | SGGF | SGGF | SGGF | YSSG | YSSG |
| variable | DY | DY | DY | GFD | GFDY |
| region | (SEQ | (SEQ | (SEQ | (SEQ | (SEQ |
| CDR3 | ID | ID | ID | ID | ID |
| | NO: | NO: | NO: | NO: | NO: |
| | 66) | 66) | 66) | 73) | 78) |
| Light | KSSQ | \|KSSQ | KSSQ | LYSS | QSVL |
| Chain | SVLY | SVLY | SVLY | DNKN | YSSD |
| variable | SSDN | SSDN | SSDN | YLAW | NKNY |
| region | KNYL | KNYL | KNYL | Y | (SEQ |
| CDR1 | A | A | A | (SEQ | ID |
| | (SEQ | (SEQ | (SEQ | ID | NO: |
| | ID | ID | ID | NO: | 79) |
| | NO: | NO: | NO: | 74) | |
| | 67) | 67) | 67) | | |
| Light | WAST | WAST | WAST | LLIY | WAS |
| Chain | RES | RES | RES | WAST | |
| variable | (SEQ | (SEQ | (SEQ | RE | |
| region | ID | ID | ID | (SEQ | |
| CDR2 | NO: | NO: | NO: | ID | |
| | 68) | 68) | 68) | NO: | |
| | | | | 75) | |
| Light | QQYY | QQYY | QQYY | QQYY | QQYY |
| Chain | STPF | STPF | STPF | STPF | STPF |
| variable | T | T | T | (SEQ | T |
| region | (SEQ | (SEQ | (SEQ | ID | (SEQ |
| CDR3 | ID | ID | ID | NO: | ID |
| | NO: | NO: | NO: | 76) | NO: |
| | 69) | 69) | 69) | | 69) |

VH (SEQ ID NO: 81):
QVQLVESGPGQVKPSQTLSLTCAISGDSVSSNSAAWNWIR
QSPSRGLEWLGRTYYRSKWYNDYAVSVKSRITINPDTSKN
QFSLQLNSVTPEDTAVYYCARRDYSSGGFDYWGQGTTVTV
SS
VL (SEQ ID NO: 82):
EIVLTQSPDSLAVSLGERATINCKSSQSVLYSSDNKNYLA
WYQQKPGQPPNLLIYWASTRESGVPDRFSGSGSGTDFTLT
INSLQAEDVAVYYCQQYYSTPFTFGPGTKVEIK

TABLE 5
MSNB459 Binding Region Sequences

| | Kabat | Chothia | AbM | Contact | IMGT |
|---|---|---|---|---|---|
| Heavy | SNSV | GDSV | GDSV | SSNS | GDSV |
| Chain | AWN | SSNS | SSNS | VAWN | SSNS |
| variable | (SEQ | V | VAWN | (SEQ | VA |
| region | ID | (SEQ | (SEQ | ID | (SEQ |
| CDR1 | NO: | ID | ID | NO: | ID |
| | 83) | NO: | NO: | 88) | NO: |
| | | 30) | 253) | | 40) |
| Heavy | RTYY | YYRS | RTYY | WLGR | TYYR |
| Chain | RSKW | KWY | RSKW | TYYR | SKWY |
| variable | YNDY | (SEQ | YND | SKWY | N |
| region | ALSV | ID | (SEQ | ND | (SEQ |
| CDR2 | KS | NO: | ID | (SEQ | ID |
| | (SEQ | 31) | NO: | ID | NO: |
| | ID | | 52) | NO: | 59) |
| | NO: | | | 54) | |
| | 84) | | | | |
| Heavy | GVVG | GVVG | GVVG | ARGV | ARGV |
| Chain | AVDY | AVDY | AVDY | VGAV | VGAV |
| variable | (SEQ | (SEQ | (SEQ | D | DY |
| region | ID | ID | ID | (SEQ | (SEQ |
| CDR3 | NO: | NO: | NO: | ID | ID |
| | 85) | 85) | 85) | NO: | NO: |
| | | | | 89) | 92) |
| Light | RSSQ | RSSQ | RSSQ | LYSN | QNLL |
| Chain | NLLY | NLLY | NLLY | GYNS | YSNG |
| variable | SNGY | SNGY | SNGY | LDWY | YNS |
| region | NSLD | NSLD | NSLD | (SEQ | (SEQ |
| CDR1 | (SEQ | (SEQ | (SEQ | ID | ID |
| | ID | ID | ID | NO: | NO: |
| | NO: | NO: | NO: | 90) | 93) |
| | 86) | 86) | 86) | | |
| Light | LGSN | LGSN | LGSN | LLIY | LGS |
| Chain | RAS | RAS | RAS | LGSN | |
| variable | (SEQ | (SEQ | (SEQ | RA | |
| region | ID | ID | ID | (SEQ | |
| CDR2 | NO: | NO: | NO: | ID | |
| | 28) | 28) | 28) | NO: | |
| | | | | 38) | |
| Light | MQGL | MQGL | MQGL | MQGL | MQGL |
| Chain | RTPL | RTPL | RTPL | RTPL | RTPL |
| variable | T | T | T | (SEQ | T |
| region | (SEQ | (SEQ | (SEQ | ID | (SEQ |
| CDR3 | ID | ID | ID | NO: | ID |
| | NO: | NO: | NO: | 91) | NO: |
| | 87) | 87) | 87) | | 87) |

VH (SEQ ID NO: 94):
EVQLVQSGPGLVKPSQTLSLTCAISGDSVSSNSVAWNWIR
QSPSRGLEWLGRTYYRSKWYNDYALSVKSRITINPDTSKN
QFSLQLNSVTPEDTAVYYCARGVVGAVDYWGQGTLVTVSS
VL (SEQ ID NO: 95):
DIVMTQSPLSLPVTPGEPASISCRSSQNLLYSNGYNSLDW
YLQKPGQSPQLLIYLGSNRASGVPDRFSGSGSGTDFTLKI
SRVEAEDVGVYYCMQGLRTPLTFGGGTKVEIK

TABLE 6
MSLNB568 Binding Region Sequences

| | Kabat | Chothia | AbM | Contact | IMGT |
|---|---|---|---|---|---|
| Heavy | SSSV | GDSV | GDSV | SSSS | GDSV |
| Chain | AWN | SSSS | SSSS | VAWN | SSSS |
| variable | (SEQ | V | VAWN | (SEQ | VA |
| region | ID | (SEQ | (SEQ | ID | (SEQ |
| CDR1 | NO: | ID | ID | NO: | ID |
| | 1) | NO: | NO: | 11) | NO: |
| | | 7) | 9) | | 17) |
| Heavy | RTYY | YYRS | RTYY | WLGR | TYYR |
| Chain | RSKW | KWY | RSKW | TYYR | SKWY |
| variable | YNDY | (SEQ | YND | SKWY | N |
| region | AISV | ID | (SEQ | ND | (SEQ |
| CDR2 | RS | NO: | ID | (SEQ | ID |
| | (SEQ | 31) | NO: | ID | NO: |
| | ID | | 52) | NO: | 59) |
| | NO: | | | 54) | |
| | 96) | | | | |
| Heavy | GVVG | GVVG | GVVG | SRGV | SRGV |
| Chain | ALDY | ALDY | ALDY | VGAL | VGAL |
| variable | (SEQ | (SEQ | (SEQ | D | DY |
| region | ID | ID | ID | (SEQ | (SEQ |
| CDR3 | NO: | NO: | NO: | ID | ID |
| | 97) | 97) | 97) | NO: | NO: |
| | | | | 100) | 104) |
| Light | RSSQ | RSSQ | RSSQ | LFTN | QSLL |
| Chain | SLLF | SLLF | SLLF | GYNS | FTNG |
| variable | TNGY | INGY | TNGY | LDWY | YNS |
| region | NSLD | NSLD | NSLD | (SEQ | (SEQ |
| CDR1 | (SEQ | (SEQ | (SEQ | ID | ID |
| | ID | ID | ID | NO: | NO: |
| | NO: | NO: | NO: | 101) | 105) |
| | 98) | 98) | 98) | | |
| Light | LVSN | LVSN | LVSN | FLIY | LVS |
| Chain | RAS | RAS | RAS | LVSN | |
| variable | (SEQ | (SEQ | (SEQ | RA | |
| region | ID | ID | ID | (SEQ | |
| CDR2 | NO: | NO: | NO: | ID | |
| | 5) | 5) | 5) | NO: | |
| | | | | 102) | |
| Light | MQAL | MQAL | MQAL | MQAL | MQAL |
| Chain | RTPY | RTPY | RTPY | RTPY | RTPY |
| variable | T | T | T | (SEQ | T |
| region | (SEQ | (SEQ | (SEQ | ID | (SEQ |
| CDR3 | ID | ID | ID | NO: | ID |
| | NO: | NO: | NO: | 103) | NO: |
| | 99) | 99) | 99) | | 99) |

VH (SEQ ID NO: 106):
QVQLQQSGPGLVKPSQTLSLTCGISGDSVSSSSVAWNWIRQS
PSRGLEWLGRTYYRSKWYNDYAISVRSRVTINPDTSKNQFSL
QLNSVTPEDTAVYYCSRGVVGALDYWGQGTLVTVSS
VL (SEQ ID NO: 107):
DIVMTQSPLSLTVTPGEPASISCRSSQSLLFTNGYNSLDWYL
QKPGQSPQFLIYLVSNRASGVPDRFSGSGSGTDFTLKISRVE
AEDVGVYYCMQALRTPYTFGQGTKVEIK

TABLE 7
MSLNB569 Binding Region Sequences

| | Kabat | Chothia | AbM | Contact | IMGT |
|---|---|---|---|---|---|
| Heavy | STRA | GDSV | GDSV | SSTR | GDSV |
| Chain | AWN | SSTR | SSTR | AAWN | SSTR |
| variable | (SEQ | A | AAWN | (SEQ | AA |
| region | ID | (SEQ | (SEQ | ID | (SEQ |
| CDR1 | NO: | ID | ID | NO: | ID |
| | 108) | NO: | NO: | 114) | NO: |
| | | 112) | 113) | | 117) |
| Heavy | RTYY | YYRS | RTYY | WLGR | TYYR |
| Chain | RSKW | KWY | RSKW | TYYR | SKWY |
| variable | YNDY | (SEQ | YND | SKWY | N |
| region | TVSV | ID | (SEQ | ND | (SEQ |
| CDR2 | KS | NO: | ID | (SEQ | ID |
| | (SEQ | 31) | NO: | ID | NO: |
| | ID | | 52) | NO: | 59) |
| | NO: | | | 54) | |
| | 109) | | | | |
| Heavy | GNVG | GNVG | GNVG | ARGN | ARGN |
| Chain | RVDF | RVDF | RVDF | VGRV | VGRV |
| variable | (SEQ | (SEQ | (SEQ | D | DF |
| region | ID | ID | ID | (SEQ | (SEQ |
| CDR3 | NO: | NO: | NO: | ID | ID |
| | 110) | 110) | 110) | NO: | NO: |
| | | | | 115) | 118) |
| Light Chain | RSSQ | RSSQ | RSSQ | LHSD | QSLL |
| variable | SLLH | SLLH | SLLH | GYNY | HSDG |
| region | SDGY | SDGY | SDGY | LDWY | YNY |
| CDR1 | NYLD | NYLD | NYLD | (SEQ | (SEQ |
| | (SEQ | (SEQ | (SEQ | ID | ID |
| | ID | ID | ID | NO: | NO: |
| | NO: | NO: | NO: | 116) | 119) |
| | 111) | 111) | 111) | | |
| Light Chain | LGSN | LGSN | LGSN | LLIY | LGS |
| variable | RAS | RAS | RAS | LGSN | |
| region | (SEQ | (SEQ | (SEQ | RA | |
| CDR2 | ID | ID | ID | (SEQ | |
| | NO: | NO: | NO: | ID | |
| | 28) | 28) | 28) | NO: | |
| | | | | 38) | |
| Light Chain | MQGL | MQGL | MQGL | MQGL | MQGL |
| variable | RTPL | RTPL | RTPL | RTPL | RTPL |
| region | T | T | T | (SEQ | T |
| CDR3 | (SEQ | (SEQ | (SEQ | ID | (SEQ |
| | ID | ID | ID | NO: | ID |
| | NO: | NO: | NO: | 91) | NO: |
| | 87) | 87) | 87) | | 87) |

VH (SEQ ID NO: 120):
QVQLQQSGPGLVKPSQTLSLTCAISGDSVSSTRAAWNWI
RQSPSRGLEWLGRTYYRSKWYNDYTVSVKSRITINPDTS
KNQFSLHLNSVTPEDSAVYFCARGNVGRVDFWGQGTLVT
VSS
VL (SEQ ID NO: 121):
EIVMTQSPLSLPVTPGEPASISCRSSQSLLHSDGYNYLD
WYLQKPGQSPQLLIYLGSNRASGVPDRFSGSGSDTDFTL
KISRVEAEDVGVYYCMQGLRTPLTFGPGTKVDIK

TABLE 8
MSLNB570 Binding Region Sequences

| | Kabat | Chothia | AbM | Contact | IMGT |
|---|---|---|---|---|---|
| Heavy | STSV | GDSV | GDSV | SSTS | GDSV |
| Chain | AWN | SSTS | SSTS | VAWN | SSTS |
| variable | (SEQ | V | VAWN | (SEQ | VA |
| region | ID | (SEQ | (SEQ | ID | (SEQ |
| CDR1 | NO: | ID | ID | NO: | ID |
| | 122) | NO: | NO: | 127) | NO: |
| | | 125) | 126) | | 130) |
| Heavy | RTYY | YYRS | RTYY | WLGR | TYYR |
| Chain | RSKW | KWY | RSKW | TYYR | SKWY |
| variable | YNDY | (SEQ | YND | SKWY | N |
| region | ALSV | ID | (SEQ | ND | (SEQ |
| CDR2 | KS | NO: | ID | (SEQ | ID |
| | (SEQ | 31) | NO: | ID | NO: |
| | ID | | 52) | NO: | 59) |
| | NO: | | | 54) | |
| | 84) | | | | |
| Heavy | GIVG | GIVG | GIVG | SRGI | SRGI |
| Chain | ALDY | ALDY | ALDY | VGAL | VGAL |
| variable | (SEQ | (SEQ | (SEQ | D | DY |
| region | ID | ID | ID | (SEQ | (SEQ |
| CDR3 | NO: | NO: | NO: | ID | ID |
| | 3) | 3) | 3) | NO: | NO: |
| | | | | 13) | 19) |
| Light Chain | RSSQ | RSSQ | RSSQ | MYSN | QSLM |
| variable | SLMY | SLMY | SLMY | GYNH | YSNG |
| region | SNGY | SNGY | SNGY | LDWY | YNH |
| CDR1 | NHLD | NHLD | NHLD | (SEQ | (SEQ |
| | (SEQ | (SEQ | (SEQ | ID | ID |
| | ID | ID | ID | NO: | NO: |
| | NO: | NO: | NO: | 128) | 131) |
| | 123) | 123) | 123) | | |
| Light Chain | LGSN | LGSN | LGSN | FLIH | LGS |
| variable | RVS | RVS | RVS | LGSN | |
| region | (SEQ | (SEQ | (SEQ | RV | |
| CDR2 | ID | ID | ID | (SEQ | |
| | NO: | NO: | NO: | ID | |
| | 124) | 124) | 124) | NO: | |
| | | | | 129) | |
| Light Chain | MQAL | MQAL | MQAL | MQAL | MQAL |
| variable | RTPY | RTPY | RTPY | RTPY | RTPY |
| region | T | T | T | (SEQ | T |
| CDR3 | (SEQ | (SEQ | (SEQ | ID | (SEQ |
| | ID | ID | ID | NO: | ID |
| | NO: | NO: | NO: | 103) | NO: |
| | 99) | 99) | 99) | | 99) |

VH (SEQ ID NO: 132):
QVQLQQSGPGLVKPSQTLSLTCAISGDSVSSTSVAWNW
IRQSPSGGLEWLGRTYYRSKWYNDYALSVKSRMTINPD
TASNQFSLQLNSVTPEDTAVYYCSRGIVGALDYWGQGT
LVTVSS
VL (SEQ ID NO: 133):
DIVMTQTPLSLPVTPGGPASISCRSSQSLMYSNGYNHL
DWYLQKPGQSPQFLIHLGSNRVSGVPDRFSGSGSGTDF
TLKISRVEAEDVGVYYCMQALRTPYTFGQGTKVDIK

TABLE 9
MSLNB571 Binding Region Sequences

| | Kabat | Chothia | AbM | Contact | IMGT |
|---|---|---|---|---|---|
| Heavy | STSV | GDSV | GDSV | SSTS | GDSV |
| Chain | AWN | SSTS | SSTS | VAWN | SSTS |
| variable | (SEQ | V | VAWN | (SEQ | VA |
| region | ID | (SEQ | (SEQ | ID | (SEQ |
| CDR1 | NO: | ID | ID | NO: | ID |
| | 122) | NO: | NO: | 127) | NO: |
| | | 125) | 126) | | 130) |
| Heavy | RIYY | YYRS | RIYY | WLGR | IYYR |
| Chain | RSKW | KWY | RSKW | IYYR | SKWY |
| variable | YNDY | (SEQ | YND | SKWY | N |
| region | ALSV | ID | (SEQ | ND | (SEQ |
| CDR2 | KS | NO: | ID | (SEQ | ID |
| | (SEQ | 31) | NO: | ID | NO: |
| | ID | | 135) | NO: | 137) |
| | NO: | | | 136) | |
| | 134) | | | | |
| Heavy | GIVG | GIVG | GIVG | SRGI | SRGI |
| Chain | ALDY | ALDY | ALDY | VGAL | VGAL |
| variable | (SEQ | (SEQ | (SEQ | D | DY |
| region | ID | ID | ID | (SEQ | (SEQ |
| CDR3 | NO: | NO: | NO: | ID | ID |
| | 3) | 3) | 3) | NO: | NO: |
| | | | | 13) | 19) |
| Light Chain | RSSQ | RSSQ | RSSQ | MYSN | QSLM |
| variable | SLMY | SLMY | SLMY | GYNH | YSNG |
| region | SNGY | SNGY | SNGY | LDWY | YNH |
| CDR1 | NHLD | NHLD | NHLD | (SEQ | (SEQ |
| | (SEQ | (SEQ | (SEQ | ID | ID |
| | ID | ID | ID | NO: | NO: |
| | NO: | NO: | NO: | 128) | 131) |
| | 123) | 123) | 123) | | |
| Light Chain | LGSN | LGSN | LGSN | FLIH | LGS |
| variable | RVS | RVS | RVS | LGSN | |
| region | (SEQ | (SEQ | (SEQ | RV | |
| CDR2 | ID | ID | ID | (SEQ | |
| | NO: | NO: | NO: | ID | |
| | 124) | 124) | 124) | NO: | |
| | | | | 129) | |
| Light Chain | MQAL | MQAL | MQAL | MQAL | MQAL |
| variable | RTPY | RTPY | RTPY | RTPY | RTPY |
| region | T | T | T | (SEQ | T |
| CDR3 | (SEQ | (SEQ | (SEQ | ID | (SEQ |
| | ID | ID | ID | NO: | ID |
| | NO: | NO: | NO: | 103) | NO: |
| | 99) | 99) | 99) | | 99) |

VH (SEQ ID NO: 138):
QVQLQQSGPGLVKPSQTLSLTCAISGDSVSSTSVA
WNWIRQSPSGGLEWLGRIYYRSKWYNDYALSVKSR
MTINPDTASNQFSLQLNSVTPEDTAVYYCSRGIVG
ALDYWGQGTLVTVSS
VL (SEQ ID NO: 133):
DIVMTQTPLSLPVTPGGPASISCRSSQSLMYSNGY
NHLDWYLQKPGQSPQFLIHLGSNRVSGVPDRFSGS
GSGTDFTLKISRVEAEDVGVYYCMQALRTPYTFGQ
GTKVDIK

TABLE 10
MSLNB590 Binding Region Sequences

| | Kabat | Chothia | AbM | Contact | IMGT |
|---|---|---|---|---|---|
| Heavy | STRA | GDSV | GDSV | SSTR | GDSV |
| Chain | AWN | SSTR | SSTR | AAWN | SSTR |
| variable | (SEQ | A | AAWN | (SEQ | AA |
| region | ID | (SEQ | (SEQ | ID | (SEQ |
| CDR1 | NO: | ID | ID | NO: | ID |
| | 108) | NO: | NO: | 114) | NO: |
| | | 112) | 113) | | 117) |
| Heavy | RTYY | YYRS | RTYY | WLGR | TYYR |
| Chain | RSKW | KWY | RSKW | TYYR | SKWY |
| variable | YNDY | (SEQ | YND | SKWY | N |
| region | TVSV | ID | (SEQ | ND | (SEQ |
| CDR2 | KS | NO: | ID | (SEQ | ID |
| | (SEQ | 31) | NO: | ID | NO: |
| | ID | | 52) | NO: | 59) |
| | NO: | | | 54) | |
| | 109) | | | | |
| Heavy | GNVG | GNVG | GNVG | ARGN | ARGN |
| Chain | RVDF | RVDF | RVDF | VGRV | VGRV |
| variable | (SEQ | (SEQ | (SEQ | D | DF |
| region | ID | ID | ID | (SEQ | (SEQ |
| CDR3 | NO: | NO: | NO: | ID | ID |
| | 110) | 110) | 110) | NO: | NO: |
| | | | | 115) | 118) |
| Light Chain | RSSQ | RSSQ | RSSQ | LHSD | QSLL |
| variable | SLLH | SLLH | SLLH | GYNY | HSDG |
| region | SDGY | SDGY | SDGY | LDWY | YNY |
| CDR1 | NYLD | NYLD | NYLD | (SEQ | (SEQ |
| | (SEQ | (SEQ | (SEQ | ID | ID |
| | ID | ID | ID | NO: | NO: |
| | NO: | NO: | NO: | 116) | 119) |
| | 111) | 111) | 111) | | |
| Light Chain | LGSN | LGSN | LGSN | LLIY | LGS |
| variable | RAS | RAS | RAS | LGSN | |
| region | (SEQ | (SEQ | (SEQ | RA | |
| CDR2 | ID | ID | ID | (SEQ | |
| | NO: | NO: | NO: | ID | |
| | 28) | 28) | 28) | NO: | |
| | | | | 38) | |
| Light Chain | MQGL | MQGL | MQGL | MQGL | MQGL |
| variable | RTPL | RTPL | RTPL | RTPL | RTPL |
| region | T | T | T | (SEQ | T |
| CDR3 | (SEQ | (SEQ | (SEQ | ID | (SEQ |
| | ID | ID | ID | NO: | ID |
| | NO: | NO: | NO: | 91) | NO: |
| | 87) | 87) | 87) | | 87) |

VH (SEQ ID NO: 120):
QVQLQQSGPGLVKPSQTLSLTCAISGDSVSSTRAAWN
WIRQSPSRGLEWLGRTYYRSKWYNDYTVSVKSRITIN
PDTSKNQFSLHLNSVTPEDSAVYFCARGNVGRVDFWG
QGTLVTVSS
VL (SEQ ID NO: 139):
EIVMTQSPLSLPVTPGEPASISCRSSQSLLHSDGYNY
LDWYLQKPGQSPQLLIYLGSNRASGVPDRFSGSGSDT
DFTLKISRVEAEDVGVYYCMQGLRTPLTFGPGTKLEI
K

TABLE 11
MSLNB591 Binding Region Sequences

| | Kabat | Chothia | AbM | Contact | IMGT |
|---|---|---|---|---|---|
| Heavy | SSSV | GDSV | GDSV | SSSS | GDSV |
| Chain | AWN | SSSS | SSSS | VAWN | SSSS |
| variable | (SEQ | V | VAWN | (SEQ | VA |
| region | ID | (SEQ | (SEQ | ID | (SEQ |
| CDR1 | NO: | ID | ID | NO: | ID |
| | 1) | NO: | NO: | 11) | NO: |
| | | 7) | 9) | | 17) |
| Heavy | RTYY | YYRS | RTYY | WLGR | TYYR |
| Chain | RSKW | KWY | RSKW | TYYR | SKWY |
| variable | YNDY | (SEQ | YND | SKWY | N |
| region | AISV | ID | (SEQ | ND | (SEQ |
| CDR2 | RS | NO: | ID | (SEQ | ID |
| | (SEQ | 31) | NO: | ID | NO: |
| | ID | | 52) | NO: | 59) |
| | NO: | | | 54) | |
| | 96) | | | | |
| Heavy | GVVG | GVVG | GVVG | SRGV | SRGV |
| Chain | ALDY | ALDY | ALDY | VGAL | VGAL |
| variable | (SEQ | (SEQ | (SEQ | D | DY |
| region | ID | ID | ID | (SEQ | (SEQ |
| CDR3 | NO: | NO: | NO: | ID | ID |
| | 97) | 97) | 97) | NO: | NO: |
| | | | | 100) | 104) |
| Light Chain | RSSQ | RSSQ | RSSQ | LFTN | QSLL |
| variable | SLLF | SLLF | SLLF | GYNS | FTNG |
| region | TNGY | TNGY | TNGY | LDWY | YNS |
| CDR1 | NSLD | NSLD | NSLD | (SEQ | (SEQ |
| | (SEQ | (SEQ | (SEQ | ID | ID |
| | ID | ID | ID | NO: | NO: |
| | NO: | NO: | NO: | 101) | 105) |
| | 98) | 98) | 98) | | |
| Light Chain | LVSN | LVSN | LVSN | FLIY | LVS |
| variable | RAS | RAS | RAS | LVSN | |
| region | (SEQ | (SEQ | (SEQ | RA | |
| CDR2 | ID | ID | ID | (SEQ | |
| | NO: | NO: | NO: | ID | |
| | 5) | 5) | 5) | NO: | |
| | | | | 102) | |
| Light Chain | MQAL | MQAL | MQAL | MQAL | MQAL |
| variable | RTPY | RTPY | RTPY | RTPY | RTPY |
| region | T | T | T | (SEQ | T |
| CDR3 | (SEQ | (SEQ | (SEQ | ID | (SEQ |
| | ID | ID | ID | NO: | ID |
| | NO: | NO: | NO: | 103) | NO: |
| | 99) | 99) | 99) | | 99) |

VH (SEQ ID NO: 106):
QVQLQQSGPGLVKPSQTLSLTCGISGDSVSSSSV
AWNWIRQSPSRGLEWLGRTYYRSKWYNDYAISVR
SRVTINPDTSKNQFSLQLNSVTPEDTAVYYCSRG
VVGALDYWGQGTLVTVSS
VL (SEQ ID NO: 140):
DIVMTQSPLSLTVTPGEPASISCRSSQSLLFTNGY
NSLDWYLQKPGQSPQFLIYLVSNRASGVPDRFSGS
GSGTDFTLKISRVEAEDVGVYYCMQALRTPYTFGQ
GTKVDIK

TABLE 12
MSLNB592 Binding Region Sequences

| | Kabat | Chothia | AbM | Contact | IMGT |
|---|---|---|---|---|---|
| Heavy | STSV | GDSV | GDSV | SSTS | GDSV |
| Chain | AWN | SSTS | SSTS | VAWN | SSTS |
| variable | (SEQ | V | VAWN | (SEQ | VA |
| region | ID | (SEQ | (SEQ | ID | (SEQ |
| CDR1 | NO: | ID | ID | NO: | ID |
| | 122) | NO: | NO: | 127) | NO: |
| | | 125) | 126) | | 130) |
| Heavy | RTYY | YYRS | RTYY | WLGR | TYYR |
| Chain | RSKW | KWY | RSKW | TYYR | SKWY |
| variable | YNDY | (SEQ | YND | SKWY | N |
| region | ALSV | ID | (SEQ | ND | (SEQ |
| CDR2 | KS | NO: | ID | (SEQ | ID |
| | (SEQ | 31) | NO: | ID | NO: |
| | ID | | 52) | NO: | 59) |
| | NO: | | | 54) | |
| | 84) | | | | |
| Heavy | GIVG | GIVG | GIVG | SRGI | SRGI |
| Chain | ALDY | ALDY | ALDY | VGAL | VGAL |
| variable | (SEQ | (SEQ | (SEQ | D | DY |
| region | ID | ID | ID | (SEQ | (SEQ |
| CDR3 | NO: | NO: | NO: | ID | ID |
| | 3) | 3) | 3) | NO: | NO: |
| | | | | 13) | 19) |
| Light | RSSQ | RSSQ | RSSQ | MYSN | QSLM |
| Chain | SLMY | SLMY | SLMY | GYNH | YSNG |
| variable | SNGY | SNGY | SNGY | LDWY | YNH |
| region | NHLD | NHLD | NHLD | (SEQ | (SEQ |
| CDR1 | (SEQ | (SEQ | (SEQ | ID | ID |
| | ID | ID | ID | NO: | NO: |
| | NO: | NO: | NO: | 128) | 131) |
| | 123) | 123) | 123) | | |
| Light | LGSN | LGSN | LGSN | FLIH | LGS |
| Chain | RAS | RAS | RAS | LGSN | |
| variable | (SEQ | (SEQ | (SEQ | RA | |
| region | ID | ID | ID | (SEQ | |
| CDR2 | NO: | NO: | NO: | ID | |
| | 28) | 28) | 28) | NO: | |
| | | | | 141) | |
| Light | MQAL | MQAL | MQAL | MQAL | MQAL |
| Chain | RTPY | RTPY | RTPY | RTPY | RTPY |
| variable | T | T | T | (SEQ | T |
| region | (SEQ | (SEQ | (SEQ | ID | (SEQ |
| CDR3 | ID | ID | ID | NO: | ID |
| | NO: | NO: | NO: | 103) | NO: |
| | 99) | 99) | 99) | | 99) |

VH (SEQ ID NO: 132):
QVQLQQSGPGLVKPSQTLSLTCAISGDSVSSTSVA
WNWIRQSPSGGLEWLGRTYYRSKWYNDYALSVKSR
MTINPDTASNQFSLQLNSVTPEDTAVYYCSRGIVG
ALDYWGQGTLVTVSS
VL (SEQ ID NO: 142):
DIVMTQSPLSLPVTPGEPASISCRSSQSLMYSNGY
NHLDWYLQKPGQSPQFLIHLGSNRASGVPDRFSGS
GSGTDFTLKISRVEAEDVGVYYCMQALRTPYTFGQ
GTKVEIK

In some embodiments, the MSLN binding agent described herein binds MSLN at the membrane-restricted region. In some embodiments, the MSLN binding agent described herein binds MSLN that is full length MSLN. In some embodiments, the MSLN binding agent described herein binds MSLN that is less than the full-length MSLN. In particular embodiments, MSLN that is bound by the present MSLN binding agent is a membrane-associated fragment of MSLN.

In particular embodiments, MSLN that is bound by the present MSLN binding agent is the membrane-associated portion of MSLN produced by processing of MSLN catalyzed by one or more enzymes. In some embodiments, MSLN that is bound by the present MSLN binding agent is the membrane-associated portion of MSLN produced by protease cleavage of MSLN. In some embodiments, MSLN that is bound by the present MSLN binding agent is the membrane-associated portion of MSLN produced by cleavage by a member of the matrix metalloproteinase (MMP) disintegrin and metalloprotease family. In some embodiments, MSLN that is bound by the present MSLN binding agent is the membrane-associated portion of MSLN produced by sheddase TACE cleavage. In some embodiments, MSLN that is bound by the present MSLN binding agent is the membrane-associated portion of MSLN produced by proteases (ADAM)10 cleavage. In some embodiments, MSLN that is bound by the present MSLN binding agent is the membrane-associated portion of MSLN produced by proteases ADAM17 cleavage. In some embodiments, MSLN that is bound by the present MSLN binding agent is the membrane-associated portion of MSLN produced by proteases b-secretase (BACE)2 cleavage. In some embodiments, MSLN that is bound by the present MSLN binding agent is the membrane-associated portion of MSLN produced by proteases BACE1 cleavage. In some embodiments, MSLN that is bound by the present MSLN binding agent is the membrane-associated portion of MSLN produced by proteases MMP15 cleavage.

In some embodiments, the MSLN binding agent does not bind any MSLN polypeptide that lacks the membrane-restricted region. In some embodiments, the MSLN binding agent does not bind to any soluble MSLN isoform. In some embodiments, the MSLN binding agent does not bind to any species of shed MSLN. In some embodiments, the MSLN binding agent does not bind to any soluble MSLN isoform and does not bind to any species of shed MSLN. In some embodiments, the MSLN binding agent does not bind to any species of shed MSLN produced by processing of MSLN by any enzymes selected from sheddase TACE, disintegrin and metalloproteinase (ADAM)10, ADAM17, b-secretase (BACE)2, BACE1, and MMP15. In some embodiments, the membrane-restricted region corresponds to residues 587-598 within an amino acid sequence of SEQ ID NO:200. In some embodiments, the membrane-restricted region corresponds to residues 592-598 within an amino acid sequence of SEQ ID NO:200.

In particular embodiments, the MSLN binding agent binds to MSLN at least one of residues corresponding to residues 587-598 within an amino acid sequence of SEQ ID NO:200. In some embodiments, the MSLN binding agent, when bound to MSLN, binds to at least one residue selected from the group consisting of L589, D590, M593, V588, S592, L597, E595, and A596 with an amino acid sequence of SEQ ID NO:200. In some embodiments, the MSLN binding molecule, when bound to MSLN, binds to at least the residue corresponding to L589 in the amino acid sequence of SEQ ID NO:200. In some embodiments, the MSLN binding molecule, when bound to MSLN, binds to at least the residue corresponding to D590 in the amino acid sequence of SEQ ID NO:200. In some embodiments, the MSLN binding molecule, when bound to MSLN, binds to at least the residue corresponding to M593 in the amino acid sequence of SEQ ID NO:200. In some embodiments, the MSLN binding molecule, when bound to MSLN, binds to at least the residue corresponding to V588 in the amino acid sequence of SEQ ID NO:200. In some embodiments, the MSLN binding molecule, when bound to MSLN, binds to at least the residue corresponding to S592 in the amino acid sequence of SEQ ID NO:200. In some embodiments, the MSLN binding molecule, when bound to MSLN, binds to at least the residue corresponding to L597 in the amino acid sequence of SEQ ID NO:200. In some embodiments, the MSLN binding molecule, when bound to MSLN, binds to at least the residue corresponding to E595 in the amino acid sequence of SEQ ID NO:200. In some embodiments, the MSLN binding molecule, when bound to MSLN, binds to at least the residue corresponding to A596 in the amino acid sequence of SEQ ID NO:200.

In some embodiments, the MSLN binding molecule, when bound to MSLN, binds to at least the residues corresponding to L589, D590, and M593 in the amino acid sequence of SEQ ID NO:200. In some embodiments, the MSLN binding molecule, when bound to MSLN, binds to at least the residues corresponding to V588, S592, E595, and L597 in the amino acid sequence of SEQ ID NO:200. In some embodiments, the MSLN binding molecule, when bound to MSLN, binds to at least the residues corresponding to V588, S592, and L597 in the amino acid sequence of SEQ ID NO:200.

In some embodiments, upon binding to the MSLN molecule, the present MSLN-binding molecule binds to the cell expressing the MSLN protein. In some embodiments, the MSLN-expressing cell is a cancer cell. In some embodiments, the cancer cell is selected from mesothelioma, serous ovarian carcinoma, and pancreatic cancer. In some embodiments, the cancer cell is mesothelioma, ovarian carcinoma, and pancreatic cancer.

7.4 Antibody Methodology

In some embodiments, the MSLN binding agents described herein are anti-MSLN antibodies. In some embodiments, the MSLN binding agents described herein comprises one or more domain or fragments derived from an antibody (e.g., an anti-MSLN antibody). In some embodiments, the MSLN binding agents described herein can be generated with methods and processes for the generation, selection, modification, and fragmentation, etc. of antibody molecules that are known in the art.

7.4.1 Polyclonal Antibodies

In some embodiments, the anti-MSLN antibodies of the present disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a MSLN polypeptide or a fusion protein thereof. It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly 1C, Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for MSLN antibody titer. If desired, the mammal can be boosted until the antibody titer increases or plateaus. Additionally or alternatively, lymphocytes may be obtained from the immunized animal for fusion and preparation of monoclonal antibodies from hybridoma as described below.

7.4.2 Monoclonal Antibodies

The antibodies of the present disclosure may alternatively be monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. In some embodiments, the immunizing antigen is a human protein or a fragment thereof. In some embodiments, the immunizing antigen is a mouse protein or a fragment thereof. In some embodiments, the immunizing antigen is a cyno protein or a fragment thereof. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In some embodiments, an antibody that binds an MSLN epitope comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2×SSC/0.1% SDS at about 50-65°) C., under highly stringent conditions (e.g., hybridization to filter-bound nucleic acid in 6×SSC at about 45° C. followed by one or more washes in 0.1×SSC/0.2% SDS at about 68°) C., or under other stringent hybridization conditions which are known to those of skill in the art. See, e.g., Current Protocols in Molecular Biology Vol. I, 6.3.1-6.3.6 and 2.10.3 (Ausubel et al. eds., 1989).

In some embodiments, an antibody that binds an MSLN epitope comprises an amino acid sequence of a HCDR or an amino acid sequence of a LCDR depicted in Tables 1-12 under stringent conditions (e.g., hybridization to filter-bound DNA in 6×SSC at about 45° C. followed by one or more washes in 0.2×SSC/0.1% SDS at about 50-65°) C., under highly stringent conditions (e.g., hybridization to filter-bound nucleic acid in 6×SSC at about 45° C. followed by one or more washes in 0.1×SSC/0.2% SDS at about 68°) C., or under other stringent hybridization conditions which are known to those of skill in the art (see, e.g., Ausubel et al., supra).

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, MSLN (e.g., a MSLN polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-MSLN antibodies can be obtained by designing a suitable antigen screening procedure to select for the phage clone of interest followed by construction of a full length anti-MSLN antibody clone using VH and/or VL sequences (e.g., the Fv sequences), or various CDR sequences from VH and VL sequences, from the phage clone of interest and suitable constant region (e.g., Fc) sequences described in Kabat et al., supra.

In another embodiment, anti-MSLN antibody is generated by using methods as described in Bowers et al., 2011, Proc Natl Acad Sci USA. 108:20455-60, e.g., the SHM-XHL™ platform (AnaptysBio, San Diego, CA). Briefly, in this approach, a fully human library of IgGs is constructed in a mammalian cell line (e.g., HEK293) as a starting library. Mammalian cells displaying immunoglobulin that binds to a target peptide or epitope are selected (e.g., by FACS sorting), then activation-induced cytidine deaminase (AID)-triggered somatic hypermutation is reproduced in vitro to expand diversity of the initially selected pool of antibodies. After several rounds of affinity maturation by coupling mammalian cell surface display with in vitro somatic hypermutation, high affinity, high specificity anti-MSLN antibodies are generated. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

7.4.3 Antibody Fragments

The present disclosure provides antibodies and antibody fragments that bind to MSLN. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from F. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to a MSLN epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to a MSLN epitope. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

In some embodiments, an MSLN binding region is an antigen binding fragment of an antibody. In some embodiments, the MSLN binding region is a portion of an intact antibody. In some embodiments, the MSLN binding region is a Fab, Fab′, F(ab′)₂, Fv, single chain antibody molecules(e.g., scFv), disulfide-linked scFv (dsscFv). In some embodiments, the MSLN binding region is a Fab. In other specific embodiments, an MSLN binding region comprises two Fabs. In other specific embodiments, an MSLN binding region comprises two Fabs in tandem. In some embodiments, the MSLN binding region is a scFv. In some embodiments, the MSLN binding region is selected from nanobodies, diabodies, tribodies, tetrabodies, minibodies, dual variable domain antibodies (DVD), single variable domain antibodies (e.g., camelid antibodies). Any of the VH and the VL domains described herein that bind MSLN can be engineered into the binding molecule in the various formats described above, and their binding to MSLN and thermostability may be assessed using the assays described herein.

In specific embodiments, an MSLN binding region comprises an scFv that binds MSLN. Any of the VH and the VL domains described herein that bind MSLN may be engineered into scFv format in either VH-linker-VL or VL-linker-VH orientation. Alternatively, the VH and VL domains may be engineered into scFv format without the use of a linker in either the VH-VL or VL-VH orientation. Accordingly, in some embodiments, the MSLN binding agent described herein comprises an scFv that binds MSLN in the format of VH-linker-VL. In some embodiments, the MSLN binding agent described herein comprises an scFv that binds MSLN in the format of VL-linker-VH. In some embodiments, the MSLN binding agent described herein comprises an scFv that binds MSLN in the format of VH-VL. In some embodiments, the MSLN binding agent described herein comprises an scFv that binds MSLN in the format of VL-VH.

In particular embodiments, the VH forming any of the scFv described herein is selected from the VH sequences disclosed in any of Tables 1 to 12. In particular embodiments, the VH forming any of the scFv described herein is selected from SEQ ID NOS:22, 45, 62, 81, 94, 106, 120, 132, and 138. In particular embodiments, the VL forming any of the scFv described herein is selected from the VL sequences disclosed in any of Tables 1 to 12. In particular embodiments, the VL forming any of the scFv described herein is selected from SEQ ID NOS:23, 46, 63, 82, 95, 107, 121, 133, 139, 140, and 142. In particular embodiments, the VH forming any of the scFv described herein is selected from SEQ ID NOS:22, 45, 62, 81, 94, 106, 120, 132, and 138, and the VL forming any of the scFv described herein is selected from SEQ ID NOS:23, 46, 63, 82, 95, 107, 121, 133, 139, 140, and 142.

In particular embodiments, the scFv comprises the VH and VL sequences disclosed in any of Tables 1 to 12. In some embodiments, the scFv comprises the VH comprising SEQ ID NO: 22 and the VL comprising SEQ ID NO: 23. In particular embodiments, the VH comprising SEQ ID NO:22 and the VL comprising SEQ ID NO:23 are fused directly with one another without a linker in the scFv. In some embodiments, the VH comprising SEQ ID NO:22 is fused to the N terminus of the VL comprising SEQ ID NO:23 in the VH-VL orientation. In other embodiments, the VH comprising SEQ ID NO:22 is fused to the C terminus of the VL comprising SEQ ID NO:23 in the VL-VH orientation. In some embodiments, the VH comprising SEQ ID NO:22 and the VL comprising SEQ ID NO:23 are linked with a linker in the scFv. In some embodiments, the VH comprising SEQ ID NO:22 and the VL comprising SEQ ID NO:23 and the linker are fused in the VH-linker-VL orientation in the scFv. In other embodiments, the VH comprising SEQ ID NO:22 and the VL comprising SEQ ID NO:23 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 45 and the VL comprising SEQ ID NO: 46. In particular embodiments, the VH comprising SEQ ID NO:45 and the VL comprising SEQ ID NO:46 are fused directly with one another without a linker in the scFv. In some embodiments, the VH comprising SEQ ID NO:45 is fused to the N terminus of the VL comprising SEQ ID NO:46 in the VH-VL orientation. In other embodiments, the VH comprising SEQ ID NO:45 is fused to the C terminus of the VL comprising SEQ ID NO:46 in the VL-VH orientation. In some embodiments, the VH comprising SEQ ID NO:45 and the VL comprising SEQ ID NO:46 are linked with a linker in the scFv. In some embodiments, the VH comprising SEQ ID NO:45 and the VL comprising SEQ ID NO:46 and the linker are fused in the VH-linker-VL orientation in the scFv. In other embodiments, the VH comprising SEQ ID NO:45 and the VL comprising SEQ ID NO:46 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 62 and the VL comprising SEQ ID NO: 63. In particular embodiments, the VH comprising SEQ ID NO:62 and the VL comprising SEQ ID NO:63 are fused directly with one another without a linker in the scFv. In some embodiments, the VH comprising SEQ ID NO:62 is fused to the N terminus of the VL comprising SEQ ID NO:63 in the VH-VL orientation. In other embodiments, the VH comprising SEQ ID NO:62 is fused to the C terminus of the VL comprising SEQ ID NO:63 in the VL-VH orientation. In some embodiments, the VH comprising SEQ ID NO:62 and the VL comprising SEQ ID NO:63 are linked with a linker in the scFv. In some embodiments, the VH comprising SEQ ID NO:62 and the VL comprising SEQ ID NO:63 and the linker are fused in the VH-linker-VL orientation in the scFv. In other embodiments, the VH comprising SEQ ID NO:62 and the VL comprising SEQ ID NO:63 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 81 and the VL comprising SEQ ID NO: 82. In particular embodiments, the VH comprising SEQ ID NO:81 and the VL comprising SEQ ID NO:82 are fused directly with one another without a linker in the scFv. In some embodiments, the VH comprising SEQ ID NO:81 is fused to the N terminus of the VL comprising SEQ ID NO:82 in the VH-VL orientation. In other embodiments, the VH comprising SEQ ID NO:81 is fused to the C terminus of the VL comprising SEQ ID NO:82 in the VL-VH orientation. In some embodiments, the VH comprising SEQ ID NO:81 and the VL comprising SEQ ID NO:82 are linked with a linker in the scFv. In some embodiments, the VH comprising SEQ ID NO:81 and the VL comprising SEQ ID NO:82 and the linker are fused in the VH-linker-VL orientation in the scFv. In other embodiments, the VH comprising SEQ ID NO:81 and the VL comprising SEQ ID NO:82 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 94 and the VL comprising SEQ ID NO: 95. In particular embodiments, the VH comprising SEQ ID NO:94 and the VL comprising SEQ ID NO:95 are fused directly with one another without a linker in the scFv. In some embodiments, the VH comprising SEQ ID NO:94 is fused to the N terminus of the VL comprising SEQ ID NO:95 in the VH-VL orientation. In other embodiments, the VH comprising SEQ ID NO:94 is fused to the C terminus of the VL comprising SEQ ID NO:95 in the VL-VH orientation. In some embodiments, the VH comprising SEQ ID NO:94 and the VL comprising SEQ ID NO:95 are linked with a linker in the scFv. In some embodiments, the VH comprising SEQ ID NO:94 and the VL comprising SEQ ID NO:95 and the linker are fused in the VH-linker-VL orientation in the scFv. In other embodiments, the VH comprising SEQ ID NO:94 and the VL comprising SEQ ID NO:95 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 106 and the VL comprising SEQ ID NO: 107. In particular embodiments, the VH comprising SEQ ID NO:106 and the VL comprising SEQ ID NO: 107 are fused directly with one another without a linker in the scFv. In some embodiments, the VH comprising SEQ ID NO: 106 is fused to the N terminus of the VL comprising SEQ ID NO: 107 in the VH-VL orientation. In other embodiments, the VH comprising SEQ ID NO: 106 is fused to the C terminus of the VL comprising SEQ ID NO: 107 in the VL-VH orientation. In some embodiments, the VH comprising SEQ ID NO:106 and the VL comprising SEQ ID NO: 107 are linked with a linker in the scFv. In some embodiments, the VH comprising SEQ ID NO: 106 and the VL comprising SEQ ID NO: 107 and the linker are fused in the VH-linker-VL orientation in the scFv. In other embodiments, the VH comprising SEQ ID NO: 106 and the VL comprising SEQ ID NO: 107 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 120 and the VL comprising SEQ ID NO: 121. In particular embodiments, the VH comprising SEQ ID NO:120 and the VL comprising SEQ ID NO: 121 are fused directly with one another without a linker in the scFv. In some embodiments, the VH comprising SEQ ID NO: 120 is fused to the N terminus of the VL comprising SEQ ID NO: 121 in the VH-VL orientation. In other embodiments, the VH comprising SEQ ID NO: 120 is fused to the C terminus of the VL comprising SEQ ID NO:121 in the VL-VH orientation. In some embodiments, the VH comprising SEQ ID NO:120 and the VL comprising SEQ ID NO: 121 are linked with a linker in the scFv. In some embodiments, the VH comprising SEQ ID NO: 120 and the VL comprising SEQ ID NO: 121 and the linker are fused in the VH-linker-VL orientation in the scFv. In other embodiments, the VH comprising SEQ ID NO: 120 and the VL comprising SEQ ID NO: 121 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 132 and the VL comprising SEQ ID NO: 133. In particular embodiments, the VH comprising SEQ ID NO:132 and the VL comprising SEQ ID NO: 133 are fused directly with one another without a linker in the scFv. In some embodiments, the VH comprising SEQ ID NO: 132 is fused to the N terminus of the VL comprising SEQ ID NO: 133 in the VH-VL orientation. In other embodiments, the VH comprising SEQ ID NO: 132 is fused to the C terminus of the VL comprising SEQ ID NO:133 in the VL-VH orientation. In some embodiments, the VH comprising SEQ ID NO: 132 and the VL comprising SEQ ID NO:133 are linked with a linker in the scFv. In some embodiments, the VH comprising SEQ ID NO: 132 and the VL comprising SEQ ID NO: 133 and the linker are fused in the VH-linker-VL orientation in the scFv. In other embodiments, the VH comprising SEQ ID NO: 132 and the VL comprising SEQ ID NO: 133 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 138 and the VL comprising SEQ ID NO: 133. In particular embodiments, the VH comprising SEQ ID NO:138 and the VL comprising SEQ ID NO: 133 are fused directly with one another without a linker in the scFv. In some embodiments, the VH comprising SEQ ID NO: 138 is fused to the N terminus of the VL comprising SEQ ID NO: 133 in the VH-VL orientation. In other embodiments, the VH comprising SEQ ID NO: 138 is fused to the C terminus of the VL comprising SEQ ID NO: 133 in the VL-VH orientation. In some embodiments, the VH comprising SEQ ID NO: 138 and the VL comprising SEQ ID NO: 133 are linked with a linker in the scFv. In some embodiments, the VH comprising SEQ ID NO: 138 and the VL comprising SEQ ID NO: 133 and the linker are fused in the VH-linker-VL orientation in the scFv. In other embodiments, the VH comprising SEQ ID NO:138 and the VL comprising SEQ ID NO: 133 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 120 and the VL comprising SEQ ID NO: 139. In particular embodiments, the VH comprising SEQ ID NO: 120 and the VL comprising SEQ ID NO: 139 are fused directly with one another without a linker in the scFv. In some embodiments, the VH comprising SEQ ID NO: 120 is fused to the N terminus of the VL comprising SEQ ID NO: 139 in the VH-VL orientation. In other embodiments, the VH comprising SEQ ID NO: 120 is fused to the C terminus of the VL comprising SEQ ID NO: 139 in the VL-VH orientation. In some embodiments, the VH comprising SEQ ID NO: 120 and the VL comprising SEQ ID NO: 139 are linked with a linker in the scFv. In some embodiments, the VH comprising SEQ ID NO: 120 and the VL comprising SEQ ID NO: 139 and the linker are fused in the VH-linker-VL orientation in the scFv. In other embodiments, the VH comprising SEQ ID NO: 120 and the VL comprising SEQ ID NO: 139 and the linker are fused in the VL-linker-VH orientation in the scFv. In some embodiments described in the present paragraph, the linker connecting the VH and VL in the scFv comprises a sequence selected from SEQ ID NOS:166-199.

In some embodiments, the scFv comprises the VH comprising SEQ ID NO: 106 and the VL comprising SEQ ID NO: 140. In particular embodiments, the VH comprising SEQ ID NO:106 and the VL comprising SEQ ID NO: 140 are fused directly with one another without a linker in the scFv. In some embodiments, the VH comprising SEQ ID NO: 106 is fused to the N terminus of the VL comprising SEQ ID NO: 140 in the VH-VL orientation. In other embodiments, the VH comprising SEQ ID NO: 106 is fused to the C terminus of the VL comprising SEQ ID NO: 140 in the VL-VH orientation. In some embodiments, the VH comprising SEQ ID NO: 106 and the VL comprising SEQ ID NO: 140 are linked with a linker in the scFv. In some embodiments, the VH comprising SEQ ID NO: 106 and the VL comprising SEQ ID NO: 140 and the linker are fused in the VH-linker-VL orientation in the scFv. In other embodiments, the VH comprising SEQ ID NO: 106 and the VL comprising SEQ ID NO: 140 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the scFv comprises the VH comprising SEQ ID NO: 132 and the VL comprising SEQ ID NO: 142. In particular embodiments, the VH comprising SEQ ID NO:132 and the VL comprising SEQ ID NO:142 are fused directly with one another without a linker in the scFv. In some embodiments, the VH comprising SEQ ID NO: 132 is fused to the N terminus of the VL comprising SEQ ID NO: 142 in the VH-VL orientation. In other embodiments, the VH comprising SEQ ID NO: 132 is fused to the C terminus of the VL comprising SEQ ID NO: 142 in the VL-VH orientation. In some embodiments, the VH comprising SEQ ID NO: 132 and the VL comprising SEQ ID NO: 142 are linked with a linker in the scFv. In some embodiments, the VH comprising SEQ ID NO:132 and the VL comprising SEQ ID NO: 142 and the linker are fused in the VH-linker-VL orientation in the scFv. In other embodiments, the VH comprising SEQ ID NO: 132 and the VL comprising SEQ ID NO: 142 and the linker are fused in the VL-linker-VH orientation in the scFv. In some embodiments described in the present paragraph, the linker connecting the VH and VL in the scFv comprises a sequence selected from SEQ ID NOS: 166-199.

In some embodiments, the VH and the VL domains identified herein may be incorporated into a scFv format and the binding and thermostability of the resulting scFv to MSLN can be assessed using known methods. In some embodiments, binding of the resulting scFv to MSLN can be assessed using ProteOn XPR36, Biacore 3000 or KinExA instrumentation, ELISA or competitive binding assays known to those skilled in the art. In some embodiments, binding of the resulting scFv to MSLN can be evaluated using purified scFvs or E. coli supernatants or lysed cells containing the expressed scFv. In some embodiments, the measured affinity of a test scFv to MSLN may vary if measured under different conditions (e.g., osmolarity, pH). Thus, measurements of affinity and other binding parameters (e.g., KD, Kon, Koff) are typically made with standardized conditions and standardized buffers. Thermostability may be evaluated by heating the test scFv at elevated temperatures, such as at 50° C., 55° C. or 60ºC for a period of time, such as 5 minutes (min), 10 min, 15 min, 20 min, 25 min or 30 min and measuring binding of the test scFv to MSLN. The scFvs retaining comparable binding to MSLN when compared to a non-heated scFv sample are referred to as being thermostable.

In recombinant expression systems, the linker is a peptide linker and may include any naturally occurring amino acid. Exemplary amino acids that can be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to MSLN.

In some embodiments, the linker is about 5-50 amino acids long. In other embodiments, the linker is about 10-40 amino acids long. In other embodiments, the linker is about 10-35 amino acids long. In other embodiments, the linker is about 10-30 amino acids long. In other embodiments, the linker is about 10-25 amino acids long. In other embodiments, the linker is about 10-20 amino acids long. In other embodiments, the linker is about 15-20 amino acids long. In other embodiments, the linker is about 16-19 amino acids long. In other embodiments, the linker is 6 amino acids long. In other embodiments, the linker is 7 amino acids long. In other embodiments, the linker is 8 amino acids long. In other embodiments, the linker is 9 amino acids long. In other embodiments, the linker is 10 amino acids long. In other embodiments, the linker is 11 amino acids long. In other embodiments, the linker is 12 amino acids long. In other embodiments, the linker is 13 amino acids long. In other embodiments, the linker is 14 amino acids long. In other embodiments, the linker is 15 amino acids long. In other embodiments, the linker is 16 amino acids long. In other embodiments, the linker is 17 amino acids long. In other embodiments, the linker is 18 amino acids long. In other embodiments, the linker is 19 amino acids long. In other embodiments, the linker is 20 amino acids long. In other embodiments, the linker is 21 amino acids long. In other embodiments, the linker is 22 amino acids long. In other embodiments, the linker is 23 amino acids long. In other embodiments, the linker is 24 amino acids long. In other embodiments, the linker is 25 amino acids long. In other embodiments, the linker is 26 amino acids long. In other embodiments, the linker is 27 amino acids long. In other embodiments, the linker is 28 amino acids long. In other embodiments, the linker is 29 amino acids long. In other embodiments, the linker is 30 amino acids long. In other embodiments, the linker is 31 amino acids long. In other embodiments, the linker is 32 amino acids long. In other embodiments, the linker is 33 amino acids long. In other embodiments, the linker is 34 amino acids long. In other embodiments, the linker is 35 amino acids long. In other embodiments, the linker is 36 amino acids long. In other embodiments, the linker is 37 amino acids long. In other embodiments, the linker is 38 amino acids long. In other embodiments, the linker is 39 amino acids long. In other embodiments, the linker is 40 amino acids long. Exemplary linkers that can be used are Gly rich linkers, Gly and Ser containing linkers, Gly and Ala containing linkers, Ala and Ser containing linkers, and other flexible linkers.

Other linker sequences can include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Exemplary linkers that may be used are shown in the following Table. Additional linkers are described for example in Int. Pat. Publ. No. WO2019/060695.

Examples of Linker Sequences

| | | SEQ |
|---|---|---|
| Linker | | ID |
| name | Amino acid sequence | NO: |
| Linker 1 | GGSEGKSSGSGSESKSTGGS | 166 |
| Linker 2 | GGGSGGGS | 167 |
| Linker 3 | GGGSGGGSGGGS | 168 |
| Linker 4 | GGGSGGGSGGGSGGGS | 169 |
| Linker 5 | GGGSGGGSGGGSGGGSGGGS | 170 |
| Linker 6 | GGGGSGGGGSGGGGS | 171 |
| Linker 7 | GGGGSGGGGSGGGGSGGGGS | 172 |
| Linker 8 | GGGGSGGGGSGGGGSGGGGSGGGGS | 173 |
| Linker 9 | GSTSGSGKPGSGEGSTKG | 174 |
| Linker 10 | IRPRAIGGSKPRVA | 175 |
| Linker 11 | GKGGSGKGGSGKGGS | 176 |
| Linker 12 | GGKGSGGKGSGGKGS | 177 |
| Linker 13 | GGGKSGGGKSGGGKS | 178 |
| Linker 14 | GKGKSGKGKSGKGKS | 179 |
| Linker 15 | GGGKSGGKGSGKGGS | 180 |
| Linker 16 | GKPGSGKPGSGKPGS | 181 |
| Linker 17 | GKPGSGKPGSGKPGSGKPGS | 182 |
| Linker 18 | GKGKSGKGKSGKGKSGKGKS | 183 |
| Linker 19 | STAGDTHLGGEDFD | 184 |
| Linker 20 | GEGGSGEGGSGEGGS | 185 |
| Linker 21 | GGEGSGGEGSGGEGS | 186 |
| Linker 22 | GEGESGEGESGEGES | 187 |
| Linker 23 | GGGESGGEGSGEGGS | 188 |
| Linker 24 | GEGESGEGESGEGESGEGES | 189 |
| Linker 25 | GSTSGSGKPGSGEGSTKG | 190 |
| Linker 26 | PRGASKSGSASQTGSAPGS | 191 |
| Linker 27 | GTAAAGAGAAGGAAAGAAG | 192 |
| Linker 28 | GTSGSSGSGSGGSGSGGGG | 193 |
| Linker 29 | GKPGSGKPGSGKPGSGKPGS | 194 |
| Linker 30 | GSGS | 195 |
| Linker 31 | APAPAPAPAP | 196 |
| Linker 32 | APAPAPAPAPAPAPAPAPAP | 197 |
| Linker 33 | AEAAAKEAAAKEAAAAKEAAAA | 198 |
| | KEAAAAKAAA | |
| Linker 34 | GTEGKSSGSGSESKST | 199 |

In particular embodiments, the scFv comprises, from the N- to C-terminus, a VH, a first linker (L1) and a VL (VH-L1-VL). In particular embodiments, the scFv comprises, from the N to C-terminus, the VL, the L1 and the VH (VL-L1-VH). In particular embodiments, the L1 comprises the amino acid sequence of SEQ ID NO: 166. In particular embodiments, the L1 comprises the amino acid sequence of SEQ ID NO: 167. In particular embodiments, the L1 comprises the amino acid sequence of SEQ ID NO: 168. In particular embodiments, the L1 comprises the amino acid sequence of SEQ ID NO: 169. In particular embodiments, the L1 comprises the amino acid sequence of SEQ ID NO: 170. In particular embodiments, the L1 comprises the amino acid sequence of SEQ ID NO: 171. In particular embodiments, the L1 comprises the amino acid sequence of SEQ ID NO: 172. In particular embodiments, the L1 comprises the amino acid sequence of SEQ ID NO: 173. In particular embodiments, the L1 comprises the amino acid sequence of SEQ ID NO: 174. In particular embodiments, the L1 comprises the amino acid sequence of SEQ ID NO: 175. In particular embodiments, the L1 comprises the amino acid sequence of SEQ ID NO: 176. In particular embodiments, the L1 comprises the amino acid sequence of SEQ ID NO: 177. In particular embodiments, the L1 comprises the amino acid sequence of SEQ ID NO: 178. In particular embodiments, the L1 comprises the amino acid sequence of SEQ ID NO: 179. In particular embodiments, the L1 comprises the amino acid sequence of SEQ ID NO: 180. In particular embodiments, the L1 comprises the amino acid sequence of SEQ ID NO: 181. In particular embodiments, the L1 comprises the amino acid sequence of SEQ ID NO: 182. In particular embodiments, the L1 comprises the amino acid sequence of SEQ ID NO: 183. In particular embodiments, the L1 comprises the amino acid sequence of SEQ ID NO: 184. In particular embodiments, the L1 comprises the amino acid sequence of SEQ ID NO: 185. In particular embodiments, the L1 comprises the amino acid sequence of SEQ ID NO: 186. In particular embodiments, the L1 comprises the amino acid sequence of SEQ ID NO: 187. In particular embodiments, the L1 comprises the amino acid sequence of SEQ ID NO: 188. In particular embodiments, the L1 comprises the amino acid sequence of SEQ ID NO: 189. In particular embodiments, the L1 comprises the amino acid sequence of SEQ ID NO: 190. In particular embodiments, the L1 comprises the amino acid sequence of SEQ ID NO: 191. In particular embodiments, the L1 comprises the amino acid sequence of SEQ ID NO: 192. In particular embodiments, the L1 comprises the amino acid sequence of SEQ ID NO: 193. In particular embodiments, the L1 comprises the amino acid sequence of SEQ ID NO: 194. In particular embodiments, the L1 comprises the amino acid sequence of SEQ ID NO: 195. In particular embodiments, the L1 comprises the amino acid sequence of SEQ ID NO: 196. In particular embodiments, the L1 comprises the amino acid sequence of SEQ ID NO: 197. In particular embodiments, the L1 comprises the amino acid sequence of SEQ ID NO: 198. In particular embodiments, the L1 comprises the amino acid sequence of SEQ ID NO: 199.

7.4.4 Multivalent Antibodies

Without being bound by any theory, it is contemplated that a multivalent antibody may be internalized (and/or catabolized) faster than a monovalent antibody by a cell expressing an antigen to which the antibodies bind, and a multivalent antibody with three or more antigen binding sites may be internalized (and/or catabolized) faster than a bivalent antibody. In some embodiments, the binding agents of the present disclosure can be a bivalent antibody with two antigen binding sites. In some embodiments, the binding agents of the present disclosure can be multivalent antibodies (which are other than of the IgM class) with three or more antigen binding sites (e.g., tetravalent antibodies). In some embodiments, the multivalent antibodies can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. In some embodiments, the multivalent antibody comprises at least one polypeptide chain (e.g., two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In specific embodiments, the multivalent binding agents disclosed herein comprises one or more bivalent sc(Fv)₂ structures. In particular embodiments, any of the VH and the VL domains identified herein may also be used to generate bivalent sc(Fv)₂ structures, such as in the bivalent format of VH-linker-VL-linker-VL-linker-VH, VH-linker-VL-linker-VH-linker-VL, VH-linker-VH-linker-VL-linker-VL, VL-linker-VH-linker-VH-linker-VL, VL-linker-VH-linker-VL-linker-VH, or VL-linker-VL-linker-VH-linker-VH. In specific embodiments, the MSLN binding agent described herein comprises an sc(Fv)₂ that binds MSLN in the format of VH-linker-VL-linker-VL-linker-VH. In some embodiments, the MSLN binding agent described herein comprises an sc(Fv)₂ that binds MSLN in the format of VH-linker-VL-linker-VH-linker-VL. In some embodiments, the MSLN binding agent described herein comprises an sc(Fv)₂ that binds MSLN in the format of VH-linker-VH-linker-VL-linker-VL. In some embodiments, the MSLN binding agent described herein comprises an sc(Fv)₂ that binds MSLN in the format of VL-linker-VH-linker-VH-linker-VL. In some embodiments, the MSLN binding agent described herein comprises an sc(Fv)₂ that binds MSLN in the format of VL-linker-VH-linker-VL-linker-VH. In some embodiments, the MSLN binding agent described herein comprises an sc(Fv)₂ that binds MSLN in the format of VL-linker-VL-linker-VH-linker-VH.

Alternatively, the use of one or more linkers in the sc(Fv)₂ structures described herein may be avoided, and any two of the VH and VL domains may be joint directly. Accordingly, in some embodiments, the MSLN binding agent described herein comprises an sc(Fv)₂ that binds MSLN in the format of VH-VL-linker-VL-linker-VH. In some embodiments, the MSLN binding agent described herein comprises an sc(Fv)₂ that binds MSLN in the format of VH-linker-VL-VL-linker-VH. In some embodiments, the MSLN binding agent described herein comprises an sc(Fv)₂ that binds MSLN in the format of VH-linker-VL-linker-VL-VH. In some embodiments, the MSLN binding agent described herein comprises an sc(Fv)₂ that binds MSLN in the format of VH-VL-VL-linker-VH. In some embodiments, the MSLN binding agent described herein comprises an sc(Fv)₂ that binds MSLN in the format of VH-VL-linker-VL-VH. In some embodiments, the MSLN binding agent described herein comprises an sc(Fv)₂ that binds MSLN in the format of VH-linker-VL-VL-VH. In some embodiments, the MSLN binding agent described herein comprises an sc(Fv)₂ that binds MSLN in the format of VH-VL-VL-VH.

In some embodiments, the MSLN binding agent described herein comprises an sc(Fv)₂ that binds MSLN in the format of VH-VL-linker-VH-linker-VL. In some embodiments, the MSLN binding agent described herein comprises an sc(Fv)₂ that binds MSLN in the format of VH-linker-VL-VH-linker-VL. In some embodiments, the MSLN binding agent described herein comprises an sc(Fv)₂ that binds MSLN in the format of VH-linker-VL-linker-VH-VL. In some embodiments, the MSLN binding agent described herein comprises an sc(Fv)₂ that binds MSLN in the format of VH-VL-VH-linker-VL. In some embodiments, the MSLN binding agent described herein comprises an sc(Fv)₂ that binds MSLN in the format of VH-VL-linker-VH-VL. In some embodiments, the MSLN binding agent described herein comprises an sc(Fv)₂ that binds MSLN in the format of VH-linker-VL-VH-VL. In some embodiments, the MSLN binding agent described herein comprises an sc(Fv)₂ that binds MSLN in the format of VH-VL-VH-VL.

In some embodiments, the MSLN binding agent described herein comprises an sc(Fv)₂ that binds MSLN in the format of VH-VH-linker-VL-linker-VL. In some embodiments, the MSLN binding agent described herein comprises an sc(Fv)₂ that binds MSLN in the format of VH-linker-VH-VL-linker-VL. In some embodiments, the MSLN binding agent described herein comprises an sc(Fv)₂ that binds MSLN in the format of VH-linker-VH-linker-VL-VL. In some embodiments, the MSLN binding agent described herein comprises an sc(Fv)₂ that binds MSLN in the format of VH-VH-VL-linker-VL. In some embodiments, the MSLN binding agent described herein comprises an sc(Fv)₂ that binds MSLN in the format of VH-VH-linker-VL-VL. In some embodiments, the MSLN binding agent described herein comprises an sc(Fv)₂ that binds MSLN in the format of VH-linker-VH-VL-VL. In some embodiments, the MSLN binding agent described herein comprises an sc(Fv)₂ that binds MSLN in the format of VH-VH-VL-VL.

In some embodiments, the MSLN binding agent described herein comprises an sc(Fv)₂ that binds MSLN in the format of VL-VH-linker-VH-linker-VL. In some embodiments, the MSLN binding agent described herein comprises an sc(Fv)₂ that binds MSLN in the format of VL-linker-VH-VH-linker-VL. In some embodiments, the MSLN binding agent described herein comprises an sc(Fv)₂ that binds MSLN in the format of VL-linker-VH-linker-VH-VL. In some embodiments, the MSLN binding agent described herein comprises an sc(Fv)₂ that binds MSLN in the format of VL-VH-VH-linker-VL. In some embodiments, the MSLN binding agent described herein comprises an sc(Fv)₂ that binds MSLN in the format of VL-VH-linker-VH-VL. In some embodiments, the MSLN binding agent described herein comprises an sc(Fv)₂ that binds MSLN in the format of VL-linker-VH-VH-VL. In some embodiments, the MSLN binding agent described herein comprises an sc(Fv)₂ that binds MSLN in the format of VL-VH-VH-VL.

In some embodiments, the MSLN binding agent described herein comprises an sc(Fv)₂ that binds MSLN in the format of VL-VH-linker-VL-linker-VH. In some embodiments, the MSLN binding agent described herein comprises an sc(Fv)₂ that binds MSLN in the format of VL-linker-VH-VL-linker-VH. In some embodiments, the MSLN binding agent described herein comprises an sc(Fv)₂ that binds MSLN in the format of VL-linker-VH-linker-VL-VH. In some embodiments, the MSLN binding agent described herein comprises an sc(Fv)₂ that binds MSLN in the format of VL-VH-VL-linker-VH. In some embodiments, the MSLN binding agent described herein comprises an sc(Fv)₂ that binds MSLN in the format of VL-VH-linker-VL-VH. In some embodiments, the MSLN binding agent described herein comprises an sc(Fv)₂ that binds MSLN in the format of VL-linker-VH-VL-VH. In some embodiments, the MSLN binding agent described herein comprises an sc(Fv)₂ that binds MSLN in the format of VL-VH-VL-VH.

In some embodiments, the MSLN binding agent described herein comprises an sc(Fv)₂ that binds MSLN in the format of VL-VL-linker-VH-linker-VH. In some embodiments, the MSLN binding agent described herein comprises an sc(Fv)₂ that binds MSLN in the format of VL-linker-VL-VH-linker-VH. In some embodiments, the MSLN binding agent described herein comprises an sc(Fv)₂ that binds MSLN in the format of VL-linker-VL-linker-VH-VH. In some embodiments, the MSLN binding agent described herein comprises an sc(Fv)₂ that binds MSLN in the format of VL-VL-VH-linker-VH. In some embodiments, the MSLN binding agent described herein comprises an sc(Fv)₂ that binds MSLN in the format of VL-VL-linker-VH-VH. In some embodiments, the MSLN binding agent described herein comprises an sc(Fv)₂ that binds MSLN in the format of VL-linker-VL-VH-VH. In some embodiments, the MSLN binding agent described herein comprises an sc(Fv)₂ that binds MSLN in the format of VL-VL-VH-VH.

In some embodiments, the two VH domains in an sc(Fv)₂ described herein are the same. In some embodiments, the two VH domains in an sc(Fv)₂ described herein are different. In some embodiments, the two VL domains in an sc(Fv)₂ described herein are the same. In some embodiments, the two VL domains in an sc(Fv)₂ described herein are different. In particular embodiments, the two VH domains forming any of the sc(Fv)₂ structures described herein are independently selected from the VH sequences disclosed in any of Tables 1 to 12.

In particular embodiments, the two VH domains forming any of the sc(Fv)₂ structures described herein are independently selected from SEQ ID NOS:22, 45, 62, 81, 94, 106, 120, 132, and 138. In particular embodiments, the two VL domains forming any of the sc(Fv)₂ structures described herein are independently selected from the VL sequences disclosed in any of Tables 1 to 12. In particular embodiments, the two VL domains forming any of the sc(Fv)₂ structures described herein are independently selected from SEQ ID NOS:23, 46, 63, 82, 95, 107, 121, 133, 139, 140, and 142. In particular embodiments, the two VH domains forming any of the sc(Fv)₂ structures described herein are independently selected from SEQ ID NOS:22, 45, 62, 81, 94, 106, 120, 132, and 138, and the two VL domains forming any of the sc(Fv)₂ structures described herein are independently selected from SEQ ID NOS:23, 46, 63, 82, 95, 107, 121, 133, 139, 140, and 142.

In recombinant expression systems, the linker is a peptide linker and may include any naturally occurring amino acid. Exemplary amino acids that can be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to MSLN.

In some embodiments, one or more linkers used in the sc(Fv)₂ structures described herein is about 5-50 amino acids long. In other embodiments, the one or more linkers is about 10-40 amino acids long. In other embodiments, the one or more linkers is about 10-35 amino acids long. In other embodiments, the one or more linkers is about 10-30 amino acids long. In other embodiments, the linker is about 10-25 amino acids long. In other embodiments, the one or more linkers is about 10-20 amino acids long. In other embodiments, the one or more linkers is about 15-20 amino acids long. In other embodiments, the one or more linkers is about 16-19 amino acids long. In other embodiments, the one or more linkers is 6 amino acids long. In other embodiments, the one or more linkers is 7 amino acids long. In other embodiments, the one or more linkers is 8 amino acids long. In other embodiments, the one or more linkers is 9 amino acids long. In other embodiments, the one or more linkers is 10 amino acids long. In other embodiments, the one or more linkers is 11 amino acids long. In other embodiments, the one or more linkers is 12 amino acids long. In other embodiments, the one or more linkers is 13 amino acids long. In other embodiments, the one or more linkers is 14 amino acids long. In other embodiments, the one or more linkers is 15 amino acids long. In other embodiments, the one or more linkers is 16 amino acids long. In other embodiments, the one or more linkers is 17 amino acids long. In other embodiments, the one or more linkers is 18 amino acids long. In other embodiments, the one or more linkers is 19 amino acids long. In other embodiments, the one or more linkers is 20 amino acids long. In other embodiments, the one or more linkers is 21 amino acids long. In other embodiments, the one or more linkers is 22 amino acids long. In other embodiments, the one or more linkers is 23 amino acids long. In other embodiments, the one or more linkers is 24 amino acids long. In other embodiments, the one or more linkers is 25 amino acids long. In other embodiments, the one or more linkers is 26 amino acids long. In other embodiments, the one or more linkers is 27 amino acids long. In other embodiments, the one or more linkers is 28 amino acids long. In other embodiments, the one or more linkers is 29 amino acids long. In other embodiments, the one or more linkers is 30 amino acids long. In other embodiments, the one or more linkers is 31 amino acids long. In other embodiments, the one or more linkers is 32 amino acids long. In other embodiments, the one or more linkers is 33 amino acids long. In other embodiments, the one or more linkers is 34 amino acids long. In other embodiments, the one or more linkers is 35 amino acids long. In other embodiments, the one or more linkers is 36 amino acids long. In other embodiments, the one or more linkers is 37 amino acids long. In other embodiments, the one or more linkers is 38 amino acids long. In other embodiments, the one or more linkers is 39 amino acids long. In other embodiments, the one or more linkers is 40 amino acids long. Exemplary linkers that can be used are Gly rich linkers, Gly and Ser containing linkers, Gly and Ala containing linkers, Ala and Ser containing linkers, and other flexible linkers.

Other linker sequences can include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Exemplary linkers that may be used are shown in the following Table. Additional linkers are described for example in Int. Pat. Publ. No. WO2019/060695.

In specific embodiments, the one or more linkers used in the sc(Fv)₂ structures described herein are independently selected from any one of SEQ ID NOS: 166 to 169. In particular embodiments, at least one of the linkers in the sc(Fv)₂ structure comprises the amino acid sequence of SEQ ID NO: 166. In particular embodiments, at least one of the linkers in the sc(Fv)₂ structure comprises the amino acid sequence of SEQ ID NO: 167. In particular embodiments, at least one of the linkers in the sc(Fv)₂ structure comprises the amino acid sequence of SEQ ID NO: 168. In particular embodiments, at least one of the linkers in the sc(Fv)₂ structure comprises the amino acid sequence of SEQ ID NO: 169. In particular embodiments, at least one of the linkers in the sc(Fv)₂ structure comprises the amino acid sequence of SEQ ID NO: 170. In particular embodiments, at least one of the linkers in the sc(Fv)₂ structure comprises the amino acid sequence of SEQ ID NO: 171. In particular embodiments, at least one of the linkers in the sc(Fv)₂ structure comprises the amino acid sequence of SEQ ID NO: 172. In particular embodiments, at least one of the linkers in the sc(Fv)₂ structure comprises the amino acid sequence of SEQ ID NO: 173. In particular embodiments, at least one of the linkers in the sc(Fv)₂ structure comprises the amino acid sequence of SEQ ID NO: 174. In particular embodiments, at least one of the linkers in the sc(Fv)₂ structure comprises the amino acid sequence of SEQ ID NO: 175. In particular embodiments, at least one of the linkers in the sc(Fv)₂ structure comprises the amino acid sequence of SEQ ID NO: 176. In particular embodiments, at least one of the linkers in the sc(Fv)₂ structure comprises the amino acid sequence of SEQ ID NO: 177. In particular embodiments, at least one of the linkers in the sc(Fv)₂ structure comprises the amino acid sequence of SEQ ID NO: 178. In particular embodiments, at least one of the linkers in the sc(Fv)₂ structure comprises the amino acid sequence of SEQ ID NO: 179. In particular embodiments, at least one of the linkers in the sc(Fv)₂ structure comprises the amino acid sequence of SEQ ID NO: 180. In particular embodiments, at least one of the linkers in the sc(Fv)₂ structure comprises the amino acid sequence of SEQ ID NO: 181. In particular embodiments, at least one of the linkers in the sc(Fv)₂ structure comprises the amino acid sequence of SEQ ID NO: 182. In particular embodiments, at least one of the linkers in the sc(Fv)₂ structure comprises the amino acid sequence of SEQ ID NO: 183. In particular embodiments, at least one of the linkers in the sc(Fv)₂ structure comprises the amino acid sequence of SEQ ID NO: 184. In particular embodiments, at least one of the linkers in the sc(Fv)₂ structure comprises the amino acid sequence of SEQ ID NO: 185. In particular embodiments, at least one of the linkers in the sc(Fv)₂ structure comprises the amino acid sequence of SEQ ID NO: 186. In particular embodiments, at least one of the linkers in the sc(Fv)₂ structure comprises the amino acid sequence of SEQ ID NO: 187. In particular embodiments, at least one of the linkers in the sc(Fv)₂ structure comprises the amino acid sequence of SEQ ID NO: 188. In particular embodiments, at least one of the linkers in the sc(Fv)₂ structure comprises the amino acid sequence of SEQ ID NO: 189. In particular embodiments, at least one of the linkers in the sc(Fv)₂ structure comprises the amino acid sequence of SEQ ID NO: 190. In particular embodiments, at least one of the linkers in the sc(Fv)₂ structure comprises the amino acid sequence of SEQ ID NO: 191. In particular embodiments, at least one of the linkers in the sc(Fv)₂ structure comprises the amino acid sequence of SEQ ID NO: 192. In particular embodiments, at least one of the linkers in the sc(Fv)₂ structure comprises the amino acid sequence of SEQ ID NO: 193. In particular embodiments, at least one of the linkers in the sc(Fv)₂ structure comprises the amino acid sequence of SEQ ID NO: 194. In particular embodiments, at least one of the linkers in the sc(Fv)₂ structure comprises the amino acid sequence of SEQ ID NO: 195. In particular embodiments, at least one of the linkers in the sc(Fv)₂ structure comprises the amino acid sequence of SEQ ID NO: 196. In particular embodiments, at least one of the linkers in the sc(Fv)₂ structure comprises the amino acid sequence of SEQ ID NO: 197. In particular embodiments, at least one of the linkers in the sc(Fv)₂ structure comprises the amino acid sequence of SEQ ID NO: 198. In particular embodiments, at least one of the linkers in the sc(Fv)₂ structure comprises the amino acid sequence of SEQ ID NO: 199.

7.4.5 Humanized Antibodies

In some embodiments, antibodies provided herein can be humanized antibodies that bind MSLN, including human and/or cynomolgus MSLN. For example, humanized antibodies of the present disclosure may comprise one or more CDRs as shown in Tables 1-12. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, V_{L}6 subgroup I (V1.61) and V_{H} subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169: 1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

7.4.6 Human Antibodies

Human anti-MSLN antibodies can be constructed by combining Fv clone variable domain sequence(s) selected from human-derived phage display libraries with known human constant domain sequences(s). Alternatively, human monoclonal anti-MSLN antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Brüggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human HCDR3 is commonly retained, as this CDR may be at the center of the antigen-binding site and may be the most important region of the antibody for antigen recognition. In some instances, however, HCDR3 and LCDR3, as well as HCDR2, LCDR2, and LCDR1 of the non-human antibody may be retained.

7.4.7 Fc Engineering

In some embodiments, the antigen binding domains that bind MSLN of the disclosure are conjugated to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). In some embodiments, the antigen binding domains that bind MSLN of the disclosure are used to make a fusion protein, wherein the fusion protein comprises the antigen binding domains that bind MSLN and an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or the fragment of the Ig constant region functions also as a half-life extending moiety as discussed herein. The antigen binding domains that bind MSLN of the disclosure may be engineered into conventional full-length antibodies using standard methods. The full-length antibodies comprising the antigen binding domain that binds MSLN may further be engineered as described herein.

Immunoglobulin heavy chain constant region comprised of subdomains CH1, hinge, CH2 and CH3. The CH1 domain spans residues A118-V215, the CH2 domain residues A231-K340 and the CH3 domain residues G341-K447 on the heavy chain, residue numbering according to the EU Index. In some instances, G341 is referred as a CH2 domain residue. Hinge is generally defined as including E216 and terminating at P230 of human IgG1. In some embodiments, the Ig Fc region comprises at least the CH2 and the CH3 domains of the Ig constant region, and therefore comprises at least a region from about A231 to K447 of Ig heavy chain constant region.

In some embodiments, the C-terminal lysine (CTL) is removed from the Ig constant region. Accordingly, in some embodiments, the Ig Fc region comprises at least a region from about A231 to G446 of Ig heavy chain constant region. In specific embodiments, the CTL is removed from the Ig constant region by endogenous circulating carboxypeptidases in the blood stream (Cai et al., (2011) Biotechnol Bioeng 108:404-412). In some embodiments, during manufacturing, CTL removal may be controlled to less than the maximum level by control of concentration of extracellular Zn²⁺, EDTA or EDTA-Fe³⁺ as described in U.S. Patent Publ. No. US20140273092. CTL content of proteins may be measured using known methods.

In other embodiments, the antigen binding fragment that binds MSLN fused to the Ig constant region has a C-terminal lysine content from about 10% to about 90%. In other embodiments, the C-terminal lysine content is from about 20% to about 80%. In other embodiments, the C-terminal lysine content is from about 40% to about 70%. In other embodiments, the C-terminal lysine content is from about 55% to about 70%. In other embodiments, the C-terminal lysine content is about 60%.

The present disclosure also provides an antigen binding domain that binds MSLN fused or conjugated to an immunoglobulin (Ig) constant region or a fragment of the Ig constant region. In some embodiments, the Ig constant region is a heavy chain constant region. In some embodiments, the Ig constant region is a light chain constant region. In some embodiments, the fragment of the Ig constant region comprises a Fc region. In some embodiments, the fragment of the Ig constant region comprises a CH2 domain. In some embodiments, the fragment of the Ig constant region comprises a CH3 domain. In some embodiments, the fragment of the Ig constant region comprises the CH2 domain and the CH3 domain. In some embodiments, the fragment of the Ig constant region comprises at least portion of a hinge, the CH2 domain and the CH3 domain. Portion of the hinge refers to one or more amino acid residues of the Ig hinge. In some embodiments, the fragment of the Ig constant region comprises the hinge, the CH2 domain and the CH3 domain.

In some embodiments, the antigen binding domain that binds MSLN is fused or conjugated to the N-terminus of the Ig constant region or the fragment of the Ig constant region. In some embodiments, the antigen binding domain that binds MSLN is fused or conjugated to the C-terminus of the Ig constant region or the fragment of the Ig constant region. In some embodiments, the antigen binding domain that binds MSLN is fused or conjugated to the Ig constant region or the fragment of the Ig constant region via a second linker (L2). In some embodiments, the L2 comprises the amino acid sequence selected from SEQ ID NOS: 166 to 199. In particular embodiments, the L2 comprises the amino acid sequence of SEQ ID NO: 166. In particular embodiments, the L2 comprises the amino acid sequence of SEQ ID NO: 167. In particular embodiments, the L2 comprises the amino acid sequence of SEQ ID NO: 168. In particular embodiments, the L2 comprises the amino acid sequence of SEQ ID NO: 169. In particular embodiments, the L2 comprises the amino acid sequence of SEQ ID NO: 170. In particular embodiments, the L2 comprises the amino acid sequence of SEQ ID NO: 171. In particular embodiments, the L2 comprises the amino acid sequence of SEQ ID NO: 172. In particular embodiments, the L2 comprises the amino acid sequence of SEQ ID NO: 173. In particular embodiments, the L2 comprises the amino acid sequence of SEQ ID NO: 174. In particular embodiments, the L2 comprises the amino acid sequence of SEQ ID NO: 175. In particular embodiments, the L2 comprises the amino acid sequence of SEQ ID NO: 176. In particular embodiments, the L2 comprises the amino acid sequence of SEQ ID NO: 177. In particular embodiments, the L2 comprises the amino acid sequence of SEQ ID NO: 178. In particular embodiments, the L2 comprises the amino acid sequence of SEQ ID NO: 179. In particular embodiments, the L2 comprises the amino acid sequence of SEQ ID NO: 180. In particular embodiments, the L2 comprises the amino acid sequence of SEQ ID NO: 181. In particular embodiments, the L2 comprises the amino acid sequence of SEQ ID NO: 182. In particular embodiments, the L2 comprises the amino acid sequence of SEQ ID NO: 183. In particular embodiments, the L2 comprises the amino acid sequence of SEQ ID NO: 184. In particular embodiments, the L2 comprises the amino acid sequence of SEQ ID NO: 185. In particular embodiments, the L2 comprises the amino acid sequence of SEQ ID NO: 186. In particular embodiments, the L2 comprises the amino acid sequence of SEQ ID NO: 187. In particular embodiments, the L2 comprises the amino acid sequence of SEQ ID NO: 188. In particular embodiments, the L2 comprises the amino acid sequence of SEQ ID NO: 189. In particular embodiments, the L2 comprises the amino acid sequence of SEQ ID NO: 190. In particular embodiments, the L2 comprises the amino acid sequence of SEQ ID NO: 191. In particular embodiments, the L2 comprises the amino acid sequence of SEQ ID NO: 192. In particular embodiments, the L2 comprises the amino acid sequence of SEQ ID NO: 193. In particular embodiments, the L2 comprises the amino acid sequence of SEQ ID NO: 194. In particular embodiments, the L2 comprises the amino acid sequence of SEQ ID NO: 195. In particular embodiments, the L2 comprises the amino acid sequence of SEQ ID NO: 196. In particular embodiments, the L2 comprises the amino acid sequence of SEQ ID NO: 197. In particular embodiments, the L2 comprises the amino acid sequence of SEQ ID NO: 198. In particular embodiments, the L2 comprises the amino acid sequence of SEQ ID NO: 199.

The antigen binding domains that bind MSLN of the disclosure fused or conjugated to Ig constant region or the fragment of the Ig constant region may be assessed for their functionality using several known assays. Binding to MSLN can be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC, CDC or ADCP.

ADCC can be assessed using an in vitro assay using MSLN expressing cells as target cells and NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

ADCP can be evaluated by using monocyte-derived macrophages as effector cells and any MSLN expressing cells as target cells which are engineered to express GFP or other labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11 CD14 macrophages using standard methods.

CDC of cells may be measured for example by plating Daudi cells at 1× 10⁵ cells/well (50 μL/well) in RPMI-B (RPMI supplemented with 1% BSA), adding 50 μL of test protein to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

In some embodiments, it may be desirable to modify an anti-MSLN antibody provided herein by Fc engineering. In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29(8): 2613-24; and Shields et al., 2001, J. Biol. Chem. 276(9): 6591-604). Other Fc variants are provided elsewhere herein.

To increase the serum half life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

In some embodiments, the Ig constant region or the fragment of the Ig constant region comprises at least one mutation that modulates a half-life of the MLSN binding agent. In some embodiments, the at least one mutation that modulates the half-life of the MLSN binding agent is selected from the group consisting of H435A, P257I/N434H, D376V/N434H, M252Y/S254T/T256E/H433K/N434F, T308P/N434A, and H435R, wherein residue numbering is according to the EU index. In some embodiments, the MSLN binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the MLSN binding agent independently selected from the group consisting of H435A, P257I/N434H, D376V/N434H, M252Y/S254T/T256E/H433K/N434F, T308P/N434A, and H435R, wherein residue numbering is according to the EU index.

In some embodiments, the Ig constant region or the fragment of the Ig constant region comprises at least one mutation that results in reduced binding of the MSLN binding agent to a FcγR.

In some embodiments, the at least one mutation that results in reduced binding of the MSLN binding agent to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the MSLN binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the MLSN binding agent independently selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the Ig constant region or the fragment of the Ig constant region comprises at least one mutation that results in enhanced binding of the MSLN binding agent to a FcγR.

In some embodiments, the at least one mutation that results in enhanced binding of the MSLN binding agent to the FcγR is selected from the group consisting of S239D/1332E, S298A/E333A/K334A, F243L/R292P/Y300L, F243L/R292P/Y300L/P396L, F243L/R292P/Y300L/V305I/P396L and G236A/S239D/1332E, wherein residue numbering is according to the EU index. In some embodiments, the FcγR is FcγRI, FcγRIIA, FcγRIIB or FcγRIII, or any combination thereof. In some embodiments, the MSLN binding agent comprises a first Ig constant region or a fragment thereof and a second Ig constant region or a fragment thereof. In some embodiments, one or both of the first Ig constant region or a fragment thereof and a second Ig constant region or a fragment thereof comprises at least one mutation that modulates a half-life of the MLSN binding agent independently selected from the group consisting of S239D/1332E, S298A/E333A/K334A, F243L/R292P/Y300L, F243L/R292P/Y300L/P396L, F243L/R292P/Y300L/V305I/P396L and G236A/S239D/1332E, wherein residue numbering is according to the EU index.

In some embodiments, the MSLN binding agent comprises at least one mutation in a CH3 domain of a first Ig constant region or in a CH3 domain of the fragment of the first Ig constant region and/or at least one mutation in a CH3 domain of a second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, T366L, F405W, K392L, T394W, T394S, Y407T, Y407A, H435R, Y436F, T366S/L368A/Y407V, L351Y/F405A/Y407V, T366I/K392M/T394W, T366L/K392L/T394W, F405A/Y407V, T366L/K392M/T394W, L351Y/Y407A, L351Y/Y407V, T366A/K409F, L351Y/Y407A, T366V/K409F, T366A/K409F, T350V/L351Y/F405A/Y407V, T350V/T366L/K392L/T394W, and H435R/L436F wherein residue numbering is according to the EU index. In some embodiments, at least one mutation in the CH3 domain is selected from the group consisting of H435R, Y436F and H435R/L436F, wherein residue numbering is according to the EU index

In some embodiments, the first Ig constant region or the fragment of the first Ig constant region and the second Ig constant region or the fragment of the second Ig constant region comprise the following mutations L234A_L235A_D265S_T350V_L351Y_F405A_Y407V in the first Ig constant region and L234A_L235A_D265S_T350V_T366L_K392L_T394W in the second Ig constant region; or L234A_L235A_D265S_T350V_T366L_K392L_T394W in the first Ig constant region and L234A_L235A_D265S_T350V_L351Y_F405A_Y407V in the second Ig constant region.

7.4.8 Alternative Binding Agents

The present disclosure encompasses non-immunoglobulin binding agents that specifically bind to the same epitope as an anti-MSLN antibody disclosed herein. In some embodiments, a non-immunoglobulin binding agent is identified as an agent that displaces or is displaced by an anti-MSLN antibody of the present disclosure in a competitive binding assay. These alternative binding agents may include, for example, any of the engineered protein scaffolds known in the art. Such scaffolds may comprise one or more CDRs as shown in Tables 1-12. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352: 95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

7.5 Multispecific Binding Proteins

In one aspect of the present disclosure, the MSLN binding agents described herein are multispecific (e.g., bispecific or trispecific) binding molecules that can bind to one or more other antigens in addition to MSLN.

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating bispecific antibodies, see, for example, Bispecific Antibodies (Kontermann ed., 2011).

In some embodiments, the multispecific protein is bispecific. In some embodiments, the multispecific protein is trispecific. In some embodiments, the multispecific protein is tetraspecific.

In specific embodiments, the MSLN binding agents described herein are bispecific antibodies. In some embodiments, bispecific antibodies described herein are monoclonal antibodies that have binding specificities for at least two different antigens. In certain embodiments, bispecific antibodies are human or humanized antibodies. In certain embodiments, one of the binding specificities is for MSLN and the other is for any other antigen. In some embodiments, one of the binding specificities is for MSLN, and the other is for another surface antigen expressed on cells expressing MSLN. In certain embodiments, bispecific antibodies may bind to two different epitopes of MSLN (i.e., bivalent for MSLN). Bispecific antibodies can be prepared as full length antibodies or antibody fragments (e.g., F(ab′)₂ bispecific antibodies).

In other embodiments, the multispecific protein is monovalent for binding to MSLN. In other embodiments, the multispecific protein is bivalent for binding to MSLN. In other embodiments, the multispecific protein is monovalent for binding to the second antigen. In other embodiments, the multispecific protein is bivalent for binding to second antigen. In other embodiments, the multispecific protein is monovalent for binding to MSLN, and is monovalent for binding to the second antigen. In other embodiments, the multispecific protein is bivalent for binding to MSLN, and is monovalent for binding to the second antigen. In other embodiments, the multispecific protein is monovalent for binding to MSLN, and is bivalent for binding to the second antigen. In other embodiments, the multispecific protein is bivalent for binding to MSLN, and is bivalent for binding to the second antigen.

In some embodiments, the first antigen binding domain that binds MSLN and/or the second antigen binding domain that binds the second antigen are independently selected from a scFv, a (scFv)₂, a Fv, a Fab, a F(ab′)₂, a Fd, a dAb or a VHH. In some embodiments, the first antigen binding domain that binds MSLN and/or the second antigen binding domain that binds the second antigen comprise the Fab. In some embodiments, the first antigen binding domain that binds MSLN and/or the second antigen binding domain that binds the second antigen comprise the F(ab′)₂. In some embodiments, the first antigen binding domain that binds MSLN and/or the second antigen binding domain that binds the second antigen comprise the VHH. In some embodiments, the first antigen binding domain that binds MSLN and/or the second antigen binding domain that binds the second antigen comprise the Fv. In some embodiments, the first antigen binding domain that binds MSLN and/or the second antigen binding domain that binds the second antigen comprise the Fd. In some embodiments, the first antigen binding domain that binds MSLN and/or the second antigen binding domain that binds the second antigen comprise the scFv. In some embodiments, the first antigen binding domain that binds MSLN comprises the Fab, and the second antigen binding domain that binds the second antigen comprises the scFv. In specific embodiments, the second antigen is a tumor antigen.

In some embodiments, the first antigen binding domain that binds MSLN is fused or conjugated to a first immunoglobulin (Ig) constant region or a fragment of the first Ig constant region and/or the second antigen binding domain that binds the tumor antigen is fused or conjugated to a second immunoglobulin (Ig) constant region or a fragment of the second Ig constant region.

In some embodiments, the fragment of the first Ig constant region and/or the fragment of the second Ig constant region comprises a Fc region. In some embodiments, the fragment of the first Ig constant region and/or the fragment of the second Ig constant region comprises a CH2 domain. In some embodiments, the fragment of the first Ig constant region and/or the fragment of the second Ig constant region comprises a CH3 domain. In some embodiments, the fragment of the first Ig constant region and/or the fragment of the second Ig constant region comprises the CH2 domain and the CH3 domain.

In some embodiments, the fragment of the first Ig constant region and/or the fragment of the second Ig constant region comprises at least portion of a hinge, the CH2 domain and the CH3 domain. In some embodiments, the fragment of the Ig constant region comprises the hinge, the CH2 domain and the CH3 domain.

In some embodiments, the multispecific protein further comprises a second linker (L2) between the first antigen binding domain that binds MSLN and the first Ig constant region or the fragment of the first Ig constant region and the second antigen binding domain that binds the tumor antigen and the second Ig constant region or the fragment of the second Ig constant region. In some embodiments, the L2 comprises the amino acid sequence of any one of SEQ ID NOs: 166 to 169.

In some embodiments, the first Ig constant region or the fragment of the first Ig constant region and the second Ig constant region or the fragment of the second Ig constant region is an IgG1, an IgG2, and IgG3 or an IgG4 isotype. In some embodiments, the first Ig constant region or the fragment of the first Ig constant region and the second Ig constant region or the fragment of the second Ig constant region is an IgG1 isotype. In some embodiments, the first Ig constant region or the fragment of the first Ig constant region and the second Ig constant region or the fragment of the second Ig constant region is an IgG2 isotype. In some embodiments, the first Ig constant region or the fragment of the first Ig constant region and the second Ig constant region or the fragment of the second Ig constant region is an IgG3 isotype. In some embodiments, the first Ig constant region or the fragment of the first Ig constant region and the second Ig constant region or the fragment of the second Ig constant region is an IgG4 isotype.

The first Ig constant region or the fragment of the first Ig constant region and the second Ig constant region or the fragment of the second Ig constant region can further be engineered as described herein. In some embodiments, the first Ig constant region or the fragment of the first Ig constant region and the second Ig constant region or the fragment of the second Ig constant region comprises at least one mutation that results in reduced binding of the multispecific protein to a FcγR.

In some embodiments, the at least one mutation that results in reduced binding of the multispecific protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the first Ig constant region or the fragment of the first Ig constant region and the second Ig constant region or the fragment of the second Ig constant region comprises at least one mutation that results in enhanced binding of the multispecific protein to a Fcγ receptor (FcγR).

In some embodiments, the at least one mutation that results in enhanced binding of the multispecific protein to the FcγR is selected from the group consisting of S239D/1332E, S298A/E333A/K334A, F243L/R292P/Y300L, F243L/R292P/Y300L/P396L, F243L/R292P/Y300L/V305I/P396L and G236A/S239D/1332E, wherein residue numbering is according to the EU index. In some embodiments, the FcγR is FcγRI, FcγRIIA, FcγRIIB or FcγRIII, or any combination thereof.

In some embodiments, the first Ig constant region or the fragment of the first Ig constant region and the second Ig constant region or the fragment of the second Ig constant region comprises at least one mutation that modulates a half-life of the multispecific protein. In some embodiments, the at least one mutation that modulates the half-life of the multispecific protein is selected from the group consisting of H435A, P257I/N434H, D376V/N434H, M252Y/S254T/T256E/H433K/N434F, T308P/N434A, and H435R, wherein residue numbering is according to the EU index.

In some embodiments, the multispecific protein comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, T366L, F405W, K392L, T394W, T394S, Y407T, Y407A, T366S/L368A/Y407V, L351Y/F405A/Y407V, T366I/K392M/T394W, T366L/K392L/T394W, F405A/Y407V, T366L/K392M/T394W, L351Y/Y407A, L351Y/Y407V, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the first Ig constant region or the fragment of the first Ig constant region and the second Ig constant region or the fragment of the second Ig constant region comprise the following mutations L234A_L235A_D265S_T350V_L351Y_F405A_Y407V in the first Ig constant region and L234A_L235A_D265S_T350V_T366L_K392L_T394W in the second Ig constant region; or L234A_L235A_D265S_T350V_T366L_K392L_T394W in the first Ig constant region and L234A_L235A_D265S_T350V_L351Y_F405A_Y407V in the second Ig constant region.

In some embodiments, the multispecific binding proteins provided herein are antibodies having a full length antibody structure. “Full length antibody” refers to an antibody having two full length antibody heavy chains and two full length antibody light chains. A full length antibody heavy chain (HC) consists of well-known heavy chain variable and constant domains VH, CH1, hinge, CH2, and CH3. A full length antibody light chain (LC) consists of well-known light chain variable and constant domains VL and CL. The full length antibody can be lacking the C-terminal lysine (K) in either one or both heavy chains. “Fab-arm” or “half molecule” refers to one heavy chain-light chain pair that specifically binds an antigen.

Full length bispecific antibodies can be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms can be engineered to favor heterodimerization over homodimerization. The resulting product is a bispecific antibody having two Fab arms or half molecules which each bind a distinct epitope, i.e. an epitope on MSLN and an epitope on CD3.

“Homodimerization” refers to an interaction of two heavy chains having identical CH3 amino acid sequences. “Homodimer” refers to an antibody having two heavy chains with identical CH3 amino acid sequences. “Heterodimerization” refers to an interaction of two heavy chains having non-identical CH3 amino acid sequences. “Heterodimer” refers to an antibody having two heavy chains with non-identical CH3 amino acid sequences.

In some embodiments, the binding proteins provided herein include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), the Biclonic (Merus) and the DuoBody (Genmab A/S).

In some embodiments, a multispecific binding protein provided herein is in the knob-and-hole format. In some embodiments, a multispecific binding protein provided herein is in a DuoBody format.

The Triomab quadroma technology can be used to generate full length bispecific antibodies provided herein. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., International Publication No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology can be used to generate full length bispecific antibodies provided herein. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies can be used to generate full length bispecific antibodies provided herein by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in International Publication Nos. WO 2009/080254, WO 2009/080251, WO 2009/018386 and WO 2009/080252.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849. Other strategies that can facilitate the removal of homodimer and/or half-molecule species include incorporating both substitutions H435R and Y436F in a heavy chain CH3 domain.

LUZ-Y technology can be utilized to generate bispecific antibodies provided herein. In this technology, a leucine zipper is added into the C terminus of the CH3 domains to drive the heterodimer assembly from parental mAbs that is removed post-purification as described in Wranik et al., (2012) J Biol Chem 287(52): 42221-9.

SEEDbody technology can be utilized to generate bispecific antibodies provided herein. SEEDbodies have, in their constant domains, select IgG residues substituted with IgA residues to promote heterodimerization as described in U.S. Patent No. US20070287170.

In addition to methods described above, binding agents provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO 2011/131746.

In some embodiments described herein, the bispecific MSLN binding agent described herein comprises a first binding region binding MSLN and a second binding region binding a second antigen, and comprises at least one substitution in an antibody CH3 constant domain. Substitutions are typically made at the DNA level to a molecule such as the constant domain of the antibody using standard methods.

The antibodies provided herein can be engineered into various well-known antibody forms.

In some embodiments, the bispecific antibody is a diabody or a cross-body.

In some embodiments, the bispecific antibody includes IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine—China).

In some embodiments, Fab fusion bispecific antibodies include F(ab)₂ (Medarex/AMGEN), Dual-Action or Bis-Fab (Genentech), Dock-and-Lock (DNL) (ImmunoMedics), Bivalent Bispecific (Biotecnoland Fab-Fv (UCB-Celltech). ScFv−, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5): 588-594.

7.6 Bispecific MSLN×CD3 Binding Proteins

As described herein, any antigen other than MSLN can be selected as the second antigen target for the present multispecific MSLN binding agent. In specific embodiments, the multispecific binding agent is a multispecific binding protein. In particular embodiments, the second antigen is expressed on an immune cell. In some embodiments, the second antigen is CD3. In specific embodiments, the second antigen is CD3ε.

In specific embodiments, the multispecific binding protein described herein is a bispecific MSLN×CD3 binding protein comprising a first antigen binding region that binds MSLN and a second antigen binding region that binds CD3ε. In some embodiments, the MSLN binding region of multispecific binding protein can be any MSLN binding region or MSLN binding protein described herein (e.g., in Section 7.3).

In particular embodiments, the CD3ε binding region of the multispecific binding protein comprises one or more binding sequences set forth in Tables 13A to 14. In some embodiments, the CD3ε binding region comprises one or more CDR sequences set forth in SEQ ID NOs: 159 and 160. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CD3ε binding region is humanized. In some embodiments, the CD3ε binding region comprises an acceptor human framework, e.g., a human immunoglobulin framework or a human consensus framework.

In some embodiments, the CD3ε binding region provided herein comprises HCDR1, HCDR2, and HCDR3 as set forth in SEQ ID NO:159. In some embodiments, the CD3ε binding region provided herein comprises LCDR1, LCDR2, and LCDR3 as set forth in SEQ ID NO:160. In some embodiments, the CD3ε binding region provided herein comprises HCDR1, HCDR2, and HCDR3 as set forth in SEQ ID NO: 159, and LCDR1, LCDR2, and LCDR3 as set forth in SEQ ID NO:160. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, the CD3ε binding region comprises an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 143, 149, and 155; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 144, 150, and 156, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 145, 151, and 157; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 146, 152, and 158; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NOs: 147 and 153; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NOs:148 and 154. In some embodiments, the CD3ε binding region is humanized. In some embodiments, the CD3ε binding region comprises an acceptor human framework, e.g., a human immunoglobulin framework or a human consensus framework.

In some specific embodiments, the CD3 binding region provided herein comprises one or more CDRs in Table 13A.

TABLE 13A
CD3B2030-N106A Binding Region Sequences

| | Kabat |
|---|---|
| Heavy Chain | RSTMH |
| Variable | (SEQ ID |
| Region CDR1 | NO: 210) |
| Heavy Chain | YINPSSA |
| Variable | YTNYNQK |
| Region CDR2 | FQG |
| | (SEQ ID |
| | NO: 211) |
| Heavy Chain | PQVHYDY |
| Variable | AGFPY |
| Region CDR3 | (SEQ ID |
| | NO: 212) |
| Light Chain | SASSSVS |
| Variable | YMN |
| Region CDR1 | (SEQ ID |
| | NO: 213) |
| Light Chain | DSSKLAS |
| Variable | (SEQ ID |
| Region CDR2 | NO: 214) |
| Light Chain | QQWSRNPPT |
| Variable | (SEQ ID |
| Region CDR3 | NO: 215) |

VH (SEQ ID NO: 235)
QVQLVQSGAEVKKPGSSVKVSCKASGYTFTRST
MHWVKQAPGQGLEWIGYINPSSAYTNYNQKFQG
RVTLTADKSTSTAYMELSSLRSEDTAVYYCASP
QVHYDYAGFPYWGQGTLVTVSS
VL (SEQ ID NO: 236)
EIVLTQSPATLSASPGERVTLSCSASSSVSYMN
WYQQKPGQAPRRWIYDSSKLASGVPARFSGSGS
GRDYTLTISSLEPEDFAVYYCQQWSRNPPTFGG
GTKVEIK

In some specific embodiments, in the CD3ε binding region provided herein, the HCDR1 comprises the amino acid sequence of SEQ ID NO: 143, the HCDR2 comprises the amino acid sequence of SEQ ID NO:144, the HCDR3 comprises the amino acid sequence of SEQ ID NO:145, the LCDR1 comprises the amino acid sequence of SEQ ID NO: 146, the LCDR2 comprises the amino acid sequence of SEQ ID NO: 147, and the LCDR3 comprises the amino acid sequence of SEQ ID NO:148.

In some specific embodiments, in the CD3ε binding region provided herein, the HCDR1 comprises the amino acid sequence of SEQ ID NO:149, the HCDR2 comprises the amino acid sequence of SEQ ID NO: 150, the HCDR3 comprises the amino acid sequence of SEQ ID NO:151, the LCDR1 comprises the amino acid sequence of SEQ ID NO: 152, the LCDR2 comprises the amino acid sequence of YAS, and the LCDR3 comprises the amino acid sequence of SEQ ID NO:154.

In some specific embodiments, in the CD3ε binding region provided herein, the HCDR1 comprises the amino acid sequence of SEQ ID NO: 155, the HCDR2 comprises the amino acid sequence of SEQ ID NO:156, the HCDR3 comprises the amino acid sequence of SEQ ID NO:157, the LCDR1 comprises the amino acid sequence of SEQ ID NO:158, the LCDR2 comprises the amino acid sequence of YAS, and the LCDR3 comprises the amino acid sequence of SEQ ID NO: 148.

In some embodiments, the CD3E binding region further comprises one or more framework regions. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FRI is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system. In some embodiments, the CD3ε binding region further comprises one or more framework regions of SEQ ID NOs: 159 and 160.

In some embodiments, the CD3ε binding region provided herein comprises a VH comprising the amino acid sequence of SEQ ID NO: 159, and a VL comprising the amino acid sequence of SEQ ID NO:160.

In certain embodiments, the CD3ε binding region provided herein comprises amino acid sequences with certain percent identity relative to any CD3 binding region provided herein. The determination of percent identity can be accomplished using mathematical algorithms known in the art or described herein.

In some embodiments, the CD3ε binding region provided herein contains substitutions (e.g., conservative substitutions), insertions, or deletions relative to the reference sequence, but the CD3ε binding region comprising that sequence retains the ability to bind to CD3ε. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the CD3ε binding region provided herein includes post-translational modifications of a reference sequence.

In some embodiments, the CD3ε binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:159, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:160.

In some embodiments, the CD3ε binding region provided herein binds to the same epitope as a CD3ε binding region comprising a VH comprising the amino acid sequence of SEQ ID NO:159, and a VL comprising the amino acid sequence of SEQ ID NO:160.

In some embodiments, the CD3E binding region provided herein specifically binds to CD3ε competitively with a CD3ε binding region comprising a VH comprising the amino acid sequence of SEQ ID NO:159, and a VL comprising the amino acid sequence of SEQ ID NO:160.

In some specific embodiments, the CD3 binding region provided herein comprises one or more CDRs in Table 13B.

TABLE 13B
CD3B450 Binding Region Sequences

| | Kabat |
|---|---|
| Heavy Chain | NNNAA |
| Variable | WS |
| Region CDR1 | (SEQ ID |
| | NO: 204) |
| Heavy Chain | RTYYRSKWL |
| Variable | YDYAVSVKS |
| Region CDR2 | (SEQ ID |
| | NO: 205) |
| Heavy Chain | GYSSSF |
| Variable | DY |
| Region CDR3 | (SEQ ID |
| | NO: 206) |
| Light Chain | TGTSSNIG |
| Variable | TYKFVS |
| Region CDR1 | (SEQ ID |
| | NO: 207) |
| Light Chain | EVSKR |
| Variable | PS |
| Region CDR2 | (SEQ ID |
| | NO: 208) |
| Light Chain | VSYAGS |
| Variable | GTLL |
| Region CDR3 | (SEQ ID |
| | NO: 209) |

VH (SEQ ID NO: 233)
QVQLQQSGPGLVKPSQTLSLTCAISGDSVENNNAAWS
WIRQSPSRGLEWLGRTYYRSKWLYDYAVSVKSRITIN
PDTSKNQFSLQLNSVTPEDTAVYYCARGYSSSFDYWG
QGTLVTVSS
VL (SEQ ID NO: 234)
QSALTQPASVSGSPGQSITISCTGTSSNIGTYKFVSW
YQQHPGKAPKVMIYEVSKRPSGVSNRFSGSKSGNTAS
LTISGLQAEDEADYYCVSYAGSGTLLFGGGTKLTVL

In some specific embodiments, in the CD3ε binding region provided herein, the HCDR1 comprises the amino acid sequence of SEQ ID NO:204, the HCDR2 comprises the amino acid sequence of SEQ ID NO:205, the HCDR3 comprises the amino acid sequence of SEQ ID NO:206, the LCDR1 comprises the amino acid sequence of SEQ ID NO:207, the LCDR2 comprises the amino acid sequence of SEQ ID NO:208, and the LCDR3 comprises the amino acid sequence of SEQ ID NO:209.

In some embodiments, the CD3ε binding region further comprises one or more framework regions. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FRI is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system. In some embodiments, the CD3ε binding region further comprises one or more framework regions of SEQ ID NOs:233 and 234.

In some embodiments, the CD3ε binding region provided herein comprises a VH comprising the amino acid sequence of SEQ ID NO:233, and a VL comprising the amino acid sequence of SEQ ID NO:234.

In some embodiments, the CD3ε binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:233, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:234.

In some embodiments, the CD3 binding region provided herein binds to the same epitope as a CD3ε binding region comprising a VH comprising the amino acid sequence of SEQ ID NO:233, and a VL comprising the amino acid sequence of SEQ ID NO:234.

In some embodiments, the CD3ε binding region provided herein specifically binds to CD3ε competitively with a CD3ε binding region comprising a VH comprising the amino acid sequence of SEQ ID NO:233, and a VL comprising the amino acid sequence of SEQ ID NO:234.

In some specific embodiments, the CD3 binding region provided herein comprises one or more CDRs in Table 13C.

TABLE 13C
CD3B2030-N106A Binding Region Sequences

| | Kabat |
|---|---|
| Heavy Chain Variable | RSTMH |
| Region CDR1 | (SEQ ID NO: 210) |
| Heavy Chain Variable | YINPSSAYTNYNQKFQG |
| Region CDR2 | (SEQ ID NO: 211) |
| Heavy Chain Variable | PQVHYDYAGFPY |
| Region CDR3 | (SEQ ID NO: 212) |
| Light Chain Variable | SASSSVSYMN |
| Region CDR1 | (SEQ ID NO: 213) |
| Light Chain Variable | DSSKLAS |
| Region CDR2 | (SEQ ID NO: 214) |
| Light Chain Variable | QQWSRNPPT |
| Region CDR3 | (SEQ ID NO: 215) |

VH (SEQ ID NO: 235)
QVQLVQSGAEVKKPGSSVKVSCKASGYTFTRSTMH
WVKQAPGQGLEWIGYINPSSAYTNYNQKFQGRVTL
TADKSTSTAYMELSSLRSEDTAVYYCASPQVHYDY
AGFPYWGQGTLVTVSS
VL (SEQ ID NO: 236)
EIVLTQSPATLSASPGERVTLSCSASSSVSYMNWY
QQKPGQAPRRWIYDSSKLASGVPARFSGSGSGRDY
TLTISSLEPEDFAVYYCQQWSRNPPTFGGGTKVEI
K

In some specific embodiments, in the CD3ε binding region provided herein, the HCDR1 comprises the amino acid sequence of SEQ ID NO:210, the HCDR2 comprises the amino acid sequence of SEQ ID NO:211, the HCDR3 comprises the amino acid sequence of SEQ ID NO:212, the LCDR1 comprises the amino acid sequence of SEQ ID NO:213, the LCDR2 comprises the amino acid sequence of SEQ ID NO:214, and the LCDR3 comprises the amino acid sequence of SEQ ID NO:215.

In some embodiments, the CD3ε binding region further comprises one or more framework regions. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FRI is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system. In some embodiments, the CD3ε binding region further comprises one or more framework regions of SEQ ID NOs:235 and 236.

In some embodiments, the CD3ε binding region provided herein comprises a VH comprising the amino acid sequence of SEQ ID NO:235, and a VL comprising the amino acid sequence of SEQ ID NO:236.

In some embodiments, the CD3ε binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:235, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:236.

In some embodiments, the CD3ε binding region provided herein binds to the same epitope as a CD3ε binding region comprising a VH comprising the amino acid sequence of SEQ ID NO:235, and a VL comprising the amino acid sequence of SEQ ID NO:236.

In some embodiments, the CD3ε binding region provided herein specifically binds to CD3ε competitively with a CD3ε binding region comprising a VH comprising the amino acid sequence of SEQ ID NO:235, and a VL comprising the amino acid sequence of SEQ ID NO:236.

In some specific embodiments, the CD3 binding region provided herein comprises one or more CDRs in Table 13D.

TABLE 13D
CD3B2051-N106A Binding Region Sequences

| | Kabat | Chothia | IMGT |
|---|---|---|---|
| Heavy Chain | RSTM | GYTFT | GYTFT |
| Variable | H | RS | RST |
| Region CDR1 | SEQ ID | SEQ ID | SEQ ID |
| | NO: 216 | NO: 222 | NO: 228 |
| Heavy Chain | YINPSSAY | NPSSAY | INPSSAYT |
| Variable | TNYNQK | SEQ ID | SEQ ID |
| Region CDR2 | FQG | NO: 223 | NO: 229 |
| | SEQ ID | | |
| | NO: 217 | | |
| Heavy Chain | PQVHYDY | PQVHYD | ASPQVH |
| Variable | AGFPY | YAGFP | YDYAG |
| Region CDR3 | SEQ ID | SEQ ID | FPY |
| | NO: 218 | NO: 224 | SEQ ID |
| | | | NO: 230 |
| Light Chain | SASSSV | SSSVSY | SSVSY |
| Variable | SYMN | SEQ ID | SEQ ID |
| Region CDR1 | SEQ ID | NO: 225 | NO: 231 |
| | NO: 219 | | |
| Light Chain | DSSKL | DSS | DSS |
| Variable | AS | | |
| Region CDR2 | SEQ ID | | |
| | NO: 220 | | |
| Light Chain | QQWSRN | WSRN | QQWSRN |
| Variable | PPT | PP | PPT |
| Region CDR3 | SEQ ID | SEQ ID | SEQ ID |
| | NO: 221 | NO: 227 | NO: 232 |

VH (SEQ ID NO: 237)
QVQLVQSGAEVKKPGSSVKVSCKASGYTFTRSTMHWVK
QAPGQGLEWMGYINPSSAYTNYNQKFQGRVTLTADKST
STAYMELSSLRSEDTAVYYCASPQVHYDYAGFPYWGQG
TLVTVSS
VL (SEQ ID NO: 238)
EIVLTQSPATLSASPGERVTLSCSASSSVSYMNWYQQK
PGQAPRRLIYDSSKLASGVPARFSGSGSGRDYTLTISS
LEPEDFAVYYCQQWSRNPPTFGGGTKVEIK

In some specific embodiments, in the CD3E binding region provided herein, the HCDR1 comprises the amino acid sequence of SEQ ID NO:216, the HCDR2 comprises the amino acid sequence of SEQ ID NO:217, the HCDR3 comprises the amino acid sequence of SEQ ID NO:218, the LCDR1 comprises the amino acid sequence of SEQ ID NO:219, the LCDR2 comprises the amino acid sequence of SEQ ID NO:220, and the LCDR3 comprises the amino acid sequence of SEQ ID NO:221.

In some specific embodiments, in the CD3ε binding region provided herein, the HCDR1 comprises the amino acid sequence of SEQ ID NO:222, the HCDR2 comprises the amino acid sequence of SEQ ID NO:223, the HCDR3 comprises the amino acid sequence of SEQ ID NO:224, the LCDR1 comprises the amino acid sequence of SEQ ID NO:225, the LCDR2 comprises the amino acid sequence of DSS, and the LCDR3 comprises the amino acid sequence of SEQ ID NO:227.

In some specific embodiments, in the CD3ε binding region provided herein, the HCDR1 comprises the amino acid sequence of SEQ ID NO:228, the HCDR2 comprises the amino acid sequence of SEQ ID NO:229, the HCDR3 comprises the amino acid sequence of SEQ ID NO:230, the LCDR1 comprises the amino acid sequence of SEQ ID NO:231, the LCDR2 comprises the amino acid sequence of DSS, and the LCDR3 comprises the amino acid sequence of SEQ ID NO:232.

In some embodiments, the CD3ε binding region further comprises one or more framework regions. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FRI is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system. In some embodiments, the CD3ε binding region further comprises one or more framework regions of SEQ ID NOs:237 and 238.

In some embodiments, the CD3ε binding region provided herein comprises a VH comprising the amino acid sequence of SEQ ID NO:237, and a VL comprising the amino acid sequence of SEQ ID NO:238.

In some embodiments, the CD3ε binding region provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:237 and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:238.

In some embodiments, the CD3ε binding region provided herein binds to the same epitope as a CD3ε binding region comprising a VH comprising the amino acid sequence of SEQ ID NO:237, and a VL comprising the amino acid sequence of SEQ ID NO:238.

In some embodiments, the CD3E binding region provided herein specifically binds to CD3ε competitively with a CD3ε binding region comprising a VH comprising the amino acid sequence of SEQ ID NO:237, and a VL comprising the amino acid sequence of SEQ ID NO:238.

In some specific embodiments, the CD3ε binding region is a scFv. In some embodiments, the CD3ε binding scFv comprises one or more amino acid substitutions, such as those stabilizing scFv.

In some embodiments, the CD3ε binding scFv comprises the VH and VL sequences disclosed in any of Tables 13A to 13D. In some embodiments, the CD3ε binding scFv comprises a VH comprising the sequence selected from SEQ ID NOS: 159, 233, 235, and 237. In some embodiments, the CD3ε binding scFv comprises a VH comprising the sequence selected from SEQ ID NOS: 160, 234, 236, and 238. In some embodiments, the CD3ε binding scFv comprises a VH comprising the sequence of SEQ ID NO: 159 and a VL comprising the sequence of SEQ ID NO: 160. In some embodiments, the CD3ε binding scFv comprises a VH comprising the sequence of SEQ ID NO:233 and a VL comprising the sequence of SEQ ID NO:234. In some embodiments, the CD3ε binding scFv comprises a VH comprising the sequence of SEQ ID NO:235 and a VL comprising the sequence of SEQ ID NO:236. In some embodiments, the CD3ε binding scFv comprises a VH comprising the sequence of SEQ ID NO:237 and a VL comprising the sequence of SEQ ID NO:238.

In specific embodiments, any of the VH and the VL domains described herein that bind CD3ε may be engineered into scFv format in either VH-linker-VL or VL-linker-VH orientation. Alternatively, the VH and VL domains may be engineered into scFv format without the use of a linker in either the VH-VL or VL-VH orientation.

In specific embodiments, the CD3ε binding scFv comprises the VH comprising SEQ ID NO:159 and the VL comprising SEQ ID NO:160. In particular embodiments, the VH comprising SEQ ID NO: 159 and the VL comprising SEQ ID NO: 160 are fused directly with one another without a linker in the scFv. In some embodiments, the VH comprising SEQ ID NO: 159 is fused to the N terminus of the VL comprising SEQ ID NO: 160 in the VH-VL orientation. In other embodiments, the VH comprising SEQ ID NO: 159 is fused to the C terminus of the VL comprising SEQ ID NO: 160 in the VL-VH orientation. In some embodiments, the VH comprising SEQ ID NO: 159 and the VL comprising SEQ ID NO: 160 are linked with a linker in the scFv. In some embodiments, the VH comprising SEQ ID NO: 159 and the VL comprising SEQ ID NO: 160 and the linker are fused in the VH-linker-VL orientation in the scFv. In other embodiments, the VH comprising SEQ ID NO: 159 and the VL comprising SEQ ID NO: 160 and the linker are fused in the VL-linker-VH orientation in the scFv. In some embodiments described in the present paragraph, the linker connecting the VH and VL in the scFv comprises a sequence selected from SEQ ID NOS: 166-199.

In specific embodiments, the CD3ε binding scFv comprises the VH comprising SEQ ID NO:233 and the VL comprising SEQ ID NO:234. In particular embodiments, the VH comprising SEQ ID NO:233 and the VL comprising SEQ ID NO:234 are fused directly with one another without a linker in the scFv. In some embodiments, the VH comprising SEQ ID NO:233 is fused to the N terminus of the VL comprising SEQ ID NO:234 in the VH-VL orientation. In other embodiments, the VH comprising SEQ ID NO:233 is fused to the C terminus of the VL comprising SEQ ID NO:234 in the VL-VH orientation. In some embodiments, the VH comprising SEQ ID NO:233 and the VL comprising SEQ ID NO:234 are linked with a linker in the scFv. In some embodiments, the VH comprising SEQ ID NO:233 and the VL comprising SEQ ID NO:234 and the linker are fused in the VH-linker-VL orientation in the scFv. In other embodiments, the VH comprising SEQ ID NO:233 and the VL comprising SEQ ID NO:234 and the linker are fused in the VL-linker-VH orientation in the scFv. In some embodiments described in the present paragraph, the linker connecting the VH and VL in the scFv comprises a sequence selected from SEQ ID NOS: 166-199.

In specific embodiments, the CD3ε binding scFv comprises the VH comprising SEQ ID NO:235 and the VL comprising SEQ ID NO:236. In particular embodiments, the VH comprising SEQ ID NO:235 and the VL comprising SEQ ID NO:236 are fused directly with one another without a linker in the scFv. In some embodiments, the VH comprising SEQ ID NO:235 is fused to the N terminus of the VL comprising SEQ ID NO:236 in the VH-VL orientation. In other embodiments, the VH comprising SEQ ID NO:235 is fused to the C terminus of the VL comprising SEQ ID NO:236 in the VL-VH orientation. In some embodiments, the VH comprising SEQ ID NO:235 and the VL comprising SEQ ID NO:236 are linked with a linker in the scFv. In some embodiments, the VH comprising SEQ ID NO:235 and the VL comprising SEQ ID NO:236 and the linker are fused in the VH-linker-VL orientation in the scFv. In other embodiments, the VH comprising SEQ ID NO:235 and the VL comprising SEQ ID NO:236 and the linker are fused in the VL-linker-VH orientation in the scFv. In some embodiments described in the present paragraph, the linker connecting the VH and VL in the scFv comprises a sequence selected from SEQ ID NOS: 166-199.

In specific embodiments, the CD3ε binding scFv comprises the VH comprising SEQ ID NO:237 and the VL comprising SEQ ID NO:238. In particular embodiments, the VH comprising SEQ ID NO:237 and the VL comprising SEQ ID NO:238 are fused directly with one another without a linker in the scFv. In some embodiments, the VH comprising SEQ ID NO:237 is fused to the N terminus of the VL comprising SEQ ID NO:238 in the VH-VL orientation. In other embodiments, the VH comprising SEQ ID NO:237 is fused to the C terminus of the VL comprising SEQ ID NO:238 in the VL-VH orientation. In some embodiments, the VH comprising SEQ ID NO:237 and the VL comprising SEQ ID NO:238 are linked with a linker in the scFv. In some embodiments, the VH comprising SEQ ID NO:237 and the VL comprising SEQ ID NO:238 and the linker are fused in the VH-linker-VL orientation in the scFv. In other embodiments, the VH comprising SEQ ID NO:237 and the VL comprising SEQ ID NO:238 and the linker are fused in the VL-linker-VH orientation in the scFv. In some embodiments described in the present paragraph, the linker connecting the VH and VL in the scFv comprises a sequence selected from SEQ ID NOS: 166-199.

In particular embodiments, the CD3ε binding scFv is in the orientation of VH-linker-VL. In some embodiments, the scFv comprises an amino acid sequence of SEQ ID NO: 161. In some embodiments, the scFv comprises an amino acid sequence of SEQ ID NO:241. In some embodiments, the scFv comprises an amino acid sequence of SEQ ID NO:243. In some embodiments, the CD3ε binding scFv is in the orientation of VL-linker-VH. In some embodiments, the scFv comprises an amino acid sequence of SEQ ID NO: 162. In some embodiments, the scFv comprises an amino acid sequence of SEQ ID NO:242. In some embodiments, the scFv comprises an amino acid sequence of SEQ ID NO:244.

TABLE 14
CD38-Binding scFvs

| Binding | | |
|---|---|---|
| domain | scFv-HL amino | scFv-LH amino |
| name | acid Sequence | acid Sequence |
| CD3W245 | EVQLVESGGGLVKPGGSLRL | DIQMTQSPSSLSASVGDRVT |
| | SCAASGFTFSRYNMNWVRQA | ITCRARQSIGTAIHWYQQKP |
| | PGKGLEWVSSISTSSNYIYY | GKAPKLLIKYASESISGVPS |
| | ADSVKGRFTFSRDNAKNSLD | RFSGSGSGTDFTLTISSLQP |
| | LQMSGLRAEDTAIYYCTRGW | EDFATYYCQQSGSWPYTFGQ |
| | GPFDYWGQGTLVTVSSGGSE | GTKLEIKGGSEGKSSGSGSE |
| | GKSSGSGSESKSTGGSDIQM | SKSTGGSEVQLVESGGGLVK |
| | TQSPSSLSASVGDRVTITCR | PGGSLRLSCAASGFTFSRYN |
| | ARQSIGTAIHWYQQKPGKAP | MNWVRQAPGKGLEWVSSIST |
| | KLLIKYASESISGVPSRFSG | SSNYIYYADSVKGRFTFSRD |
| | SGSGTDFTLTISSLQPEDFA | NAKNSLDLQMSGLRAEDTAI |
| | TYYCQQSGSWPYTFGQGTKL | YYCTRGWGPFDYWGQGTLVT |
| | EIK | VSS |
| | (Seq ID NO: 161) | (Seq ID NO: 162) |
| CD3B2030- | QVQLVQSGAEVKKPGSSVKV | EIVLTQSPATLSASPGERVT |
| N106A | SCKASGYTFTRSTMHWVKQA | LSCSASSSVSYMNWYQQKPG |
| | PGQGLEWIGYINPSSAYTNY | QAPRRWIYDSSKLASGVPAR |
| | NQKFQGRVTLTADKSTSTAY | FSGSGSGRDYTLTISSLEPE |
| | MELSSLRSEDTAVYYCASPQ | DFAVYYCQQWSRNPPTFGGG |
| | VHYDYAGFPYWGQGTLVTVS | TKVEIKGGSEGKSSGSGSES |
| | SGGSEGKSSGSGSESKSTGG | KSTGGSQVQLVQSGAEVKKP |
| | SEIVLTQSPATLSASPGERV | GSSVKVSCKASGYTFTRSTM |
| | TLSCSASSSVSYMNWYQQKP | HWVKQAPGQGLEWIGYINPS |
| | GQAPRRWIYDSSKLASGVPA | SAYTNYNQKFQGRVTLTADK |
| | RFSGSGSGRDYTLTISSLEP | STSTAYMELSSLRSEDTAVY |
| | EDFAVYYCQQWSRNPPTFGG | YCASPQVHYDYAGFPYWGQG |
| | GTKVEIK | TLVTVSS |
| | (SEQ ID NO: 241) | (SEQ ID NO: 242) |
| CD3B2051- | QVQLVQSGAEVKKPGSSVKV | EIVLTQSPATLSASPGERVT |
| N106A | SCKASGYTFTRSTMHWVKQA | LSCSASSSVSYMNWYQQKPG |
| | PGQGLEWMGYINPSSAYTNY | QAPRRLIYDSSKLASGVPAR |
| | NQKFQGRVTLTADKSTSTAY | FSGSGSGRDYTLTISSLEPE |
| | MELSSLRSEDTAVYYCASPQ | DFAVYYCQQWSRNPPTFGGG |
| | VHYDYAGFPYWGQGTLVTVS | TKVEIKGGSEGKSSGSGSES |
| | SGGSEGKSSGSGSESKSTGG | KSTGGSQVQLVQSGAEVKKP |
| | SEIVLTQSPATLSASPGERV | GSSVKVSCKASGYTFTRSTM |
| | TLSCSASSSVSYMNWYQQKP | HWVKQAPGQGLEWMGYINPS |
| | GQAPRRLIYDSSKLASGVPA | SAYTNYNQKFQGRVTLTADK |
| | RFSGSGSGRDYTLTISSLEP | STSTAYMELSSLRSEDTAVY |
| | EDFAVYYCQQWSRNPPTFGG | YCASPQVHYDYAGFPYWGQG |
| | GTKVEIK | TLVTVSS |
| | (SEQ ID NO: 243) | (SEQ ID NO: 244) |

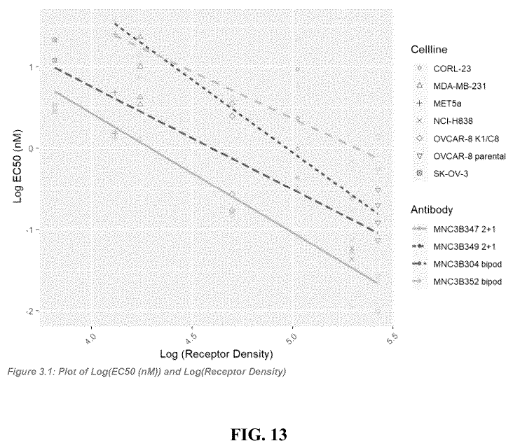

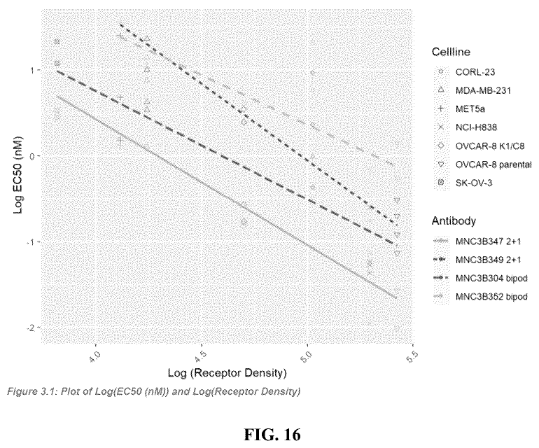

In some embodiments, the MSLN×CD3 binding protein provided herein has the “Bipod” structure as illustrated in FIG. 14 (left). In specific embodiments, the MSLN×CD3 binding protein provided herein comprises a scFv that binds CD3ε and a Fab that binds MSLN, and the binding protein further comprises a Fc region (see FIG. 17). In specific embodiments, the MSLN×CD3 binding protein provided herein comprises: (i) a first polypeptide comprising a scFv that binds CD3ε that is linked to one arm of an antibody Fc region, (ii) a second polypeptide comprising the VH domain that binds MSLN that is linked to the other arm of an antibody Fc region, and (iii) a third polypeptide comprising the VL domain that binds MSLN, wherein the VH domain and the VL domain forms a Fab that binds MSLN, and the first polypeptide and the second polypeptide forms a Fc region.

In other specific embodiments, the MSLN×CD3 binding protein provided herein comprises: (i) a first polypeptide comprising a scFv that binds MSLN that is linked to one arm of an antibody Fc region, (ii) a second polypeptide comprising the VH domain that binds CD3ε that is linked to the other arm of an antibody Fc region, and (iii) a third polypeptide comprising the VL domain that binds CD3ε, wherein the VH domain and the VL domain forms a Fab that binds MSLN, and the first polypeptide and the second polypeptide forms a Fc region.

In some embodiments, the Fc regions comprise one or more modifications that facilitate heterodimerization of the first and second polypeptides. In some embodiments, one arm of the Fc region comprises T366W substitution and the other arm of the Fc region comprises the T366S, L368A, Y407V substitutions. In some embodiments, the one arm of the Fc region comprises the H435R and Y436F substitution. In some embodiments, the second arm of the Fc region comprises the H435R and Y436F substitution. In some embodiments, the Fc region comprises one or more mutations that reduces binding affinity of the Fc domain to Fc receptors. In particular embodiments, one arm of the Fc region comprises L234A, L235A, and D265S (AAS) mutations. In particular embodiments, the second arm of the Fc region comprises L234A, L235A, and D265S (AAS) mutations. In particular embodiments, each the first and second arms of the Fc region comprises L234A, L235A, and D265S (AAS) mutations.

In some embodiments, the MSLN×CD3 binding protein provided herein comprises: (i) a first polypeptide comprising a scFv that binds CD3ε that is linked to one arm of an antibody Fc region, (ii) a second polypeptide comprising the VH domain that binds MSLN that is linked to the other arm of an antibody Fc region, and (iii) a third polypeptide comprising the VL domain that binds MSLN, wherein the VH domain and the VL domain forms a Fab that binds MSLN, and the first polypeptide and the second polypeptide forms a Fc region; further wherein the Fc region of the first polypeptide comprises the T366S, L368A, Y407V substitutions, and the Fc region of the second polypeptide comprises the T366W substitution. In specific embodiments described in this paragraph, the Fc region of the first polypeptide further comprises the L234A, L235A, and D265S (AAS) mutations. In specific embodiments described in this paragraph, the Fc region of the second polypeptide further comprises the L234A, L235A, and D265S (AAS) mutations. In specific embodiments described in this paragraph, both Fc regions of the first and second polypeptides further comprise the L234A, L235A, and D265S (AAS) mutations.

In alternative embodiments, the MSLN×CD3 binding protein provided herein comprises: (i) a first polypeptide comprising a scFv that binds CD3ε that is linked to one arm of an antibody Fc region, (ii) a second polypeptide comprising the VH domain that binds MSLN that is linked to the other arm of an antibody Fc region, and (iii) a third polypeptide comprising the VL domain that binds MSLN, wherein the VH domain and the VL domain forms a Fab that binds MSLN, and the first polypeptide and the second polypeptide forms a Fc region; further wherein the Fc region of the first polypeptide comprises the T366W substitution and the Fc region of the second polypeptide comprises the T366S, L368A, Y407V substitutions. In specific embodiments described in this paragraph, the Fc region of the first polypeptide further comprises the L234A, L235A, and D265S (AAS) mutations. In specific embodiments described in this paragraph, the Fc region of the second polypeptide further comprises the L234A, L235A, and D265S (AAS) mutations. In specific embodiments described in this paragraph, both Fc regions of the first and second polypeptides further comprise the L234A, L235A, and D265S (AAS) mutations.

In specific embodiments, the MSLN×CD3 binding protein provided herein comprises: (i) a first polypeptide comprising a scFv that binds CD3ε that is linked to one arm of an antibody Fc region, (ii) a second polypeptide comprising the VH domain that binds MSLN that is linked to the other arm of an antibody Fc region, and (iii) a third polypeptide comprising the VL domain that binds MSLN, wherein the VH domain and the VL domain forms a Fab that binds MSLN, and the first polypeptide and the second polypeptide forms a Fc region. In some embodiments, the first polypeptide comprising an amino acid sequence of SEQ ID NO: 163. In some embodiments, the first polypeptide is encoded by a nucleic acid molecule comprising the sequence of SEQ ID NO:248. In some embodiments, the second polypeptide comprising an amino acid sequence of SEQ ID NO:164. In some embodiments, the second polypeptide is encoded by a nucleic acid molecule comprising the sequence of SEQ ID NO:239. In some embodiments, the third polypeptide comprising an amino acid sequence of SEQ ID NO:165. In some embodiments, the third polypeptide is encoded by a nucleic acid sequence of SEQ ID NO:240.

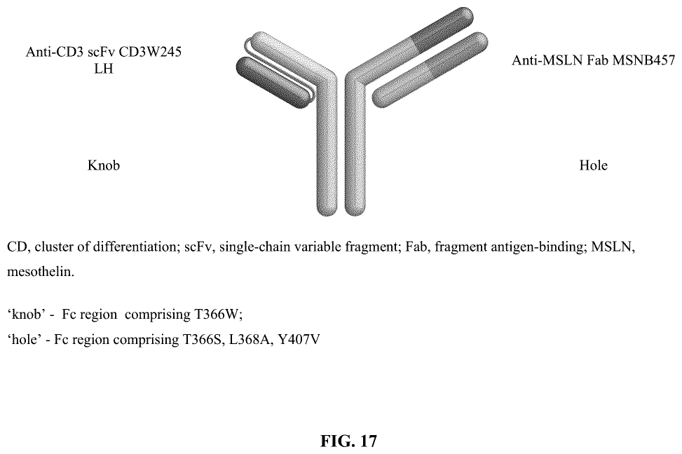

In a specific embodiment, the MSLN×CD3 binding protein provided herein comprises: (i) a first polypeptide comprising a scFv that binds CD3ε that is linked to one arm of an antibody Fc region, (ii) a second polypeptide comprising the VH domain that binds MSLN that is linked to the other arm of an antibody Fc region, and (iii) a third polypeptide comprising the VL domain that binds MSLN, wherein the VH domain and the VL domain forms a Fab that binds MSLN, and the first polypeptide and the second polypeptide forms a Fc region, wherein the first polypeptide comprises an amino acid sequence of SEQ ID NO: 163, the second polypeptide comprises an amino acid sequence of SEQ ID NO: 164, and the third polypeptide comprises an amino acid sequence of SEQ ID NO: 165 (see Table 15 or FIG. 18).

TABLE 15
Sequences of MNC3B304.

| | Amino Acids (CDRs | |
|---|---|---|
| | and Fc mutations | Encoding |
| | underlined) | Polynucleotides |
| Heavy | DIQMTQSPSSLSASVGDRVT | GACATCCAGATGACCCAGTC |
| Chain 1 | ITCRARQSIGTAIHWYQQKP | TCCATCCTCTCTGTCCGCCT |
| (1^{st} poly- | GKAPKLLIKYASESISGVPS | CTGTGGGCGACAGAGTGACC |
| peptide) | RFSGSGSGTDFTLTISSLQP | ATTACCTGCCGGGCCAGACA |
| | EDFATYYCQQSGSWPYTFGQ | GTCTATCGGCACCGCTATCC |
| | GTKLEIKGGSEGKSSGSGSE | ACTGGTATCAGCAGAAGCCT |
| | SKSTGGSEVQLVESGGGLVK | GGCAAGGCCCCTAAGCTGCT |
| | PGGSLRLSCAASGFTFSRYN | GATTAAGTACGCCTCCGAGT |
| | MNWVRQAPGKGLEWVSSIST | CCATCTCCGGCGTGCCCTCC |
| | SSNYIYYADSVKGRFTFSRD | AGATTTTCTGGCTCTGGATC |
| | NAKNSLDLQMSGLRAEDTAI | TGGCACCGACTTTACCCTGA |
| | YYCTRGWGPFDYWGQGTLVT | CAATCTCCAGCCTGCAGCCT |
| | VSSEPKSSDKTHTCPPCPAP | GAGGACTTCGCCACCTACTA |
| | EAAGGPSVFLFPPKPKDTLM | CTGTCAGCAGTCCGGCTCTT |
| | ISRTPEVTCVVVSVSHEDPE | GGCCTTACACCTTTGGTCAG |
| | VKFNWYVDGVEVHNAKTKPR | GGCACCAAGCTGGAAATCAA |
| | EEQYNSTYRVVSVLTVLHQD | GGGCGGATCTGAGGGAAAGT |
| | WLNGKEYKCKVSNKALPAPI | CCAGCGGCTCCGGCAGCGAA |
| | EKTISKAKGQPREPQVYTLP | AGCAAGTCCACCGGCGGAAG |
| | PSREEMTKNQVSLWCLVKGF | CGAGGTGCAGCTGGTTGAAT |
| | YPSDIAVEWESNGQPENNYK | CTGGCGGAGGACTGGTTAAG |
| | TTPPVLDSDGSFFLYSKLTV | CCTGGCGGCTCTCTGAGACT |
| | DKSRWQQGNVFSCSVMHEAL | GTCTTGTGCTGCTTCTGGCT |
| | HNHYTQKSLSLSPGK | TCACCTTCAGCCGGTACAAC |
| | (SEQ ID NO: 163) | ATGAACTGGGTCCGACAGGC |
| | | TCCTGGCAAAGGCCTGGAAT |
| | | GGGTGTCCTCCATCTCCACC |
| | | TCCAGCAACTACATCTACTA |
| | | CGCCGACTCCGTGAAGGGCA |
| | | GATTCACCTTCTCCAGAGAC |
| | | AACGCCAAGAACTCCCTGGA |
| | | CCTGCAGATGTCTGGCCTGA |
| | | GAGCTGAGGACACCGCTATC |
| | | TACTACTGCACCAGAGGCTG |
| | | GGGACCCTTCGATTATTGGG |
| | | GCCAGGGAACCCTGGTCACC |
| | | GTGTCATCTGAGCCCAAATC |
| | | TAGCGACAAAACTCACACAT |
| | | GCCCACCGTGCCCAGCACCT |
| | | GAAGCCGCCGGGGGACCGTC |
| | | AGTCTTCCTCTTCCCCCCAA |
| | | AACCCAAGGACACCCTCATG |
| | | ATCTCCCGGACCCCTGAGGT |
| | | CACATGCGTGGTGGTGAGCG |
| | | TGAGCCACGAAGACCCTGAG |
| | | GTCAAGTTCAACTGGTACGT |
| | | GGACGGCGTGGAGGTGCATA |
| | | ATGCCAAGACAAAGCCGCGG |
| | | GAGGAGCAGTACAACAGCAC |
| | | GTACCGTGTGGTCAGCGTCC |
| | | TCACCGTCCTGCACCAGGAC |
| | | TGGCTGAATGGCAAGGAGTA |
| | | CAAGTGCAAGGTGTCGAACA |
| | | AAGCCCTCCCAGCCCCCATC |
| | | GAGAAAACCATCTCCAAAGC |
| | | CAAAGGGCAGCCCCGAGAAC |
| | | CACAGGTGTACACCCTGCCC |
| | | CCATCCCGGGAGGAGATGAC |
| | | CAAGAACCAGGTCAGCCTGT |
| | | GGTGCCTGGTCAAAGGCTTC |
| | | TATCCCAGCGACATCGCCGT |
| | | GGAGTGGGAGAGCAATGGGC |
| | | AGCCGGAGAACAACTACAAG |
| | | ACCACGCCTCCCGTGCTGGA |
| | | CTCCGACGGCTCCTTCTTCC |
| | | TCTACAGCAAGCTCACCGTG |
| | | GACAAGAGCAGATGGCAGCA |
| | | GGGGAACGTCTTCTCATGCT |
| | | CCGTGATGCATGAGGCTCTG |
| | | CACAACCACTACACGCAGAA |
| | | GTCTCTCTCCCTGTCTCCGG |
| | | GAAAA(SEQ ID NO: 248) |
| Heavy | QVQLVESGPGQVKPSQTLSL | CAGGTGCAGCTGGTGGAATC |
| Chain 2 | TCAISGDSVSSNSAAWNWIR | TGGACCTGGCCAAGTGAAGC |
| (2^{nd} | QSPSRGLEWLGRTYYRSKWY | CCTCTCAGACCCTGTCTCTG |
| poly- | NDYAVSVKSRITINPDTSKN | ACCTGTGCCATCTCCGGCGA |
| peptide) | QFSLQLNSVTPEDTAVYYCA | CTCCGTGTCCTCTAATTCTG |
| | RRDYSSGGFDYWGQGTTVTV | CCGCCTGGAACTGGATCCGG |
| | SSASTKGPSVFPLAPSSKST | CAGTCTCCTAGTAGAGGCCT |
| | SGGTAALGCLVKDYFPEPVT | GGAATGGCTGGGCAGAACCT |
| | VSWNSGALTSGVHTFPAVLQ | ACTACCGGTCCAAGTGGTAC |
| | SSGLYSLSSVVTVPSSSLGT | AACGACTACGCCGTGTCCGT |
| | QTYICNVNHKPSNTKVDKKV | GAAGTCCCGGATCACCATCA |
| | EPKSCDKTHTCPPCPAPEAA | ATCCCGACACCTCCAAGAAC |
| | GGPSVFLFPPKPKDTLMISR | CAGTTCTCCCTGCAGCTGAA |
| | TPEVTCVVVSVSHEDPEVKF | CAGCGTGACCCCTGAGGATA |
| | NWYVDGVEVHNAKTKPREEQ | CCGCCGTGTACTACTGCGCC |
| | YNSTYRVVSVLTVLHQDWLN | AGACGGGACTATTCTTCCGG |
| | GKEYKCKVSNKALPAPIEKT | CGGCTTTGATTACTGGGGCC |
| | ISKAKGQPREPQVYTLPPSR | AGGGCACAACAGTGACCGTG |
| | EEMTKNQVSLSCAVKGFYPS | TCATCTGCCTCCACCAAGGG |
| | DIAVEWESNGQPENNYKTTP | CCCATCGGTCTTCCCCCTGG |
| | PVLDSDGSFFLVSKLTVDKS | CACCCTCCTCCAAGAGCACC |
| | RWQQGNVFSCSVMHEALHNR | TCTGGGGGCACAGCGGCCCT |
| | FTQKSLSLSPGK | GGGCTGCCTGGTCAAGGACT |
| | (SEQ ID NO: 164) | ACTTCCCCGAACCGGTGACG |
| | | GTGTCGTGGAACTCAGGCGC |
| | | CCTGACCAGCGGCGTGCACA |
| | | CCTTCCCGGCTGTCCTACAG |
| | | TCCTCAGGACTCTACTCCCT |
| | | CAGCAGCGTGGTGACCGTGC |
| | | CCTCCAGCAGCTTGGGCACC |
| | | CAGACCTACATCTGCAACGT |
| | | GAATCACAAGCCCAGCAACA |
| | | CCAAGGTGGACAAGAAAGTT |
| | | GAGCCCAAATCTTGTGACAA |
| | | AACTCACACATGCCCACCGT |
| | | GCCCAGCACCTGAAGCCGCC |
| | | GGGGGACCGTCAGTCTTCCT |
| | | CTTCCCCCCAAAACCCAAGG |
| | | ACACCCTCATGATCTCCCGG |
| | | ACCCCTGAGGTCACATGCGT |
| | | GGTGGTGAGCGTGAGCCACG |
| | | AAGACCCTGAGGTCAAGTTC |
| | | AACTGGTACGTGGACGGCGT |
| | | GGAGGTGCATAATGCCAAGA |
| | | CAAAGCCGCGGGAGGAGCAG |
| | | TACAACAGCACGTACCGTGT |
| | | GGTCAGCGTCCTCACCGTCC |
| | | TGCACCAGGACTGGCTGAAT |
| | | GGCAAGGAGTACAAGTGCAA |
| | | GGTGTCGAACAAAGCCCTCC |
| | | CAGCCCCCATCGAGAAAACC |
| | | ATCTCCAAAGCCAAAGGGCA |
| | | GCCCCGAGAACCACAGGTGT |
| | | ACACCCTGCCCCCATCCCGG |
| | | GAGGAGATGACCAAGAACCA |
| | | GGTCAGCCTGTCCTGCGCCG |
| | | TCAAAGGCTTCTATCCCAGC |
| | | GACATCGCCGTGGAGTGGGA |
| | | GAGCAATGGGCAGCCGGAGA |
| | | ACAACTACAAGACCACGCCT |
| | | CCCGTGCTGGACTCCGACGG |
| | | CTCCTTCTTCCTCGTGAGCA |
| | | AGCTCACCGTGGACAAGAGC |
| | | AGATGGCAGCAGGGGAACGT |
| | | CTTCTCATGCTCCGTGATGC |
| | | ATGAGGCTCTGCACAACCGG |
| | | TTCACGCAGAAGTCTCTCTC |
| | | CCTGTCTCCGGGAAAA |
| | | (SEQ ID NO: 239) |
| Light | EIVLTQSPDSLAVSLGERAT | GAGATCGTGCTGACCCAGTC |
| Chain | INCKSSQSVLYSSDNKNYLA | TCCTGACTCTCTGGCTGTGT |
| (3^{rd} | WYQQKPGQPPNLLIYWASTR | CTCTGGGCGAGAGAGCCACC |
| poly- | ESGVPDRFSGSGSGTDFTLT | ATCAACTGCAAGTCCTCTCA |
| peptide | INSLQAEDVAVYYCQQYYST | GTCCGTGCTGTACTCCTCCG |
| | PFTFGPGTKVEIKRTVAAPS | ACAACAAGAACTACCTGGCT |
| | VFIFPPSDEQLKSGTASVVC | TGGTATCAGCAGAAGCCTGG |
| | LLNNFYPREAKVQWKVDNAL | TCAGCCTCCTAACCTGCTGA |
| | QSGNSQESVTEQDSKDSTYS | TCTACTGGGCCTCCACCAGA |
| | LSSTLTLSKADYEKHKVYAC | GAGTCTGGTGTGCCCGATCG |
| | EVTHQGLSSPVTKSFNRGEC | CTTTTCCGGCTCTGGCTCTG |
| | (SEQ ID NO: 165) | GCACCGACTTCACCCTGACC |
| | | ATCAATTCCCTGCAGGCCGA |
| | | GGATGTGGCCGTGTACTACT |
| | | GCCAGCAGTACTACAGCACC |
| | | CCTTTCACCTTCGGACCCGG |
| | | CACCAAGGTGGAAATCAAGC |
| | | GTACTGTGGCTGCACCATCT |
| | | GTCTTCATCTTCCCGCCATC |
| | | TGATGAGCAGTTGAAATCTG |
| | | GAACTGCCTCTGTTGTGTGC |
| | | CTGCTGAATAACTTCTATCC |
| | | CAGAGAGGCCAAAGTACAGT |
| | | GGAAGGTGGATAACGCCCTC |
| | | CAATCGGGTAACTCCCAGGA |
| | | GAGTGTCACAGAGCAGGACA |
| | | GCAAGGACAGCACCTACAGC |
| | | CTCAGCAGCACCCTGACGCT |
| | | GAGCAAAGCAGACTACGAGA |
| | | AACACAAAGTCTACGCCTGC |
| | | GAAGTCACCCATCAGGGCCT |
| | | GAGCTCGCCCGTCACAAAGA |
| | | GCTTCAACAGGGGAGAGTGT |
| | | (SEQ ID NO: 240) |

In some embodiments, the MSLN×CD3 binding protein provided herein has the “2+1” structure as illustrated in FIG. 14 (right). In specific embodiments, the MSLN×CD3 binding protein provided herein comprises: (i) a first polypeptide comprising a scFv that binds a CD3&, wherein the scFv is linked with a first VH (or first VL) domain that binds MSLN on one end, and is further linked to one arm of an antibody Fc region on the other end; (ii) a second polypeptide comprising a first VL (or first VH) domain that binds MSLN, wherein the first VH (or first VL) and the first VL (or first VH) domains form a Fab that binds MSLN; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the MSLN×CD3 binding protein provided herein comprises: (i) a first polypeptide comprising a scFv that binds a CD3ε, wherein the scFv is linked with a first VH (or first VL) domain that binds MSLN on one end, and is further linked to one arm of an antibody Fc region on the other end; (ii) a second polypeptide comprising a first VL (or first VH) domain that binds MSLN, wherein the first VH (or first VL) and the first VL (or first VH) domains form a Fab that binds MSLN; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the MSLN×CD3 binding protein provided herein comprises: (i) a first polypeptide comprising a scFv that binds a MSLN, wherein the scFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds MSLN, wherein the second VH domain is linked to the other arm of an antibody Fc region, and (iv) a fourth polypeptide comprising the second VL domain that binds MSLN, wherein the second VH domain and the second VL domain form a Fab that binds MSLN, and wherein the first polypeptide and the third polypeptide forms a Fc region.

In other specific embodiments, the MSLN×CD3 binding protein provided herein comprises: (i) a first polypeptide comprising a scFv that binds a CD3ε, wherein the scFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds MSLN, wherein the second VH domain is linked to the other arm of an antibody Fc region, and (iv) a fourth polypeptide comprising the second VL domain that binds MSLN, wherein the second VH domain and the second VL domain form a Fab that binds MSLN, and wherein the first polypeptide and the third polypeptide forms a Fc region.

In other specific embodiments, the MSLN×CD3 binding protein provided herein comprises: (i) a first polypeptide comprising a scFv that binds a MSLN, wherein the scFv is linked with a first VH (or first VL) domain that binds MSLN on one end, and is further linked to one arm of an antibody Fc region on the other end; (ii) a second polypeptide comprising a first VL (or first VH) domain that binds MLSN, wherein the first VH (or first VL) and the first VL (or first VH) domains form a Fab that binds MSLN; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the MSLN×CD3 binding protein provided herein comprises: (i) a first polypeptide comprising a scFv that binds a MSLN, wherein the scFv is linked with a first VH (or first VL) domain that binds CD3ε on one end, and is further linked to one arm of an antibody Fc region on the other end; (ii) a second polypeptide comprising a first VL (or first VH) domain that binds CD3ε, wherein the first VH (or first VL) and the first VL (or first VH) domains form a Fab that binds CD3ε; (iii) a third polypeptide comprising a second VH domain that binds CD3ε, wherein the second VH domain is linked to the other arm of an antibody Fc region, and (iv) a fourth polypeptide comprising the second VL domain that binds CD3ε, wherein the second VH domain and the second VL domain form a Fab that binds CD3ε, and wherein the first polypeptide and the third polypeptide forms a Fc region.

In other specific embodiments, the MSLN×CD3 binding protein provided herein comprises: (i) a first polypeptide comprising a scFv that binds a CD3ε, wherein the scFv is linked with a first VH (or first VL) domain that binds MSLN on one end, and is further linked to one arm of an antibody Fc region on the other end; (ii) a second polypeptide comprising a first VL (or first VH) domain that binds MSLN, wherein the first VH (or first VL) and the first VL (or first VH) domains form a Fab that binds MSLN; (iii) a third polypeptide comprising a second VH domain that binds MSLN, wherein the second VH domain is linked to the other arm of an antibody Fc region, and (iv) a fourth polypeptide comprising the second VL domain that binds MSLN, wherein the second VH domain and the second VL domain form a Fab that binds MSLN, and wherein the first polypeptide and the third polypeptide forms a Fc region.

In yet particular embodiments, the MSLN×CD3 binding protein provided is in the Central-scFv format as described in U.S. Pat. No. 10,889,653, the content of which is herein incorporated by reference in its entirety.

In some embodiments, the Fc region comprises one or more amino acid mutations that facilitate the dimerization of the two arms of the Fc region. In some embodiments, the MSLN×CD3 binding protein provided herein comprises: (i) a first polypeptide comprising a scFv that binds CD3ε, a CH2, and a CH3, (ii) a second polypeptide comprising the VH domain that binds MSLN, a CH2, and a CH3, and (iii) a third polypeptide comprising the VL domain that binds MSLN, wherein the VH domain and the VL domain forms a Fab that binds MSLN, and the first polypeptide and the second polypeptide forms a Fc region.

In other embodiments, the MSLN×CD3 binding protein provided herein comprises a scFv that binds CD3 and two Fabs each bind MSLN, and the binding agent further comprises a Fc region. In some embodiments, the two Fabs are identical and are linked to each other.

In some embodiments, the MSLN×CD3 binding protein provided herein comprises: (i) a first polypeptide comprising a scFv that binds CD3ε that is linked to one arm of an antibody Fc region, (ii) a second polypeptide comprising two identical VH domains in tandem each bind MSLN that is linked to the other arm of an antibody Fc region, (iii) a third polypeptide comprising a VL domain that binds MSLN, and (iv) a fourth polypeptide comprising a VL domain that binds MSLN, wherein the two VH domains and the two VL domains form two Fabs that bind MSLN, and the first polypeptide and the second polypeptide forms a Fc region. In some embodiments, the Fc region comprises one or more amino acid mutations that facilitate the dimerization of the two arms of the Fc region.

In some embodiments, the MSLN×CD3 binding protein provided herein comprises: (i) a first polypeptide comprising a scFv that binds CD3ε, a CH2, and a CH3, (ii) a second polypeptide comprising a first VH domain that binds MSLN, a second VH domain that binds MSLN, a CH2, and a CH3, (iii) a third polypeptide comprising a first VL domain that binds MSLN, and (iv) a fourth polypeptide comprising a second VH domain that binds MSLN, wherein the first VH domain and the first VL domain forms a first Fab that binds MSLN, the second VH domain and the second VL domain forms a second Fab that binds MSLN, and the first polypeptide and the second polypeptide forms a Fc region.

7.7 Pharmaceutical Compositions

In another general aspect, provided is a pharmaceutical composition comprising the MSLN binding agent (monospecific or multispecific) provided herein and a pharmaceutically acceptable excipient. In some embodiments, the MSLN binding agent is a MSLN binding protein. In another general aspect, provided is a pharmaceutical composition comprising a nucleic acid encoding the MSLN binding protein (monospecific or multispecific) provided herein or a fragment or a portion thereof and a pharmaceutically acceptable excipient. In another general aspect, provided is a pharmaceutical composition comprising an engineered cell expressing the MSLN binding protein (monospecific or multispecific) provided herein a pharmaceutically acceptable excipient.

In another general aspect, provided herein is a method of producing a pharmaceutical composition comprising a binding agent or an antigen binding region thereof provided herein, comprising combining a binding agent or an antigen binding region thereof with a pharmaceutically acceptable carrier to obtain the pharmaceutical composition.

Methods of Use

The functional activity of MSLN binding agents (monospecific or multispecific) provided herein can be characterized by methods known in the art and as described herein. Methods for characterizing binding agents include, but are not limited to, affinity and specificity assays including Biacore, ELISA, and OctetRed analysis; binding assays to detect the binding of antibodies to target cells by FACS; binding assays to detect the binding of antibodies to the target antigen on cells. According to particular embodiments, the methods for characterizing binding agents include those described below.

An MSLN binding agent (monospecific or multispecific) of the disclosure is useful in a variety of applications including, but not limited to, therapeutic treatment methods, such as treatment of cancer. In some embodiments, the therapeutic treatment methods comprise immunotherapy for cancer. In some embodiments, the MSLN binding agent is useful for activating, promoting, increasing, and/or enhancing an immune response to a cancer or cancer cell. In some embodiments, the MSLN binding agent is useful for activating, promoting, increasing, and/or enhancing an immune response to a tumor or tumor cell. In some embodiments, the MSLN binding agent is useful for activating, promoting, increasing, and/or enhancing a T cell response to a cancer or cancer cells. In some embodiments, the MSLN binding agent is useful for activating, promoting, increasing, and/or enhancing a T cell response to a tumor or tumor cell. The methods of use may be in vitro, ex vivo, or in vivo methods.

In one aspect, provided herein is a method of directing a T cell to a target cell expressing MSLN by contacting the T cell with an effective amount of an MSLN×CD3 binding agent provided herein. In some embodiments, wherein the CD3 binding region binds the T cell. In some embodiments, the MSLN binding region binds the target cell. In some embodiments, the target cell is a tumor or cancer cell. In some embodiments, the target cell expresses MSLN at a level higher than a reference level of expression of MSLN. In some embodiments, the target cell expresses MSLN at a lower level than a reference level of expression of MSLN. In some embodiments, the target cell is not homozygous for a single nucleotide polymorphism in the MSLN gene that results in Met593 Val substitution in the encoded MSLN. In some embodiments, the target cell is does not contain an SNP in the MSLN gene that results in Met593 Val substitution in the encoded MSLN. In some embodiments, the target cell contains an SNP in the MSLN gene that results in results in Met593 Val substitution in the encoded MSLN, but is heterozygous for the SNP.

In another aspect, provided herein is a method of directing a T cell to a cancer or tumor cell, comprising contacting the T cell with an effective amount of a pharmaceutical composition comprising an MSLN×CD3 binding agent provided herein, wherein the CD3 binding region binds the T cell. In some embodiments, the directed T cell incudes cytokine release. In some embodiments, cytokine release is increased compared to a reference. In some embodiments, said cytokine release is determined by measuring IL-1β, IL-2, IL-4, IL-6, IL-8, IL-10, IL-12, IL-13, TNF-α, IFN-γ, or any combination thereof. In some embodiments, the directed T cell induces cytokine release, and release of one or more of IL-1β, IL-2, IL-4, IL-6, IL-8, IL-10, IL-12, IL-13, TNF-α, and IFN-γ is increased as compared to a reference. In some embodiments, a cytokine reference is: (a) a cytokine measured in a corresponding normal cell or issue; (b) a cytokine measured in a neighboring non-cancerous cell or tissue in the same subject; or (c) a cytokine measured in a corresponding cell or tissue measured in a cohort of healthy subjects. In some embodiments, the directed T cell induces apoptosis in the cancer or tumor cell. In some embodiments, when the T cell is directed to the cancer or tumor cell, the T cell induces differential cytotoxicity and cytokine release. That is, a method of directing a T cell to a cancer or tumor cell results in T-cell dependent cytotoxicity (TDCC) that is inversely related to T cell cytokine release. For example, in some embodiments, TDCC is increased compared to a reference and cytokine release is decreased compared to a reference. In some embodiments, said TDCC reference is: (a) TDCC measured in a corresponding normal cell or tissue; (b) TDCC measured in a neighboring non-cancerous cell or tissue in the same subject; or (c) TDCC measured in a corresponding cell or tissue measured in a cohort of healthy subjects. In some embodiments, said TDCC is determined by measuring apoptosis. In some embodiments, caspase mediated apoptosis is increased. In some embodiments, a cytokine reference is: (a) a cytokine measured in a corresponding normal cell or issue; (b) a cytokine measured in a neighboring non-cancerous cell or tissue in the same subject; or (c) a cytokine measured in a corresponding cell or tissue measured in a cohort of healthy subjects. In some embodiments, said cytokine release is determined by measuring IL-1β, IL-2, IL-4, IL-6, IL-8, IL-10, IL-12, IL-13, TNF-α, IFN-γ, or any combination thereof.

In some embodiments, the cancer or tumor cell comprises an ovarian cancer, a primary pleural and peritoneal mesothelioma, a primary pleural mesothelioma, a primary peritoneal mesothelioma, and a pancreatic ductal adenocarcinoma. In some embodiments, an ovarian cancer is high grade serous epithelial ovary cancer, fallopian tube cancer or primary peritoneal cancer. In some embodiments, a primary pleural and/or peritoneal mesothelioma do not include sarcomatoid and well-differentiated papillary histologies. In some embodiments, a pancreatic ductal adenocarcinoma does not include neuroendocrine cancer, squamous cancer or pseudopapillary cancer. In some embodiments, the cancer cell expresses MSLN. In some embodiments, the cancer cell expresses high levels of MSLN compared to a reference expression level. In some embodiments, the cancer cell expresses low levels of MSLN compared to a reference expression level. In some embodiments, said reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects. In some embodiments, said expression level of MSLN is determined by measuring the protein expression level of MSLN. In some embodiments, the cancer or tumor cell does not contain a homozygous single nucleotide polymorphism (SNP) in the MSLN gene that results in Met593 Val substitution in the encoded MSLN.

In one aspect, provided herein is a method of activating a T cell, comprising contacting the T cell with an effective amount of the MSLN×CD3 binding protein provided herein, wherein the CD3 binding region binds the T cell. In another aspect, provided herein is a method of activating a T cell, comprising contacting the T cell with a pharmaceutical composition comprising an MSLN×CD3 binding protein provided herein.

In some embodiments, T cell activation is measured by expression of certain markers by the T cells. In some embodiments, T cell activation is measured by the percentage of T cells positive for a particular marker in a population of T cells. In some embodiments, the T cell activation marker is CD25. In some embodiments, the T cell activation marker is CD69. In some embodiments, the method of activating the T cell increases expression of CD25, CD69 or both CD25 and CD69 in the T cells. In some embodiments, the method of activating the T cell increases the percentage of CD25+ T cells, CD69+ T cells, and/or CD25+/CD69+ T cell in the population of T cells.

In one aspect, provided herein is a method of targeting an antigen on the surface of a target cell, the method comprising contacting the target cell with an effective amount of an MSLN×CD3 binding protein provided herein, wherein the MSLN binding region binds to the target cell. In another aspect, provided herein is a method of targeting an antigen on the surface of a target cell, the method comprising contacting the target cell with an effective amount of a pharmaceutical composition comprising a MSLN×CD3 binding protein provided herein, wherein the MSLN binding region binds to the target cell. In some embodiments, provided herein is a method of targeting an antigen on the surface of a target cell, the method comprising contacting the target cell with an effective amount of a pharmaceutical composition comprising a MSLN×CD3 binding protein provided herein. In some embodiments, the target cell is a cancer or tumor cell that expresses MSLN. In some embodiments, the target cell expresses high levels of MSLN compared to a reference expression level. In some embodiments, the target cell expresses low levels of MSLN compared to a reference expression level. In some embodiments, said reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects. In some embodiments, said expression level of MSLN is determined by measuring the protein expression level of MSLN. In some embodiments, the cancer or tumor cell comprises an ovarian cancer, a primary pleural and peritoneal mesothelioma, a primary pleural mesothelioma, a primary peritoneal mesothelioma, and a pancreatic ductal adenocarcinoma. In some embodiments, an ovarian cancer is high grade serous epithelial ovary cancer, fallopian tube cancer or primary peritoneal cancer. In some embodiments, a primary pleural and/or peritoneal mesothelioma does not include sarcomatoid and well-differentiated papillary histologies. In some embodiments, a pancreatic ductal adenocarcinoma does not include neuroendocrine cancer, squamous cancer or pseudopapillary cancer. In some embodiments, the cancer or tumor cells does not contain a homozygous single nucleotide polymorphism (SNP) in the MSLN gene that results in Met593 Val substitution in the encoded MSLN protein.

In one aspect, provided herein is a method of killing or inhibiting the proliferation of a cancer or tumor cell, comprising contacting the cancer or tumor cell with a MSLN×CD3 binding protein provided herein. In another aspect, provided herein is a method of killing or inhibiting the proliferation of a cancer or tumor cell, comprising contacting the cancer or tumor cell with a pharmaceutical composition comprising a MSLN×CD3 binding protein provided herein. In some embodiments, the MSLN×CD3 binding protein activates a T cell. In some embodiments, the CD3 binding region activates the T cell. In some embodiments, the activated T cell induces apoptosis in the cancer or tumor cell. In some embodiments, the cancer or tumor cell comprises an ovarian cancer, a primary pleural and peritoneal mesothelioma, a primary pleural mesothelioma, a primary peritoneal mesothelioma, or a pancreatic ductal adenocarcinoma. In some embodiments, an ovarian cancer is high grade serous epithelial ovary cancer, fallopian tube cancer or primary peritoneal cancer. In some embodiments, a primary pleural and/or peritoneal mesothelioma does not include sarcomatoid and well-differentiated papillary histologies. In some embodiments, a pancreatic ductal adenocarcinoma does not include neuroendocrine cancer, squamous cancer, or pseudopapillary cancer. In some embodiments, the cancer or tumor cells does not contain a homozygous single nucleotide polymorphism (SNP) in the MSLN gene that results in Met593 Val substitution in the encoded MSLN protein. In some embodiments, the cancer or tumor cell expresses high levels of MSLN compared to a reference expression level. In some embodiments, the cancer or tumor cell expresses low levels of MSLN compared to a reference expression level. In some embodiments, said reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects. In some embodiments, said expression level of MSLN is determined by measuring the protein expression level of MSLN.

In one aspect, provided herein is a method of treating a cancer or tumor in a subject, comprising administering an effective amount of a MSLN×CD3 binding protein provided herein. In another aspect, provided herein is a method of treating a cancer or tumor in a subject, comprising administering an effective amount of a pharmaceutical composition comprising a MSLN×CD3 binding protein provided herein or the pharmaceutical composition provided herein In some embodiments, the cancer or tumor cell comprises an ovarian cancer, a primary pleural and peritoneal mesothelioma, a primary pleural mesothelioma, a primary peritoneal mesothelioma, and a pancreatic ductal adenocarcinoma. In some embodiments, an ovarian cancer is high grade serous epithelial ovary cancer, fallopian tube cancer or primary peritoneal cancer. In some embodiments, a primary pleural and/or peritoneal mesothelioma does not include sarcomatoid and well-differentiated papillary histologies. In some embodiments, a pancreatic ductal adenocarcinoma does not include neuroendocrine cancer, squamous cancer or pseudopapillary cancer. In some embodiments, the cancer or tumor cells does not contain a homozygous single nucleotide polymorphism (SNP) in the MSLN gene that results in Met593 Val substitution in the encoded MSLN protein. In some embodiments, the cancer or tumor cell expresses MSLN. In some embodiments, the cancer or tumor cell expresses high levels of MSLN compared to a reference expression level. In some embodiments, the cancer or tumor cell expresses low levels of MSLN compared to a reference expression level. In some embodiments, said reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects. In some embodiments, said expression level of MSLN is determined by measuring the protein expression level of MSLN.

In another aspect, provided herein is a use of the MSLN×CD3 binding protein provided herein in the manufacture of a medicament for treatment of a cancer or tumor in a subject thereof. In yet a further aspect, provided herein is a binding agent for use in the treatment of a cancer or tumor. In some embodiments, the cancer or tumor cell comprises an ovarian cancer, a primary pleural and peritoneal mesothelioma, a primary pleural mesothelioma, a primary peritoneal mesothelioma, and a pancreatic ductal adenocarcinoma. In some embodiments, an ovarian cancer is high grade serous epithelial ovary cancer, fallopian tube cancer or primary peritoneal cancer. In some embodiments, a primary pleural and/or peritoneal mesothelioma does not include sarcomatoid and well-differentiated papillary histologies. In some embodiments, a pancreatic ductal adenocarcinoma does not include neuroendocrine cancer, squamous cancer or pseudopapillary cancer. In some embodiments, the cancer or tumor cells does not contain a homozygous single nucleotide polymorphism (SNP) in the MSLN gene that results in Met593 Val substitution in the encoded MSLN protein. In some embodiments, the cancer or tumor cell expresses MSLN. In some embodiments, the cancer or tumor cell expresses high levels of MSLN compared to a reference expression level. In some embodiments, the cancer or tumor cell expresses low levels of MSLN compared to a reference expression level. In some embodiments, said reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects. In some embodiments, said expression level of MSLN is determined by measuring the protein expression level of MSLN.

In one aspect, provided herein is a method of diagnosing and treating a subject having a MSLN-expressing cancer or tumor, comprising (a) detecting presence or absence of a SNP in the MSLN gene in the subject that results in Met593 Val substitution in the encoded MSLN protein; (b) diagnosing the subject as likely responsive to the treatment of an MSLN×CD3 binding protein described herein if the subject is not homozygous for the SNP; and (c) administering or provide for administration of an effective amount of the MSLN×CD3 binding protein to the subject if the subject is diagnosed as likely responsive in step (b). In one aspect, provided herein is a method of treating a subject having a MSLN-expressing cancer or tumor, comprising administering or providing for administration of an effective amount of the MSLN×CD3 binding protein to the subject wherein the subject has a MSLN-expressing cancer or tumor that does not express a homozygous SNP wherein the SNP is germline homozygous SNP resulting in Met593 Val substitution in the MSLN protein. In some embodiments, the cancer or tumor cell comprises an ovarian cancer, a primary pleural and peritoneal mesothelioma, a primary pleural mesothelioma, a primary peritoneal mesothelioma, and a pancreatic ductal adenocarcinoma. In some embodiments, an ovarian cancer is high grade serous epithelial ovary cancer, fallopian tube cancer or primary peritoneal cancer. In some embodiments, a primary pleural and/or peritoneal mesothelioma does not include sarcomatoid and well-differentiated papillary histologies. In some embodiments, a pancreatic ductal adenocarcinoma does not include neuroendocrine cancer, squamous cancer or pseudopapillary cancer. In some embodiments, the cancer or tumor cell expresses MSLN. In some embodiments, the cancer or tumor cell expresses high levels of MSLN compared to a reference expression level. In some embodiments, the cancer or tumor cell expresses low levels of MSLN compared to a reference expression level. In some embodiments, said reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects. In some embodiments, said expression level of MSLN is determined by measuring the protein expression level of MSLN. In some embodiments, the subject is a subject in need thereof. In some embodiments, the subject is a human. In specific embodiments, the subject is administered an effective amount.

According to particular embodiments, the pharmaceutical compositions described herein are formulated to be suitable for the intended route of administration to a subject. For example, the pharmaceutical compositions described herein can be formulated to be suitable for intravenous, subcutaneous, or intramuscular administration.

7.8 Kits

In another general aspect, the disclosure relates to kits comprising an isolated binding agent or antigen binding region thereof provided herein and instructions for use.

In one embodiment, provided is a kit comprising the MSLN binding agent (monospecific or bispecific) provided herein. The described kits can be used to carry out the methods of using the MSLN binding fragments provided herein, or other methods known to those skilled in the art. In some embodiments, the described kits can include the binding proteins described herein and reagents for use in detecting the presence of a target antigen (e.g., MSLN and CD3) in a biological sample. Accordingly, the described kits can include one or more of the binding agents (e.g., antibodies or proteins), or an antigen binding region(s) thereof, described herein and a vessel for containing the binding agent or antigen binding region when not in use, instructions for use of the antibody or fragment, the binding agent or antigen binding region affixed to a solid support, and/or detectably labeled forms of the binding agent or antigen binding region, as described herein.

In another embodiment, provided is a kit comprising the MSLN binding agent (monospecific or bispecific) comprising a first binding region specifically binding MSLN and a second binding region specifically binding CD3εprovided herein.

In some embodiments, the kit comprises an antibody described herein and reagents for detecting the binding agent. The kit can further include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the MSLN binding agent (monospecific or bispecific) provided herein in a container and instructions for use of the kit.

In some embodiments, the MSLN binding agent in the kit is labeled.

In case of conflict, the specification, including definitions, will control. As used herein and in the appended claims, the singular forms “a,” “an,” and “the” include plural referents unless the context clearly dictates otherwise. Thus, for example, reference to “a peptide sequence” or “a treatment,” includes a plurality of such sequences, treatments, and so forth. It is further noted that the claims can be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges, unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1000, 1000-2500, 2500-5000, 5000-25,000, or 5000-50,000 includes any numerical value or range within or encompassing such values, e.g., 25, 26, 27, 28, 29 . . . 250, 251, 252, 253, 254 . . . 500, 501, 502, 503, 504 . . . , etc. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

A number of embodiments of the invention have been described. Nevertheless, it will be understood that various modifications may be made without departing from the spirit and scope of the invention. Accordingly, the descriptions in the Experimental section are intended to illustrate but not limit the scope of invention described in the claims.

### Embodiments

1. A binding agent comprising an antigen binding region that
   (a) binds to an epitope of MSLN recognized by an antibody comprising a heavy chain variable (VH) domain comprising a VH complementarity determining region 1 (HCDR1), a HCDR2, and a HCDR3 having an amino acid sequence of a HCDR1, a HCDR2, and a HCDR3, respectively, of a VH domain having an amino acid sequence of SEQ ID NO:81; and a light chain variable (VL) domain comprising a LCDR1, a LCDR2, and a LCDR3 having an amino acid sequence of a LCDR1, a LCDR2, and a LCDR3, respectively, of a VL domain having an amino acid sequence of SEQ ID NO:82;
   (b) competes for the binding to MSLN with an antibody comprising a VH domain comprising a VH complementarity determining region 1 (HCDR1), a HCDR2, and a HCDR3 having an amino acid sequence of a HCDR1, a HCDR2, and a HCDR3, respectively, of a VH domain having an amino acid sequence of SEQ ID NO:81; and a VL domain comprising a LCDR1, a LCDR2, and a LCDR3 having an amino acid sequence of a LCDR1, a LCDR2, and a LCDR3, respectively, of a VL domain having an amino acid sequence of SEQ ID NO:82; or
   (c) binds to an epitope of MSLN in the membrane-restricted region of MSLN.
2. A binding agent comprising an antigen binding region that binds to MSLN, wherein the antigen binding region comprises:
   (a) a heavy chain variable (VH) domain comprising VH complementarity determining region 1 (HCDR1), HCDR2, and HCDR3 of any one of antibodies MSNB457, MSNB71, MSNB124, MSNB133, MSNB459, MSNB568, MSNB569, MSNB570, MSNB571, MSNB590, MSNB591, or MSNB592 as set forth in Tables 1 to 12; and/or
   (b) a light chain variable (VL) domain comprising LCDR1, LCDR2, and LCDR3 of any one of antibodies MSNB457, MSNB71, MSNB124, MSNB133, MSNB459, MSNB568, MSNB569, MSNB570, MSNB571, MSNB590, MSNB591, or MSNB592 as set forth in Tables 1 to 12.
3. The binding agent of embodiment 1 or 2, wherein the antigen binding region comprises:
   (a) a VH domain comprising a HCDR1, HCDR2, and HCDR3 as set forth in SEQ ID NO:81, and a VL domain comprising a LCDR1, LCDR2, and LCDR3 as set forth in SEQ ID NO:82;
   (b) a VH domain comprising a HCDR1, HCDR2, and HCDR3 as set forth in SEQ ID NO:22, and a VL domain comprising a LCDR1, LCDR2, and LCDR3 as set forth in SEQ ID NO:23;
   (c) a VH domain comprising a HCDR1, HCDR2, and HCDR3 as set forth in SEQ ID NO:45, and a VL domain comprising a LCDR1, LCDR2, and LCDR3 as set forth in SEQ ID NO:46;
   (d) a VH domain comprising a HCDR1, HCDR2, and HCDR3 as set forth in SEQ ID NO:62, and a VL domain comprising a LCDR1, LCDR2, and LCDR3 as set forth in SEQ ID NO:63;
   (e) a VH domain comprising a HCDR1, HCDR2, and HCDR3 as set forth in SEQ ID NO:94, and a VL domain comprising a LCDR1, LCDR2, and LCDR3 as set forth in SEQ ID NO:95;
   (f) a VH domain comprising a HCDR1, HCDR2, and HCDR3 as set forth in SEQ ID NO:106, and a VL domain comprising a LCDR1, LCDR2, and LCDR3 as set forth in SEQ ID NO:107;
   (g) a VH domain comprising a HCDR1, HCDR2, and HCDR3 as set forth in SEQ ID NO:120, and a VL domain comprising a LCDR1, LCDR2, and LCDR3 as set forth in SEQ ID NO:121;
   (h) a VH domain comprising a HCDR1, HCDR2, and HCDR3 as set forth in SEQ ID NO:132, and a VL domain comprising a LCDR1, LCDR2, and LCDR3 as set forth in SEQ ID NO:133;
   (i) a VH domain comprising a HCDR1, HCDR2, and HCDR3 as set forth in SEQ ID NO:138, and a VL domain comprising a LCDR1, LCDR2, and LCDR3 as set forth in SEQ ID NO:133;
   (j) a VH domain comprising a HCDR1, HCDR2, and HCDR3 as set forth in SEQ ID NO:120, and a VL domain comprising a LCDR1, LCDR2, and LCDR3 as set forth in SEQ ID NO:139;
   (k) a VH domain comprising a HCDR1, HCDR2, and HCDR3 as set forth in SEQ ID NO:106, and a VL domain comprising a LCDR1, LCDR2, and LCDR3 as set forth in SEQ ID NO:140; or
   (l) a VH domain comprising a HCDR1, HCDR2, and HCDR3 as set forth in SEQ ID NO:132, and a VL domain comprising a LCDR1, LCDR2, and LCDR3 as set forth in SEQ ID NO:142.
4. The binding agent of embodiment 2 or 3, wherein:
   (a1) HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
   (a2) HCDR1 comprises an amino acid sequence of SEQ ID NO:70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
   (a3) HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
   (a4) HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76;
   (a5) HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69;
   (b1) HCDR1 comprises an amino acid sequence of SEQ ID NO:1; HCDR2 comprises an amino acid sequence of SEQ ID NO:2; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6;
   (b2) HCDR1 comprises an amino acid sequence of SEQ ID NO:7; HCDR2 comprises an amino acid sequence of SEQ ID NO:8; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6;
   (b3) HCDR1 comprises an amino acid sequence of SEQ ID NO:9; HCDR2 comprises an amino acid sequence of SEQ ID NO:10; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6;
   (b4) HCDR1 comprises an amino acid sequence of SEQ ID NO: 11; HCDR2 comprises an amino acid sequence of SEQ ID NO:12; HCDR3 comprises an amino acid sequence of SEQ ID NO: 13; LCDR1 comprises an amino acid sequence of SEQ ID NO:14; LCDR2 comprises an amino acid sequence of SEQ ID NO:15; and LCDR3 comprises an amino acid sequence of SEQ ID NO:16;
   (b5) HCDR1 comprises an amino acid sequence of SEQ ID NO: 17; HCDR2 comprises an amino acid sequence of SEQ ID NO: 18; HCDR3 comprises an amino acid sequence of SEQ ID NO: 19; LCDR1 comprises an amino acid sequence of SEQ ID NO:20; LCDR2 comprises an amino acid sequence of LVS; and LCDR3 comprises an amino acid sequence of SEQ ID NO:6;
   (c1) HCDR1 comprises an amino acid sequence of SEQ ID NO:24; HCDR2 comprises an amino acid sequence of SEQ ID NO:25; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29; (c2) HCDR1 comprises an amino acid sequence of SEQ ID NO:30; HCDR2 comprises an amino acid sequence of SEQ ID NO:252; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29;
   (c3) HCDR1 comprises an amino acid sequence of SEQ ID NO:32; HCDR2 comprises an amino acid sequence of SEQ ID NO:33; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29;
   (c4) HCDR1 comprises an amino acid sequence of SEQ ID NO:34; HCDR2 comprises an amino acid sequence of SEQ ID NO:65; HCDR3 comprises an amino acid sequence of SEQ ID NO:36; LCDR1 comprises an amino acid sequence of SEQ ID NO:37; LCDR2 comprises an amino acid sequence of SEQ ID NO:38; and LCDR3 comprises an amino acid sequence of SEQ ID NO:39;
   (c5) HCDR1 comprises an amino acid sequence of SEQ ID NO:40; HCDR2 comprises an amino acid sequence of SEQ ID NO:41; HCDR3 comprises an amino acid sequence of SEQ ID NO:42; LCDR1 comprises an amino acid sequence of SEQ ID NO:43; LCDR2 comprises an amino acid sequence of LGS; and LCDR3 comprises an amino acid sequence of SEQ ID NO:29;
   (d1) HCDR1 comprises an amino acid sequence of SEQ ID NO:47; HCDR2 comprises an amino acid sequence of SEQ ID NO:48; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50;
   (d2) HCDR1 comprises an amino acid sequence of SEQ ID NO:251; HCDR2 comprises an amino acid sequence of SEQ ID NO:31; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50;
   (d3) HCDR1 comprises an amino acid sequence of SEQ ID NO:51; HCDR2 comprises an amino acid sequence of SEQ ID NO:52; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50;
   (d4) HCDR1 comprises an amino acid sequence of SEQ ID NO:53; HCDR2 comprises an amino acid sequence of SEQ ID NO:54; HCDR3 comprises an amino acid sequence of SEQ ID NO:55; LCDR1 comprises an amino acid sequence of SEQ ID NO:56; LCDR2 comprises an amino acid sequence of SEQ ID NO:38; and LCDR3 comprises an amino acid sequence of SEQ ID NO:57;
   (d5) HCDR1 comprises an amino acid sequence of SEQ ID NO:58; HCDR2 comprises an amino acid sequence of SEQ ID NO:59; HCDR3 comprises an amino acid sequence of SEQ ID NO:60; LCDR1 comprises an amino acid sequence of SEQ ID NO:61; LCDR2 comprises an amino acid sequence of LGS; and LCDR3 comprises an amino acid sequence of SEQ ID NO:50;
   (e1) HCDR1 comprises an amino acid sequence of SEQ ID NO:83; HCDR2 comprises an amino acid sequence of SEQ ID NO:84; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87;
   (e2) HCDR1 comprises an amino acid sequence of SEQ ID NO:30; HCDR2 comprises an amino acid sequence of SEQ ID NO:31; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87; (e3) HCDR1 comprises an amino acid sequence of SEQ ID NO:253; HCDR2 comprises an amino acid sequence of SEQ ID NO:52; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87;
   (e4) HCDR1 comprises an amino acid sequence of SEQ ID NO:88; HCDR2 comprises an amino acid sequence of SEQ ID NO:54; HCDR3 comprises an amino acid sequence of SEQ ID NO:89; LCDR1 comprises an amino acid sequence of SEQ ID NO:90; LCDR2 comprises an amino acid sequence of SEQ ID NO:38; and LCDR3 comprises an amino acid sequence of SEQ ID NO:91;
   (e5) HCDR1 comprises an amino acid sequence of SEQ ID NO:40; HCDR2 comprises an amino acid sequence of SEQ ID NO:59; HCDR3 comprises an amino acid sequence of SEQ ID NO:92; LCDR1 comprises an amino acid sequence of SEQ ID NO:93; LCDR2 comprises an amino acid sequence of LGS; and LCDR3 comprises an amino acid sequence of SEQ ID NO:87;
   (f1) HCDR1 comprises an amino acid sequence of SEQ ID NO:1; HCDR2 comprises an amino acid sequence of SEQ ID NO:96; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
   (f2) HCDR1 comprises an amino acid sequence of SEQ ID NO:7; HCDR2 comprises an amino acid sequence of SEQ ID NO:31; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
   (f3) HCDR1 comprises an amino acid sequence of SEQ ID NO:9; HCDR2 comprises an amino acid sequence of SEQ ID NO:52; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
   (f4) HCDR1 comprises an amino acid sequence of SEQ ID NO:11; HCDR2 comprises an amino acid sequence of SEQ ID NO:54; HCDR3 comprises an amino acid sequence of SEQ ID NO:100; LCDR1 comprises an amino acid sequence of SEQ ID NO:101; LCDR2 comprises an amino acid sequence of SEQ ID NO:102; and LCDR3 comprises an amino acid sequence of SEQ ID NO:103;
   (f5) HCDR1 comprises an amino acid sequence of SEQ ID NO:17; HCDR2 comprises an amino acid sequence of SEQ ID NO:59; HCDR3 comprises an amino acid sequence of SEQ ID NO: 104; LCDR1 comprises an amino acid sequence of SEQ ID NO:105; LCDR2 comprises an amino acid sequence of LVS; and LCDR3 comprises an amino acid sequence of SEQ ID NO:99;
   (g1) HCDR1 comprises an amino acid sequence of SEQ ID NO: 108; HCDR2 comprises an amino acid sequence of SEQ ID NO: 109; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
   (g2) HCDR1 comprises an amino acid sequence of SEQ ID NO:112; HCDR2 comprises an amino acid sequence of SEQ ID NO:31; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
   (g3) HCDR1 comprises an amino acid sequence of SEQ ID NO:113; HCDR2 comprises an amino acid sequence of SEQ ID NO:52; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
   (g4) HCDR1 comprises an amino acid sequence of SEQ ID NO:114; HCDR2 comprises an amino acid sequence of SEQ ID NO:54; HCDR3 comprises an amino acid sequence of SEQ ID NO:115; LCDR1 comprises an amino acid sequence of SEQ ID NO:116; LCDR2 comprises an amino acid sequence of SEQ ID NO:38; and LCDR3 comprises an amino acid sequence of SEQ ID NO:91;
   (g5) HCDR1 comprises an amino acid sequence of SEQ ID NO:117; HCDR2 comprises an amino acid sequence of SEQ ID NO:59; HCDR3 comprises an amino acid sequence of SEQ ID NO:118; LCDR1 comprises an amino acid sequence of SEQ ID NO:119; LCDR2 comprises an amino acid sequence of LGS; and LCDR3 comprises an amino acid sequence of SEQ ID NO:87;
   (h1) HCDR1 comprises an amino acid sequence of SEQ ID NO: 122; HCDR2 comprises an amino acid sequence of SEQ ID NO:84; HCDR3 comprises an amino acid sequence of SEQ ID NO:3; LCDR1 comprises an amino acid sequence of SEQ ID NO: 123; LCDR2 comprises an amino acid sequence of SEQ ID NO:124; and LCDR3 comprises an amino acid sequence of SEQ ID NO:99;
   (h2) HCDR1 comprises an amino acid sequence of SEQ ID NO: 125; HCDR2 comprises an amino acid sequence of SEQ ID NO:31; HCDR3 comprises an amino acid sequence of SEQ ID NO:3; LCDR1 comprises an amino acid sequence of SEQ ID NO:123; LCDR2 comprises an amino acid sequence of SEQ ID NO: 124; and LCDR3 comprises an amino acid sequence of SEQ ID NO:99;
   (h3) HCDR1 comprises an amino acid sequence of SEQ ID NO:126; HCDR2 comprises an amino acid sequence of SEQ ID NO:52; HCDR3 comprises an amino acid sequence of SEQ ID NO:3; LCDR1 comprises an amino acid sequence of SEQ ID NO:123; LCDR2 comprises an amino acid sequence of SEQ ID NO:124; and LCDR3 comprises an amino acid sequence of SEQ ID NO:99;
   (h4) HCDR1 comprises an amino acid sequence of SEQ ID NO:127; HCDR2 comprises an amino acid sequence of SEQ ID NO:54; HCDR3 comprises an amino acid sequence of SEQ ID NO: 13; LCDR1 comprises an amino acid sequence of SEQ ID NO:128; LCDR2 comprises an amino acid sequence of SEQ ID NO:129; and LCDR3 comprises an amino acid sequence of SEQ ID NO:103;
   (h5) HCDR1 comprises an amino acid sequence of SEQ ID NO:130; HCDR2 comprises an amino acid sequence of SEQ ID NO:59; HCDR3 comprises an amino acid sequence of SEQ ID NO:19; LCDR1 comprises an amino acid sequence of SEQ ID NO:131; LCDR2 comprises an amino acid sequence of LGS; and LCDR3 comprises an amino acid sequence of SEQ ID NO:99;
   (i1) HCDR1 comprises an amino acid sequence of SEQ ID NO:122; HCDR2 comprises an amino acid sequence of SEQ ID NO:134; HCDR3 comprises an amino acid sequence of SEQ ID NO:3; LCDR1 comprises an amino acid sequence of SEQ ID NO: 123; LCDR2 comprises an amino acid sequence of SEQ ID NO:124; and LCDR3 comprises an amino acid sequence of SEQ ID NO:99;
   (i2) HCDR1 comprises an amino acid sequence of SEQ ID NO: 125; HCDR2 comprises an amino acid sequence of SEQ ID NO:31; HCDR3 comprises an amino acid sequence of SEQ ID NO:3; LCDR1 comprises an amino acid sequence of SEQ ID NO: 123; LCDR2 comprises an amino acid sequence of SEQ ID NO:124; and LCDR3 comprises an amino acid sequence of SEQ ID NO:99;
   (i3) HCDR1 comprises an amino acid sequence of SEQ ID NO: 126; HCDR2 comprises an amino acid sequence of SEQ ID NO:135; HCDR3 comprises an amino acid sequence of SEQ ID NO:3; LCDR1 comprises an amino acid sequence of SEQ ID NO:123; LCDR2 comprises an amino acid sequence of SEQ ID NO:124; and LCDR3 comprises an amino acid sequence of SEQ ID NO:99;
   (i4) HCDR1 comprises an amino acid sequence of SEQ ID NO:127; HCDR2 comprises an amino acid sequence of SEQ ID NO:136; HCDR3 comprises an amino acid sequence of SEQ ID NO: 13; LCDR1 comprises an amino acid sequence of SEQ ID NO:128; LCDR2 comprises an amino acid sequence of SEQ ID NO:129; and LCDR3 comprises an amino acid sequence of SEQ ID NO:103;
   (i5) HCDR1 comprises an amino acid sequence of SEQ ID NO: 130; HCDR2 comprises an amino acid sequence of SEQ ID NO:137; HCDR3 comprises an amino acid sequence of SEQ ID NO: 19; LCDR1 comprises an amino acid sequence of SEQ ID NO: 131; LCDR2 comprises an amino acid sequence of LGS; and LCDR3 comprises an amino acid sequence of SEQ ID NO:99;
   (j1) HCDR1 comprises an amino acid sequence of SEQ ID NO:108; HCDR2 comprises an amino acid sequence of SEQ ID NO: 109; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
   (j2) HCDR1 comprises an amino acid sequence of SEQ ID NO:112; HCDR2 comprises an amino acid sequence of SEQ ID NO:31; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
   (j3) HCDR1 comprises an amino acid sequence of SEQ ID NO:113; HCDR2 comprises an amino acid sequence of SEQ ID NO:52; HCDR3 comprises an amino acid sequence of SEQ ID NO:110; LCDR1 comprises an amino acid sequence of SEQ ID NO: 111; LCDR2 comprises an amino acid sequence of SEQ ID NO:28; and LCDR3 comprises an amino acid sequence of SEQ ID NO:87;
   (j4) HCDR1 comprises an amino acid sequence of SEQ ID NO:114; HCDR2 comprises an amino acid sequence of SEQ ID NO:54; HCDR3 comprises an amino acid sequence of SEQ ID NO:115; LCDR1 comprises an amino acid sequence of SEQ ID NO:116; LCDR2 comprises an amino acid sequence of SEQ ID NO:38; and LCDR3 comprises an amino acid sequence of SEQ ID NO:91;
   (j5) HCDR1 comprises an amino acid sequence of SEQ ID NO:117; HCDR2 comprises an amino acid sequence of SEQ ID NO:59; HCDR3 comprises an amino acid sequence of SEQ ID NO:118; LCDR1 comprises an amino acid sequence of SEQ ID NO:119; LCDR2 comprises an amino acid sequence of LGS; and LCDR3 comprises an amino acid sequence of SEQ ID NO:87;
   (k1) HCDR1 comprises an amino acid sequence of SEQ ID NO: 1; HCDR2 comprises an amino acid sequence of SEQ ID NO:96; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
   (k2) HCDR1 comprises an amino acid sequence of SEQ ID NO:7; HCDR2 comprises an amino acid sequence of SEQ ID NO:31; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
   (k3) HCDR1 comprises an amino acid sequence of SEQ ID NO:9; HCDR2 comprises an amino acid sequence of SEQ ID NO:52; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
   (k4) HCDR1 comprises an amino acid sequence of SEQ ID NO:11; HCDR2 comprises an amino acid sequence of SEQ ID NO:54; HCDR3 comprises an amino acid sequence of SEQ ID NO: 100; LCDR1 comprises an amino acid sequence of SEQ ID NO:101; LCDR2 comprises an amino acid sequence of SEQ ID NO:102; and LCDR3 comprises an amino acid sequence of SEQ ID NO:103;
   (k5) HCDR1 comprises an amino acid sequence of SEQ ID NO:17; HCDR2 comprises an amino acid sequence of SEQ ID NO:59; HCDR3 comprises an amino acid sequence of SEQ ID NO: 104; LCDR1 comprises an amino acid sequence of SEQ ID NO:105; LCDR2 comprises an amino acid sequence of LVS; and LCDR3 comprises an amino acid sequence of SEQ ID NO:99;
   (l1) HCDR1 comprises an amino acid sequence of SEQ ID NO:122; HCDR2 comprises an amino acid sequence of SEQ ID NO:84; HCDR3 comprises an amino acid sequence of SEQ ID NO:3; LCDR1 comprises an amino acid sequence of SEQ ID NO:123; LCDR2 comprises an amino acid sequence of SEQ ID NO:28; and LCDR3 comprises an amino acid sequence of SEQ ID NO:99;
   (l2) HCDR1 comprises an amino acid sequence of SEQ ID NO: 125; HCDR2 comprises an amino acid sequence of SEQ ID NO:31; HCDR3 comprises an amino acid sequence of SEQ ID NO:3; LCDR1 comprises an amino acid sequence of SEQ ID NO: 123; LCDR2 comprises an amino acid sequence of SEQ ID NO:28; and LCDR3 comprises an amino acid sequence of SEQ ID NO:99;
   (l3) HCDR1 comprises an amino acid sequence of SEQ ID NO:126; HCDR2 comprises an amino acid sequence of SEQ ID NO:52; HCDR3 comprises an amino acid sequence of SEQ ID NO:3; LCDR1 comprises an amino acid sequence of SEQ ID NO: 123; LCDR2 comprises an amino acid sequence of SEQ ID NO:28; and LCDR3 comprises an amino acid sequence of SEQ ID NO:99;
   (l14) HCDR1 comprises an amino acid sequence of SEQ ID NO:127; HCDR2 comprises an amino acid sequence of SEQ ID NO:54; HCDR3 comprises an amino acid sequence of SEQ ID NO:13; LCDR1 comprises an amino acid sequence of SEQ ID NO: 128; LCDR2 comprises an amino acid sequence of SEQ ID NO:141; and LCDR3 comprises an amino acid sequence of SEQ ID NO:103; or
   (l5) HCDR1 comprises an amino acid sequence of SEQ ID NO:130; HCDR2 comprises an amino acid sequence of SEQ ID NO:59; HCDR3 comprises an amino acid sequence of SEQ ID NO:19; LCDR1 comprises an amino acid sequence of SEQ ID NO:131; LCDR2 comprises an amino acid sequence of LGS; and LCDR3 comprises an amino acid sequence of SEQ ID NO:99.
5. The binding agent of any one of embodiments 1 to 4, wherein the antigen binding region comprises:
   (i) a VH comprising an amino acid sequence of SEQ ID NO:81;
   (ii) a VH comprising an amino acid sequence of SEQ ID NO:22;
   (iii) a VH comprising an amino acid sequence of SEQ ID NO:45;
   (iv) a VH comprising an amino acid sequence of SEQ ID NO:62;
   (v) a VH comprising an amino acid sequence of SEQ ID NO:94;
   (vi) a VH comprising an amino acid sequence of SEQ ID NO:106;
   (vii) a VH comprising an amino acid sequence of SEQ ID NO: 120;
   (viii) a VH comprising an amino acid sequence of SEQ ID NO:132;
   (ix) a VH comprising an amino acid sequence of SEQ ID NO:138;
   (x) a VL comprising an amino acid sequence of SEQ ID NO:82;
   (xi) a VL comprising an amino acid sequence of SEQ ID NO:23;
   (xii) a VL comprising an amino acid sequence of SEQ ID NO:46;
   (xiii) a VL comprising an amino acid sequence of SEQ ID NO:63;
   (xix) a VL comprising an amino acid sequence of SEQ ID NO:82;
   (xx) a VL comprising an amino acid sequence of SEQ ID NO:95;
   (xxi) a VL comprising an amino acid sequence of SEQ ID NO:107;
   (xxii) a VL comprising an amino acid sequence of SEQ ID NO:121;
   (xxiii) a VL comprising an amino acid sequence of SEQ ID NO:133;
   (xxiv) a VL comprising an amino acid sequence of SEQ ID NO:139;
   (xxv) a VL comprising an amino acid sequence of SEQ ID NO:140; or
   (xxvi) a VL comprising an amino acid sequence of SEQ ID NO:142.
6. The binding agent of any one of embodiments 1 to 5, wherein the antigen binding region comprises:
   (a) a VH domain comprising an amino acid sequence of SEQ ID NO:81, and a VL domain comprising an amino acid sequence of SEQ ID NO:82;
   (b) a VH domain comprising an amino acid sequence of SEQ ID NO:22, and a VL domain comprising an amino acid sequence of SEQ ID NO:23;
   (c) a VH domain comprising an amino acid sequence of SEQ ID NO:45, and a VL domain comprising an amino acid sequence of SEQ ID NO:46;
   (d) a VH domain comprising an amino acid sequence of SEQ ID NO:62, and a VL domain comprising an amino acid sequence of SEQ ID NO:63;
   (e) a VH domain comprising an amino acid sequence of SEQ ID NO:94, and a VL domain comprising an amino acid sequence of SEQ ID NO:95;
   (f) a VH domain comprising an amino acid sequence of SEQ ID NO: 106, and a VL domain comprising an amino acid sequence of SEQ ID NO:107;
   (g) a VH domain comprising an amino acid sequence of SEQ ID NO:120, and a VL domain comprising an amino acid sequence of SEQ ID NO:121;
   (h) a VH domain comprising an amino acid sequence of SEQ ID NO:132, and a VL domain comprising an amino acid sequence of SEQ ID NO:133;
   (i) a VH domain comprising an amino acid sequence of SEQ ID NO:138, and a VL domain comprising an amino acid sequence of SEQ ID NO:133;
   (j) a VH domain comprising an amino acid sequence of SEQ ID NO: 120, and a VL domain comprising an amino acid sequence of SEQ ID NO:139;
   (k) a VH domain comprising an amino acid sequence of SEQ ID NO:106, and a VL domain comprising an amino acid sequence of SEQ ID NO:140; or
   (l) a VH domain comprising an amino acid sequence of SEQ ID NO: 132, and a VL domain comprising an amino acid sequence of SEQ ID NO: 142.
7. The binding agent any one of embodiments 1 to 6, wherein the antigen binding region that binds to MSLN comprises a Fab, an scFv, a (scFv)₂, a Fv, a F(ab′)₂, a Fd, or a dAb.
8. The binding agent of embodiment 7, wherein the antigen binding region that binds to MSLN is the Fab.
9 The binding agent of embodiment 7, wherein the antigen binding region that binds to MSLN is the scFv.
10. The binding agent of embodiment 9, wherein the scFv comprises, from the N- to C-terminus, a VH, a first linker (L1) and a VL (VH-L1-VL) or the VL, the L1 and the VH (VL-L1-VH).
11. The binding agent of embodiment 10, wherein the L1 comprises
   (a) about 5 to 50 amino acids;
   (b) about 5 to 40 amino acids;
   (c) about 10 to 30 amino acids; or
   (d) about 10 to 20 amino acids.
12. The binding agent of embodiment 10 or 11, wherein the L1 comprises the amino acid sequence of SEQ ID NO: 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199.
13. The binding agent of any one of embodiments 1 to 12, wherein the binding agent is a multi-specific protein.
14. The binding agent of embodiment 13, wherein the multispecific protein is a bispecific protein.
15. The binding agent of embodiment 13, wherein the multispecific protein is a trispecific protein.
16. The binding agent of any one of embodiments 1 to15, further comprising an immunoglobulin (Ig) constant region of or a fragment of the Ig constant region.
17. The binding agent of embodiment 16, wherein the fragment of the Ig constant region comprises a Fc region.
18. The binding agent of embodiment 16, wherein the fragment of the Ig constant region comprises a CH2 domain.
19. The binding agent of embodiment 16, wherein the fragment of the Ig constant region comprises a CH3 domain.
20. The binding agent of embodiment 16, wherein the fragment of the Ig constant region comprises a CH2 domain and a CH3 domain.
21. The binding agent of embodiment 16, wherein the fragment of the Ig constant region comprises at least portion of a hinge, a CH2 domain and a CH3 domain.
22. The binding agent of embodiment 16, wherein the fragment of the Ig constant region comprises a hinge, a CH2 domain and a CH3 domain.
23. The binding agent of any one of embodiments 6 to 22, wherein the antigen binding region that binds MSLN is fused to the N-terminus of the Ig constant region or the fragment of the Ig constant region.
24. The binding agent of any one of embodiments 16 to 22, wherein the antigen binding region that binds MSLN is fused to the C-terminus of the Ig constant region or the fragment of the Ig constant region.
25. The binding agent of any one of embodiments 16 to 22, wherein the antigen binding region that binds MSLN is fused to the Ig constant region or the fragment of the Ig constant region via a second linker (L2).
26. The binding agent of embodiment 25, wherein the L2 comprises the amino acid sequence of SEQ ID NO: 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199.
27. The binding agent of any one of embodiments 16 to 26, wherein the Ig constant region or the fragment of the Ig constant region is of an IgG1, an IgG2, an IgG3 or an IgG4 isotype.
28. The binding agent of any one of embodiments 16 to 27, wherein the Ig constant region or the fragment of the Ig constant region comprises at least one mutation that results in reduced binding of the protein to a Fcγ receptor (FcγR).
29. The binding agent of embodiment 28, wherein the at least one mutation that results in reduced binding of the binding agent to the FcγR is selected from the group consisting of, L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.
30. The binding agent of embodiment 28 or 29, wherein the FcγR is FcγRI, FcγRIIA, FcγRIIB or FcγRIII, or any combination thereof.
31. The binding agent of any one of the embodiments 16 to 30, wherein the protein comprises at least one mutation in a CH3 domain of the Ig constant region or the fragment of the Ig constant region. 32. The binding agent of embodiment 31, wherein the at least one mutation in the CH3 domain is selected from the group consisting of T350V, L351Y, F405A, Y407V, T366Y, T366W, T366L, F405W, T394W, K392L, T394S, Y407T, Y407A, T366S/L368A/Y407V, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index.
33. The binding agent of embodiment 31 or 32, wherein at least one mutation in the CH3 domain is selected from the group consisting of H435R, Y436F and H435R/L436F.
34. The binding agent of any one of embodiments 1 to 33, wherein the antigen binding region specifically binds to membrane-associated MSLN and does not bind to a soluble MSLN isoform or shed MSLN.
35. The binding agent of any one of embodiments 1 to 33, wherein, when bound to MSLN, the antigen binding region binds to at least one of residues 587-598 within an amino acid sequence of SEQ ID NO:200.
36. The binding agent of any one of embodiments 1 to 34, wherein, when bound to MSLN, the antigen binding region binds to at least one residue selected from the group consisting of L589, D590, M593, V588, S592, L597, E595, and A596 with an amino acid sequence of SEQ ID NO:200.
37. The binding agent of any one of embodiments 1 to 34, wherein, when bound to MSLN, the antigen binding region binds to at least one residue selected from the group consisting of L589, D590, and M593 with an amino acid sequence of SEQ ID NO:200.
38. The binding agent of any one of embodiments 1 to 34, wherein, when bound to MSLN, the antigen binding region binds to at least one residue selected from the group consisting of V588, S592, E595, and L597 with an amino acid sequence of SEQ ID NO:200. 39. The binding agent of any one of embodiments 1 to 34, wherein, when bound to MSLN, the antigen binding region binds to at least one residue selected from the group consisting of V588, S592, and L597 with an amino acid sequence of SEQ ID NO:200.
40. The binding agent of any one of embodiments 13 to 39, wherein the multispecific protein comprises an antigen binding region that binds a second antigen other than MSLN.
41. The binding agent of embodiment 40, wherein the second antigen is cluster of differentiation 38 (CD3ε).
42. A binding agent comprising a first antigen binding region that binds to MSLN and a second antigen binding region that binds to CD3ε, wherein
   (i) the first antigen binding region comprises a VH domain comprising a HCDR1, HCDR2, and HCDR3 as set forth in SEQ ID NO:81, and a VL domain comprising a LCDR1, LCDR2, and LCDR3 as set forth in SEQ ID NO:82; and
   (ii) the second antigen binding region a VH domain comprising a HCDR1, HCDR2, and HCDR3 as set forth in SEQ ID NO:159, and a VL domain comprising a LCDR1, LCDR2, and LCDR3 as set forth in SEQ ID NO:160.
43. The binding agent of embodiment 42, wherein
   (i) in the first antigen binding region that binds to MSLN,
      (a) HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
      (b) HCDR1 comprises an amino acid sequence of SEQ ID NO:70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
      (c) HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
      (d) HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76; or
      (e) HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69; and
   (ii) in the second antigen binding region that binds to CD3&,
      (a) HCDR1 comprises an amino acid sequence of SEQ ID NO:143; HCDR2 comprises an amino acid sequence of SEQ ID NO:144; HCDR3 comprises an amino acid sequence of SEQ ID NO:145; LCDR1 comprises an amino acid sequence of SEQ ID NO:146; LCDR2 comprises an amino acid sequence of SEQ ID NO:147; and LCDR3 comprises an amino acid sequence of SEQ ID NO: 148;
      (b) HCDR1 comprises an amino acid sequence of SEQ ID NO:149; HCDR2 comprises an amino acid sequence of SEQ ID NO:150; HCDR3 comprises an amino acid sequence of SEQ ID NO: 151; LCDR1 comprises an amino acid sequence of SEQ ID NO:152; LCDR2 comprises an amino acid sequence of YAS; and LCDR3 comprises an amino acid sequence of SEQ ID NO:154; or
      (c) HCDR1 comprises an amino acid sequence of SEQ ID NO:155; HCDR2 comprises an amino acid sequence of SEQ ID NO:156; HCDR3 comprises an amino acid sequence of SEQ ID NO:157; LCDR1 comprises an amino acid sequence of SEQ ID NO:158; LCDR2 comprises an amino acid sequence of YAS; and LCDR3 comprises an amino acid sequence of SEQ ID NO:148.
44. The binding agent of embodiment 42 or 43, wherein
   (i) the first binding region comprises a VH domain comprising an amino acid sequence of SEQ ID NO:81, and a VL domain comprising an amino acid sequence of SEQ ID NO:82; and
   (ii) the second binding region comprises a VH domain comprising an amino acid sequence of SEQ ID NO:159, and a VL domain comprising an amino acid sequence of SEQ ID NO: 160.
45. The binding agent of any one of embodiments 42 to 44, wherein the first antigen binding region comprises a Fab, and the second antigen binding region comprises a scFv.
46. The binding agent of embodiment 45, wherein the scFv comprises an amino acid sequence of SEQ ID NO: 161 or SEQ ID NO: 162.
47. The binding agent of any one of embodiments 42 to 46, wherein the binding agent further comprises an immunoglobulin (Ig) constant region of or a fragment of the Ig constant region.
48. The binding agent of embodiment 47, wherein the fragment of the Ig constant region comprises a Fc region.
49. The binding agent of embodiment 47 or 48, wherein the binding agent comprises:
   (i) a first polypeptide comprising the second antigen binding region as a scFv, a CH2 domain and a CH3 domain;
   (ii) a second polypeptide comprising the VH domain of the first antigen binding region, a CH2 domain and a CH3 domain; and
   (iii) a third polypeptide comprising the VL domain of the first antigen binding region, wherein the VH domain and the VL domain of the first antigen binding region form a Fab, and the first polypeptide and the second polypeptide forms a Fc region.
50. The binding agent of embodiment 49, wherein the first polypeptide comprising an amino acid sequence of SEQ ID NO: 163, a second polypeptide comprising an amino acid sequence of SEQ ID NO:164, and a third polypeptide comprising an amino acid sequence of SEQ ID NO: 165.
51. The binding agent of any one of embodiments 42 to 50, wherein the binding agent is a bispecific antibody.
52. The binding agent of any one of embodiments 42 to 51, wherein the binding agent is a monoclonal antibody.
53. The binding agent of any one of embodiments 42 to 52, wherein the binding agent is a humanized antibody.
54. A binding agent comprises
   (i) a first polypeptide comprising a scFv that binds CD3ε, a CH2 domain and a CH3 domain;
   (ii) a second polypeptide comprising a VH domain that binds MSLN, a CH2 domain and a CH3 domain; and
   (iii) a third polypeptide comprising a VL domain that binds MSLN,
   wherein the scFv that binds CD3ε comprises a VH domain comprising a HCDR1, HCDR2, and HCDR3 as set forth in SEQ ID NO: 159, and a VL domain comprising a LCDR1, LCDR2, and LCDR3 as set forth in SEQ ID NO:160; and
   wherein the VH domain that binds MSLN comprises a HCDR1, HCDR2, and HCDR3 as set forth in SEQ ID NO:81, and the VL domain that binds MSLN comprises a LCDR1, LCDR2, and LCDR3 as set forth in SEQ ID NO:82.
55. The binding agent of embodiment 54, wherein:
   (i) the scFv that binds CD3ε comprises:
      (a) HCDR1 comprising an amino acid sequence of SEQ ID NO: 143; HCDR2 comprising an amino acid sequence of SEQ ID NO:144; HCDR3 comprising an amino acid sequence of SEQ ID NO: 145; LCDR1 comprising an amino acid sequence of SEQ ID NO:146; LCDR2 comprising an amino acid sequence of SEQ ID NO: 147; and LCDR3 comprising an amino acid sequence of SEQ ID NO: 148;
      (b) HCDR1 comprising an amino acid sequence of SEQ ID NO:149; HCDR2 comprising an amino acid sequence of SEQ ID NO:150; HCDR3 comprising an amino acid sequence of SEQ ID NO:151; LCDR1 comprising an amino acid sequence of SEQ ID NO:152; LCDR2 comprising an amino acid sequence of YAS; and LCDR3 comprising an amino acid sequence of SEQ ID NO: 154; or
      (c) HCDR1 comprising an amino acid sequence of SEQ ID NO:155; HCDR2 comprising an amino acid sequence of SEQ ID NO:156; HCDR3 comprising an amino acid sequence of SEQ ID NO: 157; LCDR1 comprising an amino acid sequence of SEQ ID NO:158; LCDR2 comprising an amino acid sequence of YAS; and LCDR3 comprising an amino acid sequence of SEQ ID NO: 148; and
   (ii) (a) in the VH domain that binds MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69;
   (b) in the VH domain that binds MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69;
   (c) in the VH domain that binds MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69;
   (d) in the VH domain that binds MSLN, HCDR1 comprises an amino acid sequence of SEQ ID NO:72; HCDR2 comprises an amino acid sequence of SEQ ID NO:54; HCDR3 comprises an amino acid sequence of SEQ ID NO:73; and in the VL domain that binds MSLN, LCDR1 comprises an amino acid sequence of SEQ ID NO:74; LCDR2 comprises an amino acid sequence of SEQ ID NO:75; and LCDR3 comprises an amino acid sequence of SEQ ID NO:76; or
   (e) in the VH domain that binds MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; and in the VL domain that binds MSLN, LCDR1 comprises an amino acid sequence of SEQ ID NO:79; LCDR2 comprises an amino acid sequence of WAS; and LCDR3 comprises an amino acid sequence of SEQ ID NO:69.
56. The binding agent of embodiment 54 or 55, wherein the scFv that binds CD3ε comprises a VH domain comprising an amino acid sequence of SEQ ID NO:159, and a VL domain comprising an amino acid sequence of SEQ ID NO: 160; the VH domain that binds MSLN comprises an amino acid sequence of SEQ ID NO:81, and the VL domain that binds MSLN comprises an amino acid sequence of SEQ ID NO:82.
57. The binding agent of any one of embodiments 54 to 56, wherein the scFv comprises an amino acid sequence of SEQ ID NO: 161 or SEQ ID NO:162.
58. A composition comprising the binding agent of any one of embodiments 1 to 40, and a pharmaceutically acceptable carrier.
59. A composition comprising the binding agent of any one of embodiments 41 to 57, and a pharmaceutically acceptable carrier.
60. A polynucleotide comprising nucleotide sequences encoding a VH, a VL, or both a VH and a VL of the binding agent of any one of embodiments 1 to 57.
61. A polynucleotide comprising nucleotide sequences encoding the first polypeptide, the second polypeptide, and/or the third polypeptide of the binding agent of any one of embodiments 49, 50, or 54 to 57.
62. The polynucleotide of embodiment 60 or 61, wherein the polynucleotide is operably linked to a promoter.
63. A vector comprising the polynucleotide of any one of embodiments 60 to 62. 64. A cell comprising the polynucleotide of any one of embodiments 60 to 62.
65. A cell comprising the vector of embodiment 63.
66. An isolated cell producing the binding agent of any one of embodiments 1 to 57.
67. A kit comprising the binding agent of any one of embodiments 1 to 57.
68. A method of making a binding agent which binds to a membrane-restricted epitope of MSLN, comprising culturing the cell of any one of embodiments 64 to 66 to express the binding agent.
69. A method of making a binding agent which binds to a membrane-restricted epitope of MSLN, comprising expressing the polynucleotide of any one of embodiments 60 to 62.
70. A method of directing a T cell to a target cell expressing MSLN, comprising contacting the T cell with an effective amount of the binding agent of any one of embodiments 41 to 57 or the composition of embodiment 59, wherein the antigen binding region that binds to CD3ε binds the T cell and the antigen binding region that binds to MSLN binds to the target cell.
71. The method of embodiment 70, wherein the T cell induces apoptosis in the target cell.
72. The method of embodiment 71, wherein the apoptosis is induced via T-cell dependent cytotoxicity (TDCC) of the directed T cell.
73. The method of any one of embodiments 70 to 72, wherein the method induces differential cytokine releases by the directed T cell; optionally wherein the cytokine is IL-1β, IL-2, IL-4, IL-6, IL-8, IL-12, IL-13, TNF-α, IFN-γ, or any combination thereof.
74. The method of any one of embodiments 70 to 73, wherein the target cell expresses MSLN at a level higher than a reference expression level of MSLN; optionally wherein the reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects.
75. The method of any one of embodiments 70 to 74, wherein the target cell is a cancer or tumor cell.
76 The method of embodiment 75, wherein the cancer or tumor is selected from mesothelioma, ovarian cancer, and pancreatic cancer.
77. The method of embodiment 76, wherein the mesothelioma is a pleural mesothelioma or a peritoneal mesothelioma, the ovarian cancer is a serous ovarian carcinoma, and the pancreatic cancer is a pancreatic ductal adenocarcinoma.
78. The method of any one of embodiments 70 to 77, wherein the target cell comprises a single nucleotide polymorphism (SNP) in the MSLN gene that result in Met593 Val substitution in the encoded MSLN protein, and wherein the target cell is not homozygous for the SNP.
79. A method of activating a population of T cells, comprising contacting the population of T cells with an effective amount of the binding agent of any one of embodiments 41 to 57 or the composition of embodiment 59, wherein said binding agent activates the population of T cells upon binding to CD3ε.
80. The method of embodiment 79, wherein activation of the population of T cells is measured by an increase in expression of T cell activation marker(s) CD25, CD69, or a combination thereof.
81 The method of embodiment 79 or 80, wherein the percentage of CD25+ T cells, the percentage of CD69+ T cells, and/or the percentage of CD25+/CD69+ T cells in the population of T cells is increased 82. The method of embodiment 79, wherein activation of the population of T cells is measured by an increase in secretion of a cytokine by the population of T cells; optionally wherein the cytokine is IL-1β, IL-2, IL-4, IL-6, IL-8, IL-12, IL-13, TNF-α, IFN-γ, or any combination thereof.
83. A method of killing or inhibiting the proliferation of a cancer or tumor cell, comprising contacting the cancer or tumor cell with an effective amount the binding agent of any one of embodiments any one of embodiments 41 to 57 or the composition of embodiment 59.
84 The method of embodiment 83, wherein the cancer or tumor is selected from mesothelioma, serous ovarian carcinoma, and pancreatic cancer.
85. The method of embodiment 84, wherein the cancer is mesothelioma, serous ovarian carcinoma, and pancreatic cancer.
86. The method of any one of embodiment 83 to 85, wherein the cancer or tumor cell expresses MSLN at a level higher than a reference expression level of MSLN; optionally wherein the reference expression level of MSLN is: (a) a predetermined expression level of MSLN; (b) an MSLN expression level in a corresponding normal cell or issue; (c) an MSLN expression level measured in a neighboring non-cancerous cell or tissue in the same subject; or (d) an MSLN expression level in a corresponding cell or tissue measured in a cohort of healthy subjects.
87. A method of treating a cancer or tumor in a subject in need thereof, comprising administering an effective amount the binding agent of any one of embodiments any one of embodiments 41 to 57 or the composition of embodiment 59 to the subject.
88. The method of embodiment 87, wherein the cancer or tumor is selected from mesothelioma, ovarian cancer, and pancreatic cancer.
89. The method of embodiment 88, wherein the mesothelioma is a pleural mesothelioma or a peritoneal mesothelioma, the ovarian cancer is a serous ovarian carcinoma, and the pancreatic cancer is a pancreatic ductal adenocarcinoma.
90 The method of any one of embodiment 87 to 89, wherein the administering is subcutaneous administration.
91. The method of any one of embodiment 87 to 90, wherein the subject is not homozygous for an SNP in the MLSN gene that results in Met593 Val substitution in the encoded MSLN.
92. A method for diagnosing and treating a subject having a MSLN-expressing cancer or tumor, comprising
   (a) detecting presence or absence of a SNP in the MSLN gene in the subject that results in Met593 Val substitution in the encoded MSLN protein;
   (b) diagnosing the subject as likely responsive to the treatment of a binding agent comprising a first antigen binding region that binds to MSLN and a second antigen binding region that binds to CD3ε if the subject is not homozygous for the SNP; and
   (c) administering or providing for administration of an effective amount of the binding agent of any one of embodiments 41 to 57 or the composition of embodiment 59 to the subject if the subject is diagnosed as likely responsive in step (b).
93. A method for treating a subject having a MSLN-expressing cancer or tumor, comprising administering or provide for administration of an effective amount of the binding agent of any one of embodiments 41 to 57 or the composition of embodiment 59 to a subject wherein the subject is not homozygous for a SNP in the MSLN gene that results in Met593 Val substitution in the encoded MSLN protein.
94. The method of embodiment 92 or 93, wherein the cancer or tumor is selected from mesothelioma, ovarian cancer, and pancreatic cancer.

### 8. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; pl or pL=picoliter(s); dl or dL=deciliter; μl or μL=microliter; ml or mL=milliliter; l or L=liter; M=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; TIW=three times a week; QM=monthly; HPLC=high performance liquid chromatography; BW=body weight; U=unit; ns=not statistically significant; PBS=phosphate-buffered saline; PCR=polymerase chain reaction; NHS=N-Hydroxysuccinimide; HSA=human serum albumin; BSA=bovine serum albumin; DMEM=Dulbeco's Modification of Eagle's Medium; GC=genome copy; EDTA=ethylenediaminetetraacetic acid.

The following experimental methods were employed throughout the Examples described herein.

### 8.1 Example 1: Targeting Intact MSLN on the Membrane

The purpose of this study was to design an antibody that binds to the C-terminal region of MSLN that is not present in shed MSLN to avoid binding to the reservoir of soluble MSLN known to be present in the serum and increased in some cancers.

MSLN was first shown to be shed from the membrane in ovarian carcinoma patients due to the sheddase TACE, which is a member of the matrix metalloproteinase (MMP) disintegrin and metalloprotease family. FIG. 1 shows that TACE cleaves MSLN at 7 to 12 amino acids distal from the GPI anchor, releasing shed MSLN into the circulation. Further, other proteases including disintegrin and metalloproteinase (ADAM)10, ADAM17, b-secretase (BACE)2, BACE1, and MMP15 have also been shown to cleave MSLN close to the cell membrane and more than one sheddase can catalyze MSLN release in the same cell.

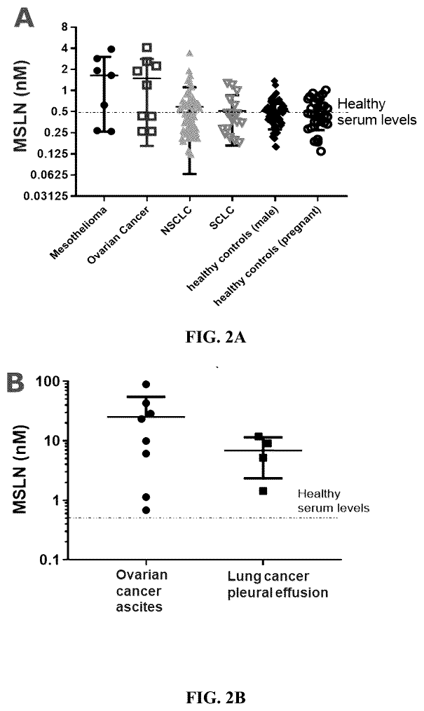

FIG. 2A shows that elevated blood levels of soluble MSLN, particularly in mesothelioma and ovarian cancers. Serosal fluids of cancer patients, in which normal mesothelial cells and circulating cancer cells can be found, contained up to 70-fold higher amounts of soluble MSLN compared to serum of healthy individuals (FIG. 2B). Tumor tissues from different indications were also shown to contain high levels of soluble MSLN, amounts that in many cases were higher than those of membrane-bound MSLN (FIG. 2D). These results show that soluble MSLN in the tumor environment could impair activity of MSLN-targeting antibody therapies by acting as a soluble “sink” impairing antibody activity.
8.1.1 Antigens Used to Generate Antibodies Against C-terminus of Human MSLN

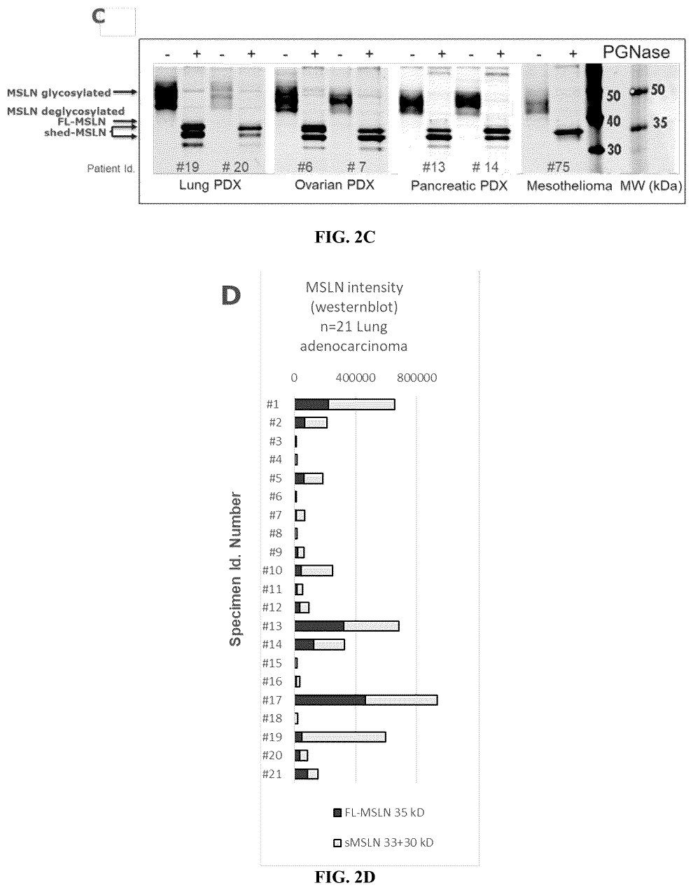

Three versions of chimeric full-length MSLN were used to create antibodies targeting the C-terminus of MSLN. FIG. 3 shows a graphical depiction of two of the immunogens. They differ in the length of sequence at the C-terminus that is derived from the human sequence. For MSNW20 the sequence of the last 52 amino acids was derived from the human isoform 2 sequence (Q13421-3), with the remainder of the sequence derived from the mouse sequence (sQ16468). For MSNW23 only the last 8 amino acids were derived from the human sequence with the rest derived from the mouse sequence. These antigens were used to immunize both transgenic Ablexis mice expressing human variable regions and wild-type mice (AJ strain). The amino acid sequences for the immunogen polypeptides are provided below:

MSNW20
(SEQ ID NO: 245)
HHHHHHGLNDIFEAQKIEWHEENLYFQSGSDAEQKACPPGKEPYK
VDEDLIFYQNWELEACVDGTMLARQMDLVNEIPFTYEQLSIFKHK
LDKTYPQGYPESLIQQLGHFFRYVSPEDIHQWNVTSPDTVKTLLK
VSKGQKMNAQAIALVACYLRGGGQLDEDMVKALGDIPLSYLCDFS
PQDLHSVPSSVMWLVGPQDLDKCSQRHLGLLYQKACSAFQNVSGL
EYFEKIKTFLGGASVKDLRALSQHNVSMDIATFKRLQVDSLVGLS
VAEVQKLLGPHVEGLKAEERHRPVRDWILRQRQDDLDTLGLGLQG
GIPNGYLVLDLSMQEALSGGGGS
MSNW23
(SEQ ID NO: 246)
HHHHHHGLNDIFEAQKIEWHEENLYFQSGSDAEQKACPPGKEPYK
VDEDLIFYQNWELEACVDGTMLARQMDLVNEIPFTYEQLSIFKHK
LDKTYPQGYPESLIQQLGHFFRYVSPEDIHQWNVTSPDTVKTLLK
VSKGQKMNAQAIALVACYLRGGGQLDEDMVKALGDIPLSYLCDFS
PQDLHSVPSSVMWLVGPQDLDKCSQRHLGLLYQKACSAFQNVSGL
EYFEKIKTFLGGASVKDLRALSQHNVSMDIATFKRLQVDSLVGLS
VAEVQKLLGPNIVDLKTEEDKSPVRDWLFRQHQKDLDRLGLGLQG
GIPNGYLVLDLSMQEALSGGGGS
MSNW10
(SEQ ID NO: 247)
EVEKTACPSGKKAREIDESLIFYKKWELEACVDAALLATQMDRVN
AIPFTYEQLDVLKHKLDELYPQGYPESVIQHLGYLFLKMSPEDIR
KWNVTSLETLKALLEVNKGHEMSPQVATLIDRFVKGRGQLDKDTL
DTLTAFYPGYLCSLSPEELSSVPPSSIWAVRPQDLDTCDPRQLDV
LYPKARLAFQNMNGSEYFVKIQSFLGGAPTEDLKALSQQNVSMDL
ATFMKLRTDAVLPLTVAEVQKLLGPHVEGLKAEERHRPVRDWILR
QRQDDLDTLGLGLQGGIPNGYLVLDLSMQEALSGSHHHHHH

Table 16 lists the immunization campaigns, antigens used, and number of clones returned at each stage of screening. Clones from which antibody sequences were successfully recovered were first screened for binding to recombinant full length human MSLN (MSNW10) and H838 cells that express MSLN on the cell surface. Binders were further screened for binding to cells in the presence of recombinant soluble MSLN lacking the C-terminal 7 AAs (MSNW11). This form of recombinant MSLN represented the product of cleavage at the closest cleavage site to the C-terminus known at the time, therefore, it theoretically represented the longest form of shed MSLN. The final concentration of shed MSLN in the assays was 100 nM. This approximates the highest concentration that was measured in tumor ascites.

TABLE 16

| | | | | | Unique |
|---|---|---|---|---|---|
| | | | V-regions | Binding | Selective |
| Strain | Antigen | Recovery | recovered | confirmed | Binders |
| Ablexis | MSNW20 | Hybridoma | 127 | 55 | 4 |
| Ablexis | MSNW20 | B-cell | 12 | 7 | 4 |
| Ablexis | MSNW20 | B-cell (repeat) | 190 | 54 | 7 |
| AJ | MSNW20 | Hybridoma | 16 | 7 | 2 |
| AJ | MSNW23 | Hybridoma | 47 | 7 | 6 |

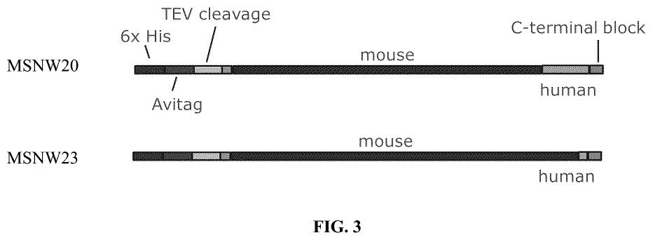

FIGS. 4A-B shows selected binding curves from the selectivity assay. FIG. 4A shows the results for the antibody MSNB124 whose binding was not competed by a consistent amount of excess recombinant shed MSLN. FIG. 4B shows the results from antibody MSNB110 whose binding was completely abrogated with the addition of shed MSLN, demonstrating that this antibody was not selective for membrane MSLN.

Table 17 gives a full list of the antibodies whose binding was not inhibited when recombinant shed MSLN was added to the cell binding assay.

TABLE 17

| Ab ID | Host Species | Immunogen | Recovery | Affinity (nM) | Tm1 | Tagg |
|---|---|---|---|---|---|---|
| MSNB71 | Ablexis Mouse | MSNW20 | Hybridoma | 0.2 | 74 | 75 |
| MSNB75 | Ablexis Mouse | MSNW20 | Hybridoma | 1.5 | 64 | 66 |
| MSNB124 | Ablexis Mouse | MSNW20 | Hybridoma | 6.4 | 70 | 82 |
| MSNB133 | Ablexis Mouse | MSNW20 | Hybridoma | 3.7 | 75 | 74 |
| MSNB451 | Ablexis Mouse | MSNW20 | Single B-cell | 16.1 | 77 | 75 |
| MSNB455 | Ablexis Mouse | MSNW20 | Single B-cell | 11.5 | 73 | 72 |
| MSNB457 | Ablexis Mouse | MSNW20 | Single B-cell | 5.6 | 68 | 79 |
| MSNB459 | Ablexis Mouse | MSNW20 | Single B-cell | 3.5 | 71 | 75 |
| MSLNB568 | Ablexis Mouse | MSNW20 | Single B-cell | ~1-5 nM | 74 | 74 |
| MSLNB569 | Ablexis Mouse | MSNW20 | Single B-cell | ~1-5 nM | 73 | 73 |
| MSLNB570 | Ablexis Mouse | MSNW20 | Single B-cell | 3.6 | 70 | 71 |
| MSLNB571 | Ablexis Mouse | MSNW20 | Single B-cell | 1.8 | 72 | 74 |
| MSLNB590 | Ablexis Mouse | MSNW20 | Single B-cell | ~1-5 nM | 70 | 76 |
| MSLNB591 | Ablexis Mouse | MSNW20 | Single B-cell | ~1-5 nM | 72 | 73 |
| MSLNB592 | Ablexis Mouse | MSNW20 | Single B-cell | ~1-5 nM | 75 | 75 |
| MSNB74 | AJ WT Mouse | MSNW20 | Hybridoma | 1.1 | nd | nd |
| MSNB112 | AJ WT Mouse | MSNW23 | Hybridoma | 3.2 | nd | nd |
| MSNB193 | AJ WT Mouse | MSNW23 | Hybridoma | 13.4 | nd | nd |
| MSNB202 | AJ WT Mouse | MSNW23 | Hybridoma | 7.3 | nd | nd |
| MSNB222 | AJ WT Mouse | MSNW23 | Hybridoma | 3.5 | nd | nd |
| MSNB236 | AJ WT Mouse | MSNW23 | Hybridoma | 26.5 | nd | nd |
| MSNB264 | AJ WT Mouse | MSNW23 | Hybridoma | 3.8 | nd | nd |
| MSNB265 | AJ WT Mouse | MSNW23 | Hybridoma | 31 | nd | nd |

Table 18 lists a subset of antibodies identified in the same screen that bind to soluble MSLN. These antibodies were later used as assay controls to differentiate the activity of a membrane selective antibody from that of a non-selective antibody. Control antibodies were also generated from published sequences for this purpose.

TABLE 18

| Ab ID | Host Species/Source | Immunogen | Recovery | Affinity (nM) | Tm1 | Tagg |
|---|---|---|---|---|---|---|
| MSNB90 | Ablexis Mouse | MSNW20 | Hybridoma | 0.33 | 70 | nd |
| MSNB95 | Ablexis Mouse | MSNW20 | Hybridoma | 3.90 | 72 | nd |
| MSNB110 | Ablexis Mouse | MSNW20 | Hybridoma | 0.22 | 69 | nd |
| MSNB9 | Publication | na | na | 0.04 | nd | nd |

Binders that lacked binding to soluble MSLN were further characterized for their affinity for recombinant full-length human MSLN by SPR, and for their Tm & Tagg by DSC (Table 17). The minimal binding affinity cutoff was 10 nM. All of the Ablexis-derived antibodies except for MSNB451 and MSNB455 had an affinity <10 nM. The affinities of five of the Ablexis-derived antibodies were not determined using SPR but, based on cell binding, these were estimated to be in the 1-5 nM range and were advanced. In additional experiments the affinities of MSLNB568, MSLNB569, MSLNB590, MSLNB591, and MSLNB592 were 0.5, 3.4, 1.2, 0.3, and 0.9 nM respectively, as determined by SPR. The criterion for minimum Tm was >65° C. and only MSNB75 failed to meet this criterion.

MSNB75 also contained a free cystine in its CDR which would be problematic for development. Table 19 lists Ablexis-derived antibodies that passed the initial criteria for affinity and Tm and were therefore advanced for more in-depth characterization. Antibodies derived from the wild-type AJ mice were not advanced due to the additional effort that would be needed for humanization.

TABLE 19

| | Campaign |
|---|---|
| Ab ID | ID |
| MSNB71 | HYB604 |
| MSNB124 | HYB604 |
| MSNB133 | HYB604 |
| MSNB457 | HYB625 |
| MSNB459 | HYB625 |
| MSLNB568 | AB331 |
| MSLNB569 | AB331 |
| MSLNB570 | AB331 |
| MSLNB571 | AB331 |
| MSLNB590 | AB331 |
| MSLNB591 | AB331 |
| MSLNB592 | AB331 |

### Binding to MSLN Comprising a Singular Nucleotide Variation/Polymorphism.

The membrane restricted region of MSLN contains a M>V singular nucleotide variation/polymorphism (SNV or SNP) caused by a A-to-G mutation. The homozygous SNVG/G is present in 11.5% of the general population and incidence of the genotype varies in different ancestry groups. The highest incidence is approximately 27% in African/African Americans, while European (non-Finnish) have an incidence of approximately 5% (data derived from gnomAD for rs1135210 SNV). Select antibodies were screened for binding to the SNVG/G variant, using SPR, including MSNB71, MSNB124, MSLNB568, MSLNB569, MSLNB570, and MSLNB591. Briefly, the α-MSLN mAbs were captured on GαH (˜30-40 RUS), followed by injection of either wild-type recombinant human MSLN or the SNV containing MSLN protein in a 3-fold serial dilution in HBSP+ buffer supplemented with 0.1 mg/mL of BSA for antigen stabilization. The results showed that the binding to the SNV could not be accurately modeled to yield an affinity value for any of the antibodies tested, since the biphasic binding was observed.

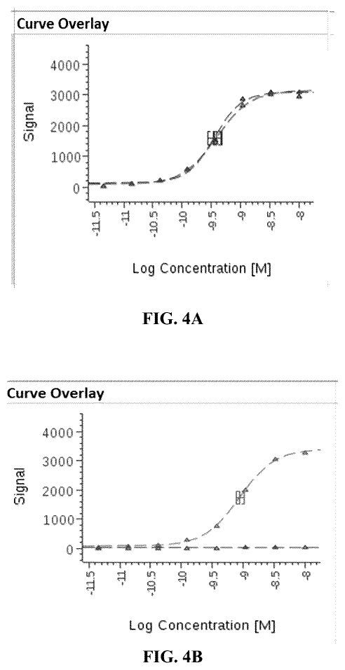

The highest affinity membrane restricted binder, MSNB71 was used for immunohistochemistry to identify cells and tissues that expressed mesothelin. FIG. 5 shows that unexpected staining of smooth muscle, cardiac muscle and peripheral neurons was observed. At the same time, a screen for off-target binding (Retrogenix) showed reactivity against Desmin, a member of the type III intermediate filament family, as well as other proteins. The binding to type III intermediate filaments was confirmed by immunoprecipitation followed by mass spectroscopy (data now shown). This study identified type III intermediate filaments Peripherin, Desmin, Vimentin and PTBP1 as potentially interacting with MSNB71. Two of these proteins, Peripherin and Desmin, had expression profiles that were consistent with the staining patterns observed in IHC. Comparison of the primary amino acid sequence of the membrane specific region of MSLN with the type III intermediate filament proteins revealed sequence similarities. In the 10 AA stretch at the C-terminus of MSLN, 7 of the 10 residues were identical to a 10 AA stretch in Peripherin and for Desmin 7 of 10 were either identical or structurally conserved. The expression of these proteins is believed to be intracellular, and a functional consequence of this off-target binding was not established. However, for antibodies suitable for further development it was considered preferable to avoid MSLN binders that demonstrated strong binding to type III intermediate filaments so as to reduce the potential for off-target binding. For this reason, commercial sources of Desmin and Peripherin recombinant protein, as well as cells expressing type III intermediate filaments were identified. C2C12 cells that express Desmin and Kelly cells that express Desmin, Vimentin and Peripherin were also identified. Using these reagents, off target binding was evaluated for select antibodies by SPR, FACS analysis of permeabilized cells, or immunofluorescent staining of permeabilized cells. Table 20 lists the properties of each antibody. Only one of the antibodies tested, MSNB457, consistently showed little or no binding to the type III intermediate filaments and met the criteria for binding affinity and stability.

TABLE 20

| | huDesmin | huPeripherin | | |
|---|---|---|---|---|
| | binding | binding | C2C12 | Kelly |
| Ab ID | SPR | SPR | permeabilized | permeabilized |
| MSNB71 | Yes | No/Low | Yes | Yes |
| MSNB124 | Yes | No/Low | Yes | Yes |
| MSNB133 | nd | nd | Yes | nd |
| MSNB457 | No/Low | No/Low | Low | Low |
| MSNB459 | nd | nd | Yes | nd |
| MSLNB568 | nd | nd | Yes | Yes |
| MSLNB569 | nd | nd | Low | Low |
| MSLNB570 | Yes | No/Low | Yes | Yes |
| MSLNB571 | Yes | No/Low | Yes | Yes |
| MSLNB590 | nd | nd | nd | nd |
| MSLNB591 | nd | nd | nd | nd |
| MSLNB592 | nd | nd | nd | nd |

8.1.2 Affinity and Selectivity of MSLN Antibodies

In order to determine the affinity and selectivity of MSLN antibodies, High Content Imaging (HCl) was employed. Affinity was assessed by measuring cell surface staining of MSLN antibodies in OVCAR-8 cells which express MSLN and potential cross-reactivity was monitored by analyzing staining intensity of the MSLN antibodies in MSLN-null C2C12 mouse myoblasts and Kelly human neuroblastoma cells.

OVCAR8 cells were cultured in RPMI 1640 medium supplemented with 10% FCS, 1% L-glutamine and 1% Gentamycin and seeded in Greiner CELLSTAR plate one day before experiment. 24 h after seeding, antibody mixture was prepared by diluting MSLN and control antibodies in Table 21 in full media containing CellMask™ Deep Red Plasma Membrane Stain (ThermoFisher, C10046) in 12 concentrations each with 3 folds dilution starting from 20 μg/ml and 20 μl of which were added directly to the cells in 20 μl. Antibodies were allowed for binding to the cell surface for 2h inside the incubator (37°) C. and the surface bound antibodies were fixed for 15 min at RT by directly adding 30 ul of 10% Formaldehyde (04018-1, Polysciences). Afterwards residual Formaldehyde was removed by washing the cells twice with PBS and the primary antibodies were stained with secondary antibodies (Alexa Fluor 488 goat anti-human IgG, Invitrogen, A11013) complemented with Hoechst 33342 (H3570, ThermoFisher) and HCS CellMask™ Deep Red Stain (ThermoFisher, H32721) in PBS for 1h at RT. After removing unbound antibodies and other dyes by washing the cells 3 times with PBS cells were imaged on the CellVoyager 7000 (Yokogawa, Tokyo, Japan) using a 40×/0.95 objective. Of each well, 9 fields were imaged. Image analysis was performed with Acapella (PerkinElmer, Waltham, MA, USA) and the results stored in Phaedra (OpenAnalytics, Antwerp, Belgium). Data analysis was done in R (www.r-project.org).

TABLE 21

| Antibody | Conc (mg/ml) | Parental protein ID |
|---|---|---|
| ITC (isotype | 10.06 | B23B62.011 |
| control) | | |
| B71 | 1.26 | MSNB71.004 |
| B124 | 5.99 | MSNB124.005 |
| B457 | 6.87 | MSNB457.003 |
| B236 | 7.99 | MSNB236.001 |
| B568 | 0.25 | MSLNB568.001 |
| B569 | 0.64 | MSLNB569.001 |
| B570 | 3.17 | MSLNB570.002 |
| B571 | 0.44 | MSLNB571.001 |

To test for cross-reactivity, Kelly and C2C12 cells, which are absent of MSLN expression, were used in the assay. Kelly cells were cultured in RPMI-1640 supplemented with 10% FCS, 2 mM L-glutamine and 50 μg/ml Gentamycin and C2C12 cells were grown in DMEM complemented with 10% FCS, 1 mM Sodium Pyruvate, 2 mM L-glutamine and 50 μg/ml Gentamycin. In order to obtain differentiated C2C12 cells growth medium was replaced by DMEM with 2% Horse serum once the cells reached 80-90% confluency. Kelly and C2C12 cells were fixed by Formaldehyde as described above for OVCAR-8 cells without preincubation with MSLN antibodies. Permeabilization, blocking and staining were performed in one step protocol as described in following. The cells were incubated with MSLN and isotype controls antibodies in Table 21 at 12 concentrations with 3 folds dilution starting from the top concentration at 20 g/ml in PBS consisting of 5% BSA (Sigma), 0.3% Triton X-100 (Sigma) for 2h at RT. After removing unbound antibodies by washing the cells twice with PBS the cells were incubated with secondary antibodies (Alexa Fluor 488 goat anti-human IgG, Invitrogen, A11013), Hoechst 33342 (H3570, ThermoFisher) and HCS CellMask™ Deep Red Stain (ThermoFisher, H32721) in PBS for 1h at RT. Image acquisition and analysis were conducted in the same way as described for the surface staining above.

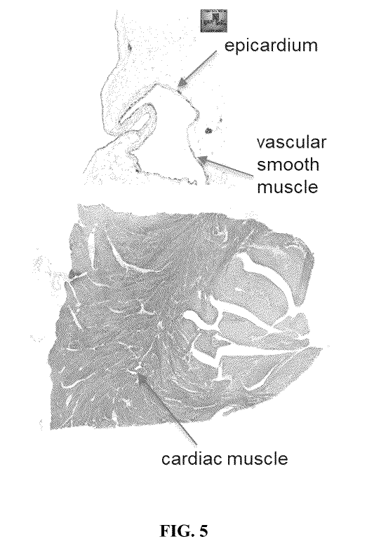

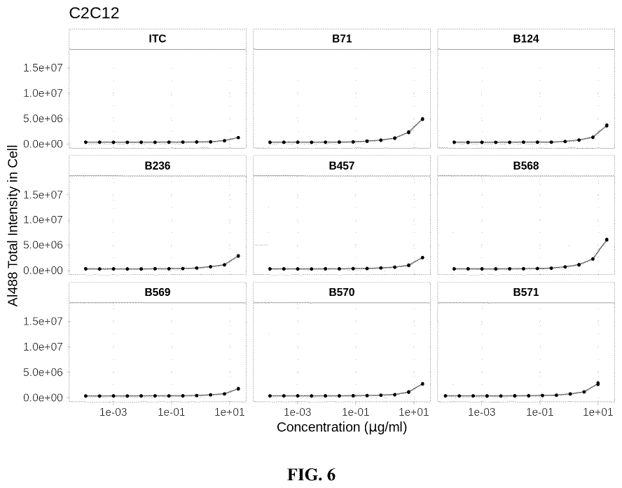

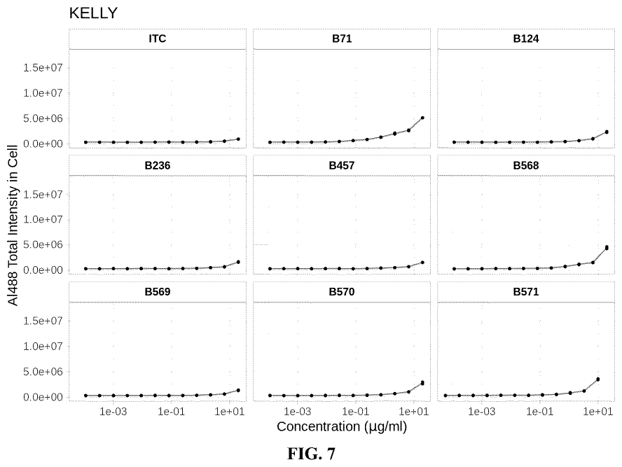

FIGS. 6, 7, and 8 show the uncorrected total intensity in the cell area as a function of antibody concentration, for C2C12, KELLY and OVCAR8 cells, respectively. In KELLY and C2C12 cells, where MSLN is absent, some aspecific binding was detected at high concentrations of B71, B124, B568, B570 and B571 whereas almost no aspecific staining was seen in B236, B457 and B569 in comparison to isotype control (ITC). In OVCAR8 we see much stronger binding with an onset at earlier concentrations due to strong binding of the MSLN antibodies to the target.

To obtain IC50 values for antibody binding, the technical background, which was calculated from ITC and ITC-biotin wells at low concentration (≤0.1 μg/ml), was first subtracted. The background value was 435,000. The data was then fitted using the four-parameter logistic using least-squares regression, keeping the lower boundary fixed at 0. Results are given in Table 22.

TABLE 22

| | Maximum Value | Upper Bound of Fit | IC₅₀ |
|---|---|---|---|
| Antibody | (arbitrary units) | (arbitrary units) | (μg/ml) |
| B236 | 14,300,000 | 13,800,000 | 0.0523 |
| B71 | 15,400,000 | 14,800,000 | 0.0713 |
| B570 | 13,900,000 | 13,600,000 | 0.0728 |
| B124 | 14,600,000 | 14,200,000 | 0.0735 |
| B571 | 14,600,000 | 14,300,000 | 0.0735 |
| B568 | 17,000,000 | 16,600,000 | 0.0764 |
| B569 | 16,000,000 | 16,200,000 | 0.166 |
| B457 | 14,700,000 | 16,600,000 | 0.501 |
| ITC | 173,000 | 282,000 | >10 |

This was done for OVCAR8 only, because the lack of a saturation plateau in the two other cell lines does not permit this type of curve fitting. For OVCAR8 the plateau is not always clearly defined, but the upper bound of the fitted curve is always similar to the maximum measured value, indicating that the fit remains realistic.

### 8.2 Example 2: Characterization of the Epitope of Select Antibodies

### Epitope Characterization by Alanine Scan

To further confirm that MSNB457 binds the membrane restricted C-terminal portion of MSLN, an alanine scan of this region was done.

Each AA in the membrane-restricted C-terminal epitope, 587LVLDLSMQEALS598 (SEQ ID NO: 203), was replaced by alanine. These mutations were made in the context of full length MSLN. Three amino acid residues, L589, D590, and M593, were found to be critical for binding. Mutation of V588, S592, or L597 to alanine resulted in a partial reduction in binding, and mutation of the other 5 had no effect on binding (see Table 23). The role of A596 was not determined.

TABLE 23

| Antigen | | | | |
|---|---|---|---|---|
| Description | ka (1/Ms) | kd (1/s) | KD (M) | SPR Comments |
| MSLN_L587A | 5.71E+05 | 6.44E−03 | 1.13E−08 | Little/no impact |
| MSLN_V588A | biphasic binding, suboptimal fit | | | Reduced binding |
| MSLN_L589A | low/no binding | | | |
| MSLN_D590A | low/no binding | | | |
| MSLN_L591A | 9.54E+05 | 3.92E−03 | 4.11E−09 | Little/no impact |
| MSLN_S592A | 1.44E+06 | 2.88E−02 | 2.01E−08 | >3 fold reduced |
| | | | | affinity |
| MSLN_M593A | low/no binding | | | |
| MSLN_Q594A | 6.21E+05 | 1.16E−03 | 1.87E−09 | Little/no impact |
| MSLN_E595A | 7.52E+05 | 3.51E−03 | 4.66E−09 | Little/no impact |
| MSLN_A596 | Not tested | | | |
| MSLN_L597A | biphasic binding, suboptimal fit | | | Reduced binding |
| MSLN_S598A | 6.31E+05 | 2.03E−03 | 3.22E−09 | Little/no impact |

To better understand the epitope diversity of the select panel of anti-MSLN mAbs, we further assessed the binding of select mAbs (MSNB71, MSNB124, MSLNB568, MSLNB569, MSLNB570, and MSLNB591) to MSLN variants containing single amino acid substitutions to alanine at positions shown to be critical for MSNB457 binding. The binding was assessed by SPR. For this assay, GAH-Fc was used to capture mAbs on a C1 sensor chip. Wild-type peptides MSNW10 (huMSLN ECD-6×His) and MSNW28 (6×HisAvis-huMSLN ECD) were injected at single-cycle kinetics (SCK) concentrations 100, 37, 11, 3.7 nM. MSLNW23 (human mature MSLN protein as shown in SEQ ID NO:247, with an amino acid substitution of D590A) and MSLN26 (human mature MSLN protein as shown in SEQ ID NO:247 with an amino acid substitution of M593A) injected at SCK concentrations 1000, 500, 250, 125 nM. MSLNW22 (human mature MSLN protein as shown in SEQ ID NO:247 with an amino acid substitution of L589A) injected at SCK concentrations 1000, 333, 111, and 37 nM. All run in HBSP+0.01% BSA Buffer.

The results suggested that the mAbs tested (MSNB71, MSNB124, MSLNB568, MSLNB569, MSLNB570, and MSLNB591) either lacked binding to the alanine variants or showed a biphasic binding profile. These data confirm that the mAbs bind to a region of the MSLN that is similar to the MSNB457 binding region, but given that there were some mAbs for which binding was not completely abrogated, there does appear to be differences in the degree to which specific amino acids contribute to the overall binding affinity.

The membrane-restricted epitope of MSNB457 in full-length membrane-associated MSLN may also be present in the “stub” that is left on the membrane after MSLN is shed from the cell. As discussed previously, the exact site of cleavage varies depending on the responsible protease and is further complicated by the lack of a specific recognition and cleavage sites for some proteases. For this reason, the length of the stub remaining on the cell after cleavage varies, and therefore the binding of antibodies directed to the stub would be expected to vary as well. Moreover, the confirmation of the membrane restricted epitope could change after cleavage, and, as a result, antibodies that bind this epitope in the context of the full-length protein may not retain after cleavage. To determine if MSNB457 retains binding to the cleaved stub, synthesized peptides corresponding to the stub region were synthesized, and cell lines expressing the stub were also synthesized. The peptides corresponded to either the last 8 or the last 12 C-terminal amino acids. Binding to these peptides was assessed via SPR and in OVCAR-8 binding assays where the peptides were assessed for their ability to compete with binding. The cell line used for overexpressing the stub was an OVCAR8 MSLN-knock-out line. Rather than generating multiple cell lines, each expressing stubs of different length, a single cell line expressing a stub that was 12 AAs in length was generated. It was understood that, as the stub gets shorter, binding could potentially be reduced, and therefore the results would not directly translate to the disease setting where stubs of multiple lengths could be present. If binding to the 12 AA stub were observed, it would be necessary to evaluate binding of progressively shorter stubs. Because the stub was displayed on the cell surface, cell lines with either a cMYC or GFP tag fused to the N-terminus of the 12 AA stub were generated.

Table 24A summarizes the results of SPR binding analysis and binding competition assays using peptides, as well as FACS analysis of binding using recombinant cell lines. Three of the mAbs tested bound to the 12 AA stub in all cases (MSNB71, MSNB124 & MSLNB570). One mAb, MSLNB571 was tested only in the SPR assay, and was shown to bind the 12 AA peptide. Interestingly, two of the mAbs, MSNB75 and MSNB457 demonstrated weak or no binding when the 12 AA peptide was presented with a free N-terminus but strong binding when a tag was added to the N-terminus. The C-terminal sequence of the cMYC and GFP proteins show no homology to the MSLN sequence upstream of the stub region. These data show that the presence of the tag sequences stabilizes the stub in a binding competent conformation that is necessary for these two mAbs to bind. The binding of the antibodies to OVCAR8 cells was not reduced with the addition of the 8 AA peptide. This was consistent with the alanine scan done with MSNB457 which showed that the DLV stretch of residues at positions −9 to −11 (from the C-terminus) are critical for binding.

TABLE 24A

| | | OVCAR8 | OVCAR8 | | | |
|---|---|---|---|---|---|---|
| | 12 AA | competition | competition | | c-MYC-12 | GFP-12 |
| | Peptide | 12 AA | 8 AA | 12 AA | AA | AA |
| Ab ID | (SPR) | Peptide | Peptide | OVCAR8 | OVCAR8 | OVCAR8 |
| MSNB71 | Yes | Yes | No | Yes | Yes | Yes |
| MSNB75 | No | No | No | No | Yes | Yes |
| MSNB124 | Yes | Partial | No | Yes | Yes | Yes |
| MSNB457 | Weak | Partial | No | Weak | Yes | Yes |
| MSLNB570 | Yes | Yes | No | Yes | Yes | Yes |
| MSLNB571 | Yes | Yes | No | n/a | n/a | n/a |

To further characterize the binding of the anti-MSLN mAbs (MSNB71, MSNB124, MSLNB568, MSLNB569, MSLNB570, and MSLNB591), we used a series of membrane proximal MSLN fragments (stub peptides) ranging from 7 to 14 AAs based on known cleavage sites within MSLN. The binding was assessed by SPR. Briefly, GAH-Fc was used to capture mAbs on C4 sensor chip followed by antigen injection starting at 30 nM (14 amino acid stub), 100 nM (10-13 amino acid stubs), or 1000 nM (7-9 amino acid stubs) progressing in a 3-fold dilution series in single cycle kinetics mode. HBSP+0.01% BSA was used in the assay.

The results suggested that none of the mAbs tested were able to bind stub peptide that was 11 AA in length or shorter (Table 24B)

TABLE 24B
Binding of mAbs to indicated MSLN fragments, KD (M) are shown.

| | | | | | 10 AA | 9 AA | 8 AA | 7 AA stub |
|---|---|---|---|---|---|---|---|---|
| | 14 AA stub | 13 AA stub | 12 AA stub | 11 AA stub | stub (a.a. | stub (a.a. | stub (a.a. | (a.a. 297- |
| | (a.a. 290- | (a.a. 291- | (a.a. 292- | (a.a. 293- | 294-303 | 295-303 | 296-303 | 303 of |
| AA ID | 303 of SEQ | 303 of SEQ | 303 of SEQ | 303 of SEQ | of SEQ ID | of SEQ ID | of SEQ ID | SEQ ID |
| Number | ID NO: 247) | ID NO: 247) | ID NO: 247) | ID NO: 247) | NO: 247) | NO: 247) | NO: 247) | NO: 247) |
| MSNB71 | 3.64E-10 | 4.69E-10 | 6.92E-10 | No | No | No | No | No |
| MSLNB568 | 1.61E-09 | 2.64E-09 | 2.33E-09 | No | No | No | No | No |
| MSLNB591 | 5.63E-10 | 1.08E-09 | 2.04E-09 | No | No | No | No | No |
| MSLNB570 | 3.67E-09 | 4.37E-09 | 7.97E-09 | No | No | No | No | No |
| MSNB124 | Poor Fit, | 1.40E-08 | Poor Fit, | No | No | No | No | No |
| | strong | | strong | | | | | |
| | binding | | binding | | | | | |
| MSLNB569 | 1.99E-09 | 5.99E-09 | 4.06E-09 | No | No | No | No | No |

### Epitope Characterization by Crystallography

The Fab portion of MSNB457, designated MSLNB703 (heavy chain: amino acids 1-225 of SEQ ID NO: 164; light chain: SEQ ID NO:165), was expressed with a His-tag to aid in purification. The purified Fab was generated to a concentration of 9.98 mg/mL in formulation buffer comprising 10 mM HEPES, 50 mM NaCl, pH 7.4. The extracellular domain (ECD) 17-mer peptide of human mesothelin (residues 582-598 of SEQ ID NO: 200) was resuspended in DMSO to a final concentration of 40 mg/mL. Fab and peptide reagents were stored at −80° C. until use.

The peptide and Fab were mixed at a molar ratio 2:1 (excess of peptide) for 2 hours on ice. The Fab-peptide complex was crystallized with the Mosquito LCP (STP Labtech) crystallization robot at 20° C. using the sitting drop vapor-diffusion method from solution containing 30% PEG1500. Crystals of the Fab-peptide complex were harvested in cryoprotectant (30% PEG1500, 30% (v/v) glycerol) and flash frozen in liquid nitrogen. X-ray diffraction data for the Fab-peptide complex were collected at −180° C. using station 17-ID, Industrial Macromolecular Crystallography Association Collaborative Access Team (IMCA-CAT), Advanced Photon Source, Argonne National Laboratory.8 Data were processed using autoPROC9 and STARANISO10, and the resolution cutoff for the highest resolution shell used the following criteria: CC(½)≥0.3, R(pim)<=0.6, and local I/sigI≥1.2. PHENIX was employed for phasing and refinement, and model building was performed using Coot. 11 The structure of the Fab-mesothelin complex was determined with anisotropic resolution cutoffs at 1.96 Å, 2.03 Å, and 2.32 Å by molecular replacement using Phaser12 and a homology model of the Fab as a search model generated using MOE13. All structures were refined using individual B-factor refinement on Fab and peptide atoms, and local NCS restraints were automatically applied. The Ramachandran Plot of the final model calculated with Molprobity 14 shows 97.38% of all residues in the favored region, 2.62% in the allowed region, and no outliers.

Results. The epitope for the binding of MSLNB703 Fab to the ECD peptide of human mesothelin was determined by X-ray crystallography. Crystals were grown in 30% PEG1500 and belong to the triclinic space group P 1 with unit cell dimensions of 60.2×79.7×86.8 Å. The asymmetric unit (ASU) contains four Fab complexes, each with a single bound peptide. The contact residues of MSLN within 4 or 5 A of MSLNB703 Fab for the four Fab/peptide complexes are shown below:
Copy 1 MSLN contact residues within 4 A: (i) G585 to Q594, (ii) A596 to L597.
Copy 1 MSLN contact residues within 5 A: (i) G585 to Q594, (ii) A596 to L597.
Copy 2 MSLN contact residues within 4 A: (i) G585, (ii) L587 to Q594, (iii) A596 to L597.
Copy 2 MSLN contact residues within 5 A: (i) G585, (ii) L587 to Q594, (iii) A596 to L597.
Copy 3 MSLN contact residues within 4 A: (i) G585, (ii) L587 to Q594, (iii) A596 to L597.
Copy 3 MSLN contact residues within 5 A: (i) G585 to Q594, (ii) A596 to L597.
Copy 4 MSLN contact residues within 4 A: (i) Y586 to M593, (ii) E595
Copy 4 MSLN contact residues within 5 A: (i) Y586 to M593, (ii) E595.

The results demonstrated that mesothelin amino acid residues within 4.0 A of residues on MSLNB703 Fab as observed in at least one out of four Fab/peptide complexes, included G585, Y586, L587, V588, L589, D590, L591, S592, M593, Q594, A596, and L597. The same residues of mesothelin were within 5.0 A of MSLNB703 Fab. An amino acid residue on mesothelin is considered to be within a given distance of MSLNB703 Fab (e.g. 4.0 A or 5.0 A) if the coordinates of any atom of the residue are within the given distance of the coordinates of any atom of the antibody.

The results also demonstrated that mesothelin amino acid residues within 4.0 A of residues on MSLNB703 Fab as observed in all four Fab/peptide complexes, included L587, V588, L589, D590, L591, S592, M593.

The contacted residues on MSLN that were within 4.0 A and 5.0 A of MSLNB703 Fab form a discontinuous epitope including 585-594 and 596-597 amino acid residues of MSLN (SEQ ID NO: 200).

The contacted residues on MSLN that were within 4.0 A and 5.0 A of MSLNB703 Fab form an epitope including 587-593 amino acid residues of MSLN (SEQ ID NO: 200).

### 8.3 Example 3: Evaluation of MSNB457 as a scFv

The MSLNB457 variable regions were formatted as scFv and characterized. First, they were formatted as scFv in the light chain-linker-heavy chain orientation and expressed in E. coli. Supernatants were evaluated in a recombinant MSLN binding ELISA assay after incubation at room temperature, 55° C., 60° C. or 65° C. The scFv derived from MSNB457 retained, 88%, 91% and 64% of its binding after incubation at these three temperatures, respectively. The reduction in binding after incubation at 65° C. compared to the room-temperature sample indicated that stability could be a liability if MSNB457 were formatted as a scFv.

The MSNB457-derived scFv in the LH orientation was then formatted as a bispecific antibody in combination with the CD3B376 CD3 binding Fab arm (described in U.S. Patent Application Publication No.: 2020/0048349, the content of which is herein incorporated by reference in its entirety) to yield two bispecific antibodies MNC3B170 and MNC3B305. These two differed in the Fc mutations used to drive heterodimerization but were otherwise identical. Both had a Tonset of 57.5ºC, Tm1 of 64.5° C. and Tagg of 66ºC. The Tonset and Tm1 values were similar to related molecules which used MSNB457 in the Fab format, MNC3B193 and MNC3B304 (Tonset 59ºC, Tm1 64° C.). However, the Tagg values for MNC3B170 and MNC3B305 were ˜10° ° C. lower than MNC3B193 & MNC3B304, suggesting that formatting MSNB457 as a scFv could increase the resulting bispecific antibody's propensity to aggregate.

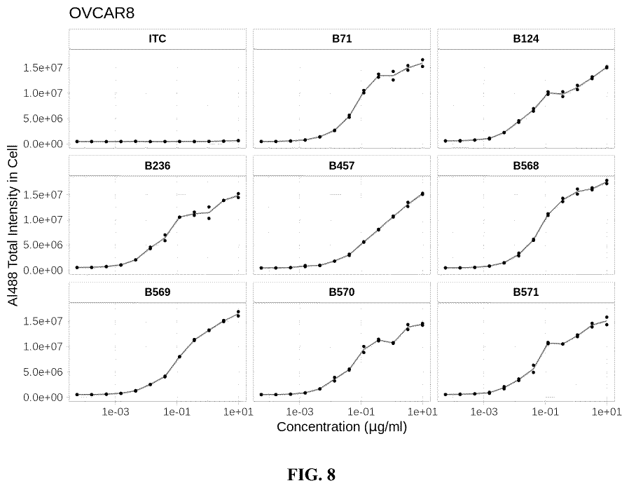

MNC3B170 and MNC3B305 were evaluated by SPR for binding to recombinant mesothelin (MSNW10). The KD of these two molecules which featured MSNB457 as a scFv was 17 nM. In comparison, MNC3B170 and MNC3B305 which featured MSNB457 as a Fab and had an average KD of ˜9 nM. This result shows that the affinity of MSNB457 for mesothelin is reduced by half when it is formatted as a scFv. This reduction of binding affinity was confirmed in cell binding assays where the binding EC50 and maximum binding of MNC3B170 was consistently lower than similar bispecific antibodies that used MSNB457 as a Fab (FIG. 9).

Because of the reduction in SPR-measured affinity for recombinant MSLN and reduction in binding to cells expressing MSLN when MSNB457 was formatted as a scFv, for the construction of bispecific antibodies, MSNB457 was used in the Fab format and paired with a CD3 targeting scFv.

MSNB457 has human germline-derived sequences and lacks substantial PTM liabilities. It binds specifically to the membrane-restricted region of full length MSLN. The thermal stability of MSNB457 is in the acceptable range and it shows little or no binding to type III intermediate filaments. For these reason MSNB457 was chosen to pair with CD3-targeting scFv to generate bispecific antibodies that would mediate T-cell redirection and kill MSLN-expressing cells.

### 8.4 Example 4: Choice of CD3-Targeted scFv

At the time that the initial bispecific antibodies were constructed, there were three choices for CD3-targeting scFv. The first two options were based on CD3W245 (described in International Patent Application Publication No.: WO 2021/240388, the content of which is herein incorporated by reference in its entirety). The VH and VL regions of CD3W245 were formatted as a scFv in either the HL or LH orientation. While the two orientations gave a similar affinity when measured by SPR, they consistently demonstrated different cell binding EC₅₀ s, with the LH orientation being 5-10-fold weaker than the HL orientation. The third option, CD3B450 (described in International Patent Application Publication No.: WO 2019/224717, the content of which is herein incorporated by reference in its entirety), had substantially weaker binding. Additional CD3 targeting scFv were characterized in combination with MSNB457. Both clones, CD3B2051-N106A and CD3B2030-N106A (described in International Patent Application No.: PCT/IB2022/052646, the content of which is herein incorporated by reference in its entirety) were reported to have a cell binding EC50 that ranges between 50 and 100 nM, which provided options that were intermediate between CD3W245 LH and CD3B450. See Table 25.

TABLE 25

| | CD3 binding | Tcell binding EC50 | |
|---|---|---|---|
| Anti-CD3 | affinity (SPR) | range to report | Cyno Cross |
| Parental/Format | (nM) | (nM) | reactivity |
| CD3W245-HL-ScFv | 0.5~1 | 1~3 | NO |
| CD3W245-LH-ScFv | 0.5~1 | 20~50 | NO |
| CD3B2030-N106A | nd | 50-100 | NO |
| CD3B2051-N106A | nd | 50-100 | NO |
| CD3B450-LH-ScFv | 50~100 | >300 | YES |

For T-cell redirecting bispecific antibodies, the optimal combination of target affinities is influenced by many factors. The receptor density of the tumor-associated antigen on tumor cells, and the normal tissue expression of the antigen are important considerations. Maximizing the binding affinity of the tumor antigen targeting arm could result in potent killing of normal cells and decrease the therapeutic window, therefore the affinity must be “tuned” to provide efficacy and while minimizing normal tissue damage. Concurrently, the affinity of the CD3-targeting arm must be tuned to favor T-cell activation in the tumor, but not in peripheral tissues where generalized activation of T-cells can elicit dose-limiting toxicities.

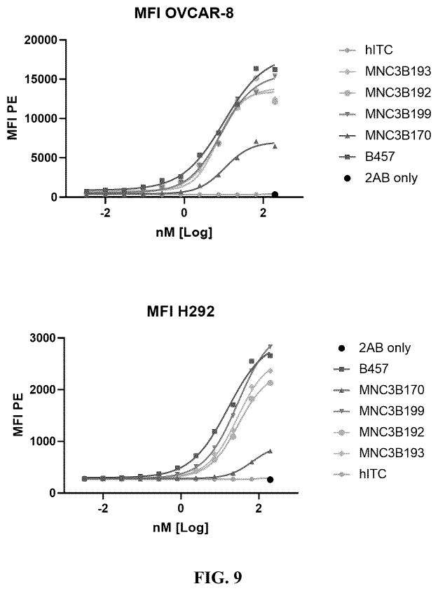

The initial screen of MSNB457 combined with different CD3-targeting scFv was done using the NCI-H838 tumor cell line (MSLN-00369). NCI-H838 is considered a high-expressing tumor line with a receptor density in the range of 200K receptors/cell. In this assay pan T-cells were used at a 5:1 E:T ratio and cytotoxicity was assessed at 48 hours. MSNB457 was combined with either the CD3W245 HL scFv (MNC3B55), the CD3W245 LH scFv (MNC3B130) or the CD3B450 LH scFv (MNC3B49). See FIG. 10.

The affinity of the CD3 targeting arm correlated with % tumor cell death. When combined with CD3W245 HL, the MSNB457-containing bispecific antibody killed with an EC50 of 0.027 nM. The activity decreased to 0.150 nM when combined with CD3W245 in the LH orientation, and when combined with CD3B450 the EC50 was >10 nM, the highest concentration used in this assay.

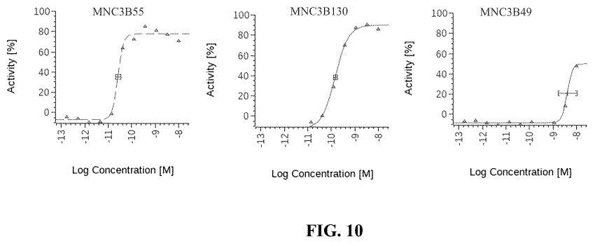

The CD3W245 HL scFv (MNC3B55) and CD3W245 LH scFv (MNC3B130) containing bispecific antibodies were evaluated in a xenograft model using the OVCAR-8 cell line. OVCAR-8 cells express ˜250K copies of mesothelin/cell. This cell line was chosen based on its behavior as a xenograft, and previous experience. As shown in FIG. 11, nearly complete tumor regression was observed for both bispecific antibodies at 1 and 0.1 mg/kg.

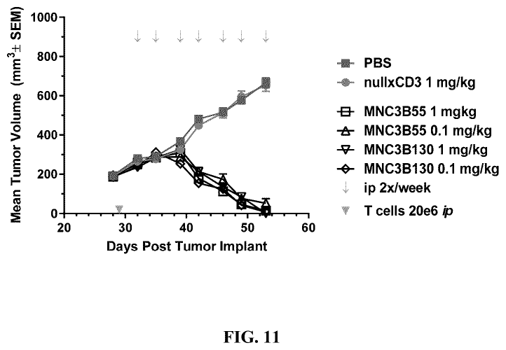

The in vitro cytotoxicity assay was repeated using the same NCI-H828 cell line at a 1:1 E:T ratio and accessing cytotoxicity at 72 hours (MSLN-00523). In this assay, the bispecific antibodies differed from the ones evaluated in the previous assay in that they included the C-terminal lysine. This difference would not be expected to affect their function, but it did result in new identifiers. Additional cris7-derived CD3 arms were also tested in this assay. See FIG. 12 and Table 26.

TABLE 26

| | MNC3B187 | MNC3B193 | MNC3B197 | MNC3B199 | MNC3B192 |
|---|---|---|---|---|---|
| CD3 Arm | CD3W245 | CD3W245 LH | B2030 | B2051 N106A | CD3B450 |
| | LH | | N106A | | |
| qAC50 [nM] | 0.0035 | 0.10 | 0.34 | 1.19 | 14.4 |
| AC90 [nM] | 0.016 | 0.25 | 2.19 | 7.32 | 28.3 |
| Max. Activity | 88 | 91 | 76 | 85 | 81 |

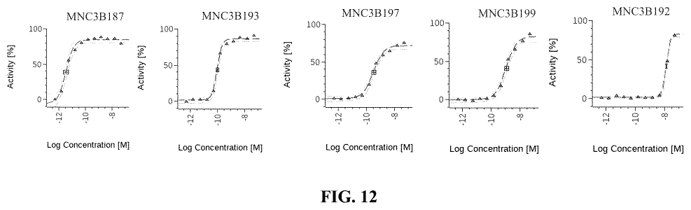

The affinity of the CD3-targeting arm also correlated with the function activity. The bispecific antibody containing the CD3W245 HL scFv was the most potent with an EC₅₀ of 0.0035 nM followed by the CD3W245 LH scFv with an EC₅₀ of 0.10 nM. The two featuring the cris7-derived scFv, MNC3B197 and MNC3B199 were intermediate in their potency at 0.34 and 1.19 nM, and the CD3B450-containing bispecific antibody showed an estimated EC₅₀ of >14.4 nM. Based on these results, the bispecific antibodies containing the CD3W245 LH scFv and B2030 N106A scFv were further characterized in cell lines with varying receptor density. For this, the cytotoxicity of the two Bipods against cell lines with a range of receptor densities was determined. Cytotoxicity EC₅₀ was then plotted against receptor density (FIG. 13). A comparison of the slope of lines fitted to the data points was used to compare selectivity. As expected, the Bipod featuring the higher affinity CD3 binder, MNC3B304, was more potent than the Bipod containing the lower affinity CD3 binder, MNC3B352. The slope of the fitted lines was almost identical, −1.264+/−0.198 vs. −1.151+/−0.247, respectively. This result demonstrated that the differences in affinity between the CD3 binding arms did not provide enhanced selectivity.

8.4.1 Monovalent vs. Bivalent Format Comparison

Initial testing of MSLN×CD3 targeting arm combinations was done using the Bipod format which enables monovalent binding to both targets. In this format the CD3-targeting arm was a scFv and the MSLN targeting arm was a Fab. It is understood in the field that bivalent targeting of the tumor antigen can have advantages. Bivalent binding can provide an avidity-driven increase in binding strength. It can also provide selectivity toward cells with higher target density. In the case where the targeted tumor cells have a higher target density than normal cells, this selectivity advantage could increase the window between efficacy and toxicity, known as the therapeutic index.

There are numerous bispecific formats that have been described that provide bivalent binding against one target and monovalent binding towards the second target. One format, termed “2+1” has been extensively described in the literature. This format is based on standard bivalent antibody format but adds a binding moiety for the second target between the Fc and Fab binding arm one of the two heavy chains. Fc heterodimerization mutations, such as knobs-into-holes, are used to generate heterodimer of the modified and unmodified heavy chains. The second binding moiety can be a Fab or scFv, although the use of a Fab necessitates the incorporation of strategies to drive proper HC-LC pairing. We chose to evaluate a 2+1 bispecific which comprises two MSLN targeting Fabs and a CD3 targeting scFv (FIG. 14).

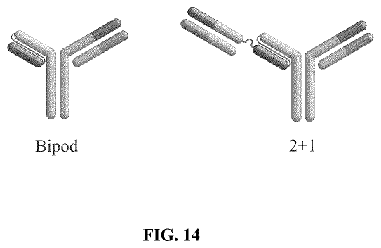

As detailed above, MSNB457 combined with the CD3W245 LH scFv in the Bipod format demonstrated potent in vivo efficacy and receptor density-dependent in vitro cytotoxicity while balancing CD3 affinity to reduce non-specific T-cell activation. BsAbs in the 2+1 format were evaluated using the same criteria. First T-cell binding was compared for Bipod and 2+1 formatted bsAbs that incorporated a CD3W245 scFv in either the LH or HL orientations. It is important to note that, unlike the Bipod, in the 2+1 format the scFv is fused to a fab at its N-terminus. The fusion of the Fab could reduce the affinity of the scFv for CD3. FIG. 15 shows the results of a T-cell binding assay done to access the binding of CD3-targeting scFv in different formats.

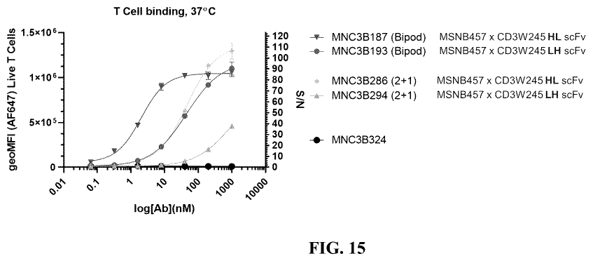

Based on the historical T-cell binding affinity of the anti-CD3 scFv (see FIG. 12), the CD3W245 HL containing Bipod, MNC3B187, showed the strongest binding with an EC50 of 2 nM. The CD3W245 LH containing Bipod, MNC3B193, gave an EC50 of 46 nM, also within the expected range. When the CD3W245 HL was formatted in the 2+1 format its EC50 was reduced to 44 nM. The CD3W245 LH formatted in the 2+1 showed an even greater reduction in binding, and an EC50 could not be calculated. Based on this data, for the purposes of comparing constructs with similar T-cell binding, the CD3W245 LH Bipod and the CD3W245 HL 2+1 was used. To compare the selectivity of the Bipod and 2+1 formats, the cytotoxicity against cell lines with a range of receptor densities was determined. Cytotoxicity EC50 was then plotted against receptor density. A comparison of the slope of lines fitted to the data points was used to compare selectivity. The bsAbs using in this assay differed from the ones used in the binding assay presented in FIG. 12 in that the Fc heterodimerization mutations were different, however the binding arms were identical. The slope of the line for the CD3W245 LH containing Biopod, MNC3B304, was similar to the slope of the CD3W245 HL 2+1 bsAb, MNC3B347. The values were −1.264+/−0.198 and −1.468+/−0.198. This difference was not statistically significant indicating that the 2+1 format did not provide an enhanced selectivity benefit. While the 2+1 format had increased potency, likely due to the increase in binding strength imparted by avidity, the Bipod was sufficiently potent in in vivo studies, and any need for increased efficacy could be addressed with dosing. See FIG. 16.

The MSNB457 mesothelin binding mAb met all criterion for binding affinity and biophysical behavior, while lacking substantial off-target binding. Bispecific antibodies in the Bipod format which combined the MSNB457 Fab with the CD3W245 LH scFv, demonstrated potent in vitro cytotoxicity and in vivo efficacy.

### 8.5 Example 5: Characterization of MSLN×CD3 Bispecific Molecules

8.5.1 MSLN SNP Frequency

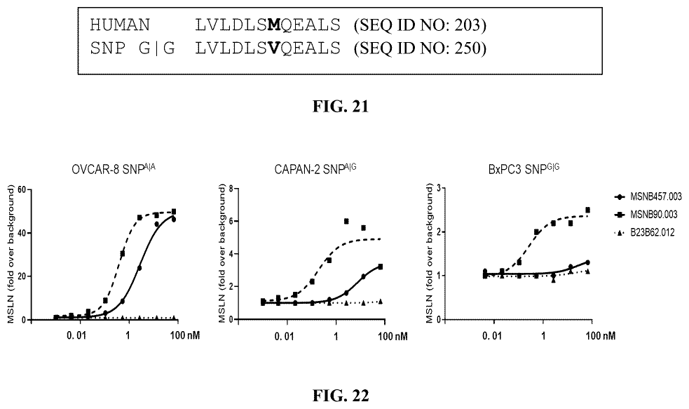

MSLN has also been described to contain SNPs. Of note, rs1135210 (GRCh38-chromosome 16:768559) is a SNP that codes for an adenosine to guanine change resulting in replacement of methionine to valine in the very C-terminal MSLN region (MSLN:p.Met593 Val). Allele frequencies for 16:768559-A-G were retrieved from gnomAD (Karczewski et al., 2020, “The mutational constraint spectrum quantified from variation in 141,456 humans,” Nature). GnomAD v.3.1.2 contains data from 75,800 individuals for this SNP (all profiled with whole-genome sequencing) (Table 27). Genotype frequencies are A|A, 0.489 (37,098); A|G, 0.396 (29,981); G|G, 0.115 (8,721). Of the total population, 11.5% (8,721 individuals of the 75,800) profiled with whole-genome sequencing in gnomAD v3.1.2 contain the homozygous SNP^{G|G} This data was accessed through Ensembl (Howe et al., 2021, Nucleic Acid Research). This SNPs analysis is relevant as homozygous SNP^{G|G} is not recognized by MNC3B304 (See FIG. 22).

TABLE 27

| | | | Allele | Homozygous | | Heterozygous | |
|---|---|---|---|---|---|---|---|
| Population | Allele count | Allele number | Frequency | SNP^{G\|G} | | SNP^{A\|G} | |
| African - African | 21521 | 41224 | 0.5221 | 5626 | (27.29%) | 10269 | (49.82%) |
| american | | | | | | | |
| Amish | 238 | 904 | 0.2633 | 29 | (6.42%) | 180 | (39.82%) |
| Ashkenazi Jewish | 805 | 3470 | 0.232 | 97 | (5.59%) | 611 | (35.22%) |
| East Asian | 1585 | 5130 | 0.309 | 278 | (10.84%) | 1029 | (40.12%) |
| European - finnish | 1695 | 10586 | 0.1601 | 149 | (2.82%) | 1397 | (26.39%) |
| European - | 15499 | 67834 | 0.2285 | 1702 | (5.02%) | 12095 | (35.66%) |
| nonfinnish | | | | | | | |
| Latino - | 4077 | 15246 | 0.2674 | 557 | (7.31%) | 2963 | (38.87%) |
| admixedAmerican | | | | | | | |
| Middle Eastern | 89 | 316 | 0.2816 | 15 | (9.49%) | 59 | (37.34%) |
| Other | 589 | 2082 | 0.2829 | 82 | (7.88%) | 425 | (40.83%) |
| South Asian | 1325 | 4808 | 0.2756 | 186 | (7.74%) | 953 | (39.64%) |
| XX (females) | 24671 | 77600 | 0.3179 | 4493 | (11.58%) | 15685 | (40.43%) |
| XY (males) | 22752 | 74000 | 0.3075 | 4228 | (11.43%) | 14296 | (38.64%) |
| Total | 47423 | 151600 | 0.3128 | 8721 | (11.51%) | 29981 | (39.55%) |

High frequencies of MSLN positivity as identified by immunohistochemistry (IHC) using antibody 5B2 have been described in mesothelioma (75%-85%), serous ovarian carcinomas (90%-95%), pancreatic cancers (50%-70%), and lung adenocarcinoma (25%-50%) (Inaguma et al., 2017, “Comprehensive immunohistochemical study of mesothelin (MSLN) using different monoclonal antibodies 5B2 and MN-1 in 1562 tumors with evaluation of its prognostic value in malignant pleural mesothelioma,” Oncotarget). In addition, MSLN is expressed to varying degrees on other solid tumors including cervical, head and neck, gastric, colorectal, esophageal, and bile duct carcinoma (Zhang et al., 2011, “Cytotoxic activity of immunotoxin SS1P is modulated by TACE-dependent mesothelin shedding,” Cancer Res).

8.5.2 Design of Platform

MNC3B304 is an IgG1 bispecific antibody that can simultaneously bind CD3ε (Uniprot ID: P07766) on T-cells and the C-terminal membrane-restricted region of MSLN (Uniprot ID: Q13421) (FIG. 17). The anti-CD3E variable region (v-region) was discovered by immunizing human antibody transgenic mice (Ablexis) with recombinant CD3εprotein followed by humanization of the light chain (LC). The anti-MSLN v-region was discovered by immunizing transgenic mice (Ablexis) with a mouse/human chimeric recombinant human MSLN protein for the purpose of directing the antibody response toward the C-terminal region.

MNC3B304 was generated by co-expression of the anti-CD3εscFv ‘knob’ heavy chain (HC) with the anti-MSLN Fab HC containing the ‘hole’ and paired with its cognate LC. The amino acid sequence for the MNC3B304 HCs and LC, as deduced from the cDNA sequence of MNC3B304, and confirmed by peptide mapping and mass spectrometry, is shown in FIG. 18 with complementarity-determining regions (CDRs) underlined according to ABM definition. The knobs-into-holes heterodimerization mutations are also underlined.

8.5.3 Intrinsic Properties of MNC3B304

Table 28 lists exemplary results from assays measuring the intrinsic properties of MNC3B304. MNC3B304 binds to recombinant full-length MSLN with a binding affinity (K_{D}) of 4.64 nM. The epitope on mrMSLN was further characterized using a series of variants wherein each of the amino acids in the stub region, 587-LVLDLSMQEALS-598 (SEQ ID NO: 203), was replaced by alanine. In this experiment, which used the parental anti-MSLN antibody MSNB457, 3 amino acid residues, L589, D590, and M593, were critical for binding. Mutation of V588, S592, E595, or L597 to alanine resulted in a partial reduction in binding, and mutation of the other 4 had no effect on binding. The role of A596 was not determined. Despite the limited size of the targeted epitope, MNC3B304 demonstrated potent in vitro activity and efficacy in tumor models. Recombinant shed MSLN lacking the 7 most C-terminal amino acids was not bound by MNC3B304, supporting its specificity for the mrMSLN region. MNC3B304 bound recombinant CD3 with K_{D} ˜0.12 nM, as measured by surface plasmon resonance (SPR). However, when formatted as a scFv in the light-heavy orientation, MNC3B304 bound at substantially lower affinity to T-cells expressing CD3 on their surface. T-cell binding by MNC3B304 did not reach saturation at concentrations as high as 1 μM; therefore, the EC₅₀ value was estimated to be >100 nM.

TABLE 28

| mAb TMP characteristic | Result |
|---|---|
| Binding affinity (human MSLN) | 4.64 nM (4.64 ± 0.33 nM) |
| Binding affinity (MSLN M593V | <100 nM |
| variant) | |
| Binding affinity (shed human | No binding detected |
| MSLN) | |
| MSLN epitope (alanine scanning) | Binding to 11 human MSLN variants with alanine mutation at different positions |
| | in the stub region was tested by SPR: D590, M593, L589 were identified as key |
| | residues in the binding epitope |
| CD3 binding | 0.12 nM (0.12 ± 0.02 nM) |
| Intact mAb (MS), Release (Da) | 128,371.5 |
| Conformational stability (DSC) | Tₘ₁ = 66.4° C.; Tₘ₂ = 72.2° C.; Tₘ₃ = 82.8° C. |
| Serum stability (aSEC-FDS) | % aggregate in human serum pH 7.4: t0 = 4.5%, 7 days at 37° C. = 8.7%; |
| | molecule is stable with low level aggregates or fragments in human serum for 7 |
| | days |
| Serum interference (SIA) | No significant SIA for binding to either MSLN or CD3. |
| Non-specific binding | No evidence of non-specific binding to irrelevant protein surfaces |
| Stability in histidine buffer (SEC) | See Table 29 |
| Intact MS after forced | Release = 128,371.5; Chem Ox = 128,449.6; pH 5 = 128,372.4; pH 8.5 = |
| degradation (Da) | 128,373.1; Thermal = 128,372.5; Physiological = 128,372.5 |
| % Monomer after forced | Release = 100%; Chem Ox = 99.4%; pH 5 = 99.7%; pH 8.5 = 97.3%; Thermal |
| degradation (SEC) | (His) = 73.3%; Thermal (acetate) = 91.4%; Physiological = 100% |
| Purity after forced degradation | Release = 100.0%; Chem Ox = 99.76%; pH 5 = 99.64%; pH 8.5 = 97.03%; |
| (GXII) | Thermal (His) = 99.75%; Physiological = 100.0% |
| MSLN binding and activity after | Release = 4.64 nM, 100%; Chem Ox = 5.22 nM, 99%; pH 5 (Iso) = 4.70 nM, |
| forced degradation (SPR) | 95%; pH 8.5 = 4.92 nM, 95%; Thermal = 4.76 nM, 97%; Physiological = |
| | 5.03 nM, 103% |
| CD3 binding and activity after | Release = 0.12 nM, 100%; Chem Ox = 0.13 nM, 100%; pH 5 (Iso) = 0.13 nM, |
| forced degradation (SPR) | 97%; pH 8.5 = 0.17 nM, 80%; Thermal = 0.14 nM, 97%; Physiological = |
| | 0.13 nM, 96% |

Table Legend: aHIC, analytical hydrophobic interaction chromatography; aSEC, analytical size exclusion chromatography; AUC, analytical ultracentrifugation; CD, cluster of differentiation; CDR, complementarity-determining region; Chem Ox, chemical oxidation; cIEF, capillary iso electric focusing; DSC, differential scanning calorimetry; Fc, fragment crystallizable region; FDS, fluorescence detection spectroscopy; GXII, capillary electrophoresis; HC, heavy chain; HCDR, heavy-chain complementarity determining region; HCLF, highly concentrated liquid formulation; HDX, hydrogen deuterium exchange; K_{D}, binding affinity; mAb, monoclonal antibody; MS, mass spectrometry; MSLN, mesothelin; N/A, not applicable; scFv, single-chain variable fragment; SEC, size exclusion chromatography; SIA, serum interference assay; SPR, surface plasmon resonance; t0, time zero; Tₘ, melting temperature; TMP, target medicinal product.

Biophysical measurements were performed to evaluate the behavior of MNC3B304 under normal and stressed conditions. Notably, MNC3B304 demonstrated a 20% reduction in binding to CD3 upon high pH (pH 8.5) stress. In addition, isomerization of D27 in heavy-chain complementarity determining region 1 (HCDR1) of the parental anti-mrMSLN antibody MSNB457 was observed in 10% of the stressed material but did not affect binding. To further probe thermal stability, MNC3B304 was incubated for 2 weeks at 4, 25, and 40° ° C. at 25 mg/mL in histidine pH 6.5 buffer (Table 29). Analysis with analytical size exclusion chromatography (aSEC) demonstrated that loss of monomer was observed only at 40° C. (Table 29).

TABLE 29

| | % Monomer | | | % HMW | | | % LMW | | |
|---|---|---|---|---|---|---|---|---|---|
| | @ 4° C. | @ 25° C. | @ 40° C. | @ 4° C. | @ 25° C. | @ 40° C. | @ 4° C. | @ 25° C. | @ 40° C. |
| | 2 weeks | 2 weeks | 2 weeks | 2 weeks | 2 weeks | 2 weeks | 2 weeks | 2 weeks | 2 weeks |
| 25 mg/mL | 100.0 | 100.0 | 97.6 | 0.0 | 0.0 | 2.2 | 0.0 | 0.0 | 0.1 |

Table Legend: aSEC, analytical size exclusion chromatography; HCLF, highly concentrated liquid formulation; HMW, high molecular weight; LMW, low molecular weight; PBS, phosphate-buffered saline. MNC3B304 was incubated for 2 weeks at 4, 25, and 40° C. at 25 mg/mL in histidine pH 6.5 buffer, followed by analysis with aSEC. The % HMW species for each condition is listed.

8.5.4 Endogenous Tumor Cell Line and Human T-cell Binding of MNC3B304

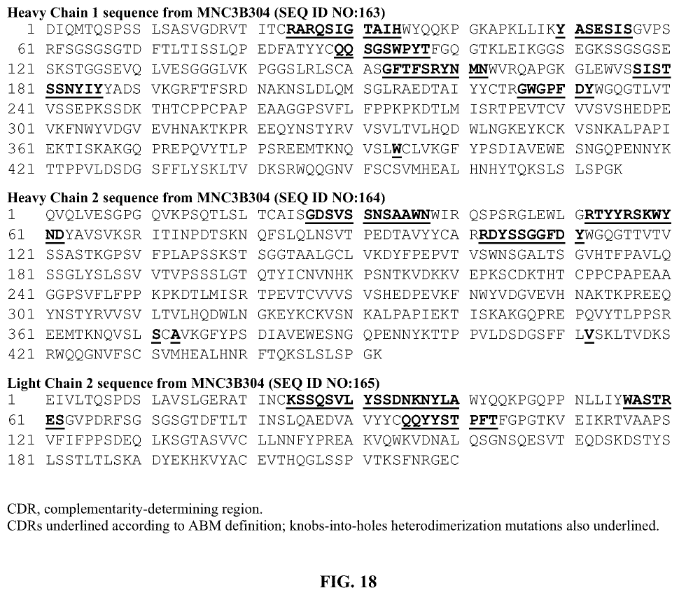

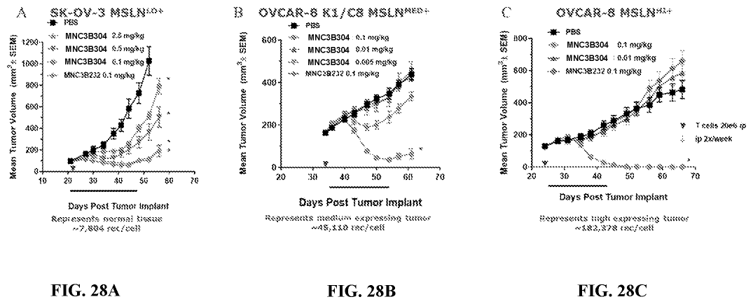

MNC3B304's in vitro binding profile was defined for a wide range of candidate cell lines across different receptor densities. MNC3B304 displayed binding to target cell lines that was consistent with receptor densities. In target cell 37° ° C. time course binding experiments, MNC3B304 showed significant and mostly stable binding to medium-high-MSLN-expressing lines (i.e., OVCAR-8 MSLN^{HI+}, OVCAR-8 K1/C8 MSLN^{MED+}; see FIG. 28), a profile that provides sufficient time to induce tumor cell death. For 1-hour dose-response binding against the same MSLN-positive cells, MNC3B304 also demonstrated significant binding but failed to show consistent saturation, preventing quantitative binding affinity determination (FIG. 19). The inability to saturate binding underscores the complex nature of MSLN targeting. The absence of binding affinity determination at 37° ° C. could be caused by receptor/antibody internalization and shedding and/or inhibition of shedding due to MNC3B304 binding at the cleavage site. Moreover, potential changes in the structure of the stub-region epitope upon shedding, and differences in MNC3B304 binding to these different forms may further the ability to observe a binding plateau.

MNC3B304 is a bispecific antibody formatted to contain the CD3W245 CD3-targeting scFv arm in the light-heavy chain orientation, which has historically been characterized as ‘medium affinity’ relative to other in-house CD3 binders. Because CD3 cell affinity can be impacted by its paired target arm, T-cell target engagement with MNC3B304 was investigated. In a 1-hour 37° C. T-cell binding assay, MNC3B304 binding did not reach saturation at concentrations as high as 1 μM; therefore, the EC50 value was estimated to be >100 nM.

8.5.5 MSNB457 Binding Epitope Resides in the mrMSLN Region

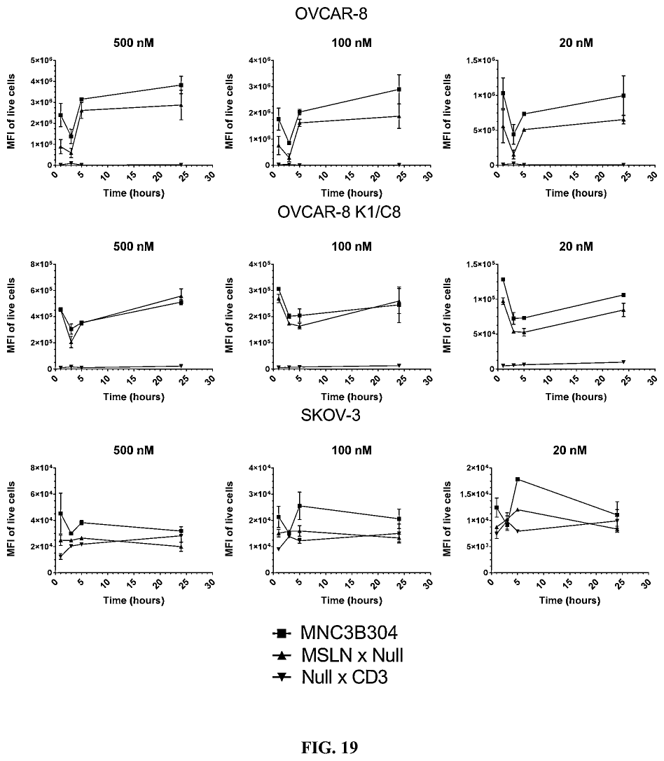

MNC3B304 targets MSLN in the C-terminal membrane-restricted stub region of MSLN that is not present in shed MSLN. Competition experiments using parental anti-mrMSLN mAb MSNB457, further confirmed that MNC3B304 does not bind soluble MSLN (FIG. 20). Shed MSLN that does not contain the SMQEALS C-terminus at 1 μM did not displace MSNB457 binding to MSLN-positive cells. However, binding was fully competed by 1 μM recombinant full-length MSLN. MSNB457 binding was reduced in the presence of 1 μM of the C-terminal 12-amino-acid peptide representing the membrane-restricted ‘stub’ region (LVLDLSMQEALS (SEQ ID NO: 203)). The observed incomplete competition by the 12 amino-acid peptide is consistent with SPR analysis showing weak binding with a fast on and off rate. [BD2021ST-067, EDMS-RIM-584499]. By comparison, the binding of MSNB90, a non-membrane-restricted control mAb (K_{D})=0.4 nM) that binds outside the stub region, was competed by both full-length and soluble MSLN, but not by the C-terminal peptide. These results demonstrated that MNC3B304 binding is membrane restricted as compared to non-membrane-restricted MSNB90.

8.5.6 MNC3B304 Does Not Bind or Kill Cells with a Homozygous MSLN:p.Met493ValSNP (SNPGIG) in the Membrane-restricted Stub Region.

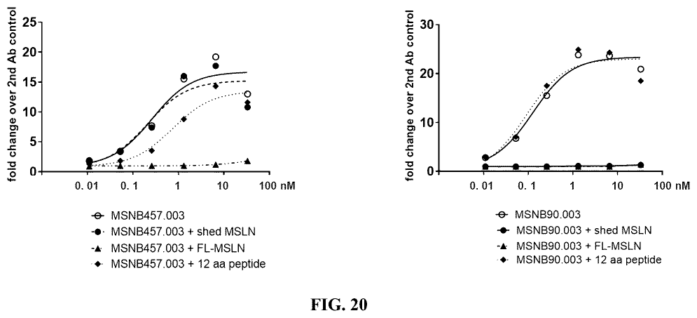

SNP rs1135210 (GRCh38-chromosome 16:768559) with a methionine to valine change (MSLN:p.Met593Val) exists in the mrMSLN region (FIG. 21). MNC3B304 binding to recombinant full-length MSLN with the M593V mutation (MSLNW14) was assessed by SPR. Weak binding was observed but failed analysis/reporting criteria. The K_{D} was estimated to be weaker than 100 nM, which represents a >20-fold reduction in binding compared to wild-type MSLN.

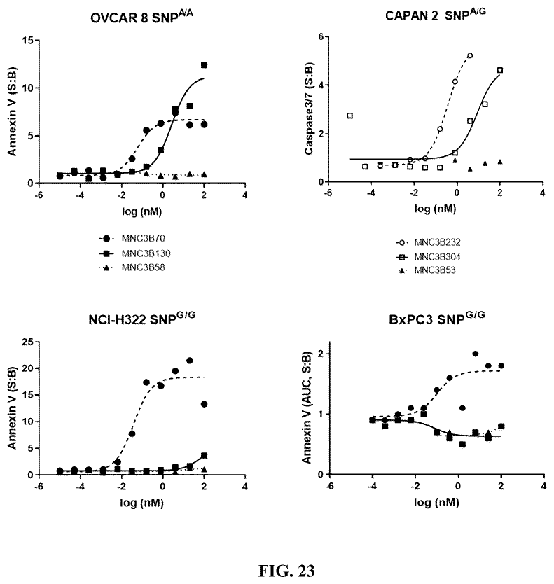

In addition, RNA sequencing (RNAseq) was performed on a panel of cell lines to characterize different MSLN isoforms as well as SNP status, identifying wild-type, heterozygous, and homozygous MSLN:p.Met593 Val cells lines that were used to assess MNC3B304 binding and T-cell-mediated killing. Both the parental MSLN binding mAb, MSNB457, an earlier version of the MSNB457×CD3-W245LH bispecific formatted on a alternative heterodimerization scaffold (Fc chain 1: T350V/L351Y/F405A/Y407V; Fc chain 2: T350V/T366L/K392/T394W) (ie, MNC3B130), and MNC3B304 were evaluated. Positive controls included MNC3B70 (MSNB9×W245LH, using alternative heterodimerization scaffold) which pairs a non-membrane restricted mesothelin binder with the CD3W245 LH binding arm, and the closely related MNC3B232 which uses knobs-in-holes (KiH) heterodimerization mutations. The negative controls were MNC3B58 and MNC3B53 (W245LH×Null) on the alternative heterodimerization scaffold and KiH formats, respectively. The methionine to valine amino acid change resulted in loss of MSNB457 (FIG. 22), MNC3B130, and MNC3B304 binding to and T-cell-mediated cytotoxicity against homozygous SNP-positive cells, while MNC3B304 binding and T-cell-mediated killing, albeit reduced, still occurred against heterozygous SNP-expressing cells (FIG. 23). The effect of homozygous SNP on the activity of MSNB457 is not fully understood, however, MSNB457 does not effectively bind to cells expressing the homozygous germline variant and much higher concentrations are needed to induce toxicity in this variant. The reduced binding by MSNB457 is consistent with lower receptor densities observed with PE-labeled MSNB457 (29,485 rec/cell) versus MSNB90 (85,316 rec/cell) (MSLN-00839).

### 8.6 Example 6: In Vitro Pharmacology Studies for MNC3B304

### 8.6.1 OVCAR-8 K1/C8 MSLN^{MED+} Cytotoxicity, T-cell Activation, and Cytokine Release Studies

OVCAR-8 K1/C8 cells were derived from OVCAR-8 ovarian cancer cells. MSLN was initially knocked out, followed by transfection of firefly luciferase. Cells were sorted to obtain MSLN monoclones with different expression levels. OVCAR-8 K1/C8 was shown to be a medium-MSLN-expressing clone (45,110 receptors/cell) representing an average level of MSLN expression from tumor cells used for experiments.

T-cell-mediated killing by MNC3B304 was assessed in -OVCAR-8 K1/C8 MSLN^{MED+} cancer cells using an impedance/×Celligence (live time-lapse) based approach. These experiments were designed to characterize the in vitro potency of MNC3B304. While MNC3B304 showed tumor cell cytotoxicity across 6 different T-cell donors, as expected the Null×CD3 control showed no activity in this assay. MNC3B304 showed significant donor-to-donor variability at the 1:3 E:T ratio, ranging from 30 to 90 percent maximal cytolysis, but did reach near 100% maximal activity at the 1:1 E:T ratio for all 6 donors. EC50 values for MNC3B304-induced cytolysis across all 6 T-cell donors at the 1:3 and 1:1 E:T ratios.

To determine the level of T-cell activation in the cell cytotoxicity assays described above, CD25 and CD69 expression were measured on T-cells by FACS. EC50 values for CD69 were consistently lower than for CD25. Overall T-cell activation comparison at the most potent E:T of 1:1 suggests CD69 was a sensitive marker for activity. CD69+ T-cell activation EC50 values at 1:1 E:T ratios demonstrated potent activity.

To further characterize T-cell activation by MNC3B304, cytokine release profiles of 11 cytokines (i.e., interferon IFN-γ, interleukin IL-1β, IL-2, IL-4, IL-6, IL-8, IL-10, IL-13, tumor necrosis factor TNF-α, IL-12p70) were determined using the V-PLEX Proinflammatory Panel 1 Human Kit (Meso-Scale discovery [MSD], K15049D-4) in OVCAR-8 K1/C8. MNC3B304-induced cytokine release was demonstrated for 4 T-cell donors at an E:T ratio of 1:1. None of the cytokine release effective concentrations were more potent than values derived for T-cell activation using CD25 or CD69, further supporting CD69 as a sensitive marker of MNC3B304 activity. 8.6.2 Influence of MSLN Receptor Density on T-cell-mediated Cytotoxicity

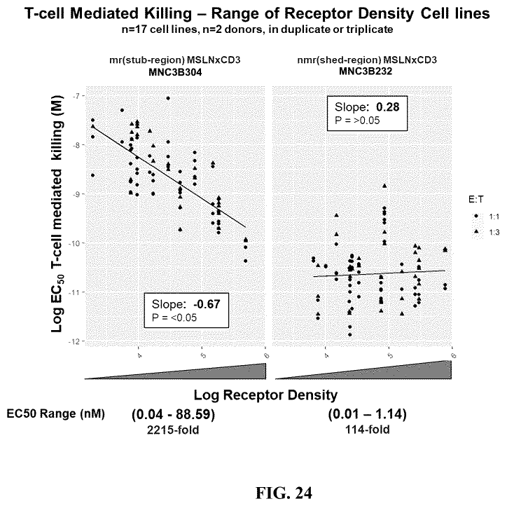

To investigate the extent of T-cell-mediated toxicity induced by mrMSLN×CD3 bispecific antibody MNC3B304 in cells with different MSLN expression levels, 17 cell lines with receptor densities ranging from 1,670 to 488,190 receptors/cell (as determined with MSNB457-PE) were used. Cytotoxicity was assessed by Caspase 7 induction using a kinetic Incucyte measurement up to 72 hours for 2 donors. Cytotoxicity was compared to tumor cell killing induced by a non-membrane-restricted bispecific antibody MNC3B232 (K_{D})=0.05 nM) paired with W245LH CD3 scFv. MNC3B304 induced killing in all cells tested with EC₅₀ values ranging from 0.04 to 88.59 nM while EC₅₀ values for MNC3B232 ranged from 0.01 to 1.14 nM (FIG. 24). For MNC3B304, the killing potency significantly correlated with receptor densities with more potent killing seen at higher expression levels, while EC50 values for MNC3B232 did not correlate with receptor densities. The correlation between increased potency and expression level for MNC3B304 was independent of donor, E:T ratio, or time point selected.

### 8.6.3 Validation of Cell Lines with Different MSLN Receptor Densities Selected to Assess MNC3B304 Differential Cytotoxicity In Vitro and In Vivo

Normal mesothelial and tumor cell expression levels were determined using primary cells isolated from serosal fluids as described in Example 1. Cell lines with expression levels corresponding to either normal mesothelial or tumor MSLN expression levels were used to further confirm differential cytotoxicity to normal and tumor cells in vivo.

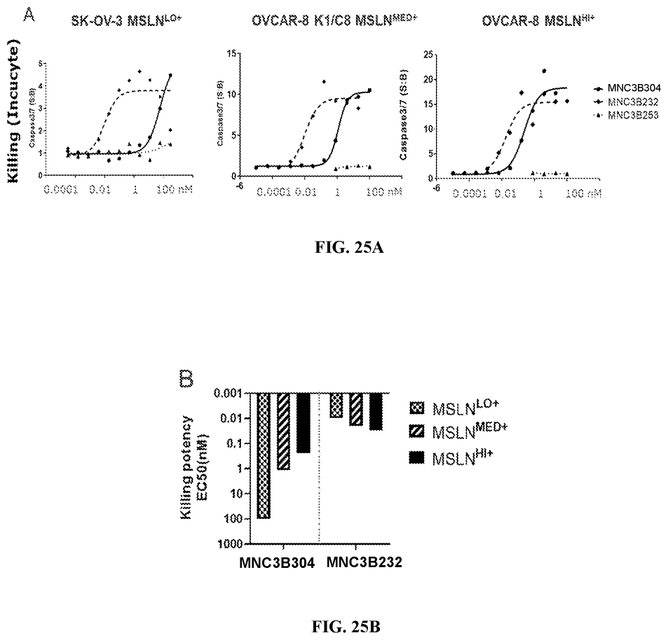

Three models with known abilities to establish tumors in vivo were selected with respectively, low, medium, and high MSLN expression: (i) SK-OV-3 MSLNLO+(7,804 receptors/cell), (ii) OVCAR-8 K1/C8 MSLN^{MED+} (45,110 receptors/cell), and (iii) OVCAR-8 MSLN^{HI+} (182,378 receptors/cell). These models were confirmed to show differential tumor cell killing in vitro. MNC3B304 cytotoxicity EC50 values as determined by Incucyte kinetic measurements (72 hours, 1:1 E:T ratios) decreased from 103 nM in MSLNLO+SK-OV-3 (normal tissue surrogate), to 1.2 nM in OVCAR-8 K1/C8 MSLNMED+, and 0.24 nM in OVCAR-8 MSLNHI+ tumor cells (FIG. 25). In contrast, the MNC3B232 nmrMSLN×CD3 bispecific antibody showed potent cytotoxicity ranging from 0.01 to 0.03 nM across these cell lines in vitro.

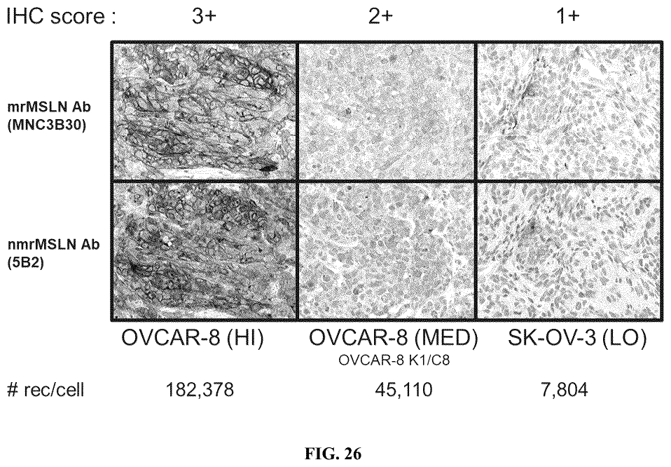

IHC was performed using mrMSLN mAb MNC3B30 and commercial nmrMSLN mAb 5B2. MNC3B30 is a rabbit IgG1 mAb originating from mrMSLN mAb MSNB71 and the only mrMSLN mAb for which an IHC assay could be established. In cell-line-derived xenografts (CDXs) from the SK-OV-3, OVCAR-8 K1/C8, and OVCAR-8 models, it was-confirmed that low, medium, and high expression levels were maintained in vivo. IHC intensity scores were 1+, 2+, and 3+ for SK-OV-3, OVCAR-8 K1/C8, and OVCAR-8 CDXs, respectively (FIG. 26).

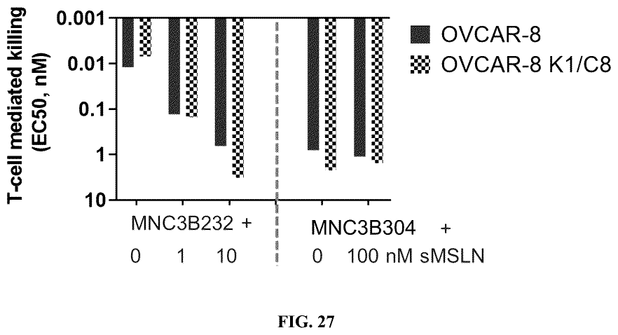

The potential of shed MSLN to interfere with in vitro antibody-mediated killing assay was assessed. A total-MSLN enzyme-linked immunosorbent assay (ELISA) detecting full-length and shed/soluble MSLN was used in combination with a full-length-MSLN-specific MSD assay on the same CDXs. Data further confirmed that shed MSLN concentrations in the tumor microenvironment increased with expression levels: 0.04 nM in MSLNLO+ SK-OV-3, 0.2 nM-in OVCAR-8 K1/C8 MSLN^{MED+}, and 3.1-nM in OVCAR-8 MSLN^{HI+} model. Western blot showed that these shed MSLN levels are as high as levels of membrane-bound full-length MSLN present in the tumors, suggesting that a non-membrane-restrictive MSLN antibody would be substantially competed by the shed MSLN levels in the tumor microenvironment of medium to high expressing tumors. In vitro cytotoxicity-experiments-against SK-OV-3, OVCAR-8 K1C8, and OVCAR-8 cell lines in the presence of 1 to 10 nM shed MSLN indeed confirmed that cytotoxicity with MNC3B232 decreased by 11- to 54-fold in MSLN^{HI+}/MSLN^{MED+} cells while cytotoxicity for membrane-restricted-MSLN-targeting bispecific antibody MNC3B304, even in the presence of 100 nM shed MSLN, was unaffected (FIG. 27).

### 8.6.4 Efficacy of MNC3B304 Against CDX Models with Different MSLN Expression Levels

The antitumor activity of mrMSLN bispecific antibody MNC3B304 and nmrMSLN bispecific antibody MNC3B232 was evaluated in the established human ovarian CDX models OVCAR-8 MSLNHI+, OVCAR-8 K1/C8 MSLNMED+, and SK-OV-3 MSLNLO+, in female immune-compromised NSG (ie, non-obese diabetic [NOD] severe combined immunodeficiency [SCID] gamma or NOD.Cg-Prkdcscid Il2rgtm 1 Wjl/SzJ) mice humanized with human donor CD3+ pan T-cells. Twice weekly treatment with MNC3B304 administered intraperitoneally (IP) was initiated after SC tumors were established. While the tolerability of MNC3B304 could not be assessed with respect to MSLN or CD3 binding to host tissues due to lack of cross-reactivity to the corresponding mouse antigens (MSLN-00922), the engrafted human T-cells expressing CD3 did bind MNC3B304. Engraftment of human T-cells can lead to body weight loss due to eventual graft-versus-host disease (GvHD); however, treatment with MNC3B304 did not result in significant body weight loss as compared to the phosphate-buffered saline (PBS)-treated control group (data not shown).

Mice bearing established SC SK-OV-3 xenografts were IP dosed with MNC3B304 twice weekly at 2.5, 0.5, and 0.1 mg/kg, or PBS for a total of 8 doses (n=10/group). Significant antitumor efficacy was observed with MNC3B304 treatment at 2.5 and 0.5 mg/kg over time (p<0.0001) with 97% and 71% ΔTGI respectively, as compared to PBS-treated control mice on Day 52 post tumor implantation (FIG. 28A). Treatment with MNC3B304 at 0.1 mg/kg resulted in 55% ΔTGI as compared to the PBS-treated control and was statistically significant (p=0.0093), but not biologically significant. In contrast, MCN3B232 elicited significant antitumor efficacy at all tested doses (2.5, 0.5, and 0.1 mg/kg) over time with (p<0.0001, 92%, 102%, and 99% ΔTGI, respectively), compared to PBS-treated control mice on Day 52 post tumor implantation.

Mice bearing established SC OVCAR-8 K1/C8 xenografts were IP dosed with MNC3B304 twice weekly at 0.1, 0.01, and 0.005 mg/kg, or PBS for a total of 6 doses (n=10/group). Significant antitumor efficacy was observed with MNC3B304 at 0.1 mg/kg over time (p<0.0001) with 150% ΔTGI as compared to PBS-treated control mice on Day 57 post tumor implantation (FIG. 28B), and 1 of 10 mice showing complete tumor regression. Treatment with MNC3B304 at 0.01 and 0.005 mg/kg did not result in statistically significant antitumor activity and 0% ΔTGI was observed as compared to the PBS-treated control. MCN3B232 did not elicit significant antitumor efficacy over time at 0.1 mg/kg.

Mice bearing established SC OVCAR-8 xenografts were IP dosed twice weekly with MNC3B304 at 0.01 and 0.1 mg/kg, or PBS for a total of 6 doses (n=10/group). Significant antitumor efficacy was observed with MNC3B304 0.1 mg/kg over time (p<0.0001) with 173% ΔTGI as compared to PBS-treated control mice on Day 45 post tumor implantation (FIG. 28C), with all animals showing complete tumor regressions. Treatment with MNC3B304 at 0.01 mg/kg resulted in 0% ΔTGI as compared to the PBS-treated control. In contrast to MNC3B304, treatment with MNC3B232 at 0.1 mg/kg did not result in a statistically significant antitumor activity and no ΔTGI was observed as compared to the PBS-treated control.

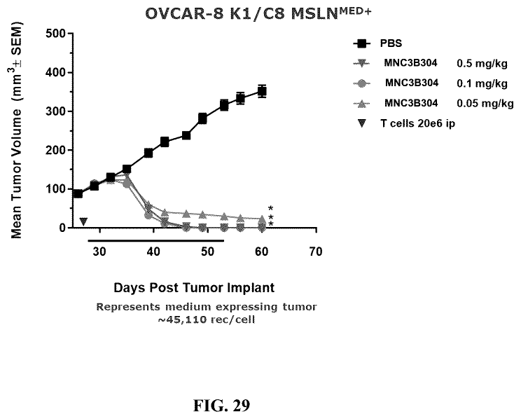

Mice bearing established SC OVCAR-8 K1/C8 xenografts were IP dosed with MNC3B304 twice weekly at 0.05, 0.1, and 0.5 mg/kg, or PBS for a total of 8 doses (n=10/group). Also, mice bearing established SC OVCAR-8 K1/C8 xenografts were IP dosed with MNC3B304 twice weekly at 0.05, 0.1, and 0.5 mg/kg, or PBS for a total of 8 doses (n=10/group). Significant antitumor efficacy was observed with MNC3B304 at 0.5, 0.1, and 0.05 mg/kg over time (p<0.0001) with 136%, 136%, and 125% ΔTGI, respectively, as compared to PBS-treated control mice on Day 56 post tumor implantation (FIG. 29). Treatment with 0.5 and 0.1 mg/kg resulted in 10 of 10 complete tumor regressions and treatment with 0.05 mg/kg resulted in 2 of 10 complete tumor regressions.

The in vivo efficacy findings suggest an inverse correlation profile for mrMSLN and nmrMSLN bispecific antibodies. MNC3B304's potency increased with increasing MSLN expression levels in vivo, while MNC3B232's potency decreased with increasing MSLN expression, likely due to the shed MSLN sink increasing with increasing level of shed MSLN from tumor. Collectively, these data suggest than an improved therapeutic window may be achieved by avoiding binding to shed MSLN.

What is claimed is:

1. A binding agent comprising an antigen binding region that
(a) binds to an epitope of MSLN recognized by an antibody comprising a heavy chain variable (VH) domain comprising a VH complementarity determining region 1 (HCDR1), a HCDR2, and a HCDR3 having an amino acid sequence of a HCDR1, a HCDR2, and a HCDR3, respectively, of a VH domain having an amino acid sequence of SEQ ID NO:81; and a light chain variable (VL) domain comprising a LCDR1, a LCDR2, and a LCDR3 having an amino acid sequence of a LCDR1, a LCDR2, and a LCDR3, respectively, of a VL domain having an amino acid sequence of SEQ ID NO:82;
(b) competes for the binding to MSLN with an antibody comprising a VH domain comprising a VH complementarity determining region 1 (HCDR1), a HCDR2, and a HCDR3 having an amino acid sequence of a HCDR1, a HCDR2, and a HCDR3, respectively, of a VH domain having an amino acid sequence of SEQ ID NO:81; and a VL domain comprising a LCDR1, a LCDR2, and a LCDR3 having an amino acid sequence of a LCDR1, a LCDR2, and a LCDR3, respectively, of a VL domain having an amino acid sequence of SEQ ID NO:82; or
(c) binds to an epitope of MSLN in the membrane-restricted region of MSLN.

2. A binding agent comprising an antigen binding region that binds to MSLN, wherein the antigen binding region comprises:
(a) a VH domain comprising a HCDR1, HCDR2, and HCDR3 as set forth in SEQ ID NO:81, and a VL domain comprising a LCDR1, LCDR2, and LCDR3 as set forth in SEQ ID NO:82;
(b) a VH domain comprising a HCDR1, HCDR2, and HCDR3 as set forth in SEQ ID NO:22, and a VL domain comprising a LCDR1, LCDR2, and LCDR3 as set forth in SEQ ID NO:23;
(c) a VH domain comprising a HCDR1, HCDR2, and HCDR3 as set forth in SEQ ID NO:45, and a VL domain comprising a LCDR1, LCDR2, and LCDR3 as set forth in SEQ ID NO:46;
(d) a VH domain comprising a HCDR1, HCDR2, and HCDR3 as set forth in SEQ ID NO:62, and a VL domain comprising a LCDR1, LCDR2, and LCDR3 as set forth in SEQ ID NO:63;
(e) a VH domain comprising a HCDR1, HCDR2, and HCDR3 as set forth in SEQ ID NO:94, and a VL domain comprising a LCDR1, LCDR2, and LCDR3 as set forth in SEQ ID NO:95;
(f) a VH domain comprising a HCDR1, HCDR2, and HCDR3 as set forth in SEQ ID NO:106, and a VL domain comprising a LCDR1, LCDR2, and LCDR3 as set forth in SEQ ID NO: 107;
(g) a VH domain comprising a HCDR1, HCDR2, and HCDR3 as set forth in SEQ ID NO:120, and a VL domain comprising a LCDR1, LCDR2, and LCDR3 as set forth in SEQ ID NO:121;
(h) a VH domain comprising a HCDR1, HCDR2, and HCDR3 as set forth in SEQ ID NO:132, and a VL domain comprising a LCDR1, LCDR2, and LCDR3 as set forth in SEQ ID NO:133;
(i) a VH domain comprising a HCDR1, HCDR2, and HCDR3 as set forth in SEQ ID NO:138, and a VL domain comprising a LCDR1, LCDR2, and LCDR3 as set forth in SEQ ID NO:133;
(j) a VH domain comprising a HCDR1, HCDR2, and HCDR3 as set forth in SEQ ID NO:120, and a VL domain comprising a LCDR1, LCDR2, and LCDR3 as set forth in SEQ ID NO:139;
(k) a VH domain comprising a HCDR1, HCDR2, and HCDR3 as set forth in SEQ ID NO:106, and a VL domain comprising a LCDR1, LCDR2, and LCDR3 as set forth in SEQ ID NO: 140; or
(l) a VH domain comprising a HCDR1, HCDR2, and HCDR3 as set forth in SEQ ID NO:132, and a VL domain comprising a LCDR1, LCDR2, and LCDR3 as set forth in SEQ ID NO:142.

3. The binding agent of claim 2, wherein:
(a1) HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(a2) HCDR1 comprises an amino acid sequence of SEQ ID NO: 70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(a3) HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(a4) HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76;
(a5) HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69;
(b1) HCDR1 comprises an amino acid sequence of SEQ ID NO:1; HCDR2 comprises an amino acid sequence of SEQ ID NO:2; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6;
(b2) HCDR1 comprises an amino acid sequence of SEQ ID NO:7; HCDR2 comprises an amino acid sequence of SEQ ID NO:8; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6;
(b3) HCDR1 comprises an amino acid sequence of SEQ ID NO:9; HCDR2 comprises an amino acid sequence of SEQ ID NO:10; HCDR3 comprises an amino acid sequence of SEQ ID NO:3; LCDR1 comprises an amino acid sequence of SEQ ID NO:4; LCDR2 comprises an amino acid sequence of SEQ ID NO:5; and LCDR3 comprises an amino acid sequence of SEQ ID NO:6;
(b4) HCDR1 comprises an amino acid sequence of SEQ ID NO: 11; HCDR2 comprises an amino acid sequence of SEQ ID NO: 12; HCDR3 comprises an amino acid sequence of SEQ ID NO: 13; LCDR1 comprises an amino acid sequence of SEQ ID NO:14; LCDR2 comprises an amino acid sequence of SEQ ID NO:15; and LCDR3 comprises an amino acid sequence of SEQ ID NO:16;
(b5) HCDR1 comprises an amino acid sequence of SEQ ID NO: 17; HCDR2 comprises an amino acid sequence of SEQ ID NO:18; HCDR3 comprises an amino acid sequence of SEQ ID NO:19; LCDR1 comprises an amino acid sequence of SEQ ID NO:20; LCDR2 comprises an amino acid sequence of LVS; and LCDR3 comprises an amino acid sequence of SEQ ID NO:6;
(c1) HCDR1 comprises an amino acid sequence of SEQ ID NO:24; HCDR2 comprises an amino acid sequence of SEQ ID NO:25; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29;
(c2) HCDR1 comprises an amino acid sequence of SEQ ID NO:30; HCDR2 comprises an amino acid sequence of SEQ ID NO:252; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29;
(c3) HCDR1 comprises an amino acid sequence of SEQ ID NO:32; HCDR2 comprises an amino acid sequence of SEQ ID NO:33; HCDR3 comprises an amino acid sequence of SEQ ID NO:26; LCDR1 comprises an amino acid sequence of SEQ ID NO:27; LCDR2 comprises an amino acid sequence of SEQ ID NO:28; and LCDR3 comprises an amino acid sequence of SEQ ID NO:29;
(c4) HCDR1 comprises an amino acid sequence of SEQ ID NO:34; HCDR2 comprises an amino acid sequence of SEQ ID NO:65; HCDR3 comprises an amino acid sequence of SEQ ID NO:36; LCDR1 comprises an amino acid sequence of SEQ ID NO:37; LCDR2 comprises an amino acid sequence of SEQ ID NO:38; and LCDR3 comprises an amino acid sequence of SEQ ID NO:39;
(c5) HCDR1 comprises an amino acid sequence of SEQ ID NO:40; HCDR2 comprises an amino acid sequence of SEQ ID NO:41; HCDR3 comprises an amino acid sequence of SEQ ID NO:42; LCDR1 comprises an amino acid sequence of SEQ ID NO:43; LCDR2 comprises an amino acid sequence of LGS; and LCDR3 comprises an amino acid sequence of SEQ ID NO:29;
(d1) HCDR1 comprises an amino acid sequence of SEQ ID NO:47; HCDR2 comprises an amino acid sequence of SEQ ID NO:48; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50;
(d2) HCDR1 comprises an amino acid sequence of SEQ ID NO:251; HCDR2 comprises an amino acid sequence of SEQ ID NO:31; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50;
(d3) HCDR1 comprises an amino acid sequence of SEQ ID NO:51; HCDR2 comprises an amino acid sequence of SEQ ID NO:52; HCDR3 comprises an amino acid sequence of SEQ ID NO:3; LCDR1 comprises an amino acid sequence of SEQ ID NO:49; LCDR2 comprises an amino acid sequence of SEQ ID NO:28; and LCDR3 comprises an amino acid sequence of SEQ ID NO:50;
(d4) HCDR1 comprises an amino acid sequence of SEQ ID NO:53; HCDR2 comprises an amino acid sequence of SEQ ID NO:54; HCDR3 comprises an amino acid sequence of SEQ ID NO:55; LCDR1 comprises an amino acid sequence of SEQ ID NO:56; LCDR2 comprises an amino acid sequence of SEQ ID NO:38; and LCDR3 comprises an amino acid sequence of SEQ ID NO:57;
(d5) HCDR1 comprises an amino acid sequence of SEQ ID NO:58; HCDR2 comprises an amino acid sequence of SEQ ID NO:59; HCDR3 comprises an amino acid sequence of SEQ ID NO:60; LCDR1 comprises an amino acid sequence of SEQ ID NO:61; LCDR2 comprises an amino acid sequence of LGS; and LCDR3 comprises an amino acid sequence of SEQ ID NO:50;
(e1) HCDR1 comprises an amino acid sequence of SEQ ID NO:83; HCDR2 comprises an amino acid sequence of SEQ ID NO:84; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87;
(e2) HCDR1 comprises an amino acid sequence of SEQ ID NO:30; HCDR2 comprises an amino acid sequence of SEQ ID NO:31; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87;
(e3) HCDR1 comprises an amino acid sequence of SEQ ID NO:253; HCDR2 comprises an amino acid sequence of SEQ ID NO:52; HCDR3 comprises an amino acid sequence of SEQ ID NO:85; LCDR1 comprises an amino acid sequence of SEQ ID NO:86; LCDR2 comprises an amino acid sequence of SEQ ID NO:28; and LCDR3 comprises an amino acid sequence of SEQ ID NO:87;
(e4) HCDR1 comprises an amino acid sequence of SEQ ID NO:88; HCDR2 comprises an amino acid sequence of SEQ ID NO:54; HCDR3 comprises an amino acid sequence of SEQ ID NO:89; LCDR1 comprises an amino acid sequence of SEQ ID NO:90; LCDR2 comprises an amino acid sequence of SEQ ID NO:38; and LCDR3 comprises an amino acid sequence of SEQ ID NO:91;
(e5) HCDR1 comprises an amino acid sequence of SEQ ID NO:40; HCDR2 comprises an amino acid sequence of SEQ ID NO:59; HCDR3 comprises an amino acid sequence of SEQ ID NO:92; LCDR1 comprises an amino acid sequence of SEQ ID NO:93; LCDR2 comprises an amino acid sequence of LGS; and LCDR3 comprises an amino acid sequence of SEQ ID NO:87;
(f1) HCDR1 comprises an amino acid sequence of SEQ ID NO:1; HCDR2 comprises an amino acid sequence of SEQ ID NO:96; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
(f2) HCDR1 comprises an amino acid sequence of SEQ ID NO:7; HCDR2 comprises an amino acid sequence of SEQ ID NO:31; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
(f3) HCDR1 comprises an amino acid sequence of SEQ ID NO:9; HCDR2 comprises an amino acid sequence of SEQ ID NO:52; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
(f4) HCDR1 comprises an amino acid sequence of SEQ ID NO: 11; HCDR2 comprises an amino acid sequence of SEQ ID NO:54; HCDR3 comprises an amino acid sequence of SEQ ID NO: 100; LCDR1 comprises an amino acid sequence of SEQ ID NO:101; LCDR2 comprises an amino acid sequence of SEQ ID NO:102; and LCDR3 comprises an amino acid sequence of SEQ ID NO:103;
(f5) HCDR1 comprises an amino acid sequence of SEQ ID NO:17; HCDR2 comprises an amino acid sequence of SEQ ID NO:59; HCDR3 comprises an amino acid sequence of SEQ ID NO: 104; LCDR1 comprises an amino acid sequence of SEQ ID NO:105; LCDR2 comprises an amino acid sequence of LVS; and LCDR3 comprises an amino acid sequence of SEQ ID NO:99;
(g1) HCDR1 comprises an amino acid sequence of SEQ ID NO:108; HCDR2 comprises an amino acid sequence of SEQ ID NO:109; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
(g2) HCDR1 comprises an amino acid sequence of SEQ ID NO: 112; HCDR2 comprises an amino acid sequence of SEQ ID NO:31; HCDR3 comprises an amino acid sequence of SEQ ID NO:110; LCDR1 comprises an amino acid sequence of SEQ ID NO: 111; LCDR2 comprises an amino acid sequence of SEQ ID NO:28; and LCDR3 comprises an amino acid sequence of SEQ ID NO:87;
(g3) HCDR1 comprises an amino acid sequence of SEQ ID NO: 113; HCDR2 comprises an amino acid sequence of SEQ ID NO:52; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
(g4) HCDR1 comprises an amino acid sequence of SEQ ID NO: 114; HCDR2 comprises an amino acid sequence of SEQ ID NO:54; HCDR3 comprises an amino acid sequence of SEQ ID NO:115; LCDR1 comprises an amino acid sequence of SEQ ID NO:116; LCDR2 comprises an amino acid sequence of SEQ ID NO:38; and LCDR3 comprises an amino acid sequence of SEQ ID NO:91;
(g5) HCDR1 comprises an amino acid sequence of SEQ ID NO: 117; HCDR2 comprises an amino acid sequence of SEQ ID NO:59; HCDR3 comprises an amino acid sequence of SEQ ID NO:118; LCDR1 comprises an amino acid sequence of SEQ ID NO:119; LCDR2 comprises an amino acid sequence of LGS; and LCDR3 comprises an amino acid sequence of SEQ ID NO:87;
(h1) HCDR1 comprises an amino acid sequence of SEQ ID NO: 122; HCDR2 comprises an amino acid sequence of SEQ ID NO:84; HCDR3 comprises an amino acid sequence of SEQ ID NO:3; LCDR1 comprises an amino acid sequence of SEQ ID NO: 123; LCDR2 comprises an amino acid sequence of SEQ ID NO: 124; and LCDR3 comprises an amino acid sequence of SEQ ID NO:99;
(h2) HCDR1 comprises an amino acid sequence of SEQ ID NO: 125; HCDR2 comprises an amino acid sequence of SEQ ID NO:31; HCDR3 comprises an amino acid sequence of SEQ ID NO:3; LCDR1 comprises an amino acid sequence of SEQ ID NO: 123; LCDR2 comprises an amino acid sequence of SEQ ID NO:124; and LCDR3 comprises an amino acid sequence of SEQ ID NO:99;
(h3) HCDR1 comprises an amino acid sequence of SEQ ID NO: 126; HCDR2 comprises an amino acid sequence of SEQ ID NO:52; HCDR3 comprises an amino acid sequence of SEQ ID NO:3; LCDR1 comprises an amino acid sequence of SEQ ID NO: 123; LCDR2 comprises an amino acid sequence of SEQ ID NO: 124; and LCDR3 comprises an amino acid sequence of SEQ ID NO:99;
(h4) HCDR1 comprises an amino acid sequence of SEQ ID NO:127; HCDR2 comprises an amino acid sequence of SEQ ID NO:54; HCDR3 comprises an amino acid sequence of SEQ ID NO:13; LCDR1 comprises an amino acid sequence of SEQ ID NO:128; LCDR2 comprises an amino acid sequence of SEQ ID NO:129; and LCDR3 comprises an amino acid sequence of SEQ ID NO:103;
(h5) HCDR1 comprises an amino acid sequence of SEQ ID NO:130; HCDR2 comprises an amino acid sequence of SEQ ID NO:59; HCDR3 comprises an amino acid sequence of SEQ ID NO: 19; LCDR1 comprises an amino acid sequence of SEQ ID NO:131; LCDR2 comprises an amino acid sequence of LGS; and LCDR3 comprises an amino acid sequence of SEQ ID NO:99;
(i1) HCDR1 comprises an amino acid sequence of SEQ ID NO:122; HCDR2 comprises an amino acid sequence of SEQ ID NO:134; HCDR3 comprises an amino acid sequence of SEQ ID NO:3; LCDR1 comprises an amino acid sequence of SEQ ID NO: 123; LCDR2 comprises an amino acid sequence of SEQ ID NO:124; and LCDR3 comprises an amino acid sequence of SEQ ID NO:99;
(i2) HCDR1 comprises an amino acid sequence of SEQ ID NO: 125; HCDR2 comprises an amino acid sequence of SEQ ID NO:31; HCDR3 comprises an amino acid sequence of SEQ ID NO:3; LCDR1 comprises an amino acid sequence of SEQ ID NO:123; LCDR2 comprises an amino acid sequence of SEQ ID NO:124; and LCDR3 comprises an amino acid sequence of SEQ ID NO:99;
(i3) HCDR1 comprises an amino acid sequence of SEQ ID NO:126; HCDR2 comprises an amino acid sequence of SEQ ID NO:135; HCDR3 comprises an amino acid sequence of SEQ ID NO:3; LCDR1 comprises an amino acid sequence of SEQ ID NO: 123; LCDR2 comprises an amino acid sequence of SEQ ID NO:124; and LCDR3 comprises an amino acid sequence of SEQ ID NO:99;
(i4) HCDR1 comprises an amino acid sequence of SEQ ID NO:127; HCDR2 comprises an amino acid sequence of SEQ ID NO:136; HCDR3 comprises an amino acid sequence of SEQ ID NO:13; LCDR1 comprises an amino acid sequence of SEQ ID NO:128; LCDR2 comprises an amino acid sequence of SEQ ID NO:129; and LCDR3 comprises an amino acid sequence of SEQ ID NO:103;
(i5) HCDR1 comprises an amino acid sequence of SEQ ID NO: 130; HCDR2 comprises an amino acid sequence of SEQ ID NO:137; HCDR3 comprises an amino acid sequence of SEQ ID NO: 19; LCDR1 comprises an amino acid sequence of SEQ ID NO:131; LCDR2 comprises an amino acid sequence of LGS; and LCDR3 comprises an amino acid sequence of SEQ ID NO:99;
(j1) HCDR1 comprises an amino acid sequence of SEQ ID NO: 108; HCDR2 comprises an amino acid sequence of SEQ ID NO:109; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
(j2) HCDR1 comprises an amino acid sequence of SEQ ID NO: 112; HCDR2 comprises an amino acid sequence of SEQ ID NO:31; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
(j3) HCDR1 comprises an amino acid sequence of SEQ ID NO:113; HCDR2 comprises an amino acid sequence of SEQ ID NO:52; HCDR3 comprises an amino acid sequence of SEQ ID NO:110; LCDR1 comprises an amino acid sequence of SEQ ID NO:111; LCDR2 comprises an amino acid sequence of SEQ ID NO:28; and LCDR3 comprises an amino acid sequence of SEQ ID NO:87;
(j4) HCDR1 comprises an amino acid sequence of SEQ ID NO:114; HCDR2 comprises an amino acid sequence of SEQ ID NO:54; HCDR3 comprises an amino acid sequence of SEQ ID NO:115; LCDR1 comprises an amino acid sequence of SEQ ID NO: 116; LCDR2 comprises an amino acid sequence of SEQ ID NO:38; and LCDR3 comprises an amino acid sequence of SEQ ID NO:91;
(j5) HCDR1 comprises an amino acid sequence of SEQ ID NO:117; HCDR2 comprises an amino acid sequence of SEQ ID NO:59; HCDR3 comprises an amino acid sequence of SEQ ID NO:118; LCDR1 comprises an amino acid sequence of SEQ ID NO:119; LCDR2 comprises an amino acid sequence of LGS; and LCDR3 comprises an amino acid sequence of SEQ ID NO:87;
(k1) HCDR1 comprises an amino acid sequence of SEQ ID NO:1; HCDR2 comprises an amino acid sequence of SEQ ID NO:96; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
(k2) HCDR1 comprises an amino acid sequence of SEQ ID NO:7; HCDR2 comprises an amino acid sequence of SEQ ID NO:31; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
(k3) HCDR1 comprises an amino acid sequence of SEQ ID NO:9; HCDR2 comprises an amino acid sequence of SEQ ID NO:52; HCDR3 comprises an amino acid sequence of SEQ ID NO:97; LCDR1 comprises an amino acid sequence of SEQ ID NO:98; LCDR2 comprises an amino acid sequence of SEQ ID NO:5; and LCDR3 comprises an amino acid sequence of SEQ ID NO:99;
(k4) HCDR1 comprises an amino acid sequence of SEQ ID NO: 11; HCDR2 comprises an amino acid sequence of SEQ ID NO:54; HCDR3 comprises an amino acid sequence of SEQ ID NO:100; LCDR1 comprises an amino acid sequence of SEQ ID NO:101; LCDR2 comprises an amino acid sequence of SEQ ID NO:102; and LCDR3 comprises an amino acid sequence of SEQ ID NO: 103;
(k5) HCDR1 comprises an amino acid sequence of SEQ ID NO:17; HCDR2 comprises an amino acid sequence of SEQ ID NO:59; HCDR3 comprises an amino acid sequence of SEQ ID NO: 104; LCDR1 comprises an amino acid sequence of SEQ ID NO:105; LCDR2 comprises an amino acid sequence of LVS; and LCDR3 comprises an amino acid sequence of SEQ ID NO:99;
(l1) HCDR1 comprises an amino acid sequence of SEQ ID NO: 122; HCDR2 comprises an amino acid sequence of SEQ ID NO:84; HCDR3 comprises an amino acid sequence of SEQ ID NO:3; LCDR1 comprises an amino acid sequence of SEQ ID NO: 123; LCDR2 comprises an amino acid sequence of SEQ ID NO:28; and LCDR3 comprises an amino acid sequence of SEQ ID NO:99;
(l2) HCDR1 comprises an amino acid sequence of SEQ ID NO:125; HCDR2 comprises an amino acid sequence of SEQ ID NO:31; HCDR3 comprises an amino acid sequence of SEQ ID NO:3; LCDR1 comprises an amino acid sequence of SEQ ID NO:123; LCDR2 comprises an amino acid sequence of SEQ ID NO:28; and LCDR3 comprises an amino acid sequence of SEQ ID NO:99;
(l3) HCDR1 comprises an amino acid sequence of SEQ ID NO: 126; HCDR2 comprises an amino acid sequence of SEQ ID NO:52; HCDR3 comprises an amino acid sequence of SEQ ID NO:3; LCDR1 comprises an amino acid sequence of SEQ ID NO: 123; LCDR2 comprises an amino acid sequence of SEQ ID NO:28; and LCDR3 comprises an amino acid sequence of SEQ ID NO:99;
(l4) HCDR1 comprises an amino acid sequence of SEQ ID NO: 127; HCDR2 comprises an amino acid sequence of SEQ ID NO:54; HCDR3 comprises an amino acid sequence of SEQ ID NO:13; LCDR1 comprises an amino acid sequence of SEQ ID NO:128; LCDR2 comprises an amino acid sequence of SEQ ID NO:141; and LCDR3 comprises an amino acid sequence of SEQ ID NO:103; or
(l5) HCDR1 comprises an amino acid sequence of SEQ ID NO: 130; HCDR2 comprises an amino acid sequence of SEQ ID NO:59; HCDR3 comprises an amino acid sequence of SEQ ID NO:19; LCDR1 comprises an amino acid sequence of SEQ ID NO:131; LCDR2 comprises an amino acid sequence of LGS; and LCDR3 comprises an amino acid sequence of SEQ ID NO:99.

4. The binding agent of any one of claims 1 to 3, wherein the antigen binding region comprises:
(a) a VH domain comprising an amino acid sequence of SEQ ID NO:81, and a VL domain comprising an amino acid sequence of SEQ ID NO:82;
(b) a VH domain comprising an amino acid sequence of SEQ ID NO:22, and a VL domain comprising an amino acid sequence of SEQ ID NO:23;
(c) a VH domain comprising an amino acid sequence of SEQ ID NO:45, and a VL domain comprising an amino acid sequence of SEQ ID NO:46;
(d) a VH domain comprising an amino acid sequence of SEQ ID NO:62, and a VL domain comprising an amino acid sequence of SEQ ID NO:63;
(e) a VH domain comprising an amino acid sequence of SEQ ID NO:94, and a VL domain comprising an amino acid sequence of SEQ ID NO:95;
(f) a VH domain comprising an amino acid sequence of SEQ ID NO:106, and a VL domain comprising an amino acid sequence of SEQ ID NO:107;
(g) a VH domain comprising an amino acid sequence of SEQ ID NO:120, and a VL domain comprising an amino acid sequence of SEQ ID NO:121;
(h) a VH domain comprising an amino acid sequence of SEQ ID NO: 132, and a VL domain comprising an amino acid sequence of SEQ ID NO:133;
(i) a VH domain comprising an amino acid sequence of SEQ ID NO:138, and a VL domain comprising an amino acid sequence of SEQ ID NO:133;
(j) a VH domain comprising an amino acid sequence of SEQ ID NO: 120, and a VL domain comprising an amino acid sequence of SEQ ID NO:139;
(k) a VH domain comprising an amino acid sequence of SEQ ID NO: 106, and a VL domain comprising an amino acid sequence of SEQ ID NO: 140; or
(l) a VH domain comprising an amino acid sequence of SEQ ID NO: 132, and a VL domain comprising an amino acid sequence of SEQ ID NO: 142.

5. The binding agent of any one of claims 1 to 4, wherein the binding agent is a bispecific protein or a multi-specific protein.

6. The binding agent of any one of claims 1 to 5, further comprising an immunoglobulin (Ig) constant region, or a fragment of the Ig constant region, wherein optionally the fragment of the Ig constant region is an Fc region or an CH3 domain.

7. The binding agent of claim 6, wherein the Ig constant region, the fragment of the Ig constant region, the Fc region, or the CH3 domain comprises at least one mutation.

8. The binding agent of claim 7, wherein the at least one mutation is selected from the group consisting of L234A/L235A/D265S, F234A/L235A, L234A/L235A, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

9. The binding agent of claim 7, wherein the at least one mutation is selected from the group consisting of T350V, L351Y, F405A, Y407V, T366Y, T366W, T366L, F405W, T394W, K392L, T394S, Y407T, Y407A, T366S/L368A/Y407V, L351Y/F405A/Y407V, T366I/K392M/T394W, F405A/Y407V, T366L/K392M/T394W, T366L/K392L/T394W, L351Y/Y407A, L351Y/Y407V, T366A/K409F, T366V/K409F, T366A/K409F, T350V/L351Y/F405A/Y407V and T350V/T366L/K392L/T394W, wherein residue numbering is according to the EU index.

10. The binding agent of claim 7, wherein at least one mutation is selected from the group consisting of H435R, Y436F and H435R/L436F.

11. The binding agent of any one of claims 1 to 10, wherein, when bound to MSLN, the antigen binding region binds to at least one of residues 587-598 within an amino acid sequence of SEQ ID NO:200.

12. The binding agent of any one of claims 1 to 10, wherein, when bound to MSLN, the antigen binding region binds to at least one residue selected from the group consisting of L589, D590, and M593 with an amino acid sequence of SEQ ID NO:200.

13. The binding agent of any one of claims 5 to 12, wherein the bispecific protein comprises an antigen binding region that binds a second antigen other than MSLN.

14. The binding agent of claim 13, wherein the second antigen is cluster of differentiation 38 (CD3ε).

15. A binding agent comprising a first antigen binding region that binds to MSLN and a second antigen binding region that binds to CD3ε, wherein
(i) the first antigen binding region comprises a VH domain comprising a HCDR1, HCDR2, and HCDR3 as set forth in SEQ ID NO:81, and a VL domain comprising a LCDR1, LCDR2, and LCDR3 as set forth in SEQ ID NO:82; and
(ii) the second antigen binding region a VH domain comprising a HCDR1, HCDR2, and HCDR3 as set forth in SEQ ID NO: 159, and a VL domain comprising a LCDR1, LCDR2, and LCDR3 as set forth in SEQ ID NO:160.

16. The binding agent of claim 15, wherein
(i) in the first antigen binding region that binds to MSLN,
(a) HCDR1 comprises an amino acid sequence of SEQ ID NO:64; HCDR2 comprises an amino acid sequence of SEQ ID NO:65; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(b) HCDR1 comprises an amino acid sequence of SEQ ID NO:70; HCDR2 comprises an amino acid sequence of SEQ ID NO:31; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(c) HCDR1 comprises an amino acid sequence of SEQ ID NO:71; HCDR2 comprises an amino acid sequence of SEQ ID NO:52; HCDR3 comprises an amino acid sequence of SEQ ID NO:66; LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(d) HCDR1 comprises an amino acid sequence of SEQ ID NO:72; HCDR2 comprises an amino acid sequence of SEQ ID NO:54; HCDR3 comprises an amino acid sequence of SEQ ID NO:73; LCDR1 comprises an amino acid sequence of SEQ ID NO:74; LCDR2 comprises an amino acid sequence of SEQ ID NO:75; and LCDR3 comprises an amino acid sequence of SEQ ID NO:76; or
(e) HCDR1 comprises an amino acid sequence of SEQ ID NO:77; HCDR2 comprises an amino acid sequence of SEQ ID NO:59; HCDR3 comprises an amino acid sequence of SEQ ID NO:78; LCDR1 comprises an amino acid sequence of SEQ ID NO:79; LCDR2 comprises an amino acid sequence of WAS; and LCDR3 comprises an amino acid sequence of SEQ ID NO:69; and
(ii) in the second antigen binding region that binds to CD3ε,
(a) HCDR1 comprises an amino acid sequence of SEQ ID NO: 143; HCDR2 comprises an amino acid sequence of SEQ ID NO:144; HCDR3 comprises an amino acid sequence of SEQ ID NO: 145; LCDR1 comprises an amino acid sequence of SEQ ID NO: 146; LCDR2 comprises an amino acid sequence of SEQ ID NO: 147; and LCDR3 comprises an amino acid sequence of SEQ ID NO:148;
(b) HCDR1 comprises an amino acid sequence of SEQ ID NO: 149; HCDR2 comprises an amino acid sequence of SEQ ID NO: 150; HCDR3 comprises an amino acid sequence of SEQ ID NO:151; LCDR1 comprises an amino acid sequence of SEQ ID NO: 152; LCDR2 comprises an amino acid sequence of YAS; and LCDR3 comprises an amino acid sequence of SEQ ID NO:154; or
(c) HCDR1 comprises an amino acid sequence of SEQ ID NO: 155; HCDR2 comprises an amino acid sequence of SEQ ID NO:156; HCDR3 comprises an amino acid sequence of SEQ ID NO:157; LCDR1 comprises an amino acid sequence of SEQ ID NO: 158; LCDR2 comprises an amino acid sequence of YAS; and LCDR3 comprises an amino acid sequence of SEQ ID NO:148.

17. The binding agent of claim 15 or 16, wherein
(i) the first binding region comprises a VH domain comprising an amino acid sequence of SEQ ID NO:81, and a VL domain comprising an amino acid sequence of SEQ ID NO:82; and
(ii) the second binding region comprises a VH domain comprising an amino acid sequence of SEQ ID NO:159, and a VL domain comprising an amino acid sequence of SEQ ID NO:160.

18. The binding agent of any one of claims 15 to 17, wherein the first antigen binding region comprises a Fab, and the second antigen binding region comprises a scFv.

19. The binding agent of claim 18, wherein the scFv comprises an amino acid sequence of SEQ ID NO:161 or SEQ ID NO:162.

20. The binding agent of any one of claims 15 to 19, wherein the binding agent further comprises an immunoglobulin (Ig) constant region, a fragment of the Ig constant region, wherein optionally the fragment of the Ig constant region is an Fc region or an CH3 domain.

21. A binding agent comprising
(i) a first polypeptide comprising a scFv that binds CD3ε, a CH2 domain and a CH3 domain;
(ii) a second polypeptide comprising a VH domain that binds MSLN, a CH2 domain and a CH3 domain; and
(iii) a third polypeptide comprising a VL domain that binds MSLN,
wherein the scFv that binds CD3ε comprises a VH domain comprising a HCDR1, HCDR2, and HCDR3 as set forth in SEQ ID NO:159, and a VL domain comprising a LCDR1, LCDR2, and LCDR3 as set forth in SEQ ID NO: 160; and
wherein the VH domain that binds MSLN comprises a HCDR1, HCDR2, and HCDR3 as set forth in SEQ ID NO:81, and the VL domain that binds MSLN comprises a LCDR1, LCDR2, and LCDR3 as set forth in SEQ ID NO:82.

22. The binding agent of claim 21, wherein:
(i) the scFv that binds CD3ε comprises:
(a) HCDR1 comprising an amino acid sequence of SEQ ID NO:143; HCDR2 comprising an amino acid sequence of SEQ ID NO:144; HCDR3 comprising an amino acid sequence of SEQ ID NO:145; LCDR1 comprising an amino acid sequence of SEQ ID NO: 146; LCDR2 comprising an amino acid sequence of SEQ ID NO: 147; and LCDR3 comprising an amino acid sequence of SEQ ID NO:148;
(b) HCDR1 comprising an amino acid sequence of SEQ ID NO: 149; HCDR2 comprising an amino acid sequence of SEQ ID NO:150; HCDR3 comprising an amino acid sequence of SEQ ID NO:151; LCDR1 comprising an amino acid sequence of SEQ ID NO:152; LCDR2 comprising an amino acid sequence of YAS; and LCDR3 comprising an amino acid sequence of SEQ ID NO:154; or
(c) HCDR1 comprising an amino acid sequence of SEQ ID NO:155; HCDR2 comprising an amino acid sequence of SEQ ID NO:156; HCDR3 comprising an amino acid sequence of SEQ ID NO: 157; LCDR1 comprising an amino acid sequence of SEQ ID NO: 158; LCDR2 comprising an amino acid sequence of YAS; and LCDR3 comprising an amino acid sequence of SEQ ID NO: 148; and
(ii) (a) in the VH domain that binds MSLN, HCDR1 comprises an amino acid sequence of SEQ ID NO:64; HCDR2 comprises an amino acid sequence of SEQ ID NO:65; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(b) in the VH domain that binds MSLN, HCDR1 comprises an amino acid sequence of SEQ ID NO:70; HCDR2 comprises an amino acid sequence of SEQ ID NO:31; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(c) in the VH domain that binds MSLN, HCDR1 comprises an amino acid sequence of SEQ ID NO:71; HCDR2 comprises an amino acid sequence of SEQ ID NO:52; HCDR3 comprises an amino acid sequence of SEQ ID NO:66; and in the VL domain that binds MSLN, LCDR1 comprises an amino acid sequence of SEQ ID NO:67; LCDR2 comprises an amino acid sequence of SEQ ID NO:68; and LCDR3 comprises an amino acid sequence of SEQ ID NO:69;
(d) in the VH domain that binds MSLN, HCDR1 comprises an amino acid sequence of SEQ ID NO:72; HCDR2 comprises an amino acid sequence of SEQ ID NO:54; HCDR3 comprises an amino acid sequence of SEQ ID NO:73; and in the VL domain that binds MSLN, LCDR1 comprises an amino acid sequence of SEQ ID NO:74; LCDR2 comprises an amino acid sequence of SEQ ID NO:75; and LCDR3 comprises an amino acid sequence of SEQ ID NO:76; or
(e) in the VH domain that binds MSLN, HCDR1 comprises an amino acid sequence of SEQ ID NO:77; HCDR2 comprises an amino acid sequence of SEQ ID NO:59; HCDR3 comprises an amino acid sequence of SEQ ID NO:78; and in the VL domain that binds MSLN, LCDR1 comprises an amino acid sequence of SEQ ID NO:79; LCDR2 comprises an amino acid sequence of WAS; and LCDR3 comprises an amino acid sequence of SEQ ID NO:69.

23. The binding agent of claim 21 or 22, wherein the scFv that binds CD3ε comprises a VH domain comprising an amino acid sequence of SEQ ID NO:159, and a VL domain comprising an amino acid sequence of SEQ ID NO: 160; the VH domain that binds MSLN comprises an amino acid sequence of SEQ ID NO:81, and the VL domain that binds MSLN comprises an amino acid sequence of SEQ ID NO:82.

24. The binding agent of any one of claims 21 to 23, wherein the scFv comprises an amino acid sequence of SEQ ID NO:161 or SEQ ID NO:162.

25. A composition comprising the binding agent of any one of claims 1 to 24, and a pharmaceutically acceptable carrier.

26. A polynucleotide comprising nucleotide sequences encoding a VH, a VL, or both a VH and a VL of the binding agent of any one of claims 1 to 24.

27. A vector comprising the polynucleotide of claim 26.

28. A cell comprising the polynucleotide of claim 26.

29. A kit comprising the binding agent of any one of claims 1 to 24.

30. A method of making a binding agent which binds to a membrane-restricted epitope of MSLN, comprising culturing the cell of claim 28 to express the binding agent.

31. A method of directing a T cell to a target cell expressing MSLN, comprising contacting the T cell with an effective amount of the binding agent of any one of claims 14 to 24 or a composition comprising the binding agent and a pharmaceutically acceptable carrier, wherein the antigen binding region that binds to CD3ε binds the T cell and the antigen binding region that binds to MSLN binds to the target cell.

32. A method of treating a cancer or tumor in a subject in need thereof, comprising administering an effective amount the binding agent of any one of claims 14 to 24 or a composition comprising the binding agent and a pharmaceutically acceptable carrier to the subject.

33. A method for diagnosing and treating a subject having a MSLN-expressing cancer or tumor, comprising
(a) detecting presence or absence of a SNP in the MSLN gene in the subject that results in Met593 Val substitution in the encoded MSLN protein;
(b) diagnosing the subject as likely responsive to the treatment of a binding agent comprising a first antigen binding region that binds to MSLN and a second antigen binding region that binds to CD3ε if the subject is not homozygous for the SNP; and
(c) administering or providing for administration of an effective amount of the binding agent of any one of claims 14 to 24 or a composition comprising the binding agent and a pharmaceutically acceptable carrier to the subject if the subject is diagnosed as likely responsive in step (b).

34. A method for treating a subject having a MSLN-expressing cancer or tumor, comprising administering or providing for administration of an effective amount of the binding agent of any one of claims 14 to 24 or a composition comprising the binding agent and a pharmaceutically acceptable carrier to a subject wherein the subject is not homozygous for a SNP in the MSLN gene that results in Met593 Val substitution in the encoded MSLN protein.

35. The method of claim 33 or 34, wherein the cancer or tumor is selected from mesothelioma, ovarian cancer, and pancreatic cancer.
